(19) United States
(12) Patent Application Publication
(10) Pub. No.: US 2023/0135729 A1
(43) Pub. Date: May 4, 2023

(54) COMPOUND WITH ANTI-SPORE ACTIVITY AND PHARMACEUTICAL COMPOSITION THEREOF

(71) Applicant: CHENGDU BIOBEL COMPANY LIMITED, Chengdu (CN)

(72) Inventors: Bing Guang, Chengdu (CN); Tai Yang, Chengdu (CN); Renhan Dong, Chengdu (CN); Jin Liu, Chengdu (CN); Jian Xie, Chengdu (CN); Chuanjun Qin, Chengdu (CN); Sheng Huang, Chengdu (CN); Xiangyang Peng, Chengdu (CN); Qing Xu, Chengdu (CN); Yongxin Lai, Chengdu (CN); Wei Zhan, Chengdu (CN); Jian Peng, Chengdu (CN); Xiaohui Wang, Chengdu (CN)

(73) Assignee: CHENGDU BIOBEL COMPANY LIMITED, Chengdu (CN)

(21) Appl. No.: 17/759,054

(22) PCT Filed: Feb. 7, 2020
(86) PCT No.: PCT/CN2020/074462

§ 371 Date: Jul. 19, 2022

(30) Foreign Application Priority Data
Jan. 20, 2020 (CN) 202010066592.2

(51) Int. Cl. C07J 41/00; C07J 9/00; A61P 31/04

(52) U.S. Cl. CPC C07J 41/0061; C07J 9/00; A61P 31/04

(57) ABSTRACT

The present disclosure provides a compound represented by formula (I), or a salt or a stereoisomer thereof. The compound is capable of effectively inhibiting germination of Clostridium difficile spores with a significant bacteriostatic activity. The compound has an excellent prospect for use in preparation of a drug for preventing and/or treating a C. difficile-caused infectious disease, recurrence of the C. difficile-caused infectious disease, or a complication of the C. difficile-caused infectious disease.

### TECHNICAL FIELD

The present disclosure belongs to the technical field of medicine, and in particular relates to a cholestane derivative with an anti-bacterial spore activity and a pharmaceutical composition thereof.

### BACKGROUND ART

Clostridium difficile, as an obligate anaerobe belonging to the genus Clostridium, is named after the difficulty of isolation and culture due to extremely high sensitivity to oxygen. The Clostridium difficile normally lives in human intestinal tract. Usually, C. difficile infection is caused by the overuse of certain antibiotics, disrupting the balance of intestinal flora, and allowing the C. difficile flora to grow faster and cause inflammations. C. difficile produces exotoxins A and B with different effects at different periods. The exotoxin A as an enterotoxin binds to mucosal cells in an initial stage, causing primary damages, which can lead to inflammations of the intestinal wall, cell infiltration, increased permeability of the intestinal wall, hemorrhage, and necrosis. The exotoxin B as a cytotoxin damages the cytoskeleton, causing cell pyknosis and necrosis, which directly damages intestinal wall cells to cause diarrhea.

C. difficile-caused infectious diseases are diseases caused by infection with C. difficile cells and/or C. difficile spores. Pseudomembranous enteritis is a common C. difficile-based infectious disease with clinical manifestations of diarrhea, abdominal pain, and symptoms of systemic toxicity. The disease has a sudden onset of symptoms and is accompanied by hypotension. The disease is also generally accompanied by fever and leukocytosis, which may even cause death. Therefore, the pseudomembranous enteritis is an extremely serious disease. In addition, the infection with C. difficile cells and/or C. difficile spores may also cause complications of the C. difficile-caused infectious disease. Common complications include pyelonephritis, meningitis, abdominal and vaginal infections, bacteremia, and gas gangrene. In recent years, the C. difficile, as an important pathogen causing nosocomial infectious diseases, is receiving increasing attention.

At present, the C. difficile-based infectious disease is mainly treated by bacteriostatic agents. Metronidazole and vancomycin are the two most commonly-used bacteriostatic agents. However, patients with the C. difficile-based infectious disease who have been treated with metronidazole and vancomycin are likely to have a relapse. Considering that blocking spore infection is an effective way to prevent and treat C. difficile infection, it is urgent to develop a novel bacteriostatic agent that inhibits the C. difficile with a high activity.

Cholic acid compounds are a class of tetracyclic fused-ring steroids with similar structures containing 23 to 25 carbon atoms, belonging to steroid acid compounds. The cholic acid compounds are commonly used in the treatment of hepatobiliary diseases; publications “The Journal of Infectious Diseases. 2013; 207: 1498-504” and “J. Med. Chem. 2018, 61, 6759-6778” reported that cholic acid derivatives had an activity of inhibiting the germination of C. difficile spores. However, the cholic acid compounds have a limited activity against the spores.

Therefore, it is of great significance to develop a novel bacteriostatic agent with a high activity against C. difficile spores.

### SUMMARY

An objective of the present disclosure is to provide a novel cholic acid compound with a high activity and capable of inhibiting germination of C. difficile spores, and a pharmaceutical composition thereof.

The present disclosure provides a compound represented by formula (I), or a salt or a stereoisomer thereof:

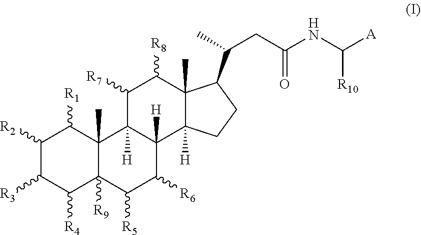

in which R₁, R₂, R₃, R₄, R₅, R₆, R₇, and R₈ are independently selected from the group consisting of H, —OH, C₁₋₅ alkoxy, —NH₂, ═O, ═S, ═NOH, —NHSO₃H or a salt thereof, —OSO₃H or a salt thereof, —OPO(OH)₂ or a salt thereof, —OCO(CH₂)ₘCOOH or a salt thereof, an amino acid ester group or a salt thereof, and —OCO(CH₂)ₘCH₃; m is an integer ranging from 0 to 9; and the amino acid ester group is a residue with a hydrogen atom removed from carboxyl on an amino acid; or

R₁ and R₂, R₅ and R₆, and/or R₇ and R₈ are ligated to form a double bond;

R₉ is selected from H, or R₉ is ligated with R₄ or R₅ to form a double bond;

R₁₀ is selected from H, linear or branched C₁₋₁₃ alkyl, —(CH₂)ₙCOOH, —(CH₂)ₙ₊₁NH₂, and —(CH₂)ₙ-B; and n is an integer ranging from 0 to 4;

A and B are independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl;

cycloalkyl includes cyclopropyl, cyclobutyl, cyclopentyl, and cyclohexyl; and

[symbol] represents a single bond or a double bond.

In some embodiments, the compound of formula (I) may have a structure represented by formula (IIA):

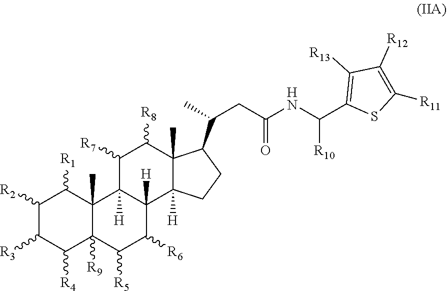

in which R₁, R₂, R₃, R₄, R₅, R₆, R₇, and R₈ are independently selected from the group consisting of H, —OH, C₁₋₅ alkoxy, —NH₂, ═O, ═S, ═NOH, —NHSO₃H or a salt thereof, —OSO₃H or a salt thereof, —OPO(OH)₂ or a salt thereof, —OCO(CH₂)ₘCOOH or a salt thereof, an amino acid ester group or a salt thereof, and —OCO(CH₂)ₘCH₃; and m is an integer ranging from 0 to 9; or

R₁ and R₂, R₅ and R₆, and/or R₇ and R₈ are ligated to form a double bond;

R₉ is selected from H, or R₉ is ligated with R₄ or R₅ to form a double bond;

R₁₀ is selected from H, linear or branched C₁₋₄ alkyl, —(CH₂)ₙCOOH, —(CH₂)ₙ₊₁NH₂, and —(CH₂)ₙ-B; and n is an integer ranging from 0 to 4;

B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl;

cycloalkyl includes cyclopropyl, cyclobutyl, cyclopentyl, and cyclohexyl; and

R₁₁, R₁₂, and R₁₃ are independently selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

In some embodiments, the compound of formula (IIA) may have a structure represented by formula (IIIA-1) or (IIIA-2):

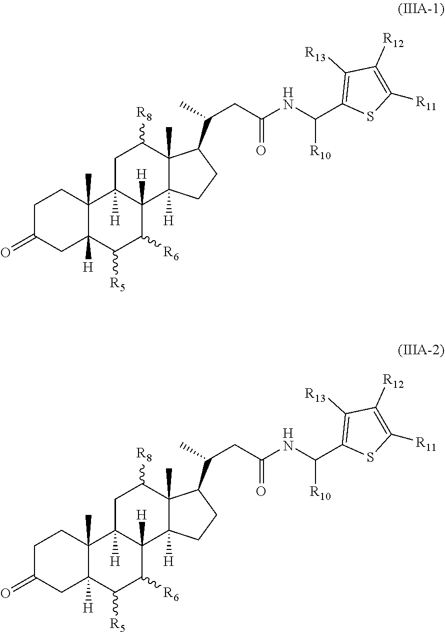

in which R₅, R₆, and R₈ are independently selected from the group consisting of H, —OH, —NH₂, —OSO₃H or a salt thereof, —OPO(OH)₂ or a salt thereof, —OCO(CH₂)ₘCOOH or a salt thereof, an amino acid ester group or a salt thereof, and —OCO(CH₂)ₘCH₃; and m is an integer ranging from 0 to 9;

R₁₀ is selected from H, linear or branched C₁₋₄ alkyl, —(CH₂)ₙCOOH, —(CH₂)ₙ₊₁NH₂, and —(CH₂)ₙ-B; and n is an integer ranging from 0 to 4;

B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;

cycloalkyl includes cyclopropyl, cyclobutyl, cyclopentyl, and cyclohexyl; and

R₁₁, R₁₂, and R₁₃ are independently selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

In some embodiments, the compound of formula (IIIA-1) may have a structure represented by formula (IVA-1):

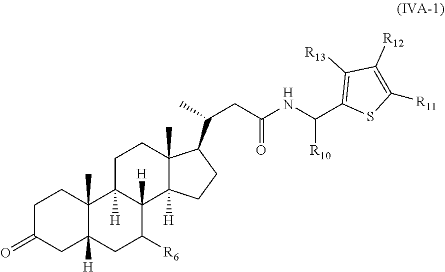

in which R₆ is selected from the group consisting of —OH, —NH₂, —OSO₃H or a salt thereof, —OPO(OH)₂ or a salt thereof, —OCO(CH₂)ₘCOOH or a salt thereof, an amino acid ester or a salt thereof, and —OCO(CH₂)ₘCH₃; the amino acid ester group is an α-amino acid ester group; and m is an integer ranging from 0 to 9;

R₁₀ is selected from H, linear or branched C₁₋₄ alkyl, —(CH₂)ₙCOOH, —(CH₂)ₙ₊₁NH₂, and —(CH₂)ₙ-B; and n is an integer ranging from 0 to 4;

B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;

cycloalkyl includes cyclopropyl, cyclobutyl, cyclopentyl, and cyclohexyl; and

R₁₁, R₁₂, and R₁₃ are independently selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

In some embodiments, the compound of formula (IIIA-1) may have a structure represented by formula (VA-1):

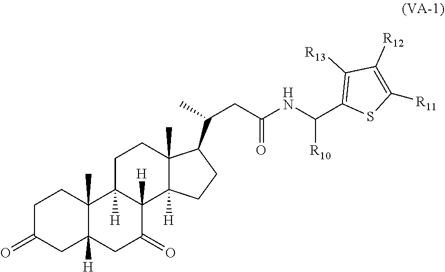

in which R₁₀ is selected from H, linear or branched C₁₋₄ alkyl, —(CH₂)ₙCOOH, —(CH₂)ₙ₊₁NH₂, and —(CH₂)ₙ-B; and n is an integer ranging from 0 to 4;

B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;

cycloalkyl includes cyclopropyl, cyclobutyl, cyclopentyl, and cyclohexyl; and

R₁₁, R₁₂, and R₁₃ are independently selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

In some embodiments, the compound of formula (IIIA-1) may have a structure represented by formula (VIA-1):

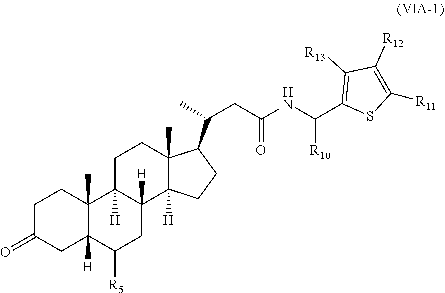

in which R₅ is selected from the group consisting of —OH, —NH₂, —OSO₃H or a salt thereof, —OPO(OH)₂ or a salt thereof, —OCO(CH₂)ₘCOOH or a salt thereof, an amino acid ester or a salt thereof, and —OCO(CH₂)ₘCH₃; the amino acid ester group is an α-amino acid ester group; and m is an integer ranging from 0 to 9;

R₁₀ is selected from H, linear or branched C₁₋₄ alkyl, —(CH₂)ₙCOOH, —(CH₂)ₙ₊₁NH₂, and —(CH₂)ₙ—B; and n is an integer ranging from 0 to 4;

B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;

cycloalkyl includes cyclopropyl, cyclobutyl, cyclopentyl, and cyclohexyl; and

R₁₁, R₁₂, and R₁₃ are independently selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

In some embodiments, the compound of formula (IIIA-1) may have a structure represented by formula (VIIA-1):

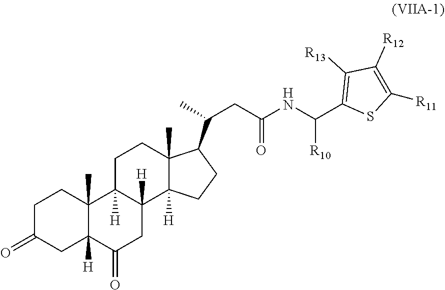

in which R₁₀ is selected from H, linear or branched C₁₋₄ alkyl, —(CH₂)ₙCOOH, —(CH₂)ₙ₊₁NH₂, and —(CH₂)ₙ-B; and n is an integer ranging from 0 to 4;

B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;

cycloalkyl includes cyclopropyl, cyclobutyl, cyclopentyl, and cyclohexyl; and

R₁₁, R₁₂, and R₁₃ are independently selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃ COOH; and n2 is an integer ranging from 0 to 3 and n3 is an integer ranging from 1 to 6.

In some embodiments, the compound of formula (IIIA-1) may have a structure represented by formula (VIIIA-1):

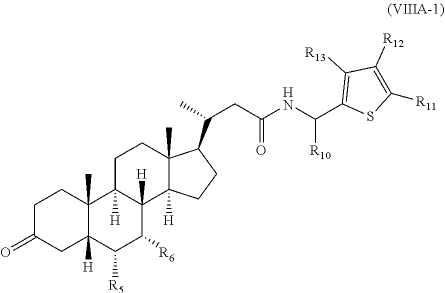

in which R₅ and R₆ are selected from the group consisting of —OH, —NH₂, —OSO₃H or a salt thereof, —OPO(OH)₂ or a salt thereof, —OCO(CH₂)ₘCOOH or a salt thereof, an amino acid ester or a salt thereof, and —OCO(CH₂)ₘCH₃; the amino acid ester group is an α-amino acid ester group; and m is an integer ranging from 0 to 9;

R₁₀ is selected from H, linear or branched C₁₋₄ alkyl, —(CH₂)ₙCOOH, —(CH₂)ₙ₊₁NH₂, and —(CH₂)ₙ—B; and n is an integer ranging from 0 to 4;

B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;

cycloalkyl includes cyclopropyl, cyclobutyl, cyclopentyl, and cyclohexyl; and

R₁₁, R₁₂, and R₁₃ are independently selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

In some embodiments, the compound of formula (IIIA-1) may have a structure represented by formula (IXA-1):

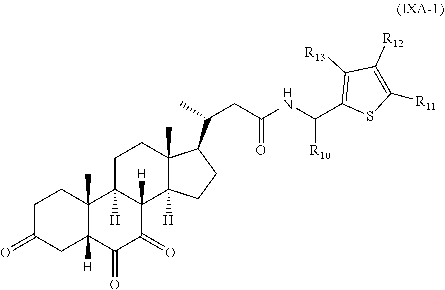

in which R₁₀ is selected from H, linear or branched C₁₋₄ alkyl, —(CH₂)ₙCOOH, —(CH₂)ₙ₊₁NH₂, and —(CH₂)ₙ-B; and n is an integer ranging from 0 to 4;

B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;

cycloalkyl includes cyclopropyl, cyclobutyl, cyclopentyl, and cyclohexyl; and

R₁₁, R₁₂, and R₁₃ are independently selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

In some embodiments, the compound of formula (IIIA-1) may have a structure represented by formula (XA-1):

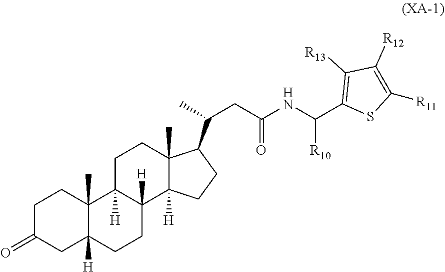

in which R₁₀ is selected from H, linear or branched C₁₋₄ alkyl, —(CH₂)ₙCOOH, —(CH₂)ₙ₊₁NH₂, and —(CH₂)ₙ—B; and n is an integer ranging from 0 to 4;

B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;

cycloalkyl includes cyclopropyl, cyclobutyl, cyclopentyl, and cyclohexyl; and

R₁₁, R₁₂, and R₁₃ are independently selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

In some embodiments, the compound of formula (WA-1) may have a structure represented by formula (XIA-1) or (XIA-2):

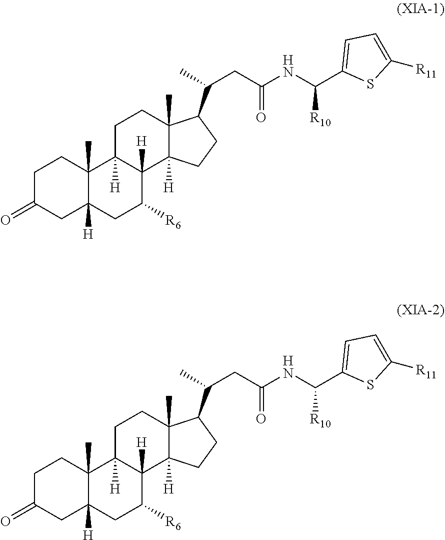

in which R₁₀ is selected from the group consisting of H, C₁₋₄ alkyl, and cyclopropyl;

in which R₆ is selected from the group consisting of —OH, —NH₂, —OSO₃H or a salt thereof, —OPO(OH)₂ or a salt thereof, —OCO(CH₂)ₘCOOH or a salt thereof, an amino acid ester or a salt thereof, and —OCO(CH₂)ₘCH₃; the amino acid ester group is an α-amino acid ester group; and m is an integer ranging from 0 to 9;

R₁₁ is selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

In some embodiments, the compound of formula (VA-1) may have a structure represented by formula (XIIA-1) or (XIIA-2):

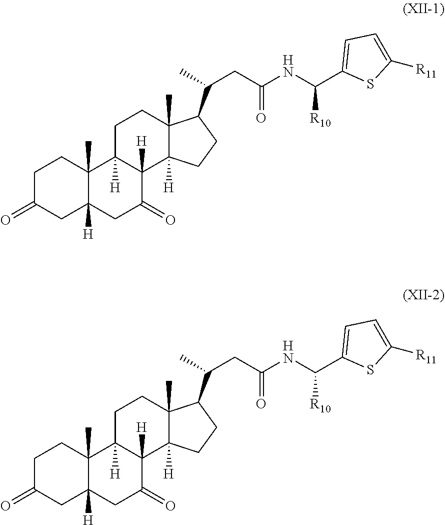

in which R₁₀ is selected from the group consisting of H, C₁₋₄ alkyl, and cyclopropyl;

R₁₁ is selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₁₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

In some embodiments, the compound of formula (IIA) may have a structure represented by formula (IIIB):

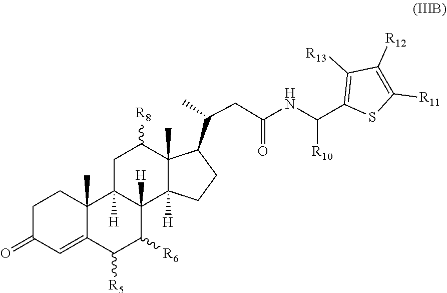

in which R₅, R₆, and R₈ are independently selected from the group consisting of H, —OH, C₁₋₅ alkoxy, —NH₂, ═O, ═S, ═NOH, —NHSO₃H or a salt thereof, —OSO₃H or a salt thereof, —OPO(OH)₂ or a salt thereof, —OCO(CH₂)ₘCOOH or a salt thereof, an amino acid ester group or a salt thereof, and —OCO(CH₂)ₘCH₃; and m is an integer ranging from 0 to 9; or

R₁₀ is selected from H, linear or branched C₁₋₄ alkyl, —(CH₂)ₙCOOH, —(CH₂)ₙ₊₁NH₂, and —(CH₂)ₙ-B; and n is an integer ranging from 0 to 4;

B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;

cycloalkyl includes cyclopropyl, cyclobutyl, cyclopentyl, and cyclohexyl; and

R₁₁, R₁₂, and R₁₃ are independently selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

In some embodiments, the compound of formula (IIA) may have a structure represented by formula (IIIC):

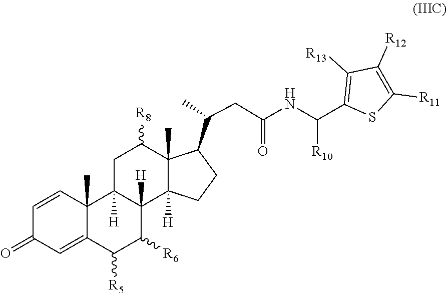

in which R₅, R₆, and R₈ are independently selected from the group consisting of H, —OH, C₁₋₅ alkoxy, —NH₂, ═O, ═S, ═NOH, —NHSO₃H or a salt thereof, —OSO₃H or a salt thereof, —OPO(OH)₂ or a salt thereof, —OCO(CH₂)ₘCOOH or a salt thereof, an amino acid ester group or a salt thereof, and —OCO(CH₂)ₘCH₃; and m is an integer ranging from 0 to 9; or

R₁₀ is selected from H, linear or branched C₁₋₄ alkyl, —(CH₂)ₙCOOH, —(CH₂)ₙ₊₁NH₂, and —(CH₂)ₙ-B; and n is an integer ranging from 0 to 4;

B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;

cycloalkyl includes cyclopropyl, cyclobutyl, cyclopentyl, and cyclohexyl; and

R₁₁, R₁₂, and R₁₃ are independently selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

In some embodiments, the compound of formula (IIIB) may have a structure represented by formula (IVB-1), (IVB-2), or (IVB-3):

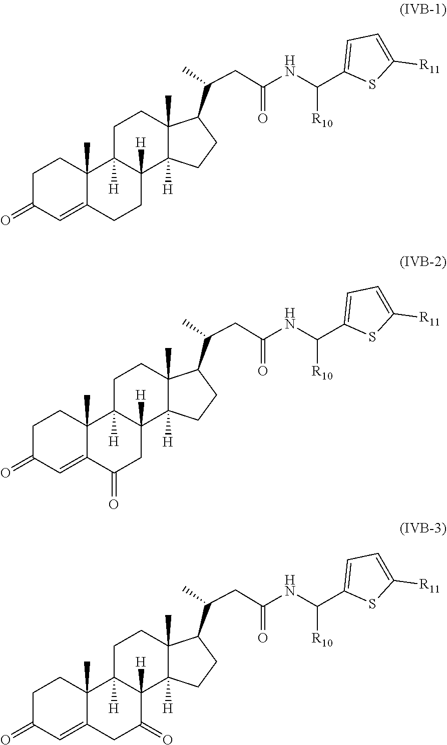

in which R₁₀ is selected from H, linear or branched C₁₋₄ alkyl, —(CH₂)ₙCOOH, —(CH₂)ₙ₊₁NH₂, and —(CH₂)ₙ-B; and n is an integer ranging from 0 to 4;

B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;

cycloalkyl includes cyclopropyl, cyclobutyl, cyclopentyl, and cyclohexyl; and

R₁₁ is selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

In some embodiments, the compound of formula (IIIB) may have a structure represented by formula (VB-1):

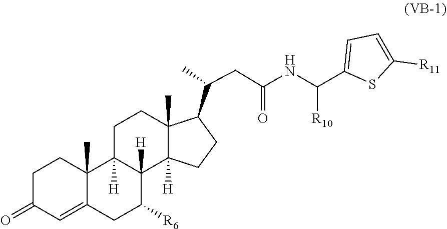

in which R₆ is selected from the group consisting of —OH, C₁₋₅ alkoxy, —NH₂, ═O, ═S, ═NOH, —NHSO₃H or a salt thereof, —OSO₃H or a salt thereof, —OPO(OH)₂ or a salt thereof, —OCO(CH₂)ₘCOOH or a salt thereof, an amino acid ester group or a salt thereof, and —OCO(CH₂)ₘCH₃; the amino acid ester group is an α-amino acid ester group; and m is an integer ranging from 0 to 9; or

R₁₀ is selected from H, linear or branched C₁₋₄ alkyl, —(CH₂)ₙCOOH, —(CH₂)ₙ₊₁NH₂, and —(CH₂)ₙ-B; and n is an integer ranging from 0 to 4;

B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;

cycloalkyl includes cyclopropyl, cyclobutyl, cyclopentyl, and cyclohexyl; and

R₁₁ is selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

In some embodiments, the compound of formula (IIIC) may have a structure represented by formula (IVC-1), (IVC-2), or (IVC-3):

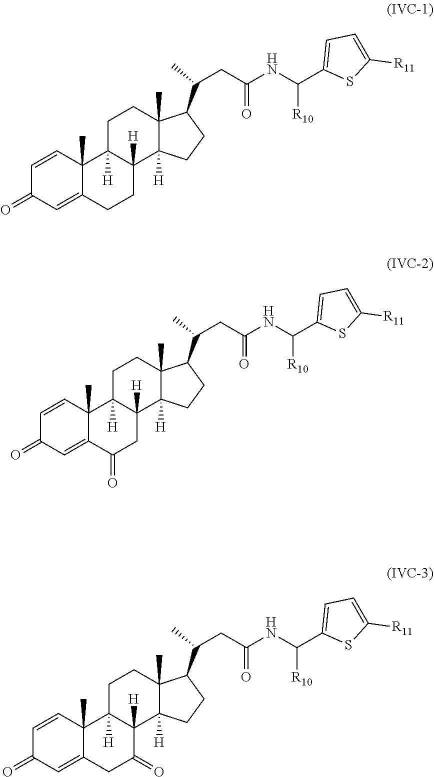

in which R₁₀ is selected from H, linear or branched C₁₋₄ alkyl, —(CH₂)ₙCOOH, —(CH₂)ₙ₊₁NH₂, and —(CH₂)ₙ-B; and n is an integer ranging from 0 to 4;

B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;

cycloalkyl includes cyclopropyl, cyclobutyl, cyclopentyl, and cyclohexyl; and

R₁₁ is selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

In some embodiments, the compound of formula (IIIC) may have a structure represented by formula (VC-1):

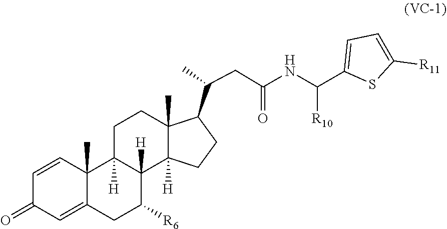

in which R₆ is selected from the group consisting of —OH, C₁₋₅ alkoxy, —NH₂, ═O, ═S, ═NOH, —NHSO₃H or a salt thereof, —OSO₃H or a salt thereof, —OPO(OH)₂ or a salt thereof, —OCO(CH₂)ₘCOOH or a salt thereof, an amino acid ester group or a salt thereof, and —OCO(CH₂)ₘCH₃; the amino acid ester group is an α-amino acid ester group; and m is an integer ranging from 0 to 9; or

R₁₀ is selected from H, linear or branched C₁₋₄ alkyl, —(CH₂)ₙCOOH, —(CH₂)ₙ₊₁NH₂, and —(CH₂)ₙ-B; and n is an integer ranging from 0 to 4;

B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;

cycloalkyl includes cyclopropyl, cyclobutyl, cyclopentyl, and cyclohexyl; and

R₁₁ is selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

In some embodiments, the compound of formula (I) may have a structure represented by formula (IID):

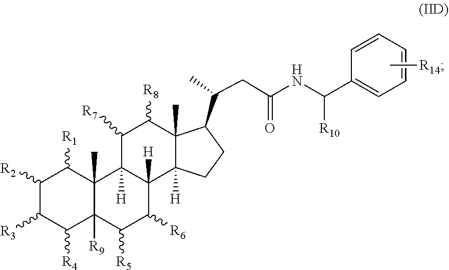

in which R₁, R₂, R₃, R₄, R₅, R₆, R₇, and R₈ are independently selected from the group consisting of H, —OH, C₁₋₅ alkoxy, —NH₂, ═O, ═S, ═NOH, —NHSO₃H or a salt thereof, —OSO₃H or a salt thereof, —OPO(OH)₂ or a salt thereof, —OCO(CH₂)ₘCOOH or a salt thereof, an amino acid ester group or a salt thereof, and —OCO(CH₂)ₘCH₃; and m is an integer ranging from 0 to 9; or

R₁ and R₂, R₅ and R₆, and/or R₇ and R₈ are ligated to form a double bond;

R₉ is selected from H, or R₉ is ligated with R₄ or R₅ to form a double bond;

R₁₀ is selected from H, linear or branched C₁₋₄ alkyl, —(CH₂)ₙCOOH, —(CH₂)ₙ₊₁NH₂, and —(CH₂)ₙ-B; and n is an integer ranging from 0 to 4;

B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl;

cycloalkyl includes cyclopropyl, cyclobutyl, cyclopentyl, and cyclohexyl; and

R₁₄ is selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

In some embodiments, the compound of formula (IID) may have a structure represented by formula (IIID):

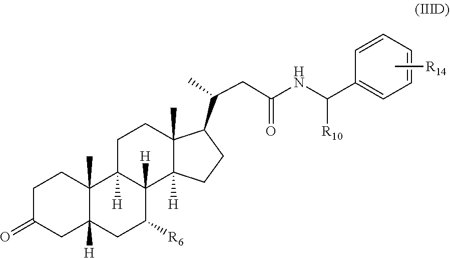

in which R₆ is selected from the group consisting of —OH, C₁₋₅ alkoxy, —NH₂, ═O, ═S, ═NOH, —NHSO₃H or a salt thereof, —OSO₃H or a salt thereof, —OPO(OH)₂ or a salt thereof, —OCO(CH₂)ₘCOOH or a salt thereof, an amino acid ester group or a salt thereof, and —OCO(CH₂)ₘCH₃; the amino acid ester group is an α-amino acid ester group; and m is an integer ranging from 0 to 9; or

R₁₀ is selected from H, linear or branched C₁₋₄ alkyl, —(CH₂)ₙCOOH, —(CH₂)ₙ₊₁NH₂, and —(CH₂)ₙ-B; and n is an integer ranging from 0 to 4;

B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;

cycloalkyl includes cyclopropyl, cyclobutyl, cyclopentyl, and cyclohexyl; and

R₁₄ is selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ2 CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

In some embodiments, the compound of formula (IID) may have a structure represented by formula (IVD):

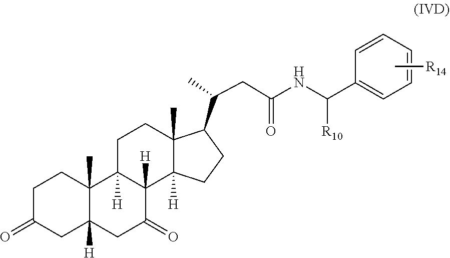

in which R₁₀ is selected from H, linear or branched C₁₋₄ alkyl, —(CH₂)ₙCOOH, —(CH₂)ₙ₊₁NH₂, and —(CH₂)ₙ-B; and n is an integer ranging from 0 to 4;

B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;

cycloalkyl includes cyclopropyl, cyclobutyl, cyclopentyl, and cyclohexyl; and

R₁₄ is selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

In some embodiments, the compound of formula (I) may have a structure represented by formula (TIE):

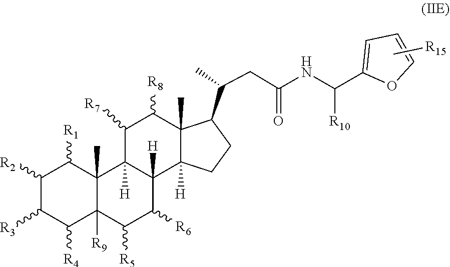

in which R₁, R₂, R₃, R₄, R₅, R₆, R₇, and R₈ are independently selected from the group consisting of H, —OH, C₁₋₅ alkoxy, —NH₂, ═O, ═S, ═NOH, —NHSO₃H or a salt thereof, —OSO₃H or a salt thereof, —OPO(OH)₂ or a salt thereof, —OCO(CH₂)ₘCOOH or a salt thereof, an amino acid ester group or a salt thereof, and —OCO(CH₂)ₘCH₃; and m is an integer ranging from 0 to 9; or

R₁ and R₂, R₅ and R₆, and/or R₇ and R₈ are ligated to form a double bond;

R₉ is selected from H, or R₉ is ligated with R₄ or R₅ to form a double bond;

R₁₀ is selected from H, linear or branched C₁₋₄ alkyl, —(CH₂)ₙCOOH, —(CH₂)ₙ₊₁NH₂, and —(CH₂)ₙ—B; and n is an integer ranging from 0 to 4;

B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl;

cycloalkyl includes cyclopropyl, cyclobutyl, cyclopentyl, and cyclohexyl; and

R₁₅ is selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

In some embodiments, the compound of formula (IIE) may have a structure represented by formula (IIIE):

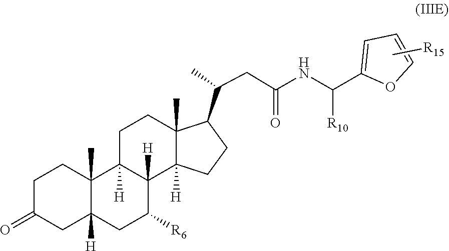

in which R₆ is selected from the group consisting of —OH, C₁₋₅ alkoxy, —NH₂, ═O, ═S, ═NOH, —NHSO₃H or a salt thereof, —OSO₃H or a salt thereof, —OPO(OH)₂ or a salt thereof, —OCO(CH₂)ₘCOOH or a salt thereof, an amino acid ester group or a salt thereof, and —OCO(CH₂)ₘCH₃; the amino acid ester group is an α-amino acid ester group; and m is an integer ranging from 0 to 9; or

R₁₀ is selected from H, linear or branched C₁₋₄ alkyl, —(CH₂)ₙCOOH, —(CH₂)ₙ₊₁NH₂, and —(CH₂)ₙ-B; and n is an integer ranging from 0 to 4;

B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;

cycloalkyl includes cyclopropyl, cyclobutyl, cyclopentyl, and cyclohexyl; and

R₁₅ is selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

In some embodiments, the compound of formula (IIE) may have a structure represented by formula (IVE):

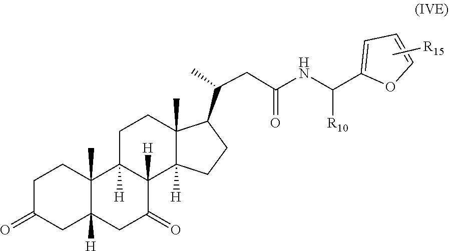

in which R₁₀ is selected from H, linear or branched C₁₋₄ alkyl, —(CH₂)ₙCOOH, —(CH₂)ₙ₊₁NH₂, and —(CH₂)ₙ—B; and n is an integer ranging from 0 to 4;

B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;

cycloalkyl includes cyclopropyl, cyclobutyl, cyclopentyl, and cyclohexyl; and

R₁₅ is selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

In some embodiments, the compound may be selected from the following compounds:

| No. | Compound structure |
|---|---|
| I-1 | 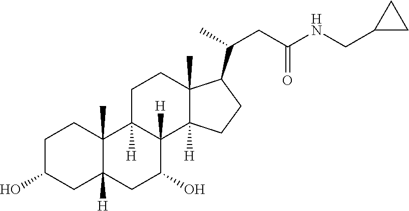 |
| I-2 | 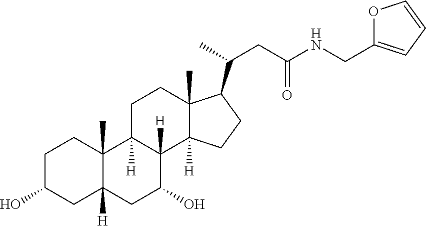 |
| I-3 | 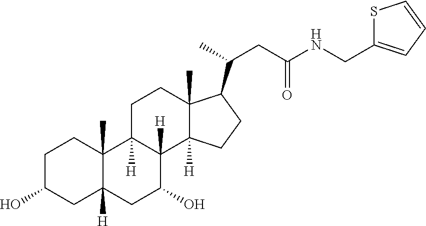 |
| I-4 | 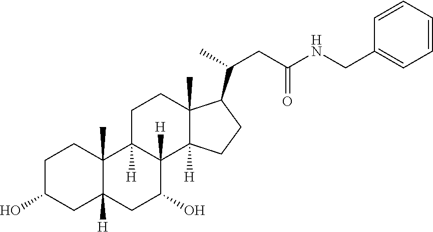 |
| I-5 | 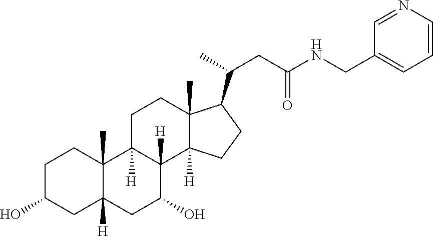 |
| I-6 | 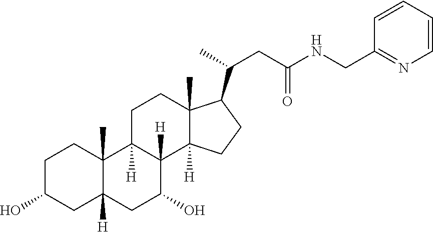 |
| I-7 | 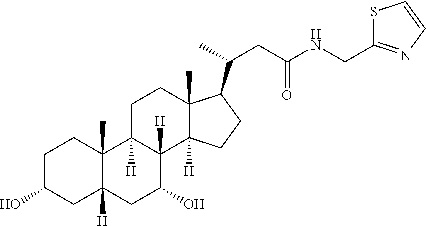 |
| I-8 | 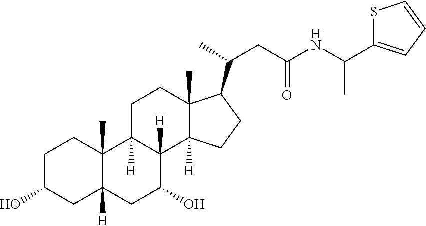 |
| I-9 | 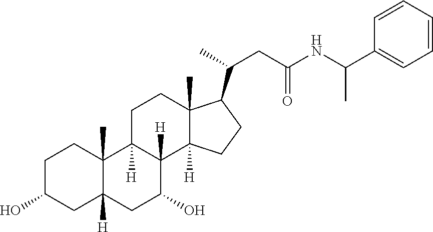 |
| I-10 | 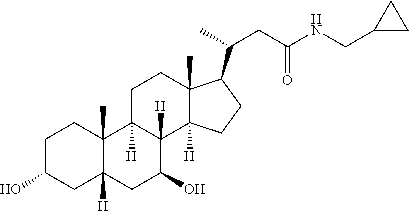 |
| I-11 | 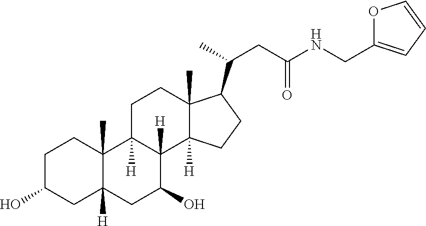 |
| I-12 | 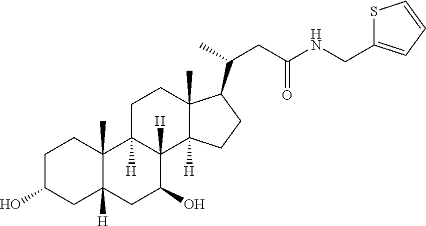 |
| I-13 | 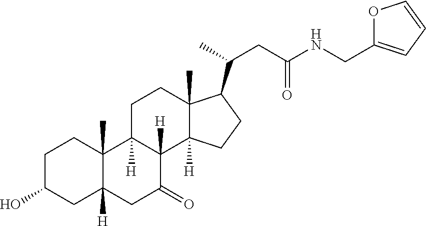 |
| I-14 | 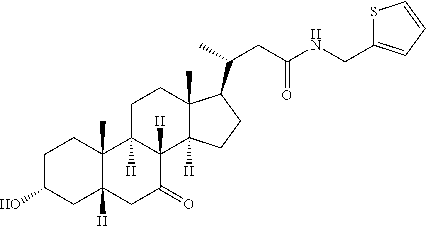 |
| I-15 | 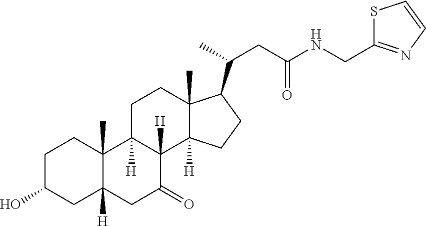 |
| I-16 | 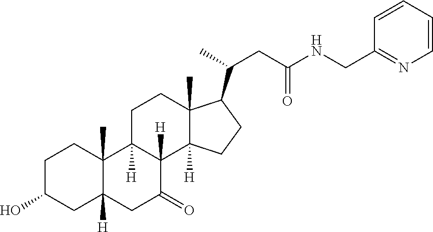 |
| I-17 | 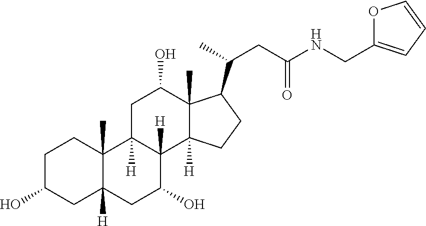 |
| I-18 | 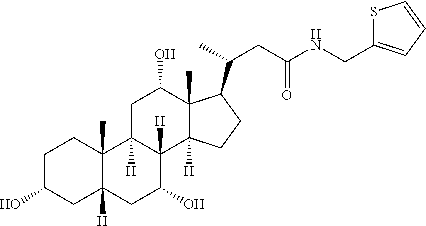 |
| I-19 | 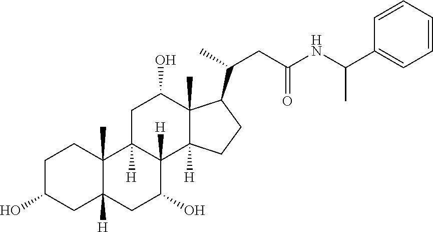 |
| I-20 | 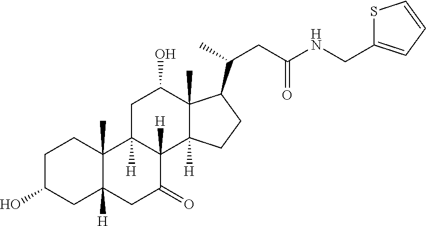 |
| I-21 | 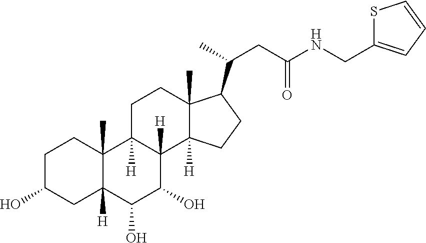 |
| I-22 | 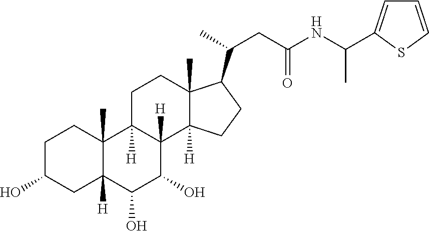 |
| I-23 | 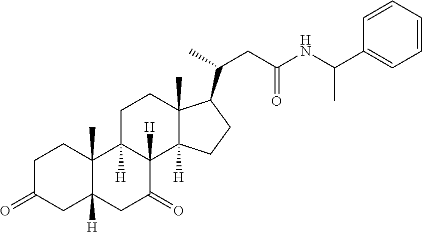 |
| I-24 | 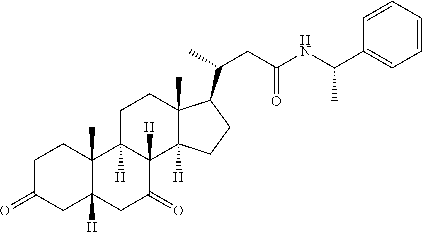 |
| I-25 | 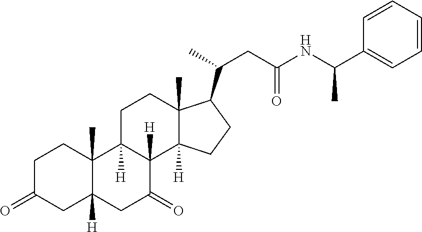 |
| I-26 | 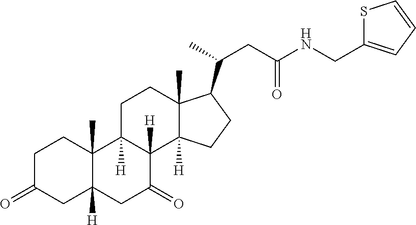 |
| I-27 | 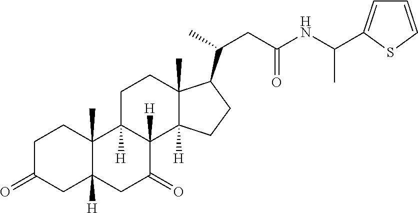 |
| I-28 | 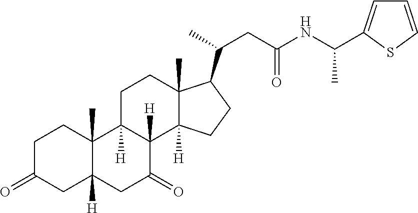 |
| I-29 | 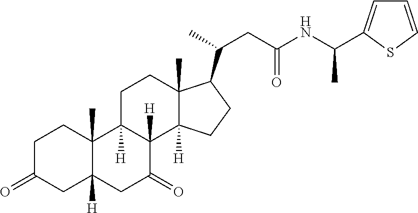 |
| I-30 | 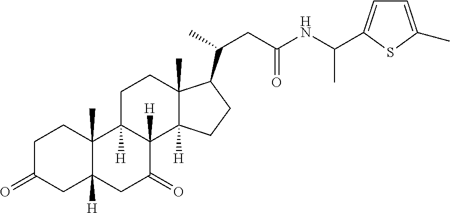 |
| I-31 | 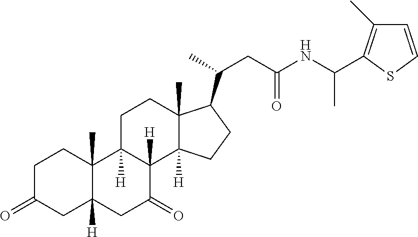 |
| I-32 | 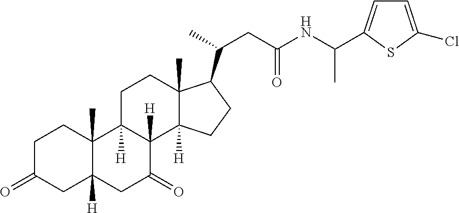 |
| I-33 | 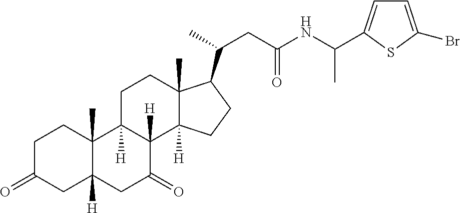 |
| I-34 | 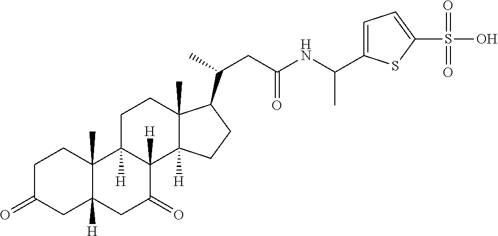 |
| I-35 | 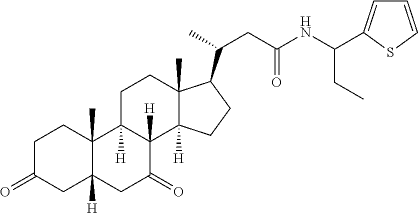 |
| I-36 | 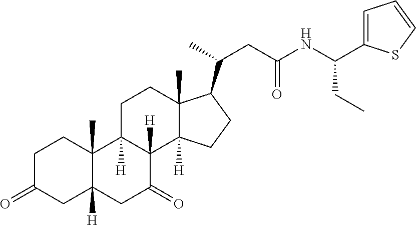 |
| I-37 | 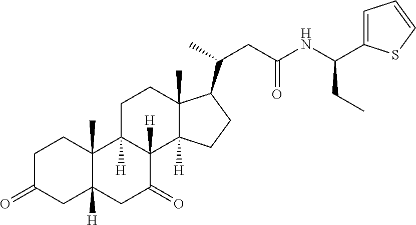 |
| I-38 | 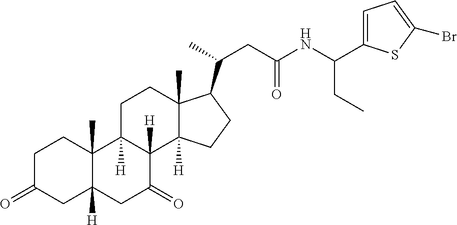 |
| I-39 | 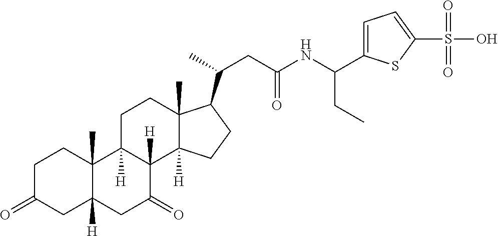 |
| I-40 | 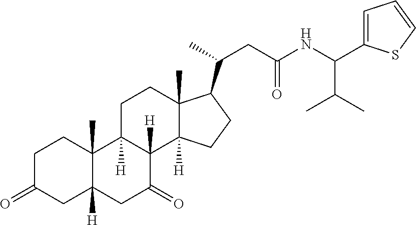 |
| I-41 | 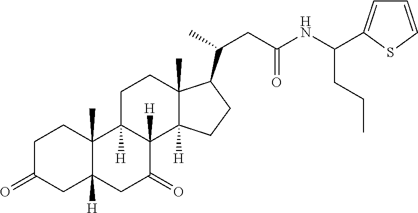 |
| I-42 | 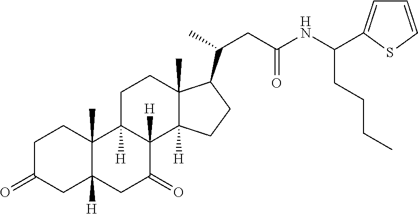 |
| I-43 | 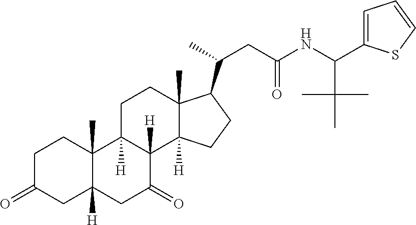 |
| I-44 | 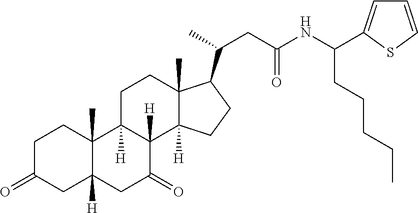 |
| I-45 | 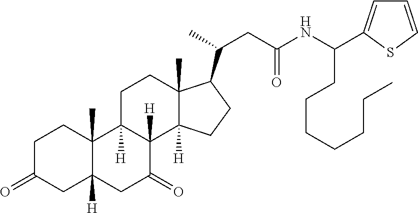 |
| I-46 | 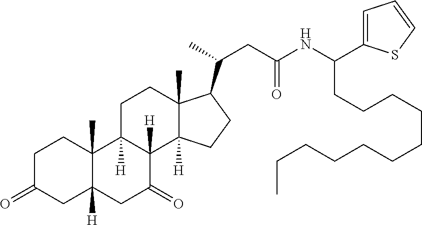 |
| I-47 | 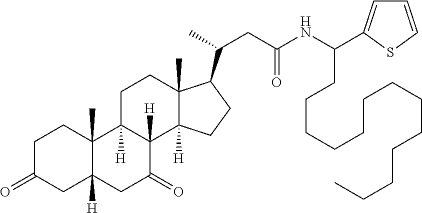 |
| I-48 | 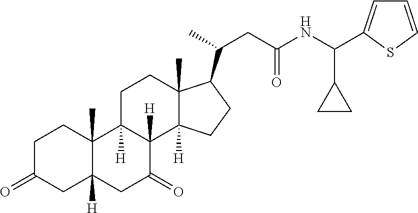 |
| I-49 | 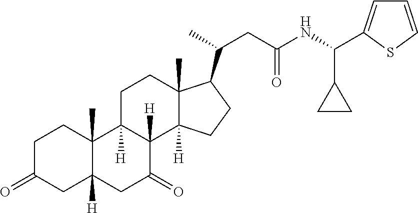 |
| I-50 | 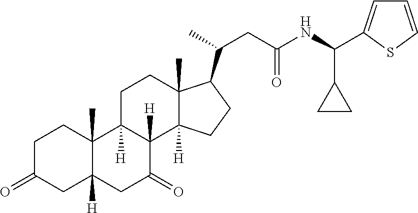 |
| I-51 | 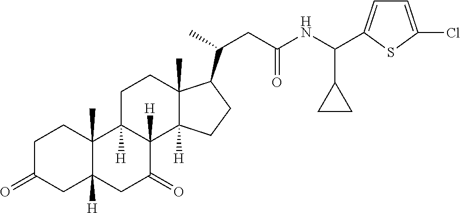 |
| I-52 | 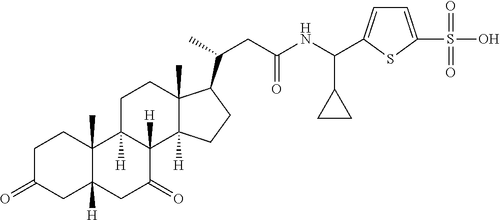 |
| I-53 | 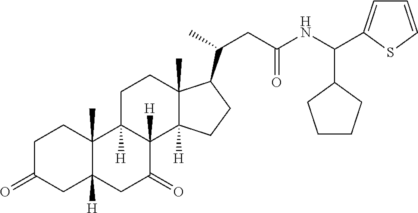 |
| I-54 | 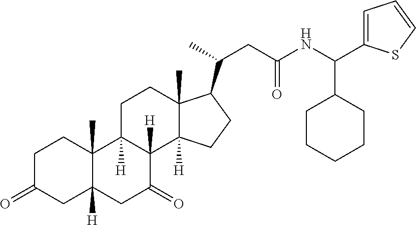 |
| I-55 | 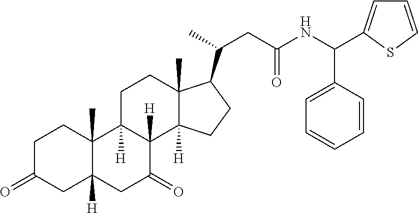 |
| I-56 | 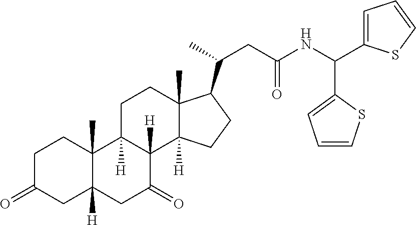 |
| I-57 | 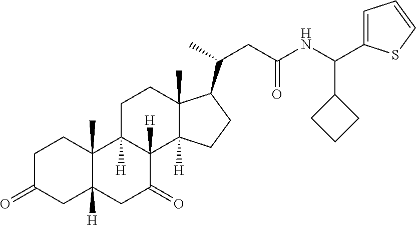 |
| I-58 | 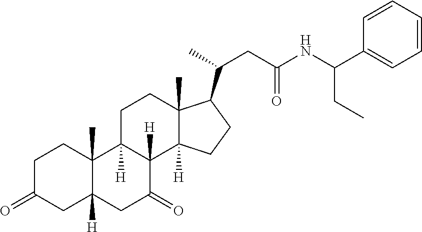 |
| I-59 | 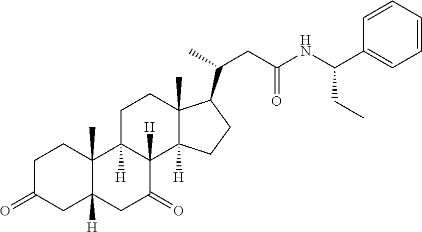 |
| I-60 | 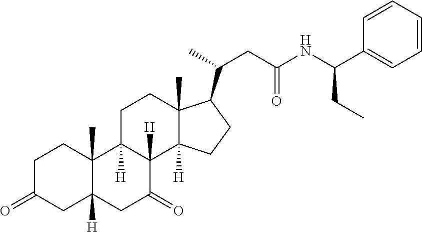 |
| I-61 | 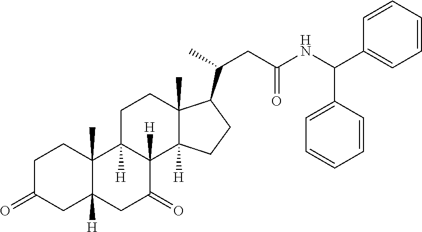 |
| I-62 | 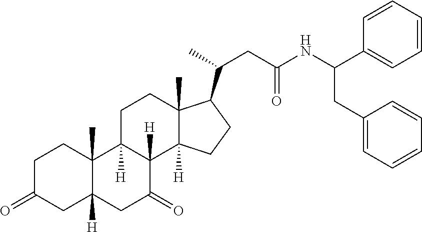 |
| I-63 | 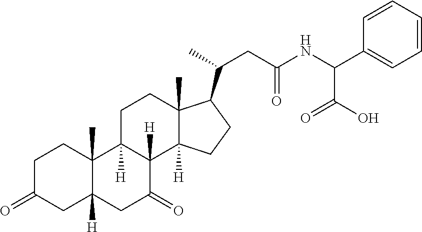 |
| I-64 | 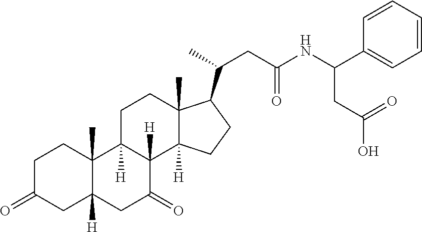 |
| I-65 | 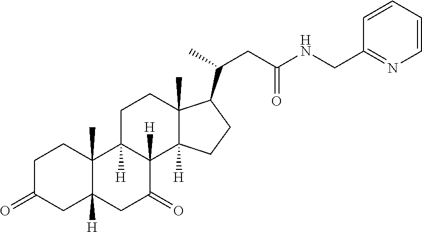 |
| I-66 | 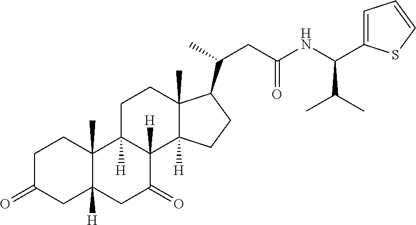 |
| I-67 | 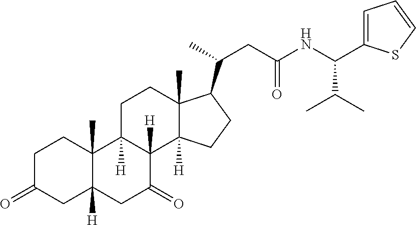 |
| I-68 | 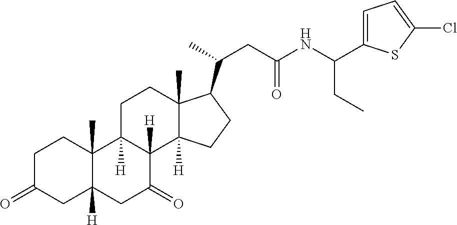 |
| I-69 | 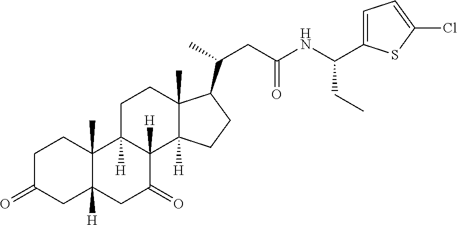 |
| I-70 | 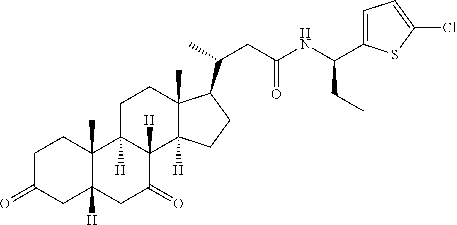 |
| I-71 | 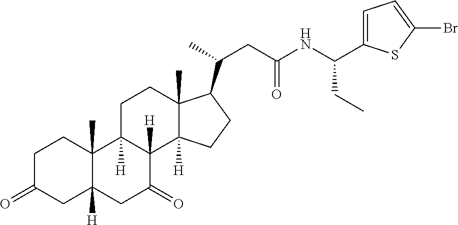 |
| I-72 | 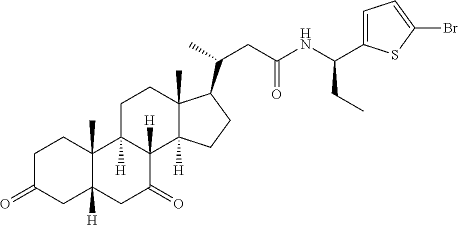 |
| I-73 | 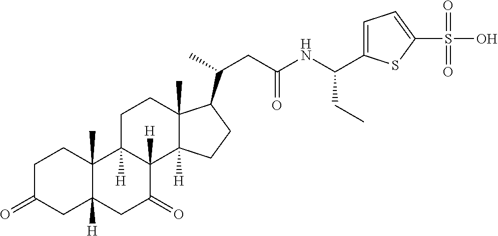 |
| I-74 | 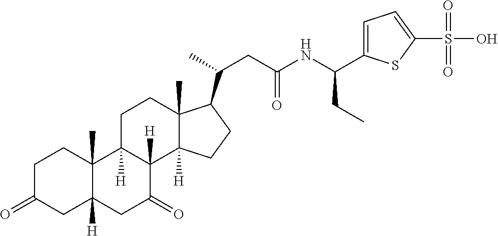 |
| I-75 | 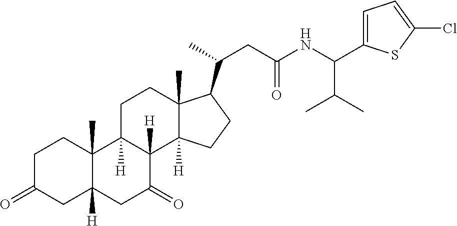 |
| I-76 | 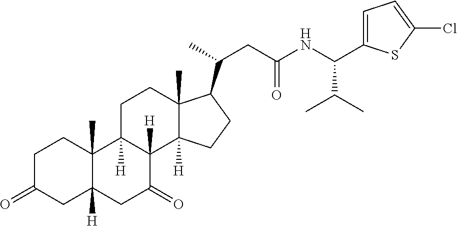 |
| I-77 | 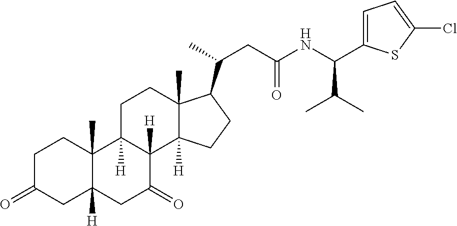 |
| I-78 | 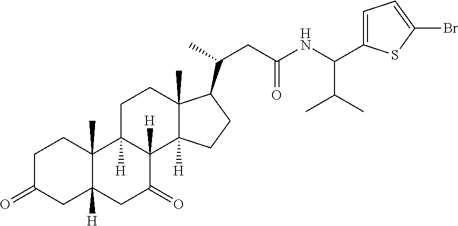 |
| I-79 | 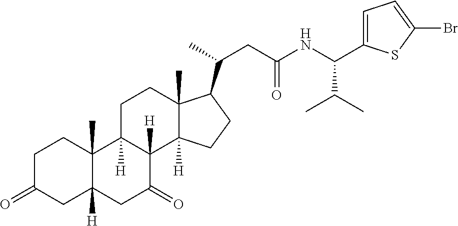 |
| I-80 | 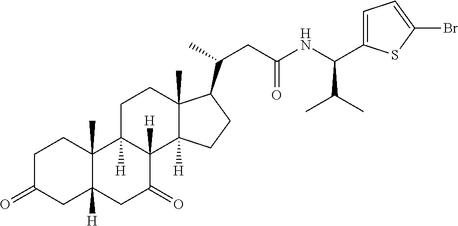 |
| I-81 | 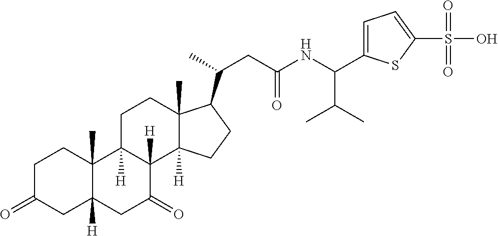 |
| I-82 | 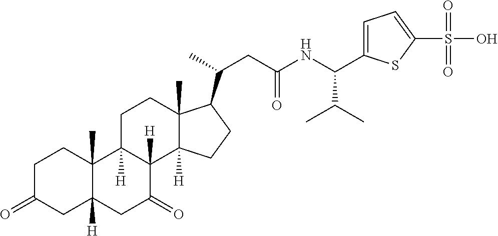 |
| I-83 | 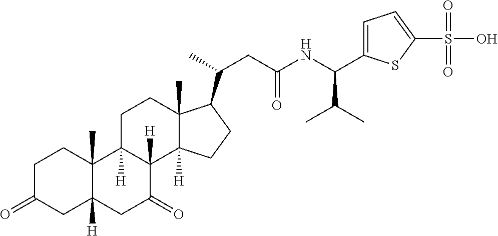 |
| I-84 | 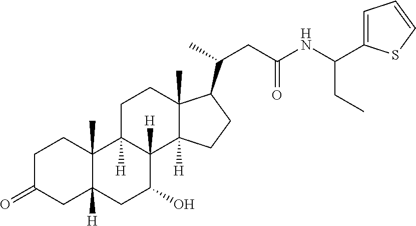 |
| I-85 | 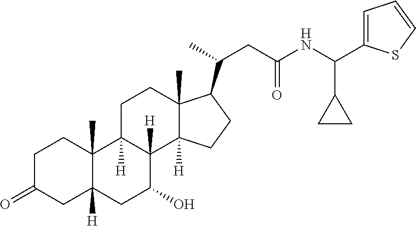 |
| I-86 | 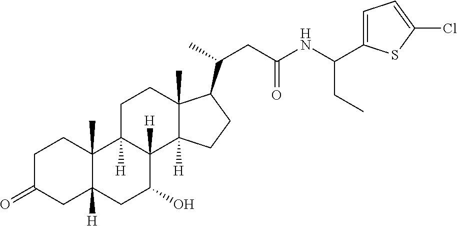 |
| I-87 | 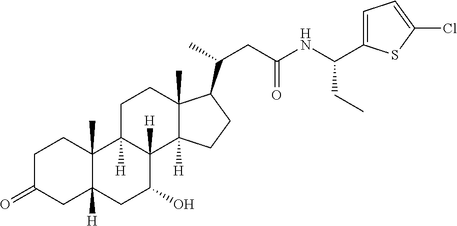 |
| I-88 | 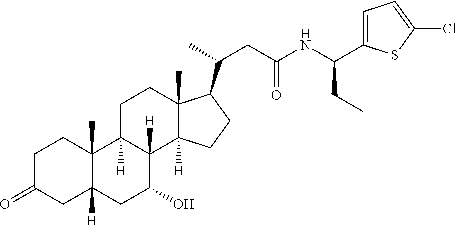 |
| I-89 | 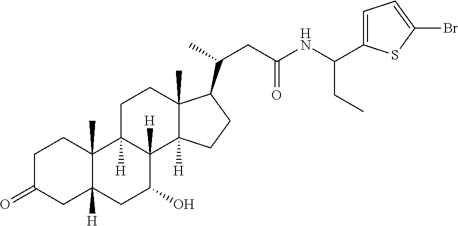 |
| I-90 | 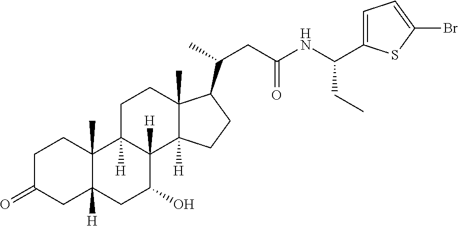 |
| I-91 | 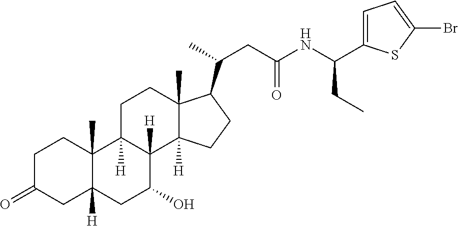 |
| I-92 | 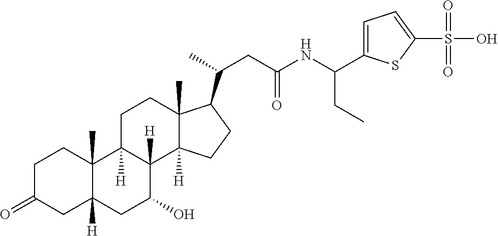 |
| I-93 | 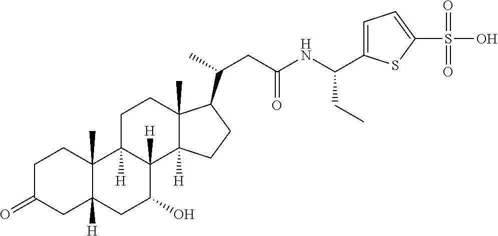 |
| I-94 | 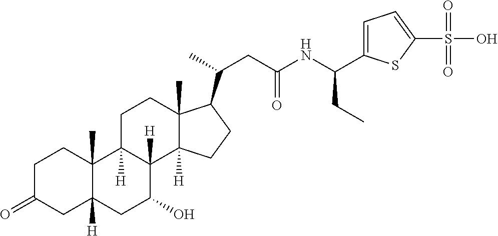 |
| I-95 | 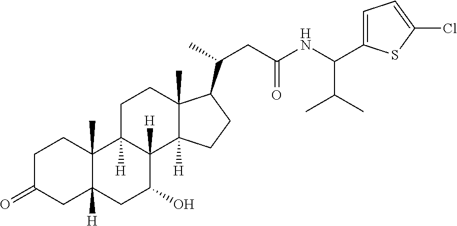 |
| I-96 | 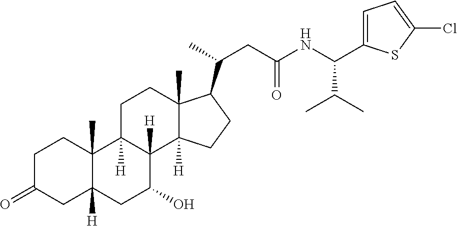 |
| I-97 | 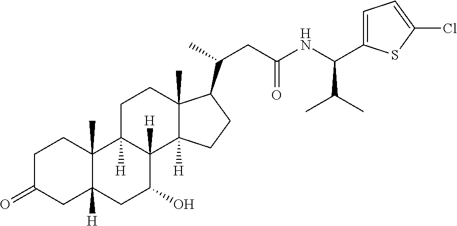 |
| I-98 | 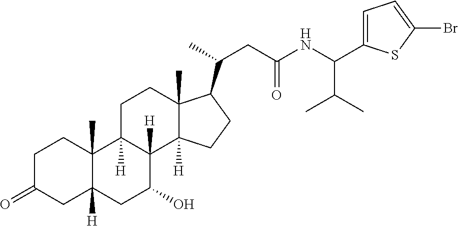 |
| I-99 | 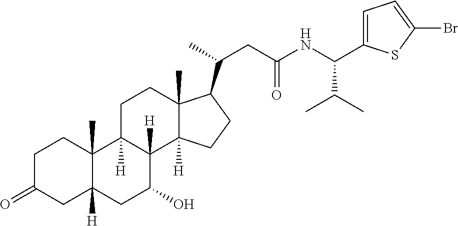 |
| I-100 | 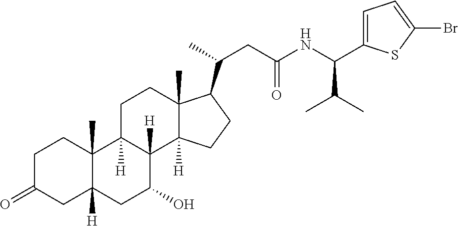 |
| I-101 | 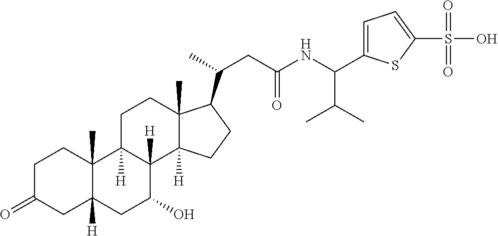 |
| I-102 | 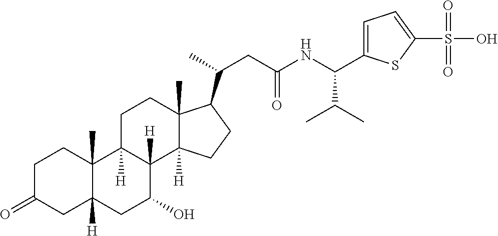 |
| I-103 | 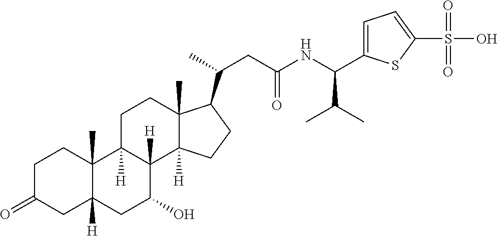 |
| I-104 | 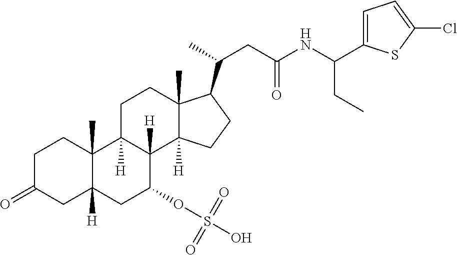 |
| I-104-1 | 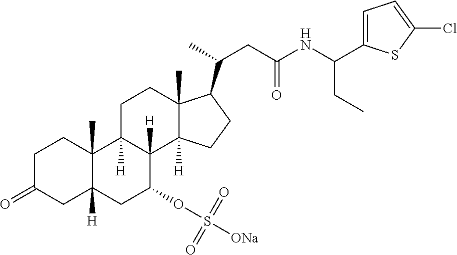 |
| I-105 | 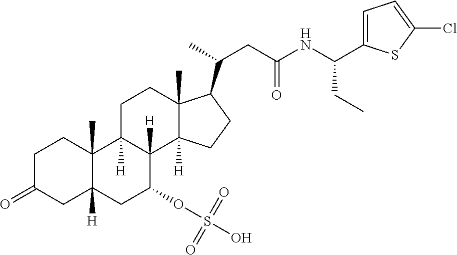 |
| I-105-1 | 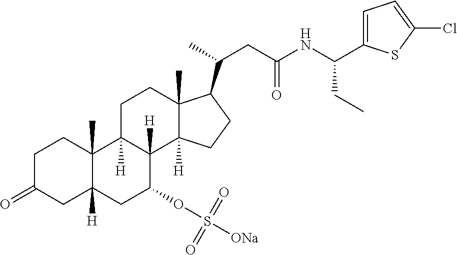 |
| I-106 | 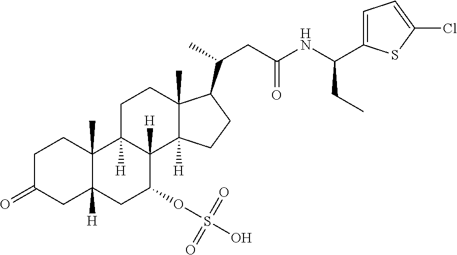 |
| I-106-1 | 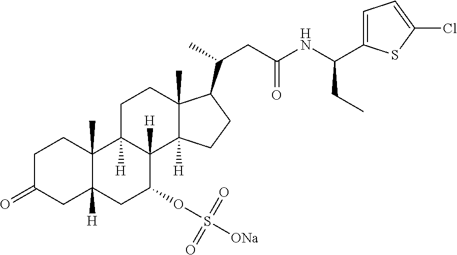 |
| I-107 | 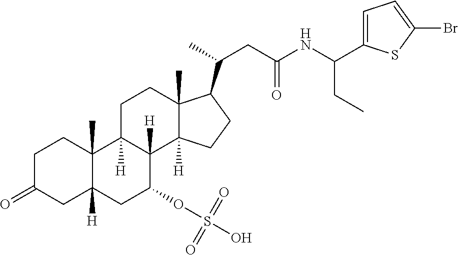 |
| I-107-1 | 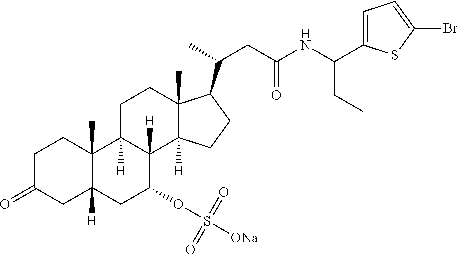 |
| I-108 | 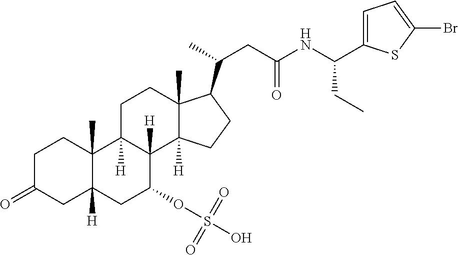 |
| I-108-1 | 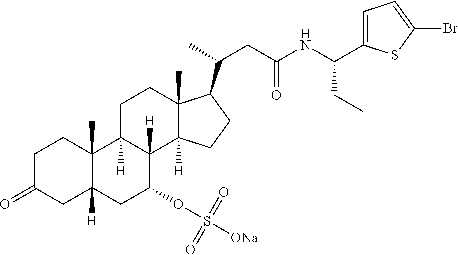 |
| I-109 | 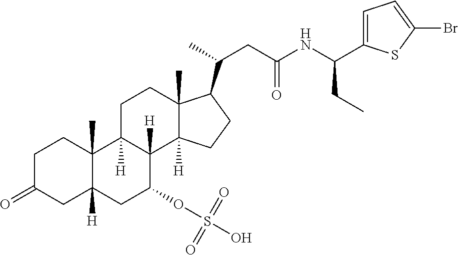 |
| I-109-1 | 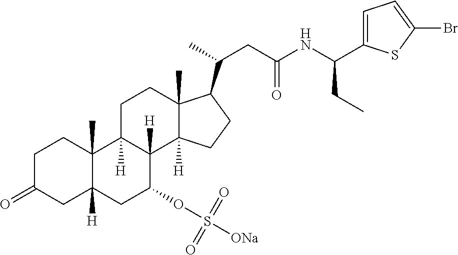 |
| I-110 | 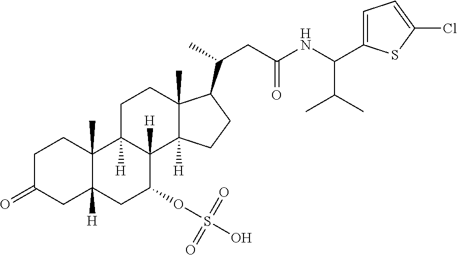 |
| I-110-1 | 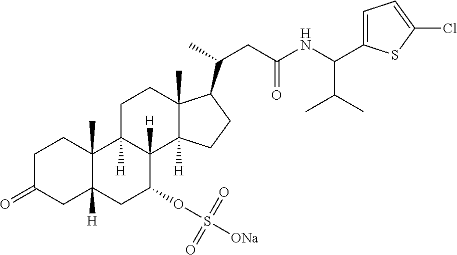 |
| I-111 | 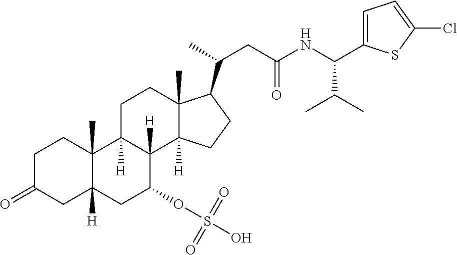 |
| I-111-1 | 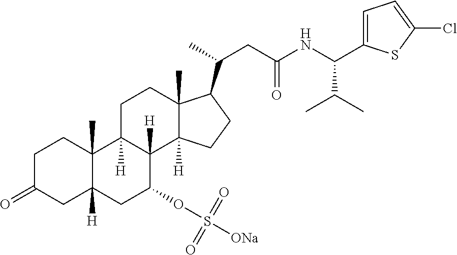 |
| I-112 | 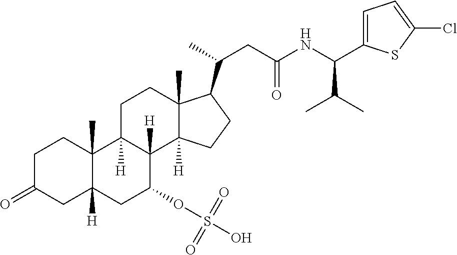 |
| I-112-1 | 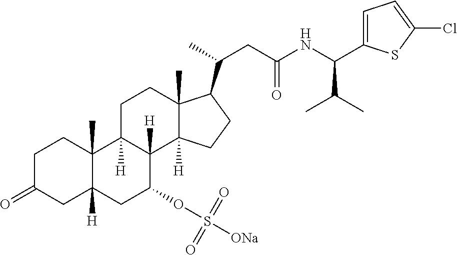 |
| I-113 | 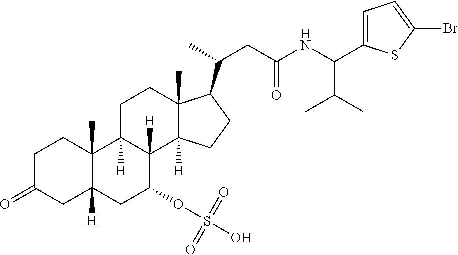 |
| I-113-1 | 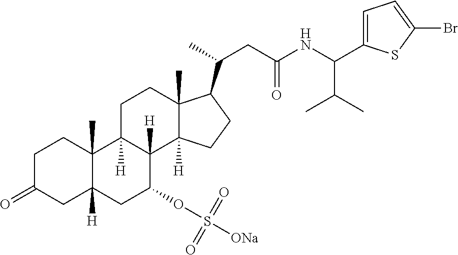 |
| I-114 | 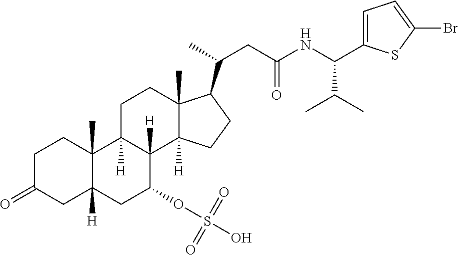 |
| I-114-1 | 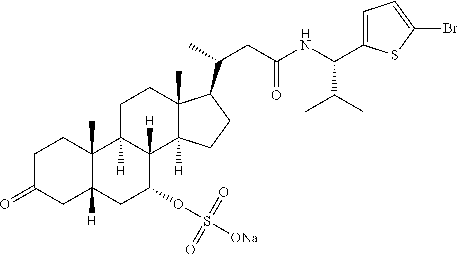 |
| I-115 | 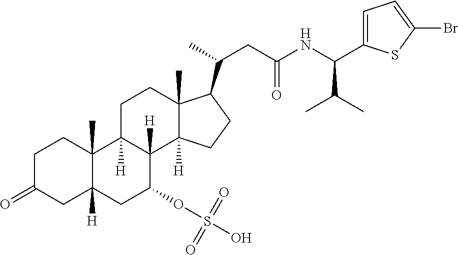 |
| I-115-1 | 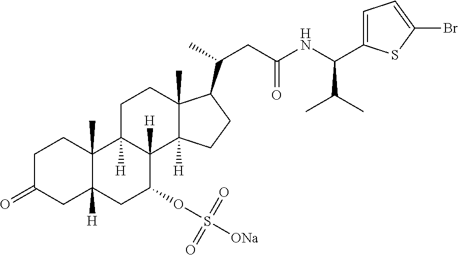 |
| I-116 | 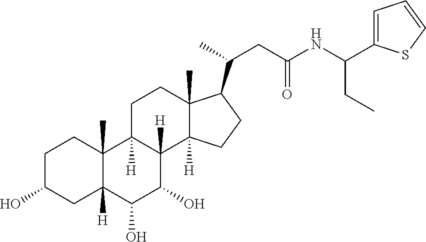 |
| I-117 | 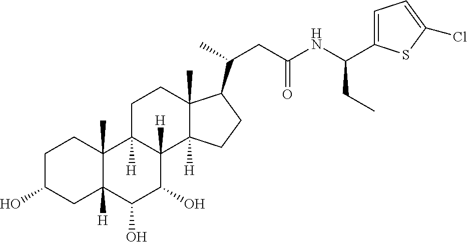 |
| I-118 | 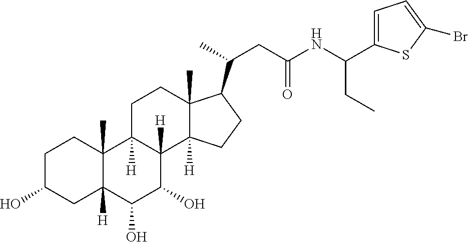 |
| I-119 | 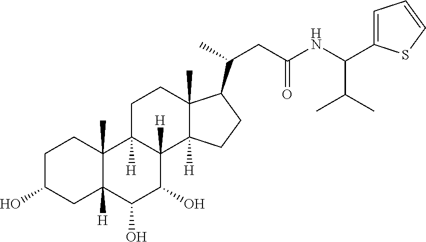 |
| I-120 | 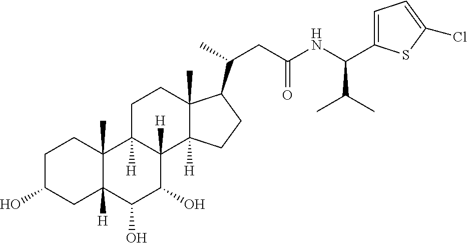 |
| I-121 | 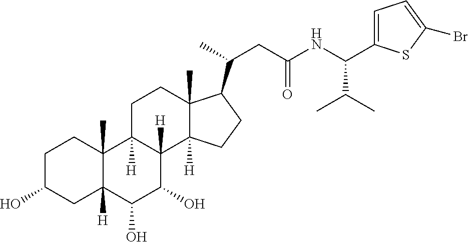 |
| I-122 | 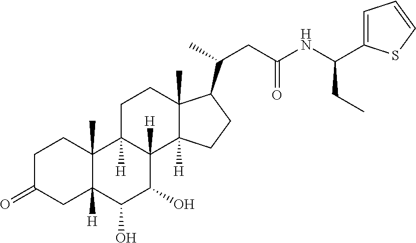 |
| I-123 | 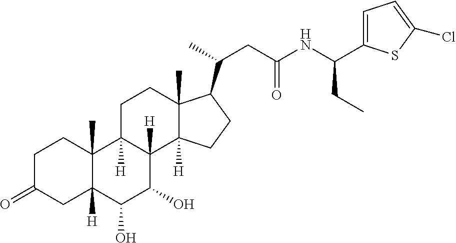 |
| I-124 | 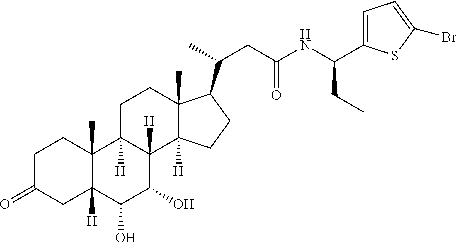 |
| I-125 | 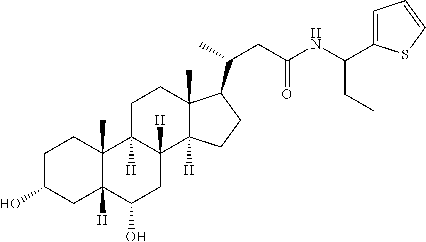 |
| I-126 | 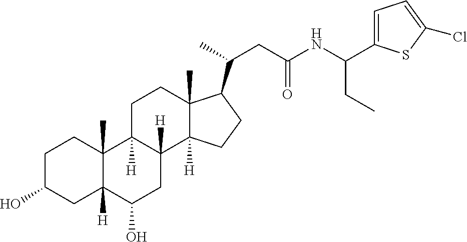 |
| I-127 | 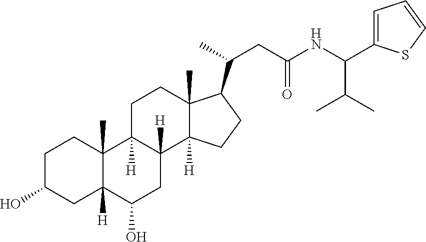 |
| I-128 | 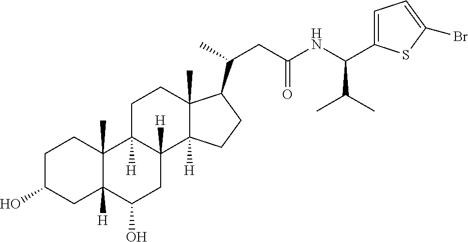 |
| I-129 | 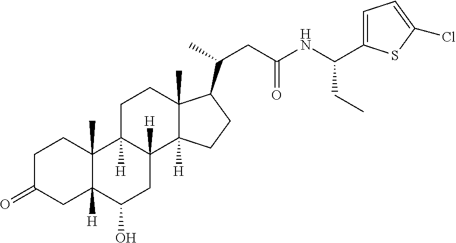 |
| I-130 | 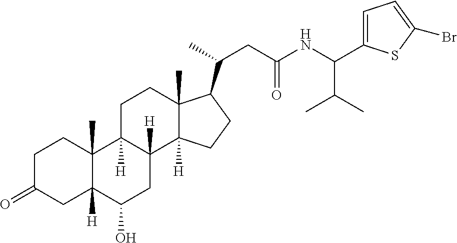 |
| I-131 | 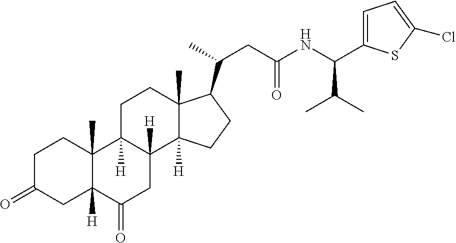 |
| I-132 | 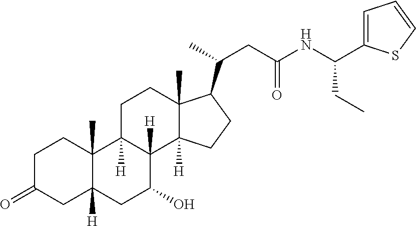 |
| I-133 | 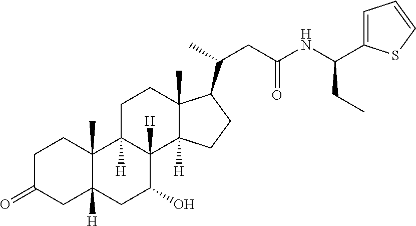 |
| I-134 | 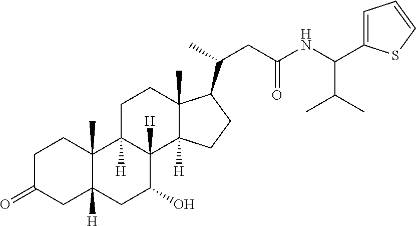 |
| I-135 | 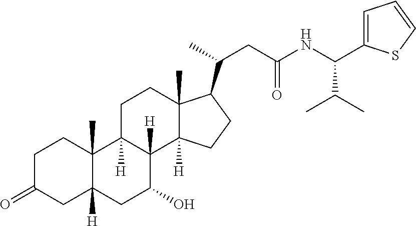 |
| I-136 | 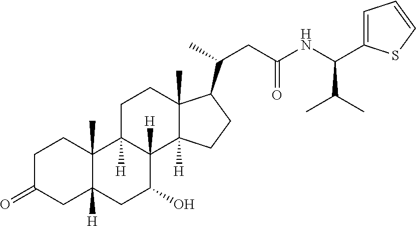 |
| I-137 | 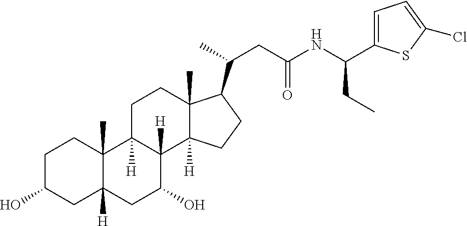 |
| I-138 | 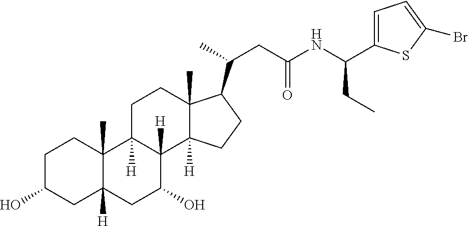 |
| I-139 | 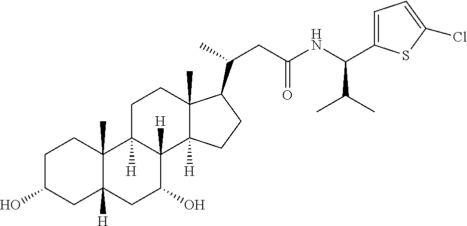 |
| I-140 | 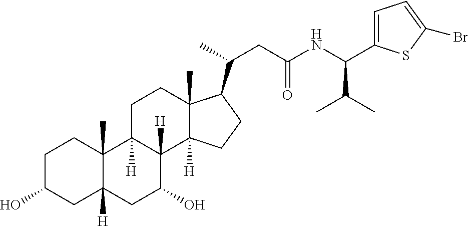 |
| I-141 | 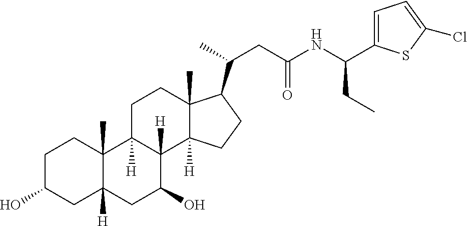 |
| I-142 | 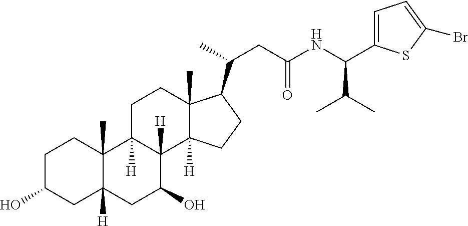 |
| I-143 | 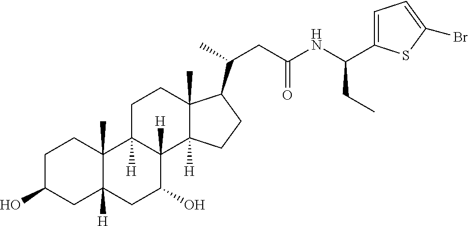 |
| I-144 | 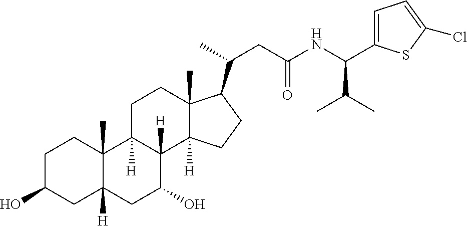 |
| I-145 | 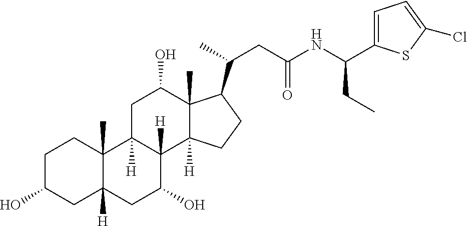 |
| I-146 | 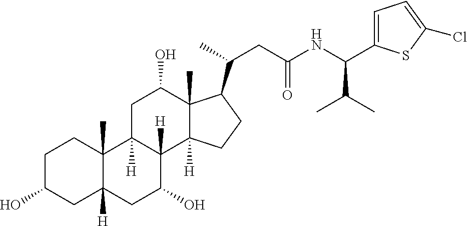 |
| I-147 | 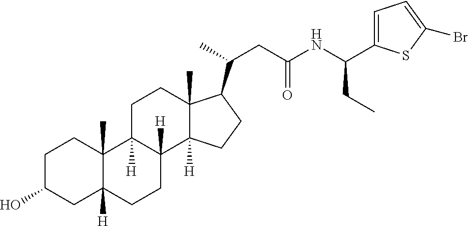 |
| I-148 | 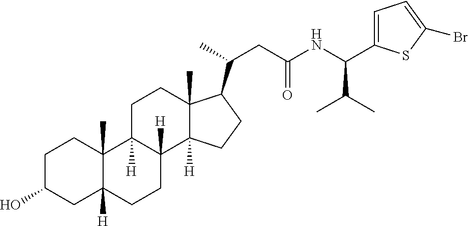 |
| I-149 | 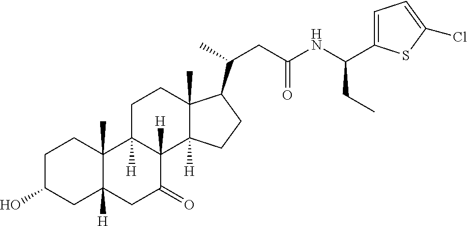 |
| I-150 | 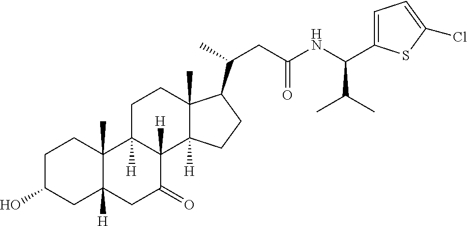 |
| I-151 | 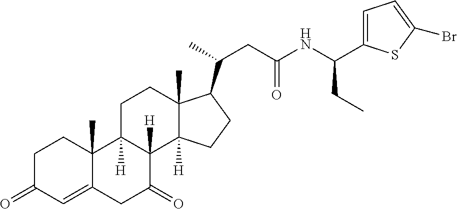 |
| I-152 | 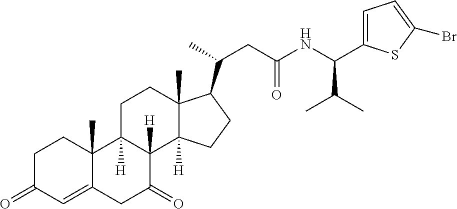 |
| I-153 | 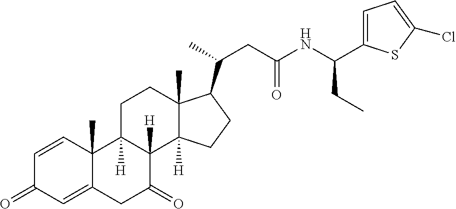 |
| I-154 | 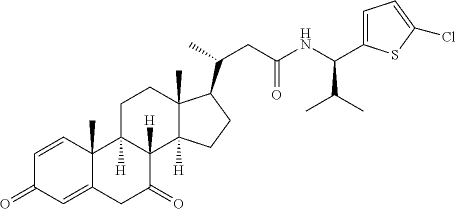 |
| I-155-1 | 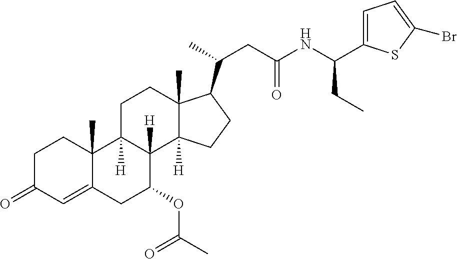 |
| I-155-2 | 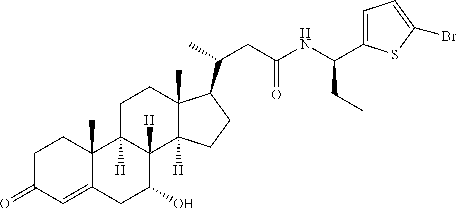 |
| I-156-1 | 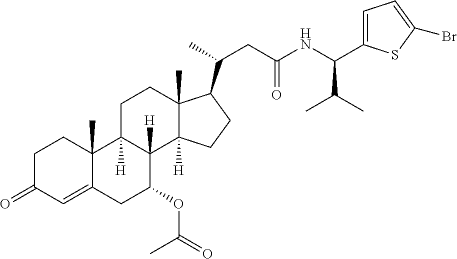 |
| I-156-2 | 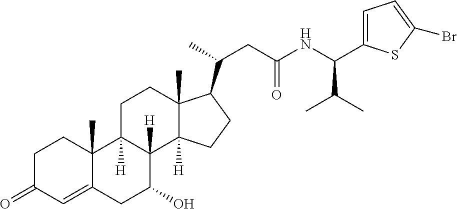 |
| I-157-1 | 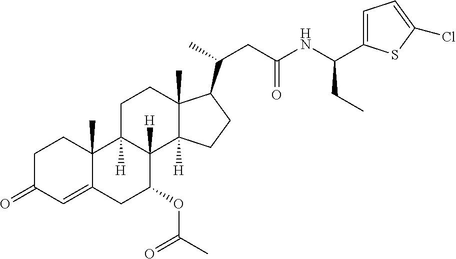 |
| I-157-2 | 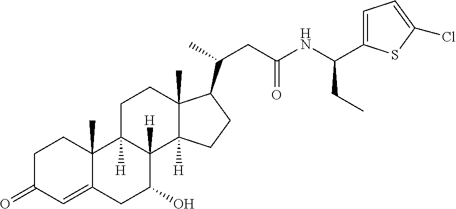 |
| I-158-1 | 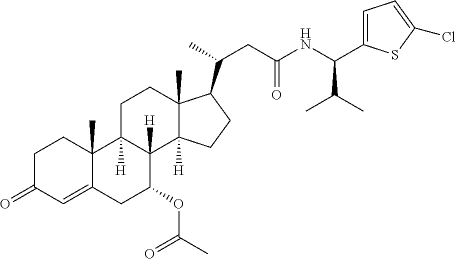 |
| I-158-2 | 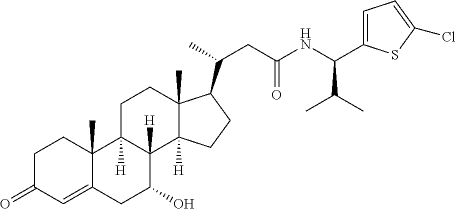 |
| I-159-1 | 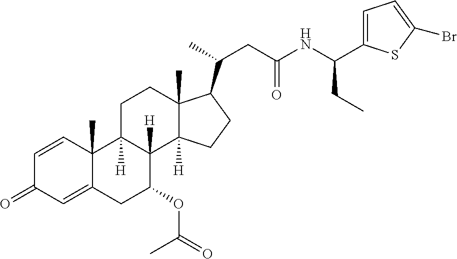 |
| I-159-2 | 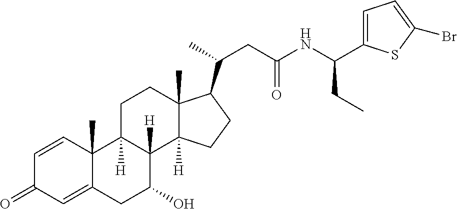 |
| I-160-1 | 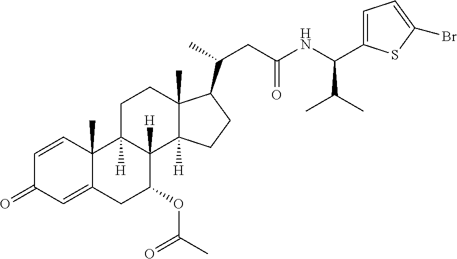 |
| I-160-2 | 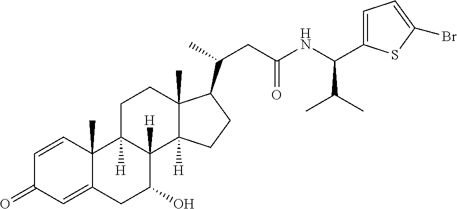 |
| I-161-1 | 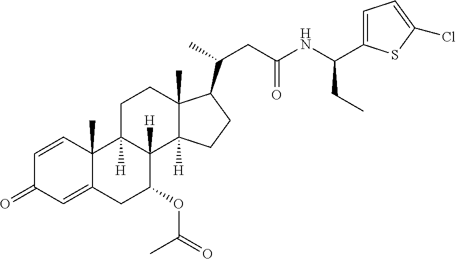 |
| I-161-2 | 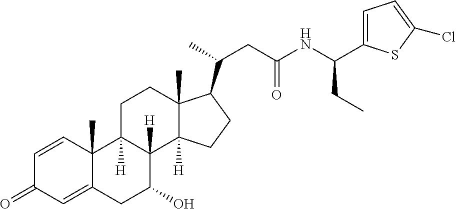 |
| I-162-1 | 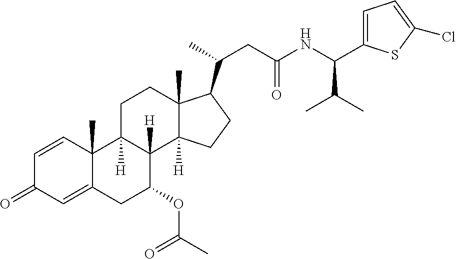 |
| I-162-2 | 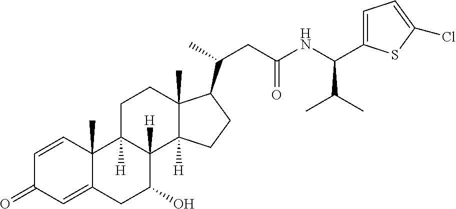 |
| I-163 | 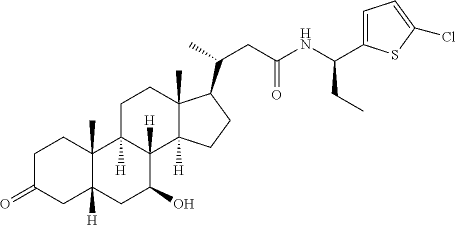 |
| I-164 | 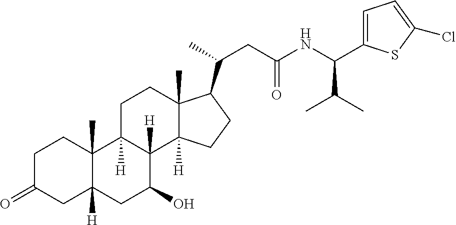 |
| I-165 | 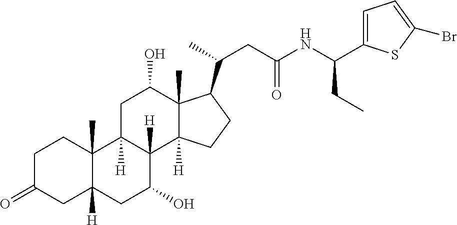 |
| I-166 | 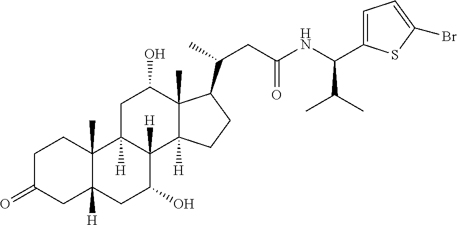 |
| I-167 | 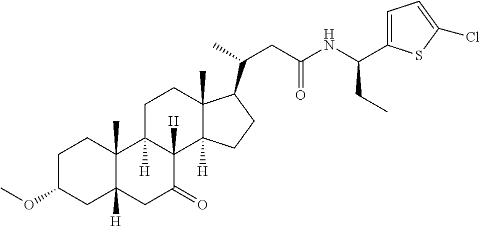 |
| I-168 | 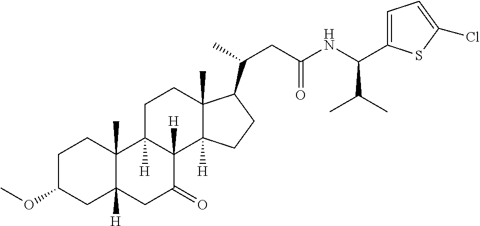 |
| I-169 | 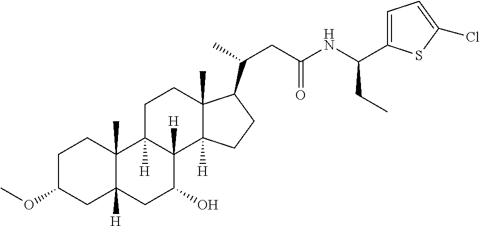 |
| I-170 | 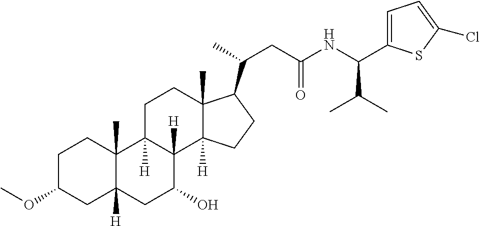 |
| I-171 | 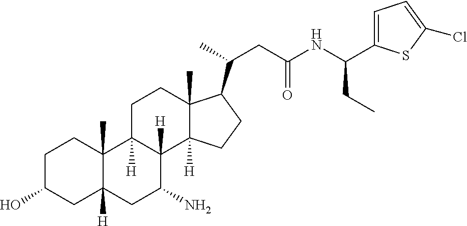 |
| I-172 | 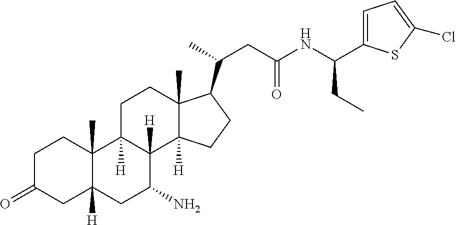 |
| I-173 | 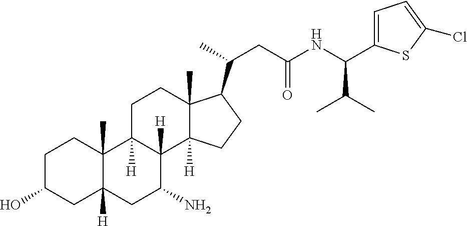 |
| I-174 | 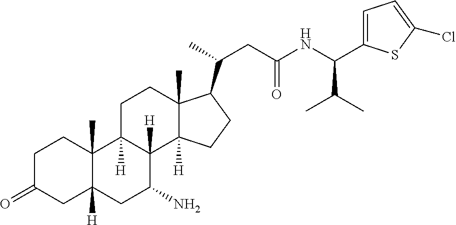 |
| I-175 | 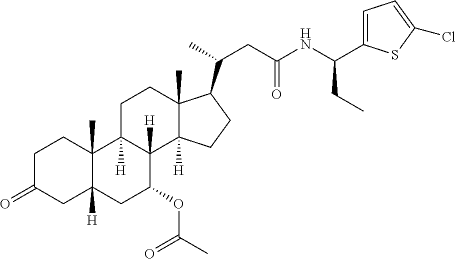 |
| I-176 | 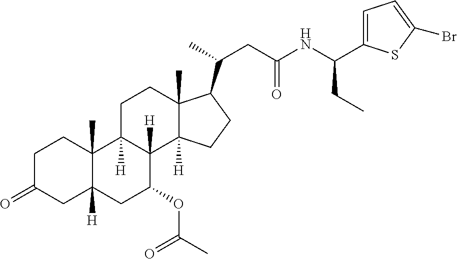 |
| I-177 | 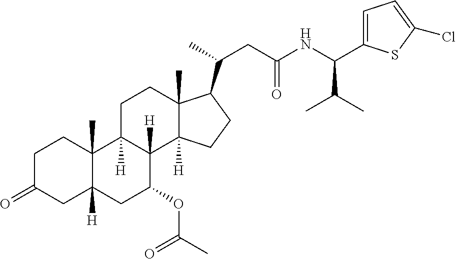 |
| I-178 | 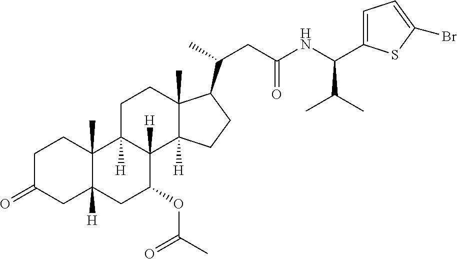 |
| I-179 | 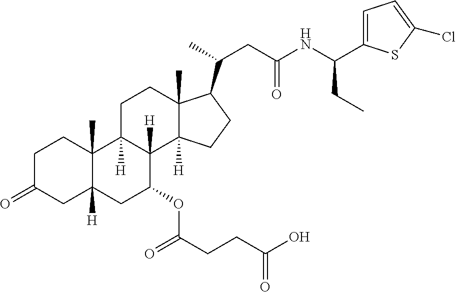 |
| I-180 | 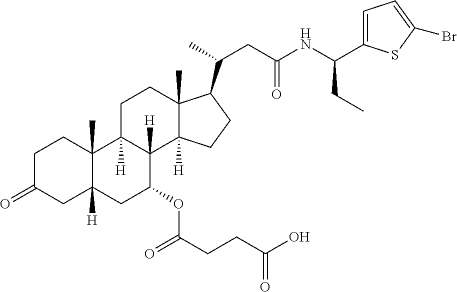 |
| I-181 | 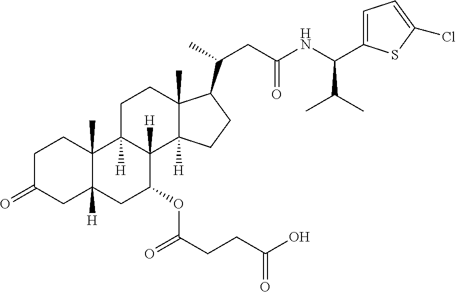 |
| I-182 | 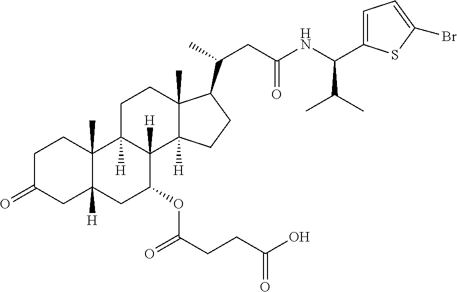 |
| I-183 | 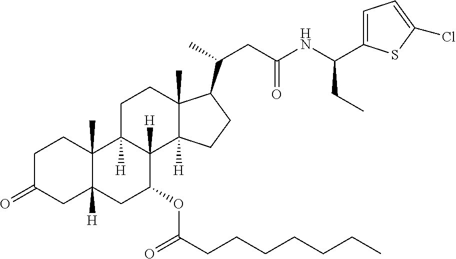 |
| I-184 | 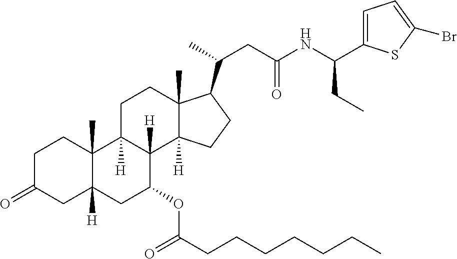 |
| I-185 | 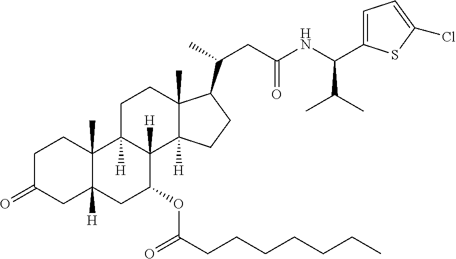 |
| I-186 | 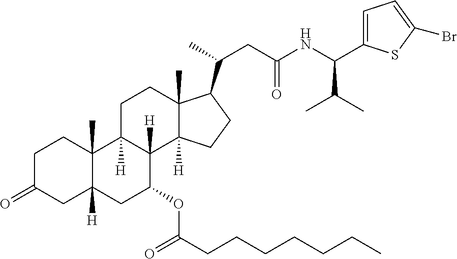 |
| I-187 | 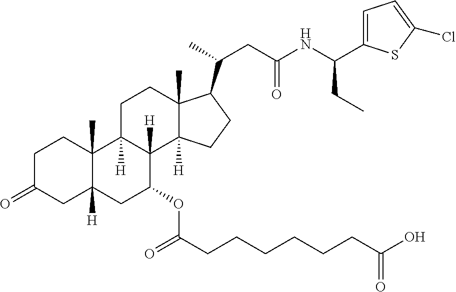 |
| I-188 | 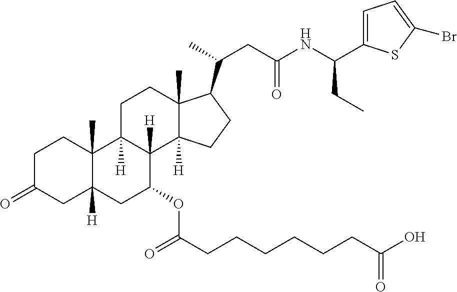 |
| I-189 | 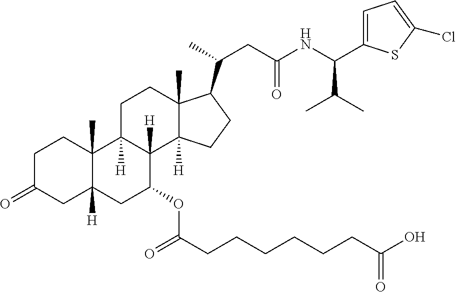 |
| I-190 | 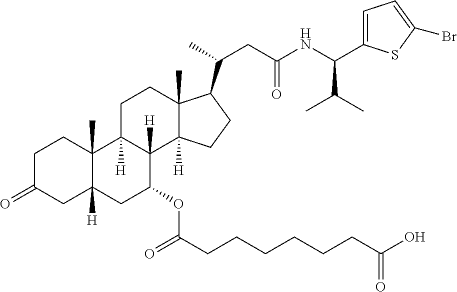 |
| I-191 | 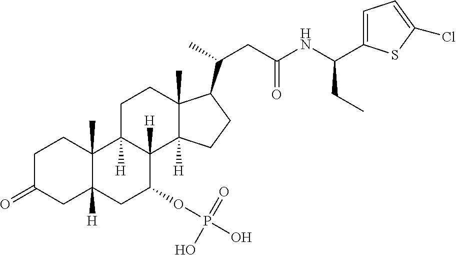 |
| I-191-1 | 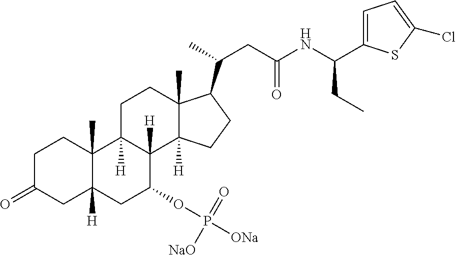 |
| I-192 | 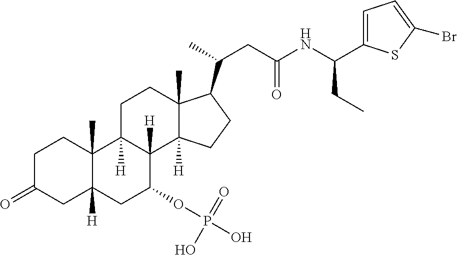 |
| I-192-1 | 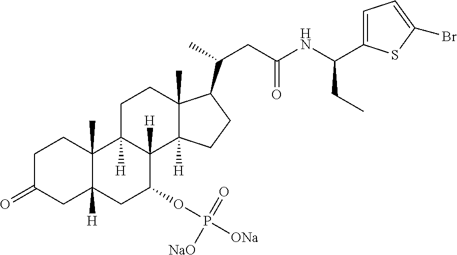 |
| I-193 | 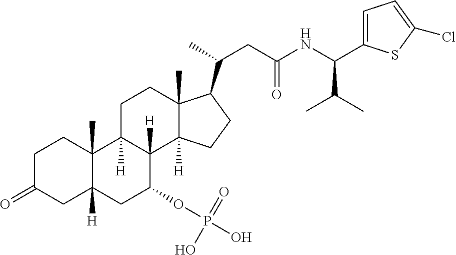 |
| I-193-1 | 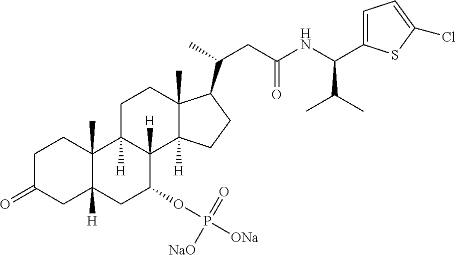 |
| I-194 | 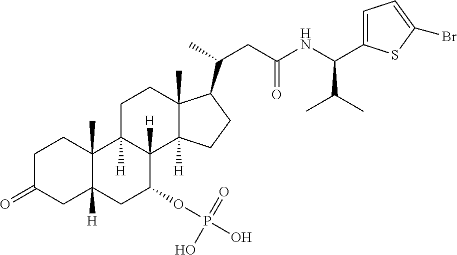 |
| I-194-1 | 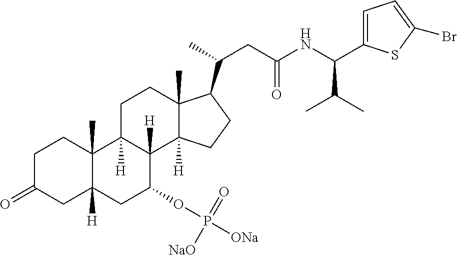 |
| I-195 | 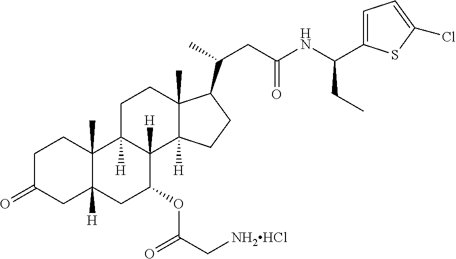 |
| I-196 | 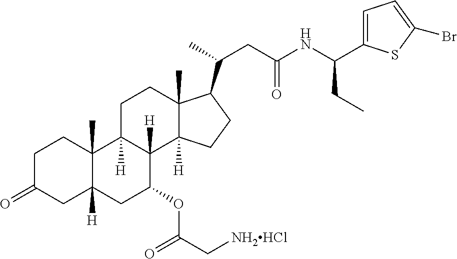 |
| I-197 | 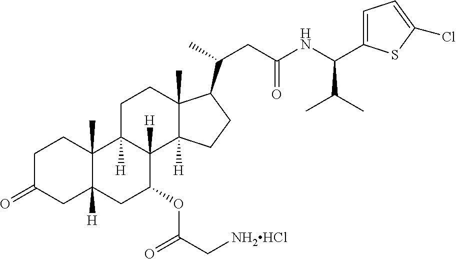 |
| I-198 | 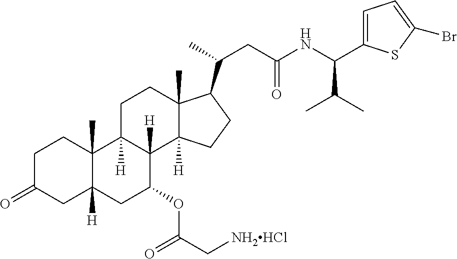 |
| I-199 | 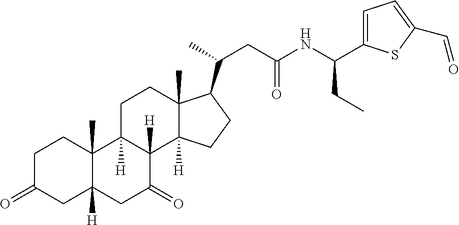 |
| I-200 | 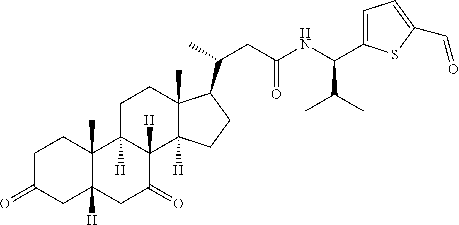 |
| I-201 | 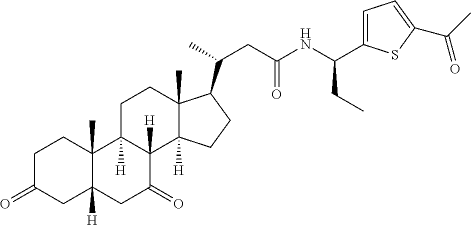 |
| I-202 | 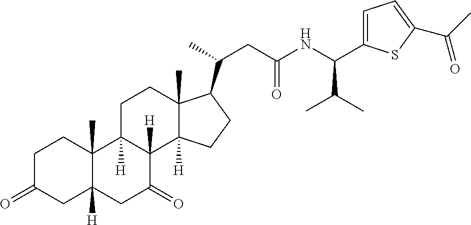 |
| I-203 | 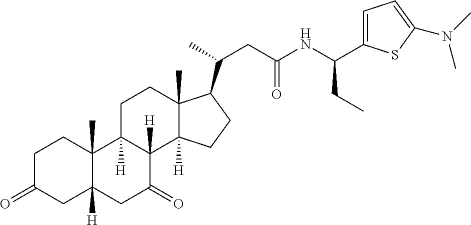 |
| I-204 | 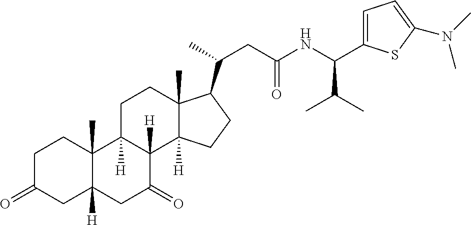 |
| I-205 | 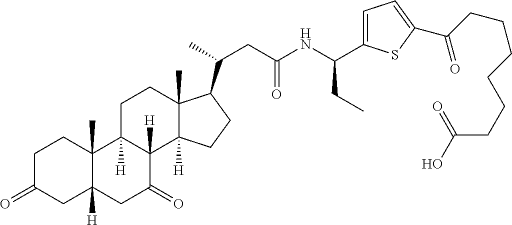 |
| I-206 | 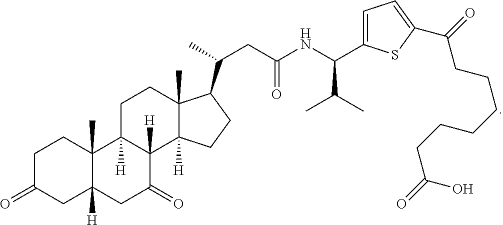 |

The present disclosure further provides use of the compound, or the salt or the stereoisomer thereof in preparation of a bacteriostatic agent.

In some embodiments, the bacteriostatic agent may be a drug for inhibiting germination of C. difficile spores.

In some embodiments, the drug may be capable of preventing and/or treating a C. difficile-caused infectious disease, recurrence of the C. difficile-caused infectious disease, or a complication of the C. difficile-caused infectious disease.

In some embodiments, the C. difficile-caused infectious disease, the recurrence of the C. difficile-caused infectious disease, or the complication of the C. difficile-caused infectious disease may be caused by infection with the C. difficile spores; and

the complication of the C. difficile-caused infectious disease is a gastrointestinal infection syndrome caused by the infection with the C. difficile spores.

In some embodiments, the gastrointestinal infection syndrome may be selected from the group consisting of pseudomembranous enteritis, diverticulitis, and antibiotic-associated diarrhea.

The present disclosure further provides a drug for inhibiting C. difficile spores, in which the drug is a preparation prepared with the compound, or the salt or the stereoisomer thereof as an active ingredient, and a pharmaceutically acceptable excipients.

In some embodiments, the pharmaceutically acceptable excipients may be any one or more selected from the group consisting of a diluent, a filler, a colorant, a glidant, a lubricant, a binder, a stabilizer, a suspending agent, and a buffer.

In some embodiments, the preparation may be an oral preparation; and

preferably, the oral preparation may be selected from the group consisting of a granule, a capsule, a tablet, and a pill.

In some embodiments, a unit preparation of the drug may include 5 mg to 2,500 mg of the active ingredient.

In the present disclosure, the unit preparation refers to 1 preparation unit, such as 1 tablet, 1 capsule, 1 bag of granules, 1 bag of pills, and 1 capsule filled with the pills.

In the present disclosure, the “α-amino acid ester group” refers to a residue with a hydrogen atom removed from carboxyl of α-amino acid, such as —OCOCH₂NH₂.

Experiments have shown that the compound is capable of effectively inhibiting germination of C. difficile spores with a significant bacteriostatic activity. The compound has an excellent prospect for use in preparation of a drug for preventing and/or treating a C. difficile-caused infectious disease, recurrence of the C. difficile-caused infectious disease, or a complication of the C. difficile-caused infectious disease.

Obviously, according to the above-mentioned content of the present disclosure and on the basis of common technical knowledges and common methods in the field, various other modifications, substitutions or alterations can be made without departing from the above-mentioned basic technical idea of the present disclosure.

The above-mentioned content of the present disclosure will be further described in detail below through specific embodiments in the form of examples. However, it should not be construed that the subject of the present disclosure is limited to the following examples. Instead, embodiments based on the content of the present disclosure should fall within the scope of the present disclosure.

### DETAILED DESCRIPTION OF THE EMBODIMENTS

Unless otherwise specified, in the present disclosure, raw materials and equipment used are all known commercially available products obtained by purchasing.

1. Synthesis of Nor-Cholic Acid Intermediates:

In the present disclosure, decarbonized (nor) cholic acids include decarbonized cholic acid (nor-CA), decarbonized chenodeoxycholic acid (nor-CDCA), decarbonized ursodeoxycholic acid (nor-UDCA), decarbonized hyodeoxycholic acid (nor-HCA), decarbonized lithocholic acid (nor-LCA), and decarbonized hyodeoxycholic acid (nor-HDCA); these compounds are synthesized with reference to the method described in “Journal of Lipid Research. 1988, 29: 1387”.

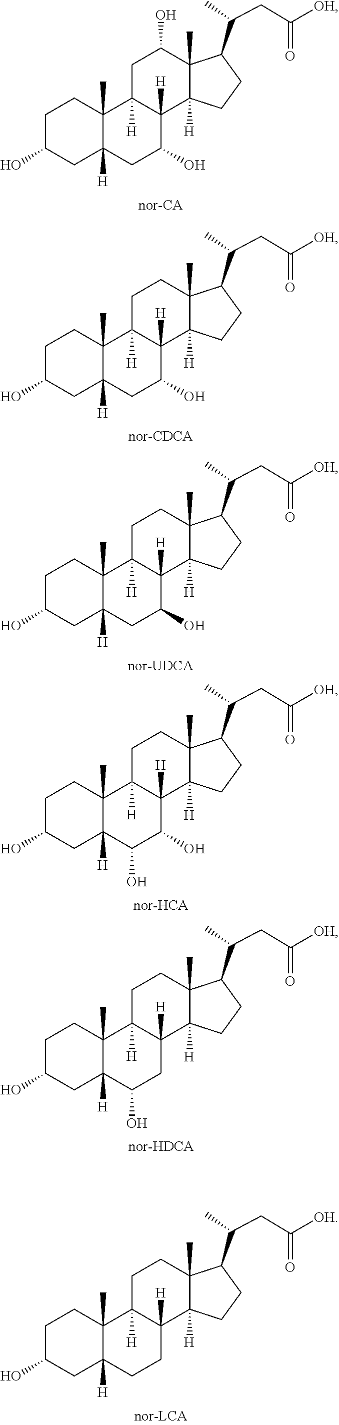

Taking the nor-UDCA as an example, a synthesis method included:

Step 1: 20 g of UDCA was mixed with a formic acid solution and a catalytic amount of perchloric acid, stirred overnight, concentrated under reduced pressure, and an obtained residue was treated with a toluene/petroleum ether (1:1) mixture system to obtain a white solid.

Step 2: the white solid without purification was mixed and dissolved with 100 mL of trifluoroacetic acid/trifluoroacetic anhydride; 5 equivalents of sodium nitrite was gradually added in batches at room temperature, a resulting mixture was slowly heated under stirring until 40° C. to 50° C., and cooled to room temperature after 30 min; the reaction was quenched by ice water/toluene, a resulting aqueous layer was extracted with toluene, toluene layers were combined, and concentrated under reduced pressure to obtain an oily substance.

Step 3: 100 ml of a mixed solution of ethanol/20% sodium hydroxide was added to the oily substance, heated under reflux until an end of the reaction, and crystallized by cooling to obtain a nor-cyano intermediate.

Step 4: the intermediate was stirred by reflux in an isopropanol/sodium hydroxide system for 24 h until cyano was hydrolyzed into carboxyl; after concentrating a solvent under reduced pressure, an acidity was adjusted to separate out a white solid. The white solid was further purified by recrystallization or column chromatography to obtain the nor-UDCA with an HPLC purity of greater than 95%, and a yield of 55% to 68%.

Similarly, the nor-CA, nor-CDCA, nor-HCA, nor-LCA, and nor-HDCA were prepared according to the above steps using corresponding CA, CDCA, HCA, LCA, and HDCA as raw materials.

2. Synthesis of Oxidized (Dehydrogenated) Nor-Cholic Acid Intermediates:

An oxidized nor-CDCA intermediate was prepared using nor-CDCA as a raw material:

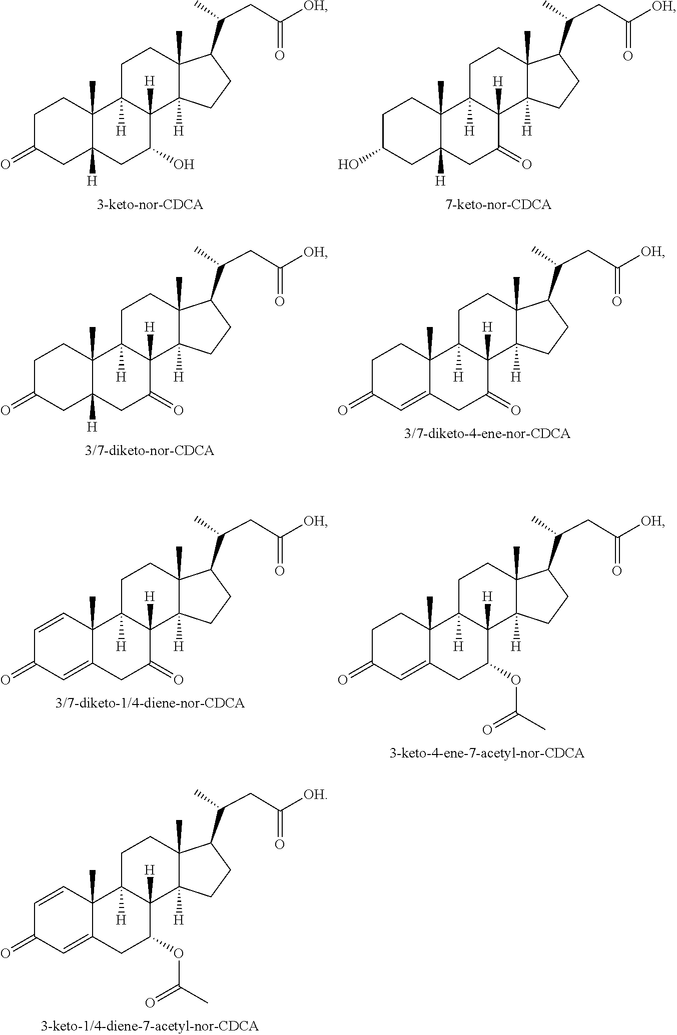

(1) Preparation of 3-keto-decarbonized chenodeoxycholic acid (3-keto-nor-CDCA):

10 g of a nor-CDCA raw material was dissolved in 50 mL of toluene, 1.5 equivalents of freshly-prepared silver carbonate/diatomite was added, and a reaction was conducted at 80° C. until the raw material disappeared; after filtration, a resulting product was concentrated under reduced pressure, and subjected to silica gel column chromatography or recrystallization to obtain the 3-keto-nor-CDCA with an HPLC purity of greater than 95%, and a yield of 75%.

Further, the 3-keto-nor-CDCA was reduced with sodium borohydride, and two products were obtained after separation by silica gel column chromatography, namely the nor-CDCA and 3β-nor-CDCA.

(2) Preparation of 7-keto-decarbonized chenodeoxycholic acid (7-keto-nor-CDCA):

10 g of a nor-CDCA raw material was dissolved in 50 mL of an acetic acid aqueous solution, 1.2 equivalents of NBS was added, and a reaction was conducted at room temperature until the raw material disappeared; a resulting product was subjected to silica gel column chromatography or recrystallization to obtain the 7-keto-nor-CDCA with an HPLC purity of greater than 95%, and a yield of 88%.

(3) Preparation of 3,7-diketo-decarbonized chenodeoxycholic acid (3,7-diketo-nor-CDCA):

10 g of a nor-CDCA raw material was dissolved in 50 mL of acetone, 4 equivalents of CrO₃ was added, and a reaction was conducted at room temperature until the completely disappeared; after filtration, a resulting product was concentrated, and subjected to silica gel column chromatography or recrystallization to obtain the 3,7-diketo-nor-CDCA with an HPLC purity of greater than 95%, and a yield of 71%.

(4) Preparation of 3,7-diketo-4-ene-decarbonized chenodeoxycholic acid (3,7-diketo-4-ene-nor-CDCA):

10 g of a 7-keto-nor-CDCA raw material was dissolved in 50 mL of toluene, 1.0 equivalent of 2-iodoxybenzoic acid (IBX) was added, and a reaction was conducted at room temperature until the raw material disappeared; a resulting product showed strong fluorescence by thin layer chromatography (TLC) detection in spots, and was subjected to silica gel column chromatography or recrystallization to obtain the 3,7-diketo-4-ene-nor-CDCA with an HPLC purity of greater than 95%, and a yield of 64%.

(5) Preparation of 3,7-diketo-1,4-diene-decarbonized chenodeoxycholic acid (3,7-diketo-1,4-diene-nor-CDCA):

The 3,7-diketo-4-ene-nor-CDCA was further reacted with the IBX by the above method to obtain the 3,7-diketo-1,4-diene-nor-CDCA.

(6) Preparation of 3-keto-4-ene-7-acetyl-decarbonized chenodeoxycholic acid (3-keto-4-ene-7-acetyl-nor-CDCA):

10 g of a 3-keto-nor-CDCA raw material was reacted with an esterifying agent such as acetic anhydride to obtain a 7-hydroxy ethyl-esterified derivative. The derivative was further reacted with the IBX by the above method to obtain the 3-keto-4-ene-7-acetyl-nor-CDCA.

(7) Preparation of 3-keto-1,4-diene-7-acetyl-decarbonized chenodeoxycholic acid (3-keto-1,4-diene-7-acetyl-nor-CDCA):

A 3-keto-4-ene-7-acetyl-nor-CDCA derivative was further reacted with the IBX by the above method to obtain the 3-keto-1,4-diene-7-acetyl-nor-CDCA.

With reference to the above method for preparing the oxidized nor-CDCA intermediate, the following oxidized decarbonized cholic acid intermediates were prepared separately: 3-keto-decarbonized ursodeoxycholic acid (3-keto-nor-UDCA), 3-keto-decarbonized cholic acid (3-keto-nor-CA), 7-keto-decarbonized cholic acid (7-keto-nor-CA), 3-keto-decarbonized lithocholic acid (3-keto-nor-LCA), 3-keto-decarbonized hyodeoxycholic acid (3-keto-nor-HCA), 3-keto-decarbonized hyodeoxycholic acid (3-keto-nor-HDCA), and 3,7-diketo-decarbonized hyodeoxycholic acid (3,7-diketo-nor-HDCA).

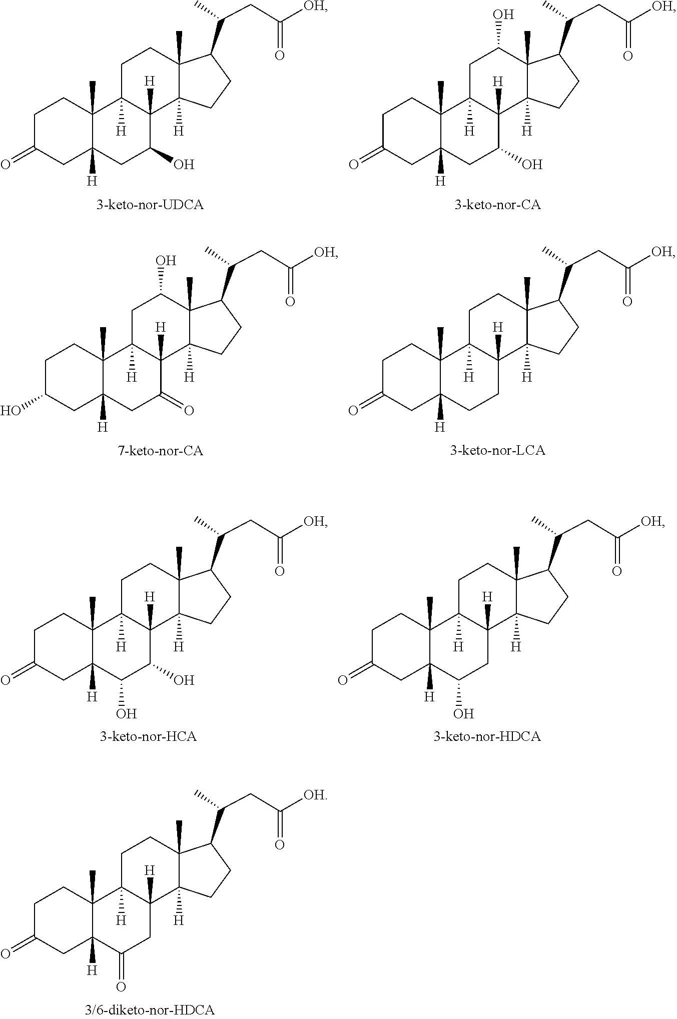

3. Synthesis of Other Cholic Acid Intermediates:

The decarbonized cholic acid intermediates with structures shown below were prepared using the above decarbonized cholic acid intermediates as raw materials:

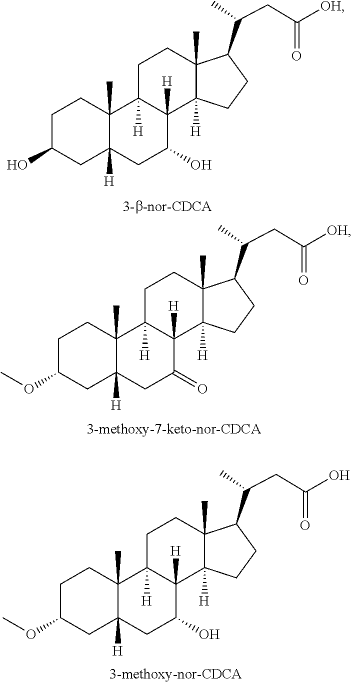

(1) Preparation of 3-β-decarbonized chenodeoxycholic acid (3β-nor-CDCA):

The 3-keto-nor-CDCA was reduced with sodium borohydride, and two products were obtained after separation by silica gel column chromatography, namely the nor-CDCA and the 3-β-nor-CDCA.

(2) Preparation of 3-methoxy-7-keto-decarbonized chenodeoxycholic acid (3-methoxy-7-keto-nor-CDCA)

The 7-keto-nor-CDCA in tetrahydrofuran (THF) solvent was added sodium cyanide and methyl iodide for reaction until raw materials were depleted, and ammonium chloride was added to terminate the reaction. The 3-methoxy-7-keto-nor-CDCA was isolated by silica gel column chromatography.

(3) Preparation of 3-methoxy-decarbonized chenodeoxycholic acid (3-methoxy-nor-CDCA):

The 3-methoxy-7-keto-nor-CDCA was reduced with sodium borohydride, and separated by silica gel column chromatography to obtain the 3-methoxy-nor-CDCA.

4. Synthesis of Thiophenine Intermediates:

(R/S)-1-(thienyl-2)-propylamine (I-A-1), (R)-1-(thienyl-2)-propylamine (I-A-1-R), and (S)-1-(thienyl-2)-propylamine (I-A-1-S) were prepared separately following the following synthetic routes.

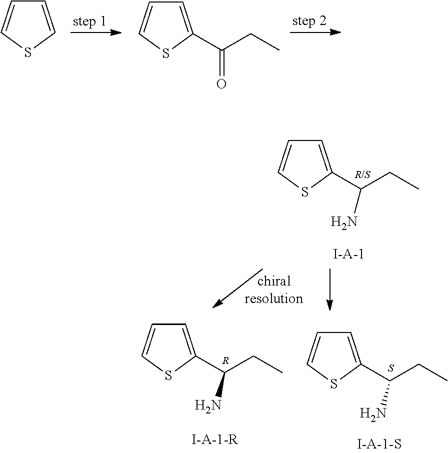

Step 1: propionic acid (4.42 mmol), trifluoroacetic acid (2.5 mL) and thienyl 4.42 mmol were mixed for a reaction, under stirring, 4.42 mmol 85% H₃PO₄ was added dropwise until the reaction was completed, and a resulting product was poured into ice water to precipitate a white solid.

Step 2: the solid was dissolved and dispersed in toluene, 2 equivalents of ammonium acetate and 2 equivalents of sodium borocyanide were added, and a reaction was completed under heating; after cooling, the reaction was quenched by acid water, an acid water layer was separated, and adjusted with a diluted sodium hydroxide aqueous solution; after extraction with methyl tert-butyl ether, a resulting product was dried and concentrated, a residue was crystallized with a DL tartaric acid/acetone water system to obtain racemic-1-(thienyl-2)-propylamine tartrate with an HPLC purity of greater than 98%. The salt was extracted and distributed with an ethyl acetate/diluted aqueous sodium hydroxide solution; an ethyl acetate layer was dried and concentrated to dryness under reduced pressure to obtain a colorless oily substance, namely the (R/S)-1-(thienyl-2)-propylamine (I-A-1), with a total yield of 52%.

Step 3 (chiral resolution): 14.1 g of the racemic thienyl propylamine (100 mmol, 1.0 eq) and 70 ml of absolute ethanol were added to a 250 ml there-necked flask, and heated to 60° C.; a tartaric acid ethanol solution (prepared by dissolving 15.0 g of D-type tartaric acid into 70 ml of absolute ethanol, 1.0 eq) was slowly added dropwise to a reaction solution, kept at about 60° C., and stirred for 0.5 h; after stirring, a product was cooled to room temperature, and a white solid was precipitated, and then filtered to obtain 28 g of solid (wet product); the solid was repeatedly recrystallized with anhydrous ethanol, dried by blasting at 60° C. to obtain 7.5 g of (R)-thienyl propylamine-D-tartrate.

7.5 g of the solid was partitioned with a 5% sodium hydroxide solution and ethyl acetate; an ethyl acetate layer was washed 1 to 2 times with water, dried over anhydrous sodium sulfate, and filtered to remove the sodium sulfate; a filtrate was concentrated under reduced pressure at 60° C. and evaporated to dryness to obtain 3.6 g of the (R)-1-(thienyl-2)-propylamine (I-A-1-R), with an enantiomeric excess (ee) of greater than 98% determined by HPLC.

All crystallization mother liquors were combined, and after recovering thienyl propylamine, crystallization was conducted by a same method with L-tartaric acid to obtain 2.8 g of the (S)-1-(thienyl-2)-propylamine (I-A-1-S), with an ee of greater than 98% determined by HPLC.

Referring to the synthetic methods of I-A-1, I-A-1-R and I-A-1-S, the rest of thiophenine derivatives were prepared by switching to different substituted thienyl raw materials and different acids. For example:

(R/S)-1-(thienyl-2)-ethylamine (I-A-2), (R)-1-(thienyl-2)-ethylamine (I-A-2-R) and S-1-(thienyl-2)-ethylamine (I-A-2-S);

(R/S)-1-(5-chloro-thienyl-2)-ethylamine (I-A-3), (R)-1-(5-chloro-thienyl-2)-ethylamine (I-A-3-R) and S-1-(5-chloro-thienyl-2)-ethylamine (I-A-3-S);

(R/S)-1-(5-bromo-thienyl-2)-ethylamine (I-A-4), (R)-1-(5-bromo-thienyl-2)-ethylamine (I-A-4-R) and S-1-(5-bromo-thienyl-2)-ethylamine (I-A-4-S);

(R/S)-1-(thienyl-2)-cyclopropylmethylamine (I-A-5), (R)-1-(thienyl-2)-cyclopropylmethylamine (I-A-5-R) and (S)-1-(thienyl-2)-cyclopropylmethylamine (I-A-5-S);

(R/S)-1-(5-chloro-thienyl-2)-propylamine (I-A-6), (R)-1-(5-chloro-thienyl-2)-propylamine (I-A-6-R) and (S)-1-(5-chloro-thienyl-2)-propylamine (I-A-6-S);

(R/S)-1-(5-bromo-thienyl-2)-propylamine (I-A-7), (R)-1-(5-bromo-thienyl-2)-propylamine (I-A-7-R) and (S)-1-(5-bromo-thienyl-2)-propylamine (I-A-7-S);

(R/S)-2-methyl-1-(thienyl-2)-1-propylamine (I-A-8), (R)-2-methyl-1-(thienyl-2)-1-propylamine (I-A-8-R) and (S)-2-methyl-1-(thienyl-2)-1-propylamine (I-A-8-S);

(R/S)-2-methyl-1-(5-chloro-thienyl-2)-1-propylamine (I-A-9), (R)-2-methyl-1-(5-chloro-thienyl-2)-1-propanamine (I-A-9-R) and (S)-2-methyl-1-(5-chloro-thienyl-2)-1-propanamine (I-A-9-S);

(R/S)-2-methyl-1-(5-bromo-thienyl-2)-1-propylamine (I-A-10), (R)-2-methyl-1-(5-bromo-thienyl-2)-1-propylamine (I-A-10-R) and (S)-2-methyl-1-(5-bromo-thienyl-2)-1-propylamine (I-A-10-S);

(R/S)-1-(thienyl-2)-butylamine (I-A-11), (R/S)-1-(thienyl-2)-pentylamine (I-A-12), (R/S)-1-(thienyl-2)-hexylamine (I-A-13), (R/S)-1-(thienyl-2)-octylamine (I-A-14), (R/S)-1-(thienyl-2)-dodecylamine (I-A-15), (R/S)-1-(thienyl-2)-tetradecylamine (I-A-16), (R/S)-1-(thienyl-2)-cyclopentylmethylamine (I-A-17), (R/S)-1-(thienyl-2)-cyclobutylmethylamine (I-A-18), (R/S)-1-(thienyl-2)-cyclohexylmethylamine (I-A-19), (R/S)-2-dimethyl-1-(thienyl-2)-propylamine (I-A-20), (R/S)-1-(thienyl-2)-benzylamine (I-A-21), bis-(thienyl-2)-methylamine (I-A-22), and (R/S)-1-(thienyl-2)-cyclobutylmethylamine (I-A-23).

The raw materials in the following examples can be obtained by referring to the above method or directly purchased.

### Example 1 Synthesis of Compound I-1

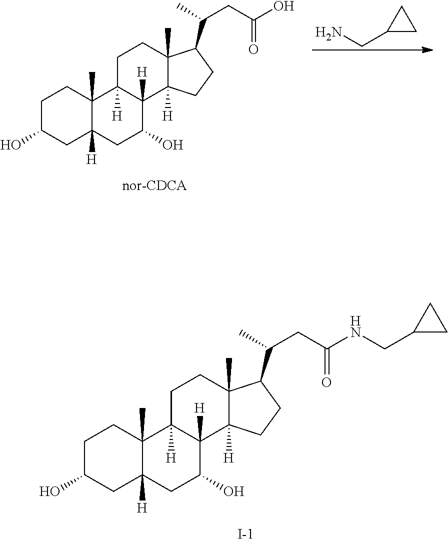

5.5 g of nor-CDCA (14.5 mmol) was dissolved in 110 ml of N,N-dimethylformamide (DMF), and 1.3 g of cyclopropylmethylamine (18.3 mmol) and 2.3 g of N,N-diisopropylethylamine (DIPEA, 17.8 mmol) were added; after stirring and dissolving, a reaction system was cooled by ice-salt bath to 0° C. to 5° C., and 5.7 g of O-benzotriazole-1-yl-N,N,N′,N′-tetramethyluronium tetrafluoroborate (TBTU, 17.8 mmol) was added to the reaction system in stages. After the TBTU was added completely, a reaction was conducted at 0° C. to 5° C. for 1 h, and the reaction system was heated to room temperature and stirred overnight.

The complete reaction of cholic acid raw materials was monitored by thin layer chromatography (TLC), water (110 ml) and ethyl acetate (110 ml) in a same volume as the DMF were added to the reaction system, and allowed to stand for stratification. An ethyl acetate layer was washed twice with one-fifth volume (22 ml) of a 10% sodium carbonate solution, twice with one-fifth volume (22 ml) of 5% hydrochloric acid, and then twice with one-fifth volume (22 ml) of saturated brine. The ethyl acetate layer was dried over 10 g of anhydrous sodium sulfate, the sodium sulfate was removed by filtration, the ethyl acetate was concentrated to dryness, and a residue was purified by silica gel column chromatography to obtain a total of 5.1 g of the compound I-1 with an HPLC purity of greater than 98%, and a yield of 81.7%.

¹HNMR(CDCl₃, 400 MHz): δ3.88 (s, 1H), 3.16 (s, 1H), 2.55-2.40 (m, 1H), 2.32-2.13 (m, 1H), 2.05-1.97 (m, 4H), 1.97-1.67 (m, 11H), 1.58-1.36 (m, 6H), 1.35-1.16 (m, 2H), 1.01 (d, J=6.4 Hz, 3H), 0.99-0.95 (m, 1H), 0.93 (s, 3H), 0.74 (s, 3H), 0.53 (d, J=7.7 Hz, 2H), 0.23 (d, J=5.0 Hz, 2H);

ESI-MS m/z: 430.31[M−1]⁻

### Example 2 Synthesis of Compound I-2

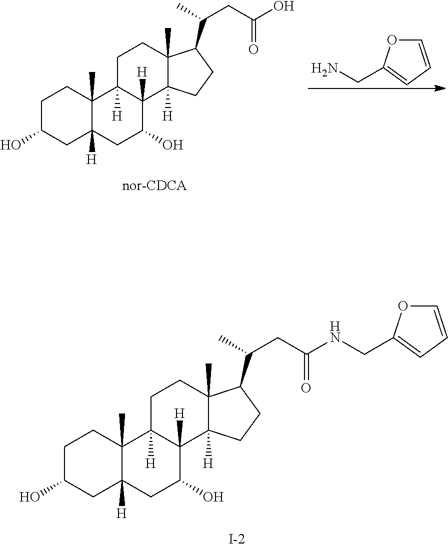

The compound I-2 was obtained by a synthetic method similar to that in Example 1 using nor-CDCA and 2-furfurylamine as raw materials.

¹HNMR(CDCl₃, 400 MHz): δ7.37 (s, 1H), 6.34 (s, 1H), 6.24 (d, J=3.1 Hz, 1H), 5.82 (t, J=5.6 Hz, 1H), 4.46 (d, J=5.2 Hz, 2H), 3.87 (brs, 1H), 3.48 (tt, J=10.6, 4.5 Hz, 1H), 2.42 (dd, J=13.9, 3.5 Hz, 1H), 2.21 (q, J=12.5 Hz, 1H), 2.05-1.93 (m, 3H), 1.89-1.59 (m, 8H), 1.57-1.23 (m, 7H), 1.23-1.09 (m, 3H), 1.07-1.02 (m, 1H), 0.99 (d, J=6.5 Hz, 3H), 0.92 (s, 3H), 0.72 (s, 3H);

ESI-MS m/z: 456.22[M−1]⁻

### Example 3 Synthesis of Compound I-3

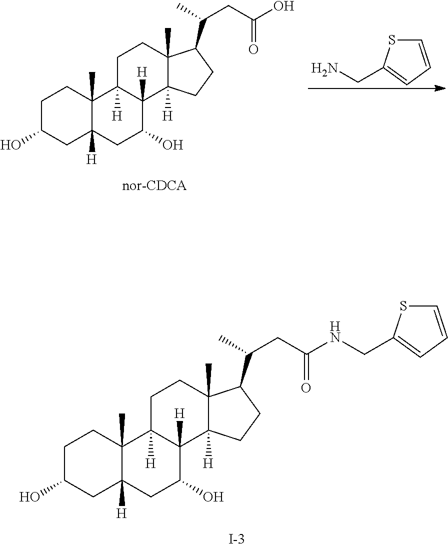

The compound I-3 was obtained by a synthetic method similar to that in Example 1 using nor-CDCA and 2-thiophenemethylamine as raw materials.

¹HNMR (CDCl₃, 400 MHz): 37.25 (d, J=4.9 Hz, 1H), 7.01-6.95 (m, 2H), 5.82 (brs, 1H), 4.64 (t, J=6.1 Hz, 2H), 3.87 (d, J=3.2 Hz, 1H), 3.55-3.42 (m, 1H), 2.48-2.39 (m, 1H), 2.29-2.14 (m, 1H), 2.07-1.95 (m, 3H), 1.94-1.60 (m, 7H), 1.57-1.47 (m, 3H), 1.47-1.25 (m, 5H), 1.23-1.11 (m, 3H), 1.05-1.03 (m, 1H), 1.01 (d, J=6.4 Hz, 3H), 0.93 (s, 3H), 0.73 (s, 3H);

ESI-MS m/z: 472.28[M−1]⁻

### Example 4 Synthesis of Compound I-4

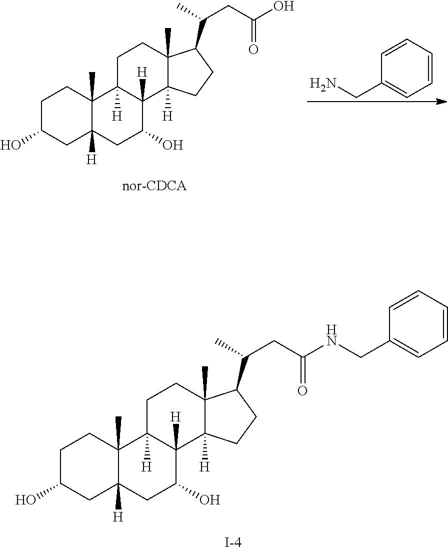

The compound I-4 was obtained by a synthetic method similar to that in Example 1 using nor-CDCA and benzylamine as raw materials.

¹H NMR (CDCl₃, 400 MHz) δ 7.40-7.26 (m, 5H), 5.87 (s, 1H), 4.54-4.40 (m, 2H), 3.87 (d, J=3.3 Hz, 1H), 3.55-3.42 (m, 1H), 2.50-2.41 (m, 1H), 2.33-2.14 (m, 1H), 2.12-1.95 (m, 4H), 1.94-1.81 (m, 4H), 1.78-1.59 (m, 4H), 1.57-1.46 (m, 3H), 1.47-1.25 (m, 4H), 1.28-1.09 (m, 3H), 1.01 (d, J=6.6 Hz, 3H), 0.92 (s, 3H), 0.72 (s, 3H).

ESI-MS m/z: 466.30[M−1]⁻

### Example 5 Synthesis of Compound I-5

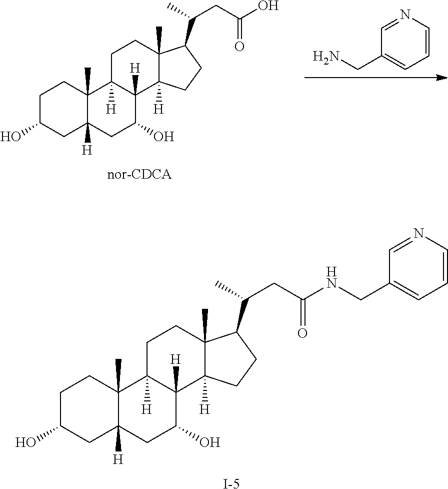

The compound I-5 was obtained by a synthetic method similar to that in Example 1 using nor-CDCA and 3-(aminomethyl)pyridine as raw materials.

ESI-MS m/z: 467.32[M−1]⁻

### Example 6 Synthesis of Compound I-6

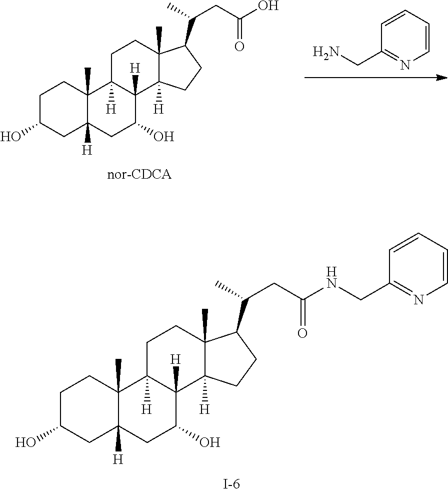

The compound I-6 was obtained by a synthetic method similar to that in Example 1 using nor-CDCA and 2-(aminomethyl)pyridine as raw materials.

¹H NMR (CDCl₃, 400 MHz): δ 8.56 (d, J=5.0 Hz, 1H), 7.80-7.70 (m, 1H), 7.40-7.33 (m, 1H), 7.32-7.23 (m, 1H), 6.85 (s, 1H), 4.61 (d, J=5.0 Hz, 2H), 3.87 (q, J=3.1 Hz, 1H), 3.55-3.42 (m, 1H), 2.52-2.42 (m, 1H), 2.31-2.15 (m, 1H), 2.09-1.95 (m, 4H), 1.95-1.78 (m, 4H), 1.57-1.46 (m, 3H), 1.46-1.37 (m, 2H), 1.41-1.31 (m, 2H), 1.34-1.18 (m, 2H), 1.22-1.09 (m, 2H), 1.00 (d, J=6.2 Hz, 3H), 1.06-0.88 (m, 1H), 0.92 (s, 3H), 0.73 (s, 3H). ESI-MS m/z: 467.32[M−1]⁻

### Example 7 Synthesis of Compound I-7

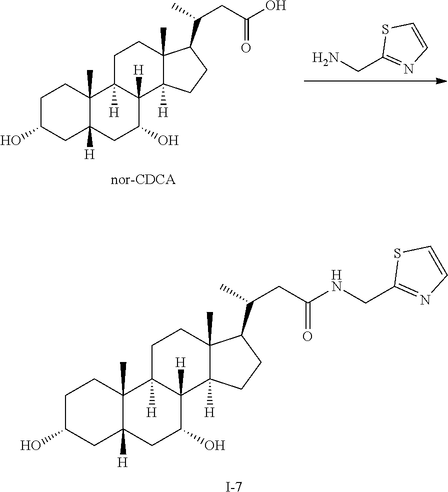

The compound I-7 was obtained by a synthetic method similar to that in Example 1 using nor-CDCA and 2-thiazolylmethylamine as raw materials.

¹HNMR(CDCl₃, 400 MHz): δ7.76-7.70 (m, 1H), 7.37-7.29 (m, 1H), 4.82-4.75 (m, 2H), 3.87 (d, J=3.6 Hz, 1H), 3.54-3.41 (m, 1H), 2.51-2.40 (m, 1H), 2.29-2.15 (m, 1H), 2.09-1.94 (m, 3H), 1.92-1.79 (m, 5H), 1.76-1.60 (m, 4H), 1.56-1.12 (m, 10H), 1.01 (d, J=6.5 Hz, 3H), 1.05-0.87 (m, 1H), 0.92 (s, 3H), 0.72 (s, 3H).

ESI-MS m/z: 473.25[M−1]⁻

### Example 8 Synthesis of Compound I-8

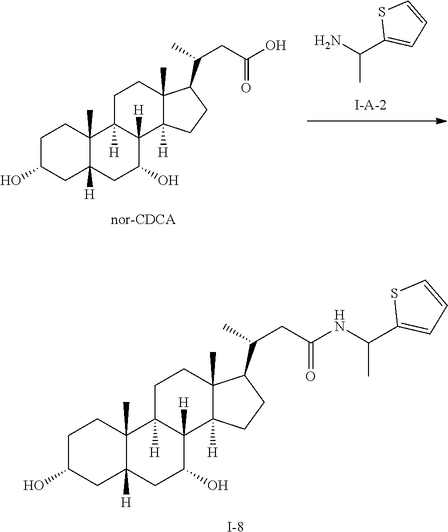

The compound I-8 was obtained by a synthetic method similar to that in Example 1 using nor-CDCA and I-A-2 as raw materials.

¹HNMR(CDCl₃, 400 MHz): δ 7.22 (d, J=4.8 Hz, 1H), 7.01-6.94 (m, 2H), 5.73-5.65 (m, 1H), 5.15-5.39 (m, 1H), 3.87 (q, J=3.0 Hz, 1H), 3.55-3.39 (m, 1H), 2.46-2.30 (m, 1H), 2.31-2.14 (m, 1H), 2.09-1.95 (m, 4H), 1.94-1.80 (m, 3H), 1.79-1.68 (m, 2H), 1.64-1.46 (m, 5H), 1.54 (s, 3H), 1.47-1.37 (m, 2H), 1.39-1.27 (m, 1H), 1.28 (s, 3H), 1.21-1.12 (m, 2H), 1.06-0.95 (m, 4H), 0.92 (s, 3H), 0.92-0.83 (m, 1H), 0.72 (s, 3H).

ESI-MS m/z: 486.29[M−1]⁻

### Example 9 Synthesis of Compound I-9

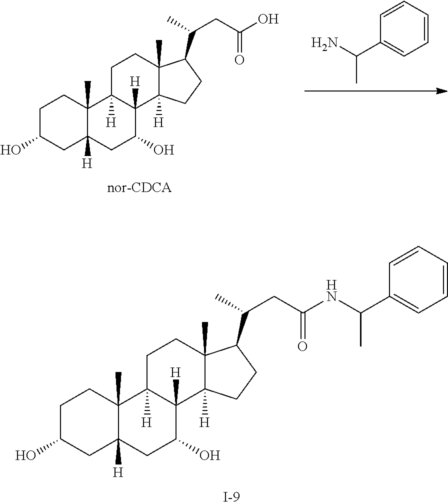

The compound I-9 was obtained by a synthetic method similar to that in Example 1 using nor-CDCA and α-phenethylamine as raw materials.

¹HNMR(CDCl₃, 400 MHz): δ7.40-7.24 (m, 5H), 5.76 (s, 1H), 5.17 (m, 1H), 3.86 (d, J=3.3 Hz, 1H), 3.55-3.42 (m, 1H), 2.47-2.30 (m, 1H), 2.31-2.14 (m, 1H), 2.12-1.93 (m, 4H), 1.89-1.80 (m, 2H), 1.80-1.68 (m, 4H), 1.56-1.41 (m, 7H), 1.44-1.28 (m, 5H), 1.25-1.10 (m, 3H), 1.06-0.93 (m, 4H), 0.92 (s, 3H), 0.71 (d, J=3.7 Hz, 3H).

ESI-MS m/z: 480.30[M−1]⁻

### Example 10 Synthesis of Compound I-10

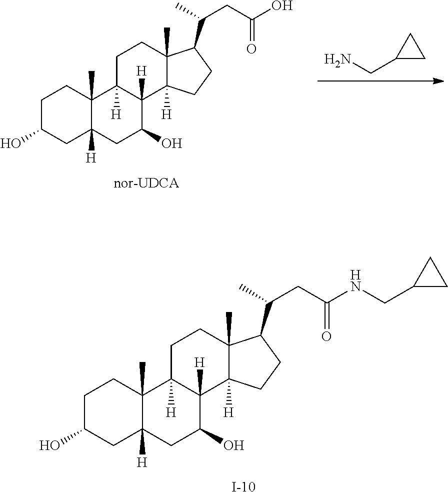

The compound I-10 was obtained by a synthetic method similar to that in Example 1 using nor-UDCA and cyclopropylmethylamine as raw materials.

¹HNMR(CDCl₃, 400 MHz): δ 5.52 (s, 1H), 3.68-3.55 (m, 2H), 3.14 (s, 2H), 2.48-2.33 (m, 1H), 2.09-1.96 (m, 2H), 1.89-1.54 (m, 10H), 1.56-1.51 (m, 3H), 1.51-1.37 (m, 5H), 1.34-1.05 (m, 5H), 1.07-0.89 (m, 1H), 1.01 (d, J=6.1 Hz, 3H), 0.97 (s, 3H), 0.75 (s, 3H), 0.52 (d, J=7.6 Hz, 2H), 0.22 (d, J=4.8 Hz, 2H).

ESI-MS m/z: 430.30[M−1]⁻

### Example 11 Synthesis of Compound I-11

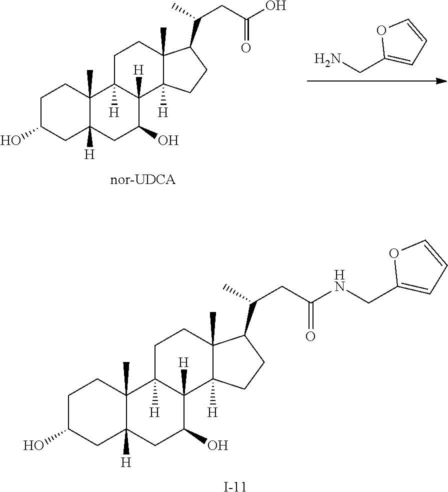

The compound I-11 was obtained by a synthetic method similar to that in Example 1 using nor-UDCA and 2-furfurylamine as raw materials.

¹HNMR(d6-DMSO, 400 MHz): δ 7.68-7.61 (m, 2H), 7.28 (s, 1H), 6.27-6.20 (m, 1H), 6.12 (d, J=3.1 Hz, 1H), 4.36-4.19 (m, 2H), 3.44-3.33 (m, 2H), 2.30-2.20 (m, 1H), 1.97-1.87 (m, 2H), 1.86-1.63 (m, 4H), 1.57-1.36 (m, 4H), 1.39-1.19 (m, 4H), 1.22-1.05 (m, 4H), 1.08-0.88 (m, 3H), 0.88-0.86 (m, 4H), 0.85 (s, 3H), 0.62 (s, 3H).

ESI-MS m/z: 456.22[M−1]⁻

### Example 12 Synthesis of Compound I-12

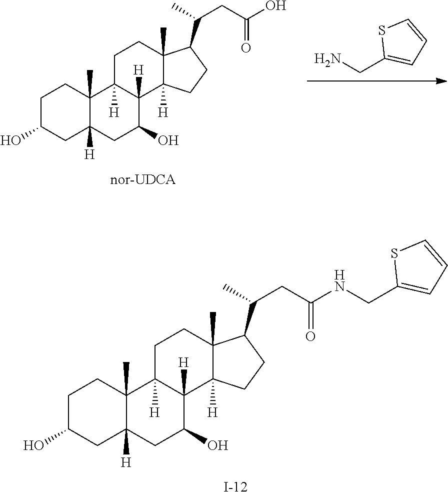

The compound I-12 was obtained by a synthetic method similar to that in Example 1 using nor-UDCA and 2-thiophenemethylamine as raw materials.

¹HNMR(d6-DMSO, 400 MHz): δ 7.66-7.58 (m, 1H), 7.58-7.52 (m, 2H), 7.17-7.08 (m, 1H), 6.91-6.81 (m, 2H), 4.55-4.38 (m, 2H), 3.46-3.50 (m, 2H), 2.32-2.21 (m, 1H), 1.97-1.86 (m, 1H), 1.83-1.63 (m, 3H), 1.56-1.44 (m, 5H), 1.42-1.25 (m, 6H), 1.25-1.10 (m, 7H), 1.10-0.82 (m, 7H), 0.63 (s, 3H).

ESI-MS m/z: 472.28 [M−1]⁻

### Example 13 Synthesis of Compound I-13

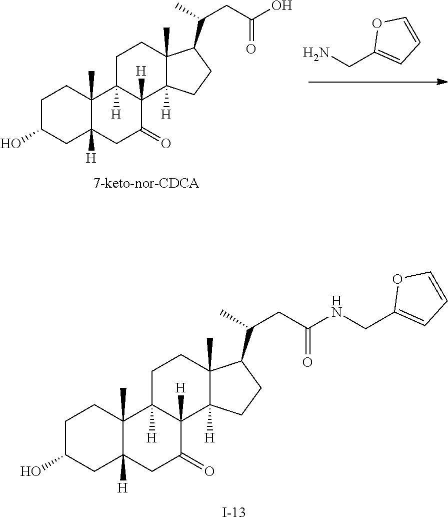

The compound I-13 was obtained by a synthetic method similar to that in Example 1 using 7-keto-nor-CDCA and 2-furfurylamine as raw materials.

¹HNMR(CDCl₃, 400 MHz): δ 7.37 (s, 1H), 6.37-6.31 (m, 1H), 6.24 (d, J=3.2 Hz, 1H), 5.76 (s, 1H), 4.46 (d, J=5.2 Hz, 2H), 3.68-3.56 (m, 1H), 2.92-2.83 (m, 1H), 2.48-2.36 (m, 2H), 2.28-2.16 (m, 1H), 2.03-1.85 (m, 3H), 1.85-1.61 (m, 8H), 1.58-1.43 (m, 3H), 1.47-1.22 (m, 3H), 1.21 (s, 3H), 1.19-0.89 (m, 3H), 0.99 (d, J=6.3 Hz, 3H), 0.72 (s, 3H).

ESI-MS m/z: 454.26 [M−1]⁻

### Example 14 Synthesis of Compound I-14

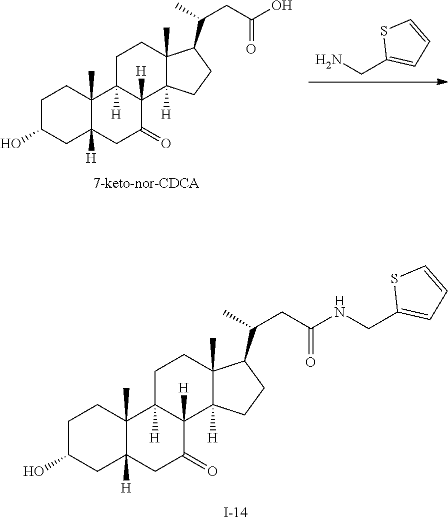

The compound I-14 was obtained by a synthetic method similar to that in Example 1 using 7-keto-nor-CDCA and 2-thiophenemethylamine as raw materials.

¹HNMR(CDCl₃, 400 MHz): δ 7.31-7.20 (m, 1H), 7.01-6.93 (m, 2H), 6.17-5.81 (m, 1H, 4.71-4.56 (m, 2H), 3.67-3.55 (m, 1H), 2.92-2.82 (m, 1H), 2.50-2.36 (m, 2H), 2.24-2.14 (m, 1H), 2.10-1.80 (m, 5H), 2.02-1.90 (m, 2H), 1.74-1.65 (m, 2H), 1.56-1.25 (m, 6H), 1.21 (s, 3H), 1.26-0.92 (m, 5H), 1.00 (d, J=6.5 Hz, 3H), 0.72 (s, 3H).

ESI-MS m/z: 470.28 [M−1]⁻

### Example 15 Synthesis of Compound I-15

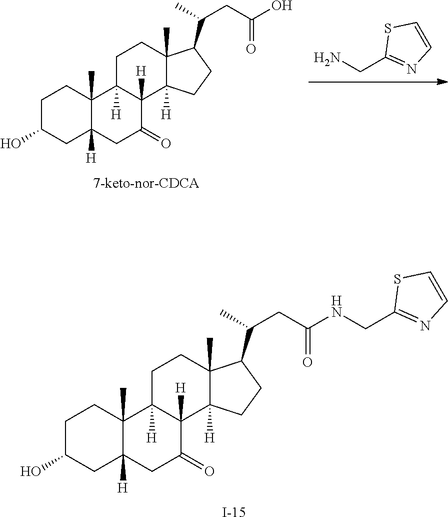

The compound I-15 was obtained by a synthetic method similar to that in Example 1 using 7-keto-nor-CDCA and 2-thiazolylmethanamine as raw materials.

¹HNMR(CDCl₃, 400 MHz): δ 7.72 (d, J=3.2 Hz, 1H), 7.31 (d, J=3.3 Hz, 1H), 6.47 (s, 1H), 4.78 (t, J=5.8 Hz, 2H), 3.68-3.55 (m, 1H), 2.92-2.82 (m, 1H), 2.52-2.36 (m, 2H), 2.25-2.20 (m, 1H), 2.06-2.96 (m, 3H), 1.96-1.66 (m, 8H), 1.58-1.39 (m, 3H), 1.10-1.23 (m, 3H), 1.21 (s, 3H), 1.20-1.08 (m, 3H), 1.00 (d, J=6.4 Hz, 3H), 0.71 (s, 3H).

ESI-MS m/z: 471.28 [M−1]⁻

### Example 16 Synthesis of Compound I-16

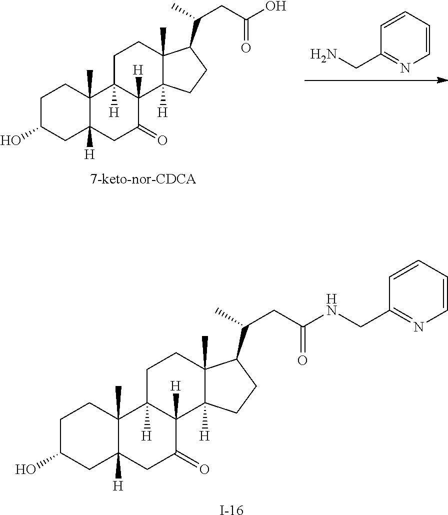

The compound I-16 was obtained by a synthetic method similar to that in Example 1 using 7-keto-nor-CDCA and 2-aminomethyl-pyridine as raw materials.

¹HNMR(CDCl₃, 400 MHz): δ 8.55 (d, J=4.8 Hz, 1H), 7.70 (t, J=6.8 Hz, 1H), 7.37-7.20 (m, 2H), 6.82 (t, J=5.0 Hz, 1H), 4.62-4.55 (m, 2H), 3.68-3.55 (m, 1H), 2.92-2.82 (m, 1H), 2.53-2.35 (m, 2H), 2.27-2.14 (m, 1H), 2.08-1.78 (m, 9H), 1.76-1.66 (m, 2H), 1.52-1.43 (m, 3H), 1.42-1.22 (m, 4H), 1.21 (s, 3H), 1.21-1.08 (m, 3H), 0.99 (d, J=6.4 Hz, 3H), 0.71 (s, 3H).

ESI-MS m/z: 465.30 [M−1]⁻

### Example 17 Synthesis of Compound I-17

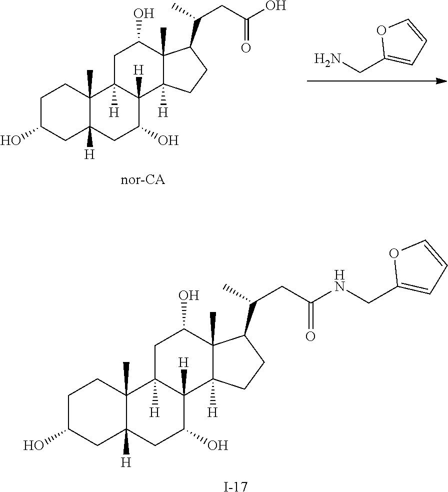

The compound I-17 was obtained by a synthetic method similar to that in Example 1 using nor-CA and 2-furfurylamine as raw materials.

¹HNMR(CDCl₃, 400 MHz): δ 7.37 (s, 1H), 6.34 (s, 1H), 6.25 (d, J=3.2 Hz, 1H), 6.09 (s, 1H), 4.50-4.43 (m, 2H), 4.02 (s, 1H), 3.87 (s, 1H), 3.55-3.44 (m, 1H), 2.49-2.40 (m, 1H), 2.29-2.18 (m, 2H), 2.19-1.89 (m, 5H), 1.89-1.75 (m, 2H), 1.71-1.66 (m, 1H), 1.63-1.51 (m, 4H), 1.47-1.25 (m, 5H), 1.24-1.09 (m, 1H), 1.05 (d, J=6.3 Hz, 3H), 0.99-0.88 (m, 1H), 0.91 (s, 3H), 0.75 (s, 3H).

ESI-MS m/z: 472.29 [M−1]⁻

### Example 18 Synthesis of Compound I-18

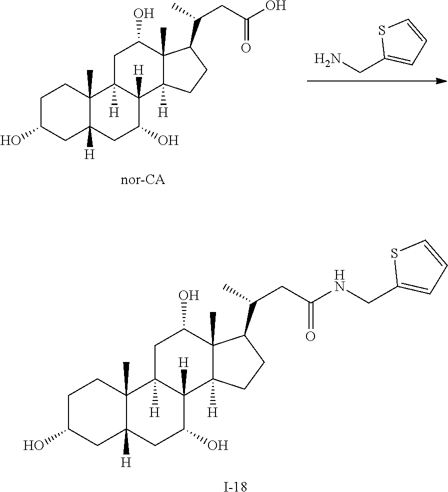

The compound I-18 was obtained by a synthetic method similar to that in Example 1 using nor-CA and 2-thiophenemethylamine as raw materials.

¹HNMR(CDCl₃, 400 MHz): δ 7.21 (d, J=4.9 Hz, 1H), 6.99-6.90 (m, 2H), 6.45 (s, 1H), 4.80-4.67 (m, 2H), 4.00 (s, 1H), 3.84 (s, 1H), 3.44 (s, 1H), 2.44-2.35 (m, 1H), 2.24-2.16 (m, 2H), 1.99-1.62 (m, 12H), 1.61-1.55 (m, 3H), 1.43-1.38 (m, 3H), 1.18-1.08 (m, 1H), 1.05 (, J=6.3 Hz, 3H), 0.90 (s, 3H), 0.73 (s, 3H).

ESI-MS m/z: 488.25 [M−1]⁻

### Example 19 Synthesis of Compound I-19

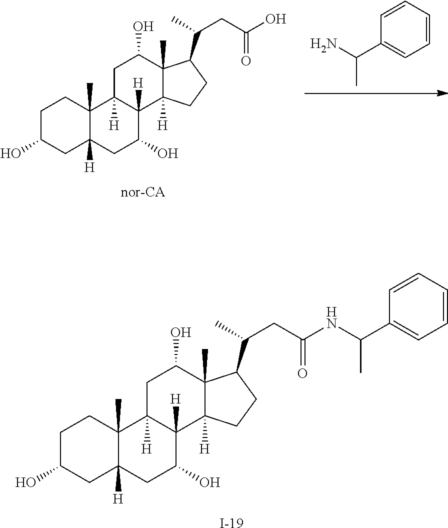

The compound I-19 was obtained by a synthetic method similar to that in Example 1 using nor-CA and α-phenethylamine as raw materials.

¹HNMR(CDCl₃, 400 MHz): δ 7.36-7.22 (m, 5H), 6.38-6.11 (m, 1H), 5.22-5.08 (m, 1H), 3.98 (d, J=8.4 Hz, 1H), 3.84 (s, 1H), 3.49-3.38 (m, 1H), 2.42-2.30 (m, 1H), 2.28-2.15 (m, 2H), 1.98-1.65 (m, 11H), 1.61-1.35 (m, 8H), 1.35-1.24 (m, 1H), 1.16-0.95 (m, 5H), 0.90 (s, 3H), 0.72 (d, J=4.0 Hz, 3H).

ESI-MS m/z: 496.35 [M−1]⁻

### Example 20 Synthesis of Compound I-20

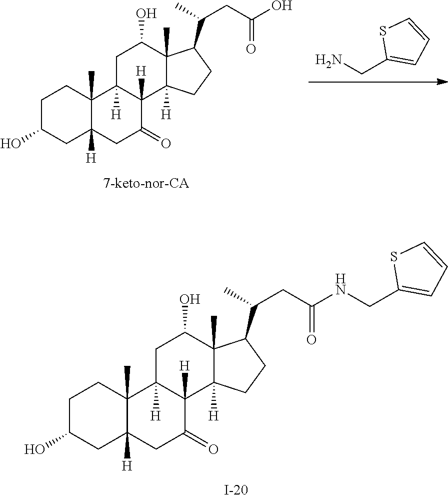

The compound I-20 was obtained by a synthetic method similar to that in Example 1 using 7-keto-nor-CA and 2-thiophenemethylamine as raw materials.

¹HNMR(CDCl₃, 400 MHz): δ 7.22 (d, J=5.1 Hz, 1H), 7.00-6.91 (m, 2H), 6.55 (s, 1H), 4.70-4.52 (m, 2H), 4.03 (s, 1H), 3.62-3.51 (m, 1H), 2.89-2.79 (m, 1H), 2.53-2.30 (m, 2H), 2.31-2.16 (m, 2H), 2.09-1.78 (m, 4H), 1.77-1.65 (m, 3H), 1.67-1.52 (m, 2H), 1.40-1.18 (m, 5H), 1.19 (s, 3H), 1.18-1.09 (m, 1H), 1.05 (d, J=6.0 Hz, 3H), 1.05-0.83 (m, 2H), 0.74 (s, 3H).

ESI-MS m/z: 486.26 [M−1]⁻

### Example 21 Synthesis of Compound I-21

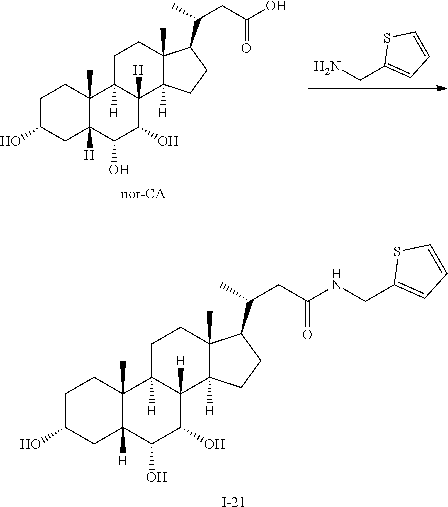

The compound I-21 was obtained by a synthetic method similar to that in Example 1 using nor-HCA and 2-thiophenemethylamine as raw materials.

¹HNMR(CDCl₃, 400 MHz): δ 7.25-7.18 (m, 1H), 6.98-6.91 (m, 2H), 6.36 (s, 1H), 4.57 (s, 2H), 3.86 (s, 2H), 3.51-3.40 (m, 1H), 2.47-2.39 (m, 1H), 2.09-1.98 (m, 2H), 1.90-1.78 (m, 3H), 1.74-1.69 (m, 5H), 1.57-1.45 (m, 4H), 1.43-1.28 (m, 2H), 1.32-1.14 (m, 4H), 1.11-1.03 (m, 1H), 1.00 (d, J=6.3 Hz, 3H), 0.92 (s, 3H), 0.72 (s, 3H).

ESI-MS m/z: 488.27 [M−1]⁻

### Example 22 Synthesis of Compound I-22

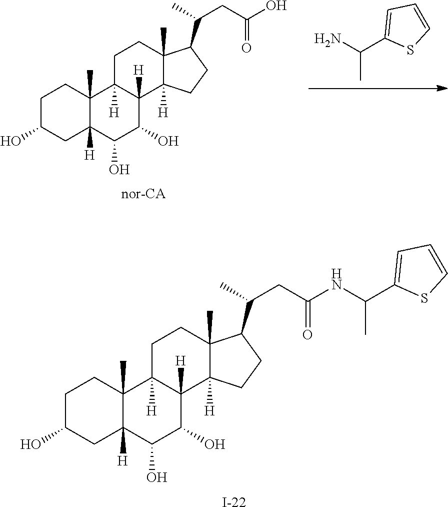

The compound I-22 was obtained by a synthetic method similar to that in Example 1 using nor-HCA and I-A-2 as raw materials.

¹HNMR(CDCl₃, 400 MHz): δ 7.23 (d, J=5.1 Hz, 1H), 7.04-6.95 (m, 2H), 5.47-5.40 (m, 1H), 3.91-3.84 (m, 2H), 3.51-3.39 (m, 1H), 2.50 (s, 1H), 2.30-1.39 (m, 17H) 1.39-1.24 (m, 2H), 1.22-1.16 (m, 4H), 1.11-0.74 (m, 2H), 1.02 (d, J=6.3 Hz, 3H), 0.92 (s, 3H), 0.72 (d, J=3.5 Hz, 3H).

ESI-MS m/z: 502.29 [M−1]⁻

### Example 23 Synthesis of Compound I-23

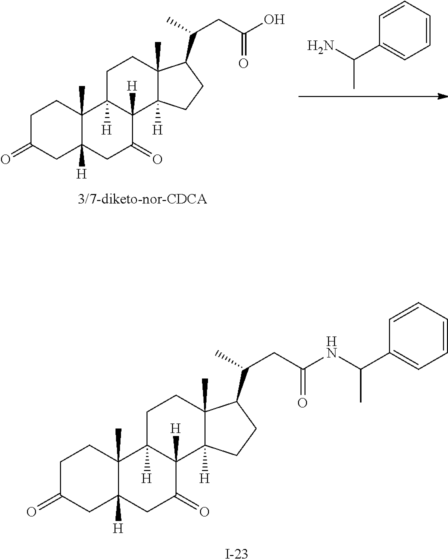

The compound I-23 was obtained by a synthetic method similar to that in Example 1 using 3,7-diketo-nor-CDCA and α-phenethylamine as raw materials.

¹HNMR(CDCl₃, 400 MHz): δ 7.41-7.24 (m, 5H), 5.74 (s, 1H), 5.24-5.11 (m, 1H), 2.95-2.85 (m, 1H), 2.57-2.37 (m, 2H), 2.33-2.17 (m, 6H), 2.12-1.96 (m, 3H), 1.96-1.81 (m, 4H), 1.80-1.42 (m, 8H), 1.45-1.23 (m, 1H), 1.32 (s, 3H), 1.26-1.08 (m, 2H), 1.04-0.93 (m, 3H), 0.75 (d, J=2.8 Hz, 3H).

ESI-MS m/z: 478.20[M+1]⁺

### Example 24 Synthesis of Compound I-24

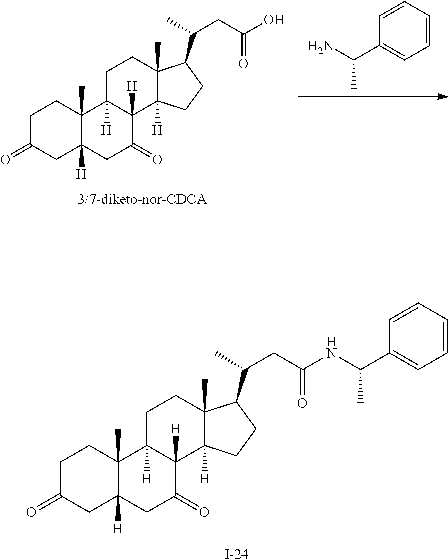

The compound I-24 was obtained by a synthetic method similar to that in Example 1 using 3,7-diketo-nor-CDCA and (S)-α-phenethylamine as raw materials.

¹HNMR(CDCl₃, 400 MHz): δ 7.39-7.31 (m, 4H), 7.31-7.23 (m, 1H), 5.82 (s, 1H), 5.23-5.11 (m, 1H), 2.94-2.84 (m, 1H), 2.57-2.38 (m, 2H), 2.32-2.14 (m, 5H), 2.17-1.81 (m, 6H), 1.81-1.66 (m, 1H), 1.68-1.54 (m, 2H), 1.50 (d, J=6.9 Hz, 3H), 1.55-1.34 (m, 3H), 1.31 (s, 3H), 1.32-1.10 (m, 3H), 1.13-0.95 (m, 1H), 0.94 (d, J=6.5 Hz, 3H), 0.74 (s, 3H).

ESI-MS m/z: 476.17[M−1]⁻

### Example 25 Synthesis of Compound I-25

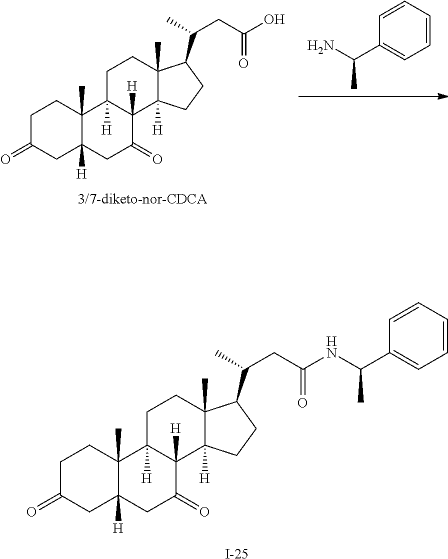

The compound I-25 was obtained by a synthetic method similar to that in Example 1 using 3, 7-diketo-nor-CDCA and (R)-α-phenethylamine as raw materials.

¹HNMR(CDCl₃, 400 MHz): δ 7.41-7.18 (m, 5H), 5.52 (s, 1H), 3.61-3.50 (m, 2H), 2.95-2.79 (m, 3H), 2.52 (t, J=11.3 Hz, 1H), 2.41-2.25 (m, 4H), 2.25-2.13 (m, 4H), 2.13-2.02 (m, 2H), 2.01-1.83 (m, 4H), 1.75-1.44 (m, 5H), 1.32 (s, 3H), 1.31-0.93 (m, 2H), 0.96 (d, J=6.4 Hz, 3H), 0.74 (s, 3H).

ESI-MS m/z: 478.33[M+1]⁺

### Example 26 Synthesis of Compound I-26

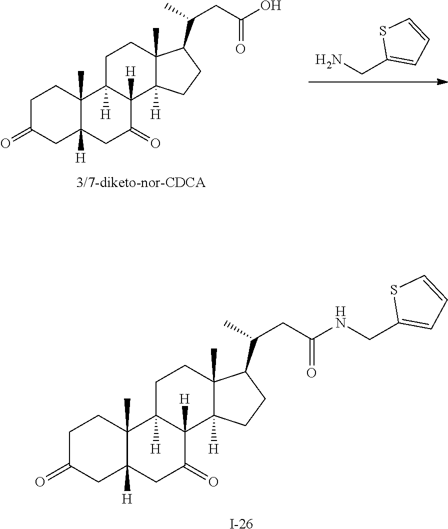

The compound I-26 was obtained by a synthetic method similar to that in Example 1 using 3,7-diketo-nor-CDCA and 2-thiophenemethylamine as raw materials.

¹HNMR(CDCl₃, 400 MHz): δ7.24 (d, J=4.8, 1H), 7.02-6.94 (m, 2H), 4.73-4.56 (m, 2H), 2.95-2.85 (m, 1H), 2.58-2.40 (m, 2H), 2.33-2.17 (m, 4H), 2.16-1.85 (m, 4H), 1.84-1.58 (m, 3H), 1.58-1.45 (m, 2H), 1.40-1.12 (m, 7H), 1.33 (s, 3H), 1.02 (d, J=6.4 Hz, 3H), 0.94-0.83 (m, 2H), 0.76 (s, 3H).

ESI-MS m/z: 468.29[M−1]⁻

### Example 27 Synthesis of Compound I-27

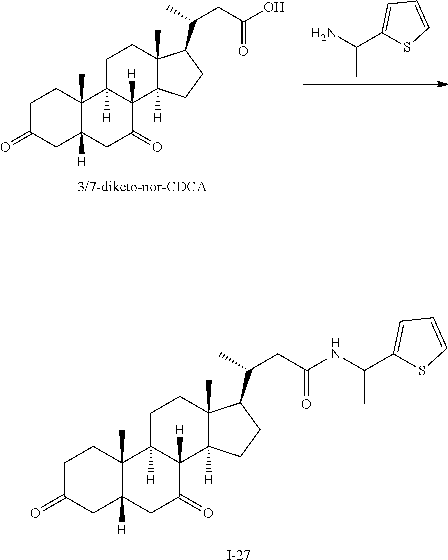

The compound I-27 was obtained by a synthetic method similar to that in Example 1 using 3,7-diketo-nor-CDCA and I-A-2 as raw materials.

¹HNMR(CDCl₃, 400 MHz): δ7.22 (d, J=4.8 Hz, 1H), 7.01-6.94 (m, 2H), 5.52-5.40 (m, 1H), 2.95-2.85 (m, 1H), 2.58-2.47 (m, 1H), 2.49-2.38 (m, 1H), 2.34-2.17 (m, 6H), 2.20-2.03 (m, 3H), 2.05-1.81 (m, 3H), 1.81-1.64 (m, 2H), 1.64-1.45 (m, 6H), 1.41-1.33 (m, 1H), 1.32 (s, 3H), 1.26-1.10 (m, 2H), 1.05-0.96 (m, 4H), 0.76 (s, 3H).

ESI-MS m/z: 482.20[M−1]⁻

### Example 28 Synthesis of Compound I-28

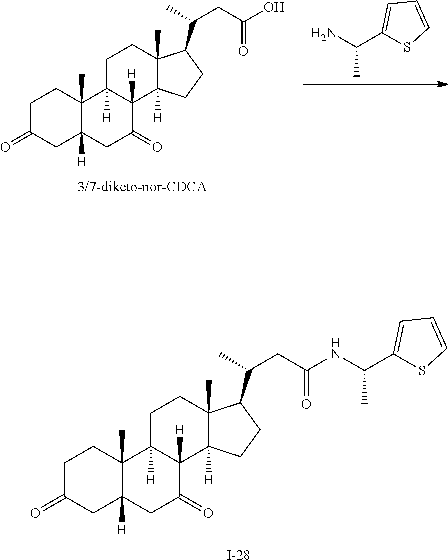

The compound I-28 was obtained by a synthetic method similar to that in Example 1 using 3,7-diketo-nor-CDCA and I-A-2 as raw materials.

¹HNMR(CDCl₃, 400 MHz): δ 7.22 (d, J=4.8 Hz, 1H), 7.00-6.94 (m, 2H), 5.53-5.41 (m, 1H), 2.95-2.85 (m, 1H), 2.59-2.49 (m, 1H), 2.50-2.39 (m, 1H), 2.34-2.17 (m, 6H), 2.20-2.03 (m, 3H), 2.05-1.81 (m, 2H), 1.81-1.64 (m, 3H), 1.64-1.45 (m, 6H), 1.41-1.33 (m, 1H), 1.32 (s, 3H), 1.26-1.10 (m, 2H), 1.05-0.96 (m, 4H), 0.76 (s, 3H).

ESI-MS m/z: 482.20[M−1]⁻

### Example 29 Synthesis of Compound I-29

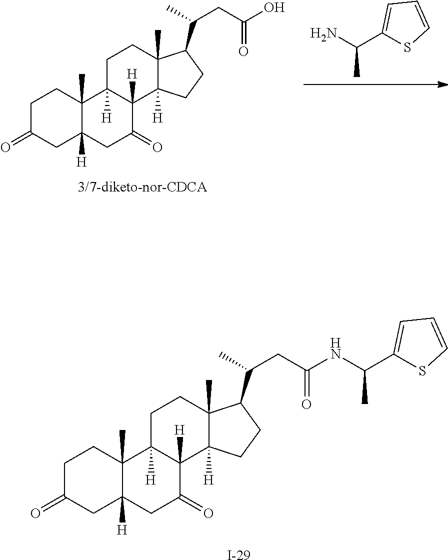

The compound I-29 was obtained by a synthetic method similar to that in Example 1 using 3,7-diketo-nor-CDCA and I-A-2-R as raw materials.

¹HNMR(CDCl₃, 400 MHz): δ 7.22 (d, J=4.8 Hz, 1H), 7.01-6.94 (m, 2H), 5.51-5.40 (m, 1H), 2.95-2.85 (m, 1H), 2.58-2.47 (m, 1H), 2.49-2.38 (m, 1H), 2.35-2.16 (m, 6H), 2.20-2.03 (m, 3H), 2.04-1.84 (m, 3H), 1.80-1.65 (m, 2H), 1.64-1.33 (m, 7H), 1.32 (s, 3H), 1.26-1.10 (m, 2H), 1.05-0.96 (m, 4H), 0.76 (s, 3H).

ESI-MS m/z: 482.20[M−1]⁻

### Example 30 Synthesis of Compound I-30

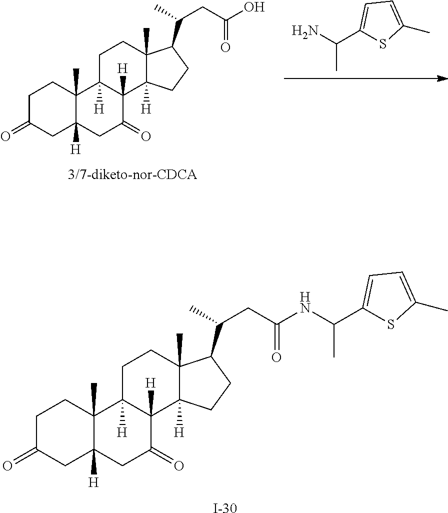

The compound I-30 was obtained by a synthetic method similar to that in Example 1 using 3,7-diketo-nor-CDCA and (R/S)-1-(5-methyl-thienyl-2) ethylamine as raw materials.

¹HNMR(CDCl₃, 400 MHz): δ 6.75 (d, J=3.4 Hz, 1H), 6.60 (s, 1H), 5.72-5.66 (m, 1H), 5.42-5.27 (m, 1H), 2.95-2.85 (m, 1H), 2.58-2.47 (m, 1H), 2.46 (s, 3H), 2.46-2.37 (m, 1H), 2.33-2.15 (m, 5H), 2.13-2.04 (m, 2H), 2.01-1.84 (m, 2H), 1.79-1.64 (m, 2H), 1.64-1.45 (m, 7H), 1.32 (s, 3H), 1.40-0.95 (m, 5H), 1.02-0.98 (m, 3H), 0.76 (s, 3H).

ESI-MS m/z: 496.26[M−1]⁻

### Example 31 Synthesis of Compound I-31

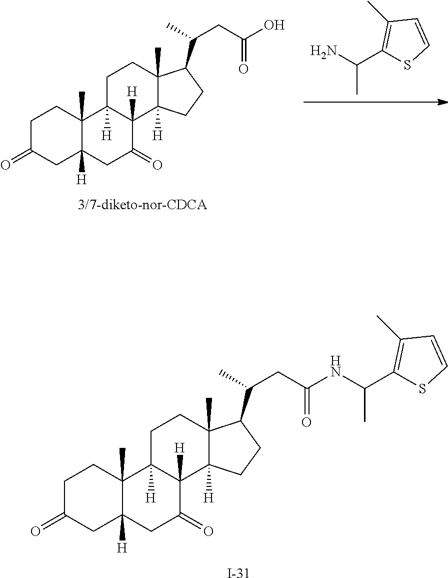

The compound I-31 was obtained by a synthetic method similar to that in Example 1 using 3,7-diketo-nor-CDCA and (R/S)-1-(3-methyl-thienyl-2) ethylamine as raw materials.

¹HNMR(CDCl₃, 400 MHz): δ 7.12 (d, J=5.2 Hz, 1H), 6.83 (d, J=5.0 Hz, 1H), 5.59 (d, J=7.9 Hz, 1H), 5.46 (q, J=6.5 Hz, 1H), 2.95-2.85 (m, 1H), 2.57-2.47 (m, 1H), 2.33-2.17 (m, 9H), 2.15-1.85 (m, 6H), 1.78-1.67 (m, 2H), 1.60-1.45 (m, 4H), 1.41-1.33 (m, 1H), 1.32 (s, 3H), 1.22-1.08 (m, 3H), 1.08-0.90 (m, 1H), 1.03-0.94 (m, 3H), 0.75 (s, 3H).

ESI-MS m/z: 496.26[M−1]⁻

### Example 32 Synthesis of Compound I-32

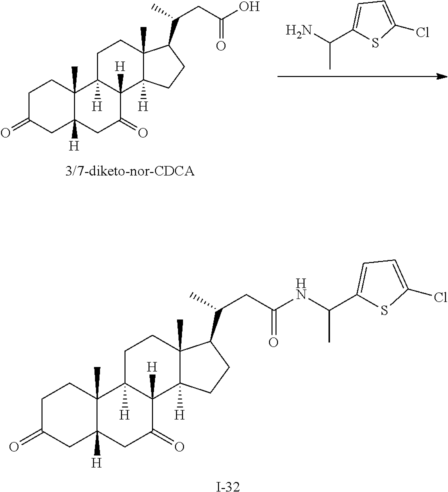

The compound I-32 was obtained by a synthetic method similar to that in Example 1 using 3,7-diketo-nor-CDCA and I-A-3 as raw materials.

¹HNMR(CDCl₃, 400 MHz): δ 6.77-6.71 (m, 2H), 5.80 (s, 1H), 5.38-5.25 (m, 1H), 2.95-2.85 (m, 1H), 2.57-2.47 (m, 1H), 2.46-2.36 (m, 1H), 2.35-2.15 (m, 5H), 2.15-1.99 (m, 3H), 2.01-1.80 (m, 3H), 1.82-1.64 (m, 2H), 1.60-1.44 (m, 6H), 1.43-1.32 (m, 1H), 1.32 (s, 3H), 1.32-1.08 (m, 3H), 1.06-0.92 (m, 1H), 1.02-0.96 (m, 3H), 0.75 (s, 3H).

ESI-MS m/z: 516.14[M−1]⁻

### Example 33 Synthesis of Compound I-33

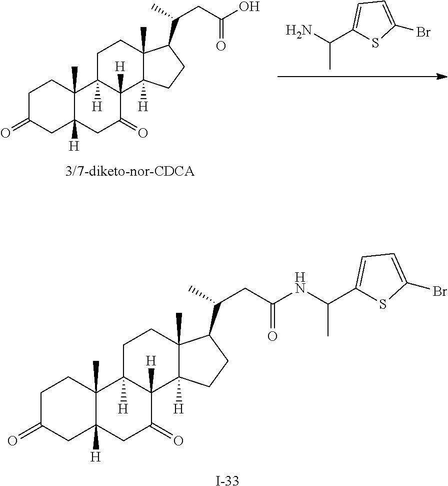

The compound I-33 was obtained by a synthetic method similar to that in Example 1 using 3,7-diketo-nor-CDCA and I-A-4 as raw materials.

ESI-MS m/z: 560.06[M−1]⁻

### Example 34 Synthesis of Compound I-34

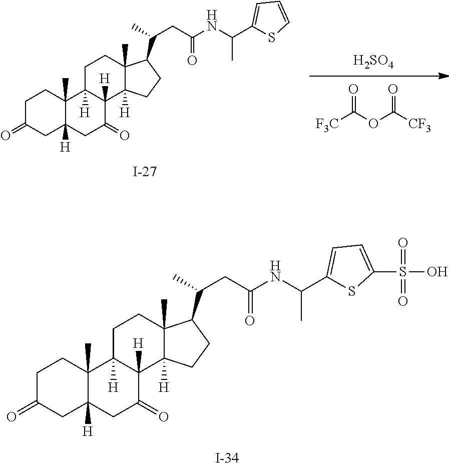

0.3 g of the compound I-27 (0.62 mmol) was dissolved in a 100 ml single-neck bottle by 50 ml of acetonitrile, 0.11 g of concentrated sulfuric acid (1.12 mmol) was slowly added dropwise, cooled down to 10° C. in an ice-water bath; 0.37 g of trifluoroacetic anhydride (1.76 mmol) was slowly added dropwise to the system, and the system was heated in an oil bath to 50° C. to allow reaction for 3 h.

The complete reaction of raw materials was monitored by TLC, 100 ml of water and 100 ml of ethyl acetate were added to the reaction system, and allowed to stand for stratification; an aqueous layer was extracted once with 100 ml of ethyl acetate, and organic layers were combined and washed once with 50 ml of saturated brine; the organic layer was dried with anhydrous sodium sulfate, filtered to remove sodium sulfate, and ethyl acetate was concentrated; an obtained oily substance was crystallized with a small amount of the ethyl acetate, and filtered to obtain 0.25 g of the compound I-34.

ESI-MS m/z: 562.22[M−1]−

The I-34 and various organic or inorganic amines can be prepared into various corresponding salts.

### Example 35 Synthesis of Compound I-35

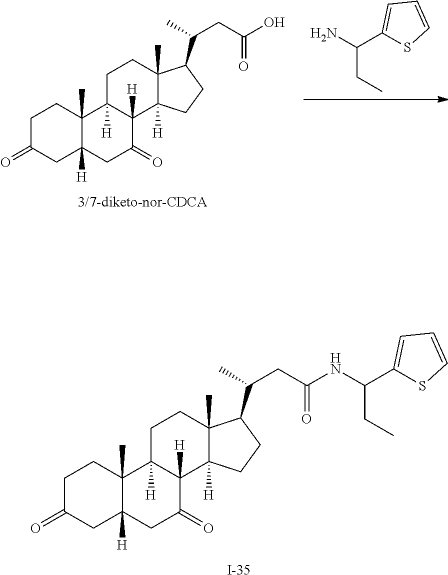

The compound I-35 was obtained by a synthetic method similar to that in Example 1 using 3,7-diketo-nor-CDCA and I-A-1 as raw materials.

¹HNMR(CDCl₃, 400 MHz): δ 7.25-7.18 (m, 1H), 7.00-6.94 (m, 2H), 5.67 (d, J=8.6 Hz, 1H), 5.25 (q, J=7.6 Hz, 1H), 2.95-2.85 (m, 1H), 2.57-2.38 (m, 2H), 2.33-2.18 (m, 4H), 2.22-2.01 (m, 4H), 2.01-1.87 (m, 4H), 1.84-1.45 (m, 6H), 1.32 (s, 3H), 1.43-1.21 (m, 2H), 1.24-1.10 (m, 2H), 1.10-0.85 (m, 1H), 1.03-0.97 (m, 6H), 0.75 (s, 3H).

ESI-MS m/z: 596.33[M−1]⁻

### Example 36 Synthesis of Compound I-36

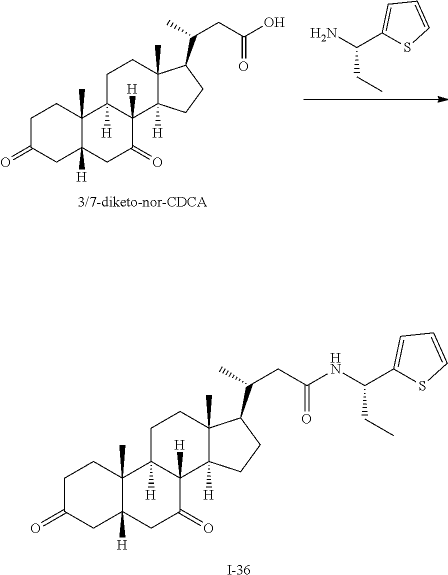

The compound I-36 was obtained by a synthetic method similar to that in Example 1 using 3,7-diketo-nor-CDCA and I-A-1-S as raw materials.

¹HNMR(CDCl₃, 400 MHz): δ7.25-7.18 (m, 1H), 7.00-6.94 (m, 2H), 5.67 (d, J=8.6 Hz, 1H), 5.25 (q, J=7.6 Hz, 1H), 2.96-2.86 (m, 1H), 2.57-2.37 (m, 2H), 2.33-2.18 (m, 4H), 2.22-2.01 (m, 4H), 2.01-1.87 (m, 4H), 1.84-1.45 (m, 6H), 1.32 (s, 3H), 1.43-1.21 (m, 2H), 1.24-1.10 (m, 2H), 1.10-0.85 (m, 1H), 1.03-0.97 (m, 6H), 0.75 (s, 3H).

ESI-MS m/z: 596.33[M−1]⁻

### Example 37 Synthesis of Compound I-37

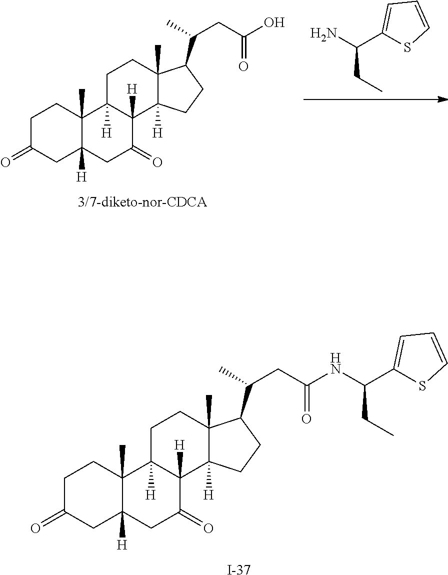

The compound I-37 was obtained by a synthetic method similar to that in Example 1 using 3,7-diketo-nor-CDCA and I-A-1-R as raw materials.

¹HNMR(CDCl₃, 400 MHz): δ7.25-7.18 (m, 1H), 7.00-6.94 (m, 2H), 5.67 (d, J=8.6 Hz, 1H), 5.25 (q, J=7.6 Hz, 1H), 2.94-2.84 (m, 1H), 2.58-2.38 (m, 2H), 2.33-2.18 (m, 4H), 2.22-2.01 (m, 4H), 2.01-1.87 (m, 4H), 1.84-1.45 (m, 6H), 1.32 (s, 3H), 1.43-1.21 (m, 2H), 1.24-1.10 (m, 2H), 1.10-0.85 (m, 1H), 1.03-0.97 (m, 6H), 0.75 (s, 3H).

ESI-MS m/z: 596.33[M−1]⁻

### Example 38 Synthesis of Compound I-38

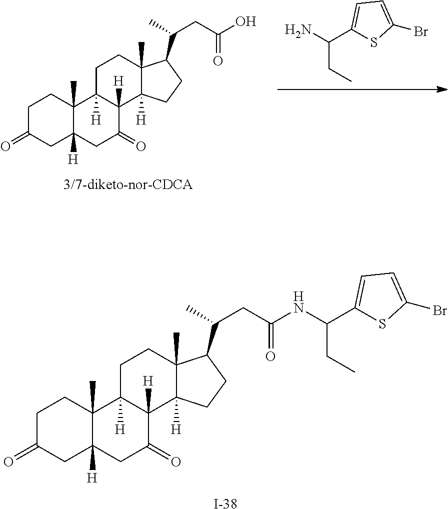

The compound I-38 was obtained by a synthetic method similar to that in Example 1 using 3,7-diketo-nor-CDCA and I-A-7 as raw materials.

¹HNMR(CDCl₃, 400 MHz): δ 6.90 (d, J=3.7 Hz, 1H), 6.73 (d, J=3.7 Hz, 1H), 5.68-5.57 (m, 1H), 5.14 (q, J=7.6 Hz, 1H), 2.95-2.85 (m, 1H), 2.57-2.38 (m, 3H), 2.34-2.15 (m, 7H), 2.15-2.03 (m, 1H), 2.01-1.72 (m, 6H), 1.56-1.48 (m, 3H), 1.38-1.27 (m, 1H), 1.32 (s, 3H), 1.26-1.11 (m, 2H), 1.09-0.94 (m, 8H), 0.75 (s, 3H).

ESI-MS m/z: 574.17[M−1]⁻

### Example 39 Synthesis of Compound I-39

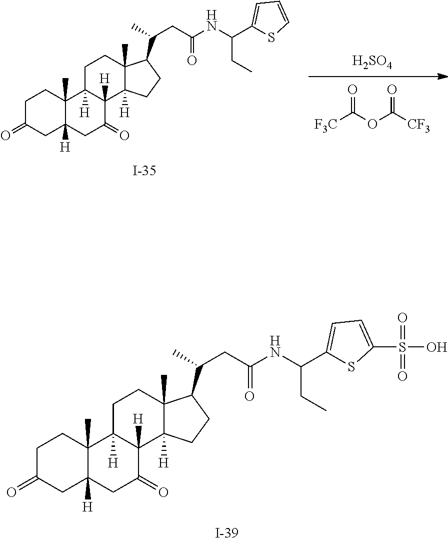

The compound I-39 was obtained by a synthetic method similar that in Example 34 using I-35 as raw materials.

¹HNMR(CDCl₃, 400 MHz): δ 7.18 (s, 1H), 6.93 (s, 2H), 5.21-5.14 (m, 1H), 2.94-2.15 (m, 10H), 2.15-1.70 (m, 8H), 1.70-0.70 (m, 21H); 2.94-2.15 (m, 10H), 2.15-1.70 (m, 8H), 1.70-0.70 (m, 21H);

ESI-MS m/z: 576.21[M−1]⁻

### Example 40 Synthesis of Compound I-40

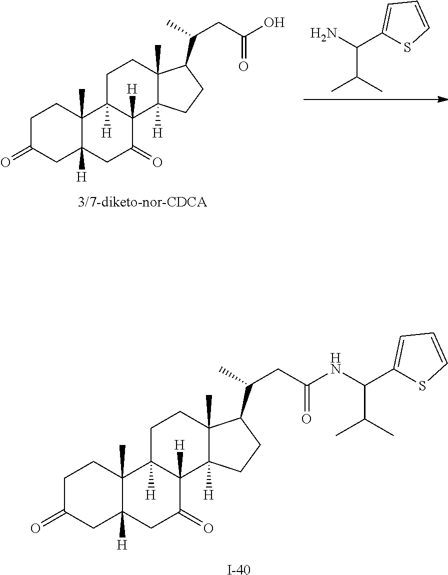

The compound I-40 was obtained by a synthetic method similar to that in Example 1 using 3,7-diketo-nor-CDCA and I-A-8 as raw materials.

¹HNMR(CDCl₃, 400 MHz): δ 7.28 (s, 1H), 6.79-6.67 (m, 2H), 5.69 (d, J=9.0 Hz, 1H), 5.06-4.97 (m, 1H), 2.95-2.85 (m, 1H), 2.58-2.40 (m, 2H), 2.36-2.10 (m, 7H), 2.10-1.97 (m, 3H), 1.97-1.74 (m, 3H), 1.74-1.45 (m, 6H), 1.33 (s, 3H), 1.42-1.10 (m, 3H), 1.10-0.93 (m, 10H), 0.75 (s, 3H).

ESI-MS m/z: 510.25[M−1]⁻

### Example 41 Synthesis of Compound I-41

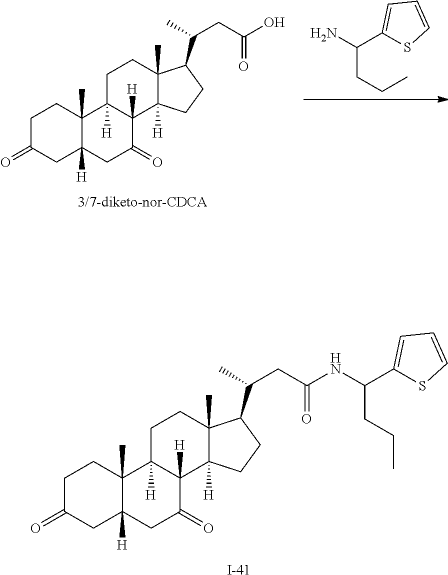

The compound I-41 was obtained by a synthetic method similar to that in Example 1 using 3,7-diketo-nor-CDCA and I-A-11 as raw materials.

¹HNMR(CDCl₃, 400 MHz): δ 7.21 (d, J=4.8 Hz, 1H), 7.00-6.95 (m, 2H), 5.67 (s, 1H), 5.33 (q, J=7.7 Hz, 1H), 2.95-2.85 (m, 1H), 2.57-2.37 (m, 3H), 2.33-2.15 (m, 6H), 2.14-1.79 (m, 8H), 1.78-1.33 (m, 7H), 1.32 (s, 3H), 1.26-1.09 (m, 2H), 1.06-0.93 (m, 7H), 0.75 (s, 3H).

ESI-MS m/z: 510.22[M−1]⁻

### Example 42 Synthesis of Compound I-42

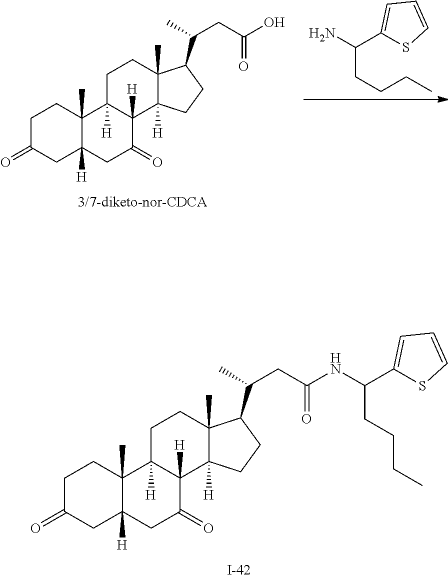

The compound I-42 was obtained by a synthetic method similar to that in Example 1 using 3,7-diketo-nor-CDCA and I-A-12 as raw materials.

¹HNMR(CDCl₃, 400 MHz): δ 7.21 (d, J=4.7 Hz, 1H), 6.99-6.94 (m, 2H), 5.69 (t, J=8.9 Hz, 1H), 5.36-5.26 (m, 1H), 2.94-2.85 (m, 2H), 2.57-2.34 (m, 3H), 2.33-2.14 (m, 6H), 2.15-1.96 (m, 4H), 1.98-1.87 (m, 2H), 1.82-1.71 (m, 1H), 1.70-1.47 (m, 4H), 1.32 (s, 3H), 1.51-1.22 (m, 3H), 1.23-1.09 (m, 2H), 1.09-0.82 (m, 9H), 0.75 (s, 3H).

ESI-MS m/z: 524.32[M−1]⁻

### Example 43 Synthesis of Compound I-43

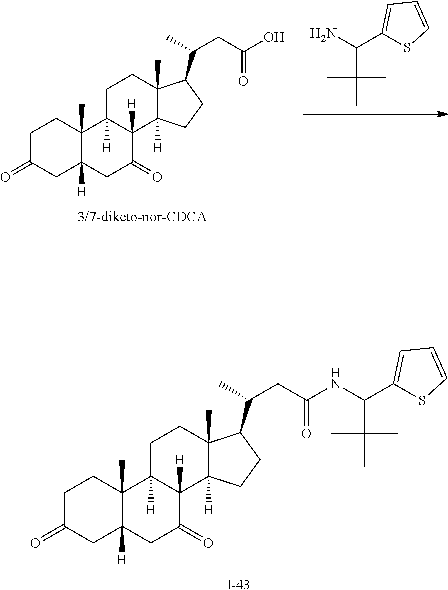

The compound I-43 was obtained by a synthetic method similar to that in Example 1 using 3,7-diketo-nor-CDCA and I-A-20 as raw materials.

¹HNMR(CDCl₃, 400 MHz): δ 7.21-7.14 (m, 1H), 6.98-6.91 (m, 2H), 5.88-5.77 (m, 1H), 5.24-5.15 (m, 1H), 2.95-2.85 (m, 1H), 2.57-2.36 (m, 2H), 2.33-2.13 (m, 6H), 2.14-1.74 (m, 7H), 1.67-1.44 (m, 3H), 1.32 (s, 3H), 1.41-1.16 (m, 2H), 1.19-1.08 (m, 2H), 1.01 (s, 9H), 0.89 (d, J=6.4 Hz, 3H), 0.73 (d, J=6.8 Hz, 3H).

ESI-MS m/z: 524.32[M−1]⁻

### Example 44 Synthesis of Compound I-44

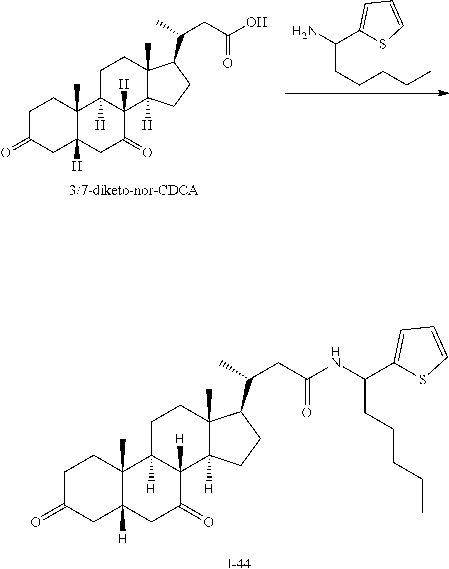

The compound I-44 was obtained by a synthetic method similar to that in Example 1 using 3,7-diketo-nor-CDCA and I-A-13 as raw materials.

¹HNMR(CDCl₃, 400 MHz): δ 7.20 (d, J=4.7 Hz, 1H), 6.99-6.93 (m, 2H), 5.82-5.59 (m, 1H), 5.31 (q, J=7.7 Hz, 1H), 2.95-2.85 (m, 1H), 2.57-2.37 (m, 3H), 2.34-2.12 (m, 8H), 2.11-1.84 (m, 8H), 1.84-1.64 (m, 2H), 1.64-1.44 (m, 4H), 1.32 (s, 3H), 1.23-1.11 (m, 3H), 1.09-0.84 (m, 9H), 0.75 (s, 3H).

ESI-MS m/z: 538.22[M−1]⁻

### Example 45 Synthesis of Compound I-45

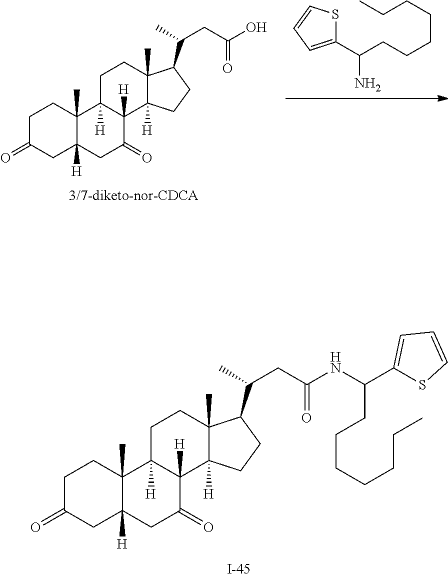

The compound I-45 was obtained by a synthetic method similar to that in Example 1 using 3,7-diketo-nor-CDCA and I-A-14 as raw materials.

¹HNMR(CDCl₃, 400 MHz): δ 7.21 (d, J=4.6 Hz, 1H), 6.96 (s, 2H), 5.80-5.57 (m, 1H), 5.31 (q, J=7.7 Hz, 1H), 2.95-2.85 (m, 1H), 2.57-2.37 (m, 2H), 2.33-2.18 (m, 2H), 2.14-1.79 (m, 9H), 1.70-1.42 (m, 4H), 1.42-1.31 (m, 4H), 1.32 (s, 3H), 1.35-1.24 (m, 8H), 1.26-1.09 (m, 2H), 1.08-0.84 (m, 7H), 0.75 (s, 3H).

ESI-MS m/z: 566.35[M−1]⁻

### Example 46 Synthesis of Compound I-46

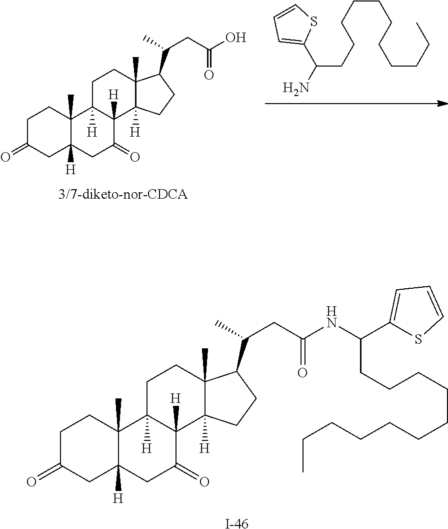

The compound I-46 was obtained by a synthetic method similar to that in Example 1 using 3,7-diketo-nor-CDCA and I-A-15 as raw materials.

ESI-MS m/z: 623.41[M−1]⁻

### Example 47 Synthesis of Compound I-47

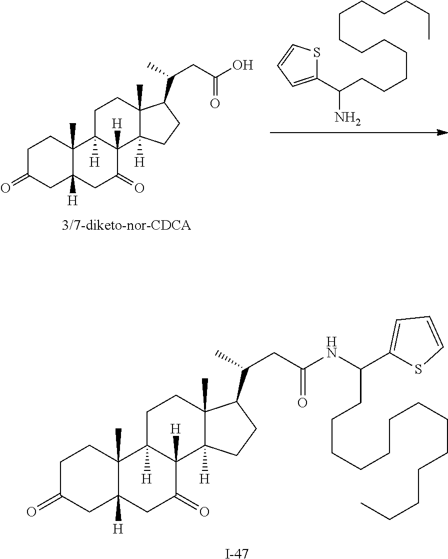

The compound I-47 was obtained by a synthetic method similar to that in Example 1 using 3,7-diketo-nor-CDCA and I-A-16 as raw materials.

¹HNMR(CDCl₃, 400 MHz): δ 7.21 (dd, J=4.4, 1.8 Hz, 1H), 6.97 (s, 2H), 5.72-5.60 (m, 1H), 5.31 (q, J=7.7 Hz, 1H), 2.95-2.85 (m, 1H), 2.57-2.37 (m, 2H), 2.33-2.15 (m, 6H), 2.15-2.01 (m, 1H), 2.01-1.79 (m, 5H), 1.69-1.45 (m, 4H), 1.45-1.23 (m, 27H), 1.23-1.09 (m, 4H), 1.07-0.85 (m, 7H), 0.75 (s, 3H).

ESI-MS m/z: 650.45[M−1]⁻

### Example 48 Synthesis of Compound I-48

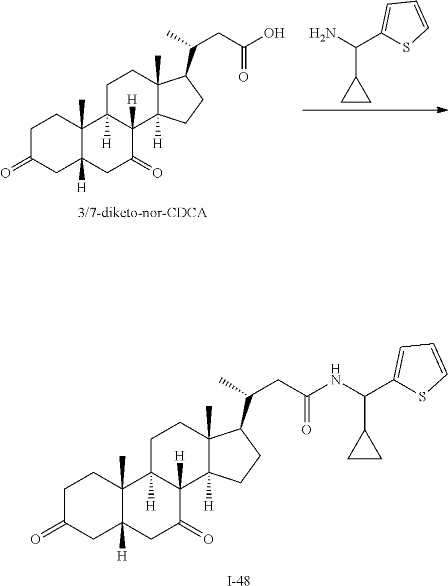

The compound I-48 was obtained by a synthetic method similar to that in Example 1 using 3,7-diketo-nor-CDCA and I-A-5 as raw materials.

ESI-MS m/z: 508.28[M−1]⁻

### Example 49 Synthesis of Compound I-49

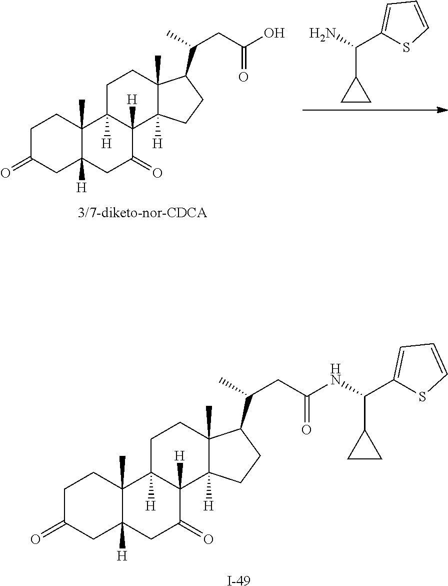

The compound I-49 was obtained by a synthetic method similar to that in Example 1 using 3,7-diketo-nor-CDCA and I-A-5-S as raw materials.

ESI-MS m/z: 508.28[M−1]⁻

### Example 50 Synthesis of Compound I-50

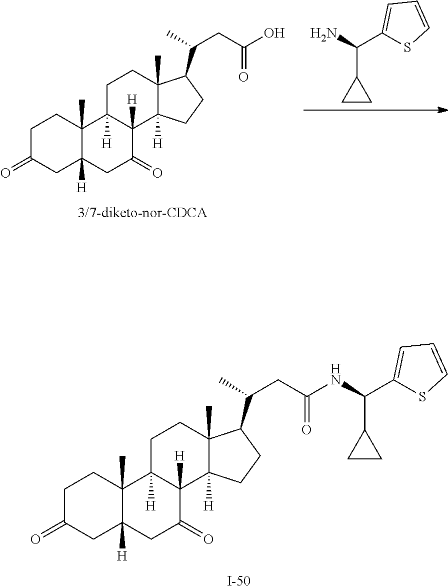

The compound I-50 was obtained by a synthetic method similar to that in Example 1 using 3,7-diketo-nor-CDCA and I-A-5-R as raw materials.

ESI-MS m/z: 508.28[M−1]⁻

### Example 51 Synthesis of Compound I-51

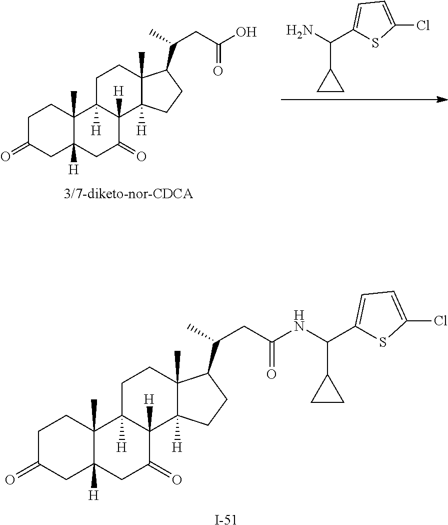

The compound I-51 was obtained by a synthetic method similar to that in Example 1 using 3,7-diketo-nor-CDCA and (R/S)-1-(5-chloro-thienyl-2)-cyclopropylamine as raw materials.

ESI-MS m/z: 542.20[M−1]⁻

### Example 52 Synthesis of Compound I-52

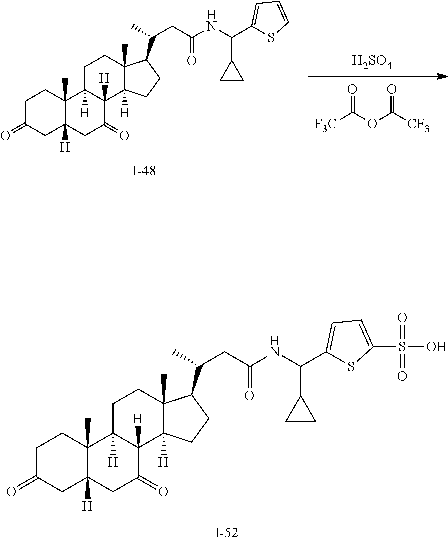

The compound I-52 was obtained by a synthetic method similar that in Example 34 using the compound I-48 as raw materials.

ESI-MS m/z: 611.20[M+Na]⁺

### Example 53 Synthesis of Compound I-53

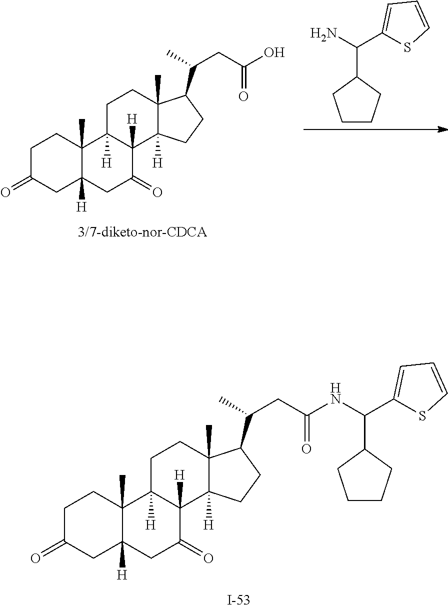

The compound I-53 was obtained by a synthetic method similar to that in Example 1 using 3,7-diketo-nor-CDCA and I-A-17 as raw materials.

ESI-MS m/z: 536.29[M−1]⁻

### Example 54 Synthesis of Compound I-54

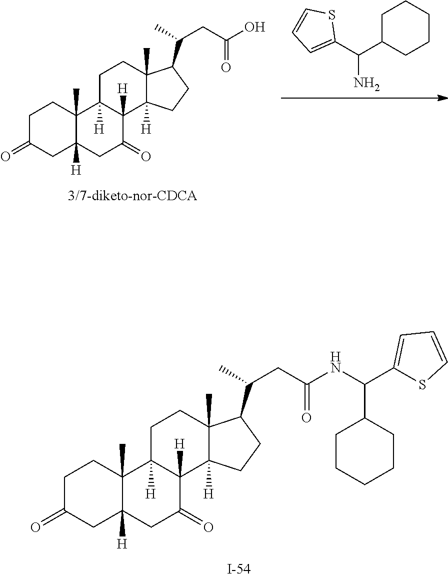

The compound I-54 was obtained by a synthetic method similar to that in Example 1 using 3,7-diketo-nor-CDCA and I-A-19 as raw materials.

¹HNMR(CDCl₃, 400 MHz): 37.20 (s, 1H), 6.96 (s, 2H), 5.64 (brs, 1H), 5.15 (m, 1H), 2.89 (m, 1H), 2.68-2.39 (m, 2H), 2.38-1.42 (m, 20H), 1.42-0.80 (m, 18H), 0.75 (s, 3H);

ESI-MS m/z: 550.28[M−1]⁻

### Example 55 Synthesis of Compound I-55

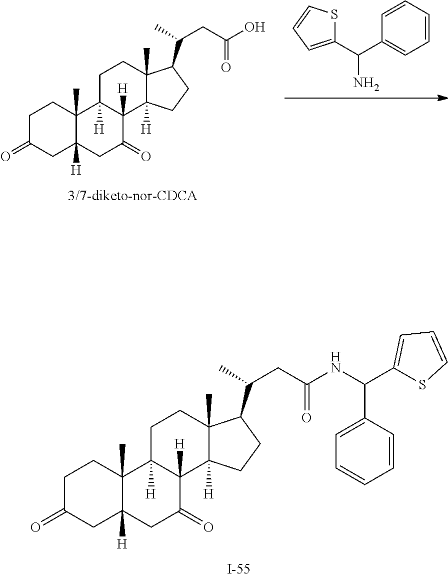

The compound I-55 was obtained by a synthetic method similar to that in Example 1 using 3,7-diketo-nor-CDCA and I-A-21 as raw materials.

¹HNMR(CDCl₃, 400 MHz): δ 7.42-7.30 (m, 5H), 7.25 (d, J=5.2 Hz, 1H), 6.96 (t, J=4.4 Hz, 1H), 6.81 (s, 1H), 6.50 (d, J=7.8 Hz, 1H), 6.11 (d, J=8.2 Hz, 1H), 2.95-2.85 (m, 1H), 2.57-2.44 (m, 2H), 2.31-2.18 (m, 5H), 2.15-2.05 (m, 3H), 2.02-1.79 (m, 4H), 1.70-1.58 (m, 3H), 1.32 (s, 3H), 1.30-1.12 (m, 4H), 1.08-0.96 (m, 3H), 0.75 (d, J=2.6 Hz, 3H).

ESI-MS m/z: 544.30[M−1]⁻

### Example 56 Synthesis of Compound I-56

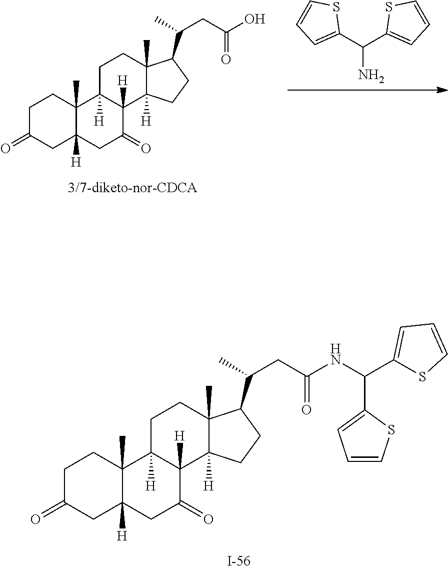

The compound I-56 was obtained by a synthetic method similar to that in Example 1 using 3,7-diketo-nor-CDCA and I-A-22 as raw materials.

¹HNMR(CDCl₃, 400 MHz): δ 7.28 (s, 2H), 7.03-6.95 (m, 4H), 6.78 (d, J=8.2 Hz, 1H), 6.14 (d, J=8.3 Hz, 1H), 2.95-2.85 (m, 1H), 2.57-2.43 (m, 2H), 2.35-2.17 (m, 5H), 2.15-2.03 (m, 3H), 2.02-1.79 (m, 4H), 1.63-1.51 (m, 4H), 1.51-1.33 (m, 2H), 1.32 (s, 2H), 1.30-1.12 (m, 3H), 1.02 (d, J=6.4 Hz, 3H), 1.09-0.84 (m, 1H), 0.76 (s, 3H).

ESI-MS m/z: 550.23[M−1]⁻

### Example 57 Synthesis of Compound I-57

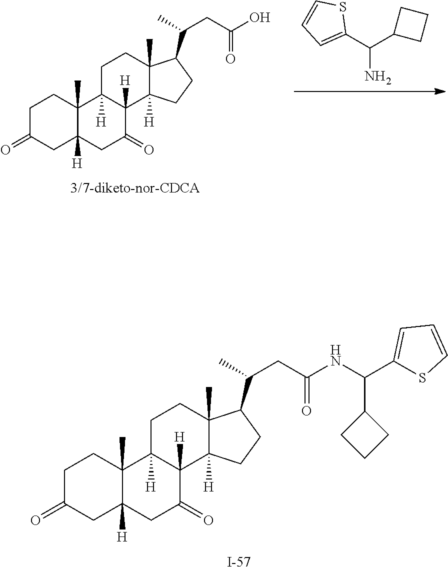

The compound I-57 was obtained by a synthetic method similar to that in Example 1 using 3,7-diketo-nor-CDCA and I-A-23 as raw materials.

ESI-MS m/z: 522.31[M−1]⁻

### Example 58 Synthesis of Compound I-58

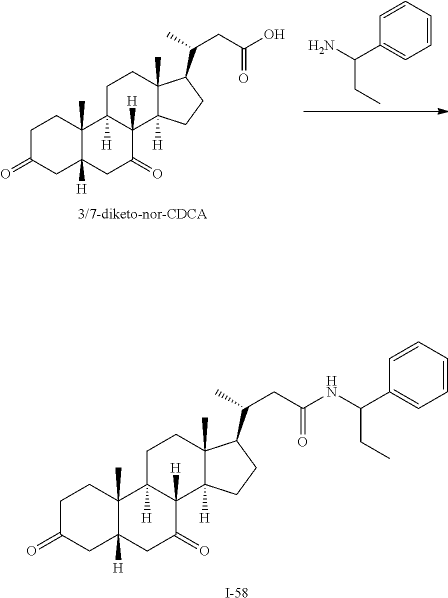

The compound I-58 was obtained by a synthetic method similar to that in Example 1 using 3,7-diketo-nor-CDCA and (R/S)-1-phenylpropan-1-amine as raw materials.

¹HNMR(CDCl₃, 400 MHz): δ 7.39-7.23 (m, 5H), 5.73 (s, 1H), 4.92 (q, J=7.6 Hz, 1H), 2.95-2.85 (m, 1H), 2.56-2.46 (m, 1H), 2.46-2.36 (m, 1H), 2.33-2.18 (m, 4H), 2.14-1.97 (m, 4H), 1.97-1.71 (m, 4H), 1.70-1.43 (m, 6H), 1.32 (s, 3H), 1.22-1.09 (m, 3H), 1.02 (d, J=6.5 Hz, 3H), 0.92 (t, J=7.3 Hz, 3H), 1.08-0.88 (m, 2H), 0.75 (s, 3H).

ESI-MS m/z: 490.30[M−1]⁻

### Example 59 Synthesis of Compound I-59

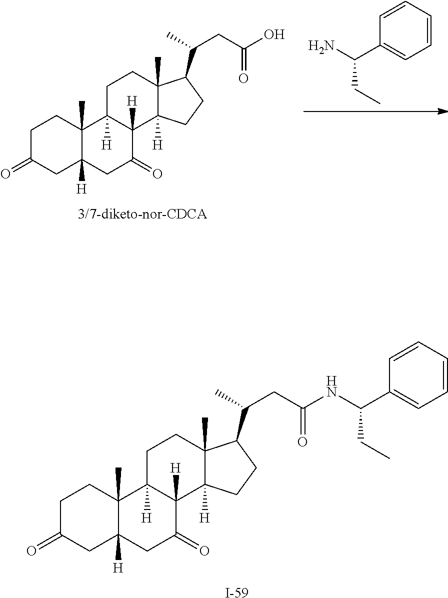

The compound I-59 was obtained by a synthetic method similar to that in Example 1 using 3,7-diketo-nor-CDCA and (S)-1-phenylpropan-1-amine as raw materials.

¹HNMR(CDCl₃, 400 MHz): δ 7.39-7.23 (m, 5H), 5.73 (s, 1H), 4.92 (q, J=7.6 Hz, 1H), 2.95-2.85 (m, 1H), 2.55-2.45 (m, 1H), 2.45-2.35 (m, 1H), 2.35-2.18 (m, 5H), 2.15-1.95 (m, 3H), 1.96-1.71 (m, 4H), 1.71-1.44 (m, 6H), 1.32 (s, 3H), 1.23-1.09 (m, 3H), 1.02 (d, J=6.5 Hz, 3H), 0.92 (t, J=7.3 Hz, 3H), 1.08-0.88 (m, 2H), 0.75 (s, 3H).

ESI-MS m/z: 490.30[M−1]⁻

### Example 60 Synthesis of Compound I-60

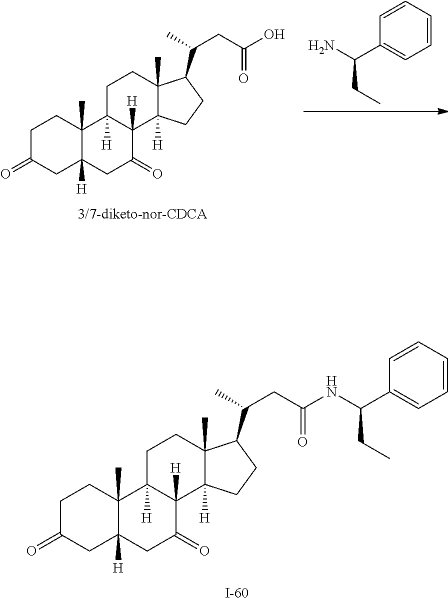

The compound I-60 was obtained by a synthetic method similar to that in Example 1 using 3,7-diketo-nor-CDCA and (R)-1-phenylpropan-1-amine as raw materials.

¹HNMR(CDCl₃, 400 MHz): δ 7.39-7.23 (m, 5H), 5.73 (s, 1H), 4.92 (q, J=7.6 Hz, 1H), 2.95-2.85 (m, 1H), 2.57-2.46 (m, 1H), 2.46-2.36 (m, 1H), 2.33-2.18 (m, 4H), 2.14-1.96 (m, 4H), 1.96-1.71 (m, 4H), 1.71-1.44 (m, 6H), 1.32 (s, 3H), 1.22-1.09 (m, 3H), 1.02 (d, J=6.5 Hz, 3H), 0.92 (t, J=7.3 Hz, 3H), 1.08-0.88 (m, 2H), 0.75 (s, 3H).

ESI-MS m/z: 490.30[M−1]⁻

### Example 61 Synthesis of Compound I-61

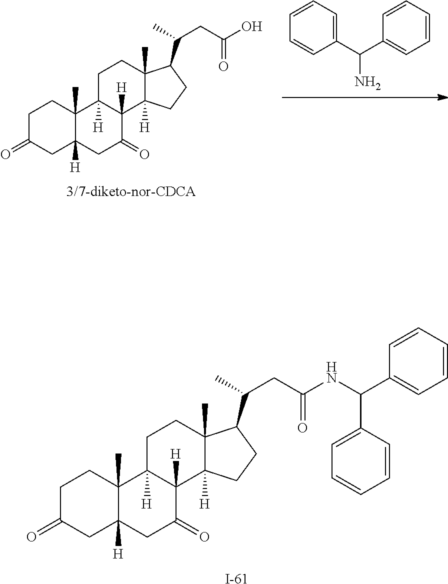

The compound I-61 was obtained by a synthetic method similar to that in Example 1 using 3,7-diketo-nor-CDCA and diphenylmethylamine as raw materials.

¹HNMR(CDCl₃, 400 MHz): δ 7.39-7.21 (m, 10H), 6.30 (d, J=7.8 Hz, 1H), 6.12-6.00 (m, 1H), 2.95-2.85 (m, 1H), 2.57-2.45 (m, 2H), 2.33-2.20 (m, 4H), 2.20-2.05 (m, 4H), 2.05-1.87 (m, 4H), 1.72-1.43 (m, 4H), 1.43-1.33 (m, 1H), 1.32 (s, 3H), 1.30-1.11 (m, 3H), 1.01 (d, J=6.5 Hz, 3H), 1.10-0.91 (m, 1H), 0.75 (s, 3H).

ESI-MS m/z: 538.30[M−1]⁻

### Example 62 Synthesis of Compound I-62

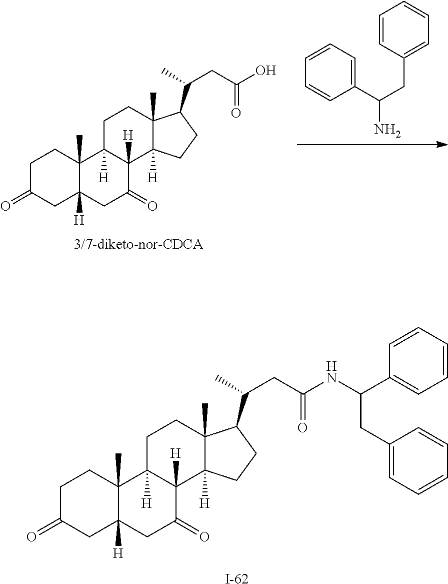

The compound I-62 was obtained by a synthetic method similar to that in Example 1 using 3,7-diketo-nor-CDCA and 1,2-diphenylethylamine as raw materials.

¹HNMR(CDCl₃, 400 MHz): δ 7.37-7.15 (m, 8H), 7.10 (t, J=7.8 Hz, 2H), 5.74 (s, 1H), 5.39-5.25 (m, 1H), 3.20-3.04 (m, 2H), 2.95-2.85 (m, 1H), 2.56-2.45 (m, 1H), 2.40-2.03 (m, 8H), 2.01-1.87 (m, 2H), 1.87-1.66 (m, 2H), 1.66-1.54 (m, 2H), 1.52-1.42 (m, 2H), 1.32 (s, 3H), 1.34-0.92 (m, 6H), 0.88-0.78 (m, 3H), 0.71 (s, 3H).

ESI-MS m/z: 552.34[M−1]⁻

### Example 63 Synthesis of Compound I-63

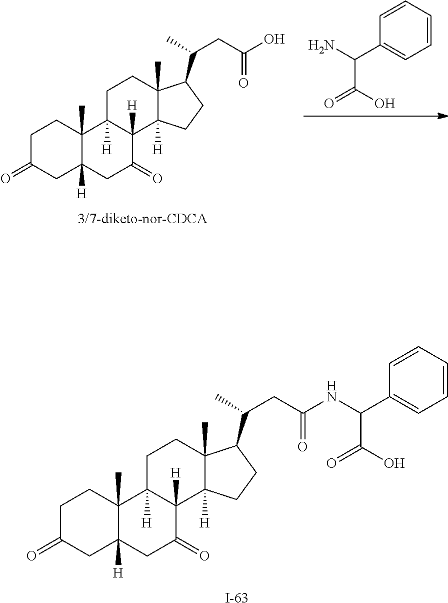

The compound I-63 was obtained by a synthetic method similar to that in Example 1 using 3,7-diketo-nor-CDCA and 2-amino-2-phenylacetic acid as raw materials.

¹HNMR(CDCl₃, 400 MHz): δ 7.43-7.33 (m, 5H), 6.62 (s, 1H), 5.60 (d, J=6.8 Hz, 1H), 2.94-2.84 (m, 1H), 2.56-2.39 (m, 2H), 2.34-2.16 (m, 5H), 2.17-2.02 (m, 5H), 2.01-1.81 (m, 5H), 1.72-1.54 (m, 2H), 1.32 (s, 3H), 1.24-1.09 (m, 4H), 1.05-0.94 (m, 4H), 0.72 (s, 3H).

ESI-MS m/z: 506.46[M−1]⁻

### Example 64 Synthesis of Compound I-64

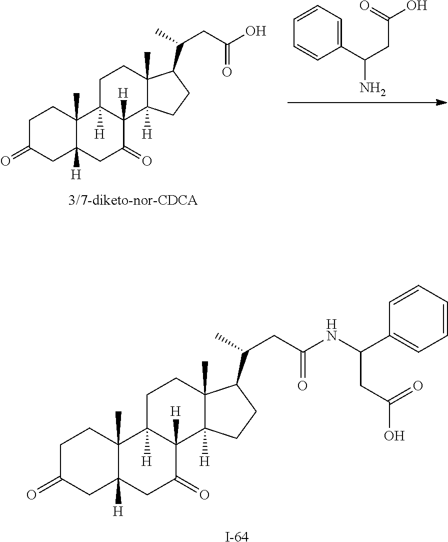

The compound I-64 was obtained by a synthetic method similar to that in Example 1 using 3,7-diketo-nor-CDCA and 3-amino-3-phenylpropionic acid as raw materials.

¹HNMR(CDCl₃, 400 MHz): δ7.39-7.24 (m, 5H), 6.77-6.50 (m, 1H), 5.47 (q, J=6.8 Hz, 1H), 4.18-4.10 (m, 1H), 3.05-2.83 (m, 3H), 2.57-2.38 (m, 2H), 2.34-2.15 (m, 6H), 2.14-1.96 (m, 6H), 1.96-1.77 (m, 2H), 1.63-1.44 (m, 3H), 1.32 (s, 3H), 1.40-1.11 (m, 3H), 1.09-0.88 (m, 4H), 0.73 (s, 3H).

ESI-MS m/z: 520.19[M−1]⁻

### Example 65 Synthesis of Compound I-65

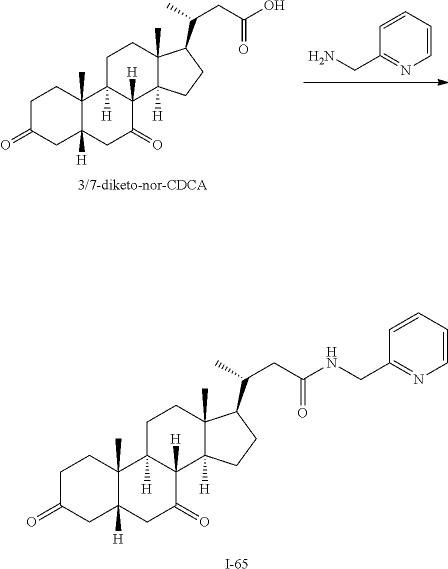

The compound I-65 was obtained by a synthetic method similar to that in Example 1 using 3,7-diketo-nor-CDCA and 2-aminomethyl-pyridine as raw materials.

¹HNMR(CDCl₃, 400 MHz): δ8.56 (d, J=4.9 Hz, 1H), 7.77-7.67 (m, 1H), 7.37-7.20 (m, 2H), 6.83-6.76 (m, 1H), 4.63-4.56 (m, 2H), 2.95-2.85 (m, 1H), 2.57-2.44 (m, 2H), 2.30-2.20 (m, 5H), 2.20-1.96 (m, 4H), 1.98-1.84 (m, 4H), 1.73-1.45 (m, 5H), 1.45-1.31 (m, 1H), 1.32 (s, 3H), 1.27-1.12 (m, 2H), 1.01 (d, J=6.5 Hz, 3H), 0.76 (s, 3H).

ESI-MS m/z: 463.18[M−1]⁻

### Example 66 Synthesis of Compound I-66

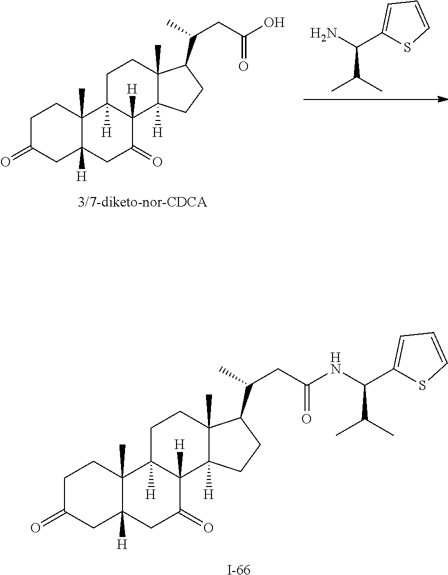

The compound I-66 was obtained by a synthetic method similar to that in Example 1 using 3,7-diketo-nor-CDCA and I-A-8-R as raw materials.

¹HNMR(CDCl₃, 400 MHz): δ 7.28 (s, 1H), 6.79-6.67 (m, 2H), 5.81-5.57 (m, 1H), 5.06-4.97 (m, 1H), 2.95-2.86 (m, 1H), 2.58-2.40 (m, 2H), 2.36-2.10 (m, 7H), 2.10-1.97 (m, 3H), 1.97-1.74 (m, 3H), 1.74-1.45 (m, 6H), 1.33 (s, 3H), 1.42-1.10 (m, 3H), 1.10-0.93 (m, 10H), 0.75 (s, 3H).

ESI-MS m/z: 510.19[M−1]⁻

### Example 67 Synthesis of Compound I-67

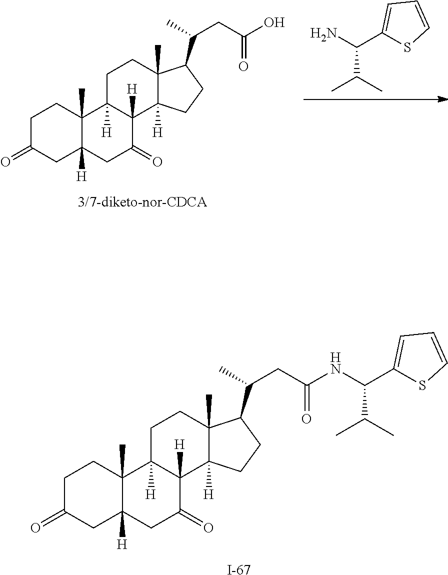

The compound I-67 was obtained by a synthetic method similar to that in Example 1 using 3,7-diketo-nor-CDCA and I-A-8-S as raw materials.

¹HNMR(CDCl₃, 400 MHz): δ 7.28 (s, 1H), 6.79-6.67 (m, 2H), 5.81-5.57 (m, 1H), 5.07-4.97 (m, 1H), 2.96-2.86 (m, 1H), 2.58-2.38 (m, 3H), 2.38-2.10 (m, 6H), 2.10-1.97 (m, 3H), 1.97-1.74 (m, 3H), 1.74-1.45 (m, 6H), 1.33 (s, 3H), 1.42-1.10 (m, 3H), 1.10-0.93 (m, 10H), 0.75 (s, 3H).

ESI-MS m/z: 510.19[M−1]⁻

### Example 68 Synthesis of Compound I-68

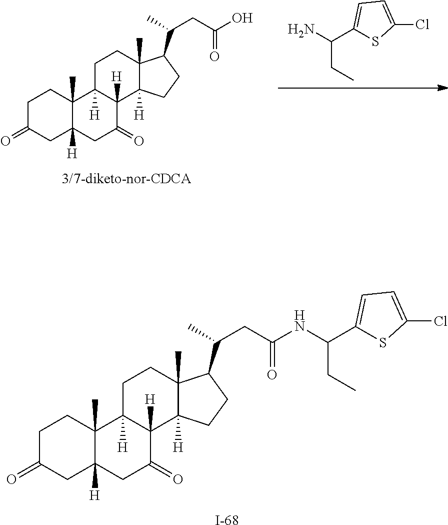

The compound I-68 was obtained by a synthetic method similar to that in Example 1 using 3,7-diketo-nor-CDCA and I-A-6 as raw materials.

¹HNMR(CDCl₃, 400 MHz): δ 6.78-6.70 (m, 2H), 5.71 (d, J=8.8 Hz, 1H), 5.10 (q, J=7.6 Hz, 1H), 2.95-2.85 (m, 1H), 2.57-2.36 (m, 3H), 2.36-2.14 (m, 7H), 2.14-2.01 (m, 4H), 2.01-1.90 (m, 2H), 1.90-1.68 (m, 3H), 1.68-1.44 (m, 2H), 1.32 (s, 3H), 1.30-1.10 (m, 3H), 1.09-0.87 (m, 7H), 0.74 (s, 3H).

ESI-MS m/z: 530.23[M−1]⁻

### Example 69 Synthesis of Compound I-69

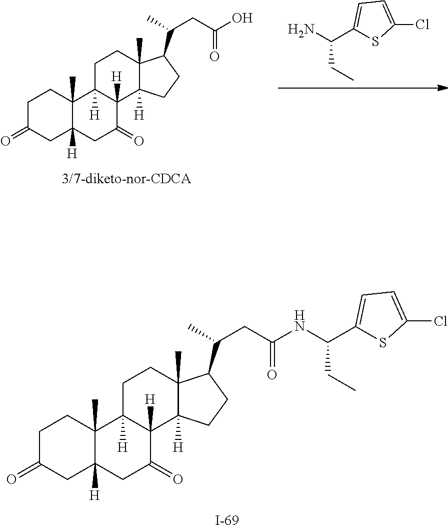

The compound I-69 was obtained by a synthetic method similar to that in Example 1 using 3,7-diketo-nor-CDCA and I-A-6-S as raw materials.

¹HNMR(CDCl₃, 400 MHz): δ 6.78-6.70 (m, 2H), 5.71 (d, J=8.8 Hz, 1H), 5.10 (q, J=7.6 Hz, 1H), 2.94-2.84 (m, 1H), 2.58-2.38 (m, 3H), 2.36-2.14 (m, 8H), 2.14-2.01 (m, 3H), 2.01-1.90 (m, 2H), 1.90-1.68 (m, 3H), 1.68-1.44 (m, 2H), 1.32 (s, 3H), 1.30-1.10 (m, 3H), 1.09-0.87 (m, 7H), 0.74 (s, 3H).

ESI-MS m/z: 554.22[M+Na]⁺

### Example 70 Synthesis of Compound I-70

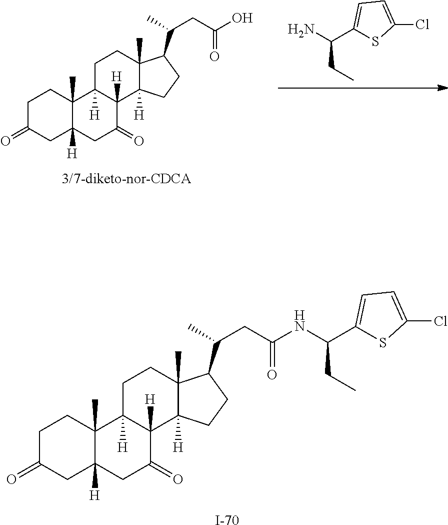

The compound I-70 was obtained by a synthetic method similar to that in Example 1 using 3,7-diketo-nor-CDCA and I-A-6-R as raw materials.

¹HNMR(CDCl₃, 400 MHz): δ 6.78-6.70 (m, 2H), 5.71 (d, J=8.8 Hz, 1H), 5.10 (q, J=7.6 Hz, 1H), 2.95-2.85 (m, 1H), 2.57-2.37 (m, 3H), 2.36-2.14 (m, 8H), 2.14-2.01 (m, 3H), 2.01-1.90 (m, 2H), 1.90-1.68 (m, 3H), 1.68-1.44 (m, 2H), 1.32 (s, 3H), 1.30-1.10 (m, 3H), 1.09-0.87 (m, 7H), 0.74 (s, 3H).

ESI-MS m/z: 554.22[M+Na]⁺

### Example 71 Synthesis of Compound I-71

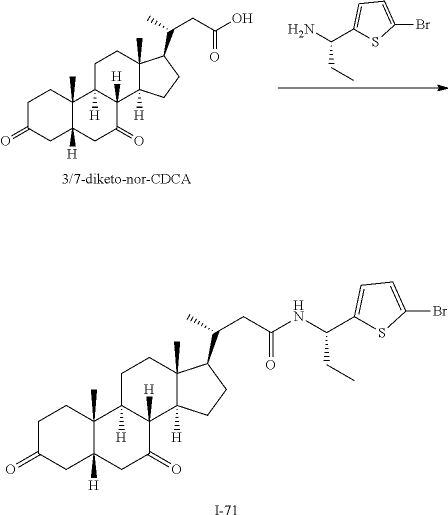

The compound I-71 was obtained by a synthetic method similar to that in Example 1 using 3,7-diketo-nor-CDCA and I-A-7-S as raw materials.

¹HNMR(CDCl₃, 400 MHz): δ 6.91 (d, J=3.7 Hz, 1H), 6.73 (d, J=3.8, 1H), 5.62 (s, 1H), 5.14 (q, J=7.7 Hz, 1H), 2.95-2.85 (m, 1H), 2.58-2.38 (m, 2H), 2.36-2.16 (m, 6H), 2.16-2.03 (m, 4H), 2.03-1.70 (m, 5H), 1.70-1.50 (m, 5H), 1.33 (s, 3H), 1.30-1.12 (m, 2H), 1.15-0.94 (m, 7H), 0.76 (s, 3H).

ESI-MS m/z: 598.10[M+Na]⁺

### Example 72 Synthesis of Compound I-72

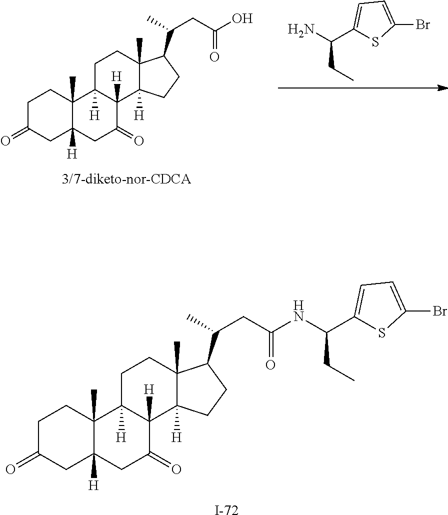

The compound I-72 was obtained by a synthetic method similar to that in Example 1 using 3,7-diketo-nor-CDCA and I-A-7-R as raw materials.

¹HNMR(CDCl₃, 400 MHz): δ 6.91 (d, J=3.7 Hz, 1H), 6.73 (d, J=3.8, 1H), 5.62 (s, 1H), 5.14 (q, J=7.7 Hz, 1H), 2.96-2.86 (m, 1H), 2.58-2.38 (m, 2H), 2.35-2.15 (m, 6H), 2.15-2.03 (m, 4H), 2.03-1.71 (m, 5H), 1.70-1.50 (m, 5H), 1.33 (s, 3H), 1.30-1.12 (m, 2H), 1.15-0.94 (m, 7H), 0.76 (s, 3H).

ESI-MS m/z: 598.10[M+Na]⁺

### Example 73 Synthesis of Compound I-73

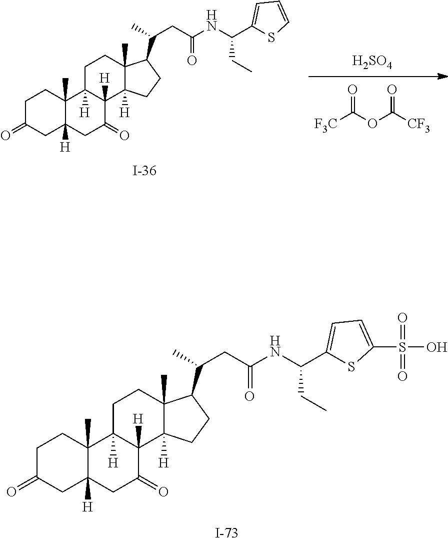

The compound I-73 was obtained by a synthetic method similar that in Example 34 using the compound I-36 as raw materials.

ESI-MS m/z: 599.14[M+Na]⁺

### Example 74 Synthesis of Compound I-74

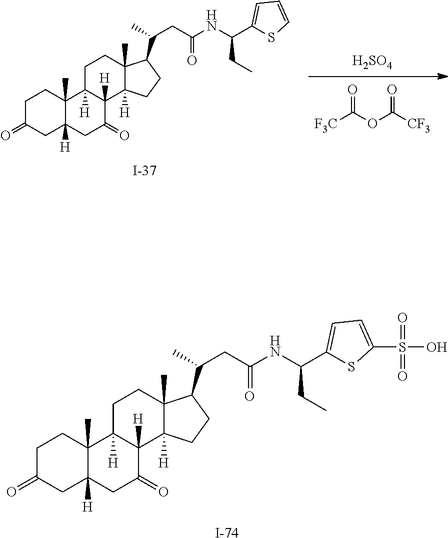

The compound I-74 was obtained by a synthetic method similar that in Example 34 using the compound I-37 as raw materials.

ESI-MS m/z: 599.140[M+Na]⁺

### Example 75 Synthesis of Compound I-75

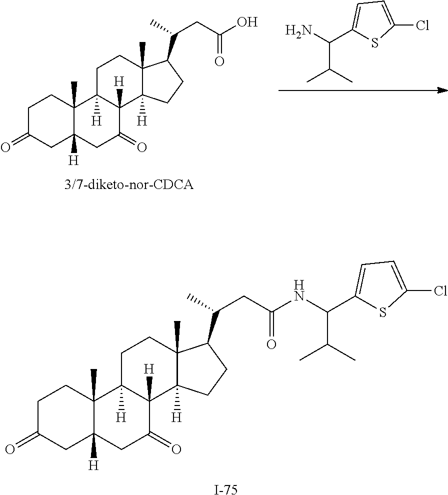

The compound I-75 was obtained by a synthetic method similar to that in Example 1 using 3,7-diketo-nor-CDCA and I-A-9 as raw materials.

¹HNMR(CDCl₃, 400 MHz): δ 6.79-6.67 (m, 2H), 5.71 (s, 1H), 5.07-4.96 (m, 1H), 2.95-2.85 (m, 1H), 2.58-2.40 (m, 2H), 2.36-2.20 (m, 5H), 2.20-2.06 (m, 3H), 2.06-1.74 (m, 4H), 1.71-1.45 (m, 6H), 1.32 (s, 3H), 1.44-1.14 (m, 3H), 1.14-0.85 (m, 10H), 0.75 (d, J=4.0 Hz, 3H).

ESI-MS m/z: 568.22[M+Na]⁺

### Example 76 Synthesis of Compound I-76

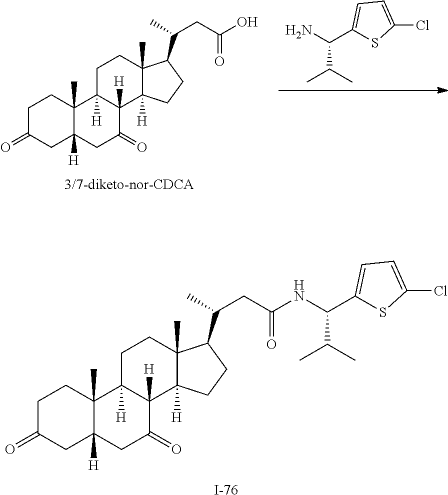

The compound I-76 was obtained by a synthetic method similar to that in Example 1 using 3,7-diketo-nor-CDCA and I-A-9-S as raw materials.

¹HNMR(CDCl₃, 400 MHz): δ 6.79-6.67 (m, 2H), 5.71 (s, 1H), 5.07-4.96 (m, 1H), 2.85-2.86 (m, 1H), 2.58-2.40 (m, 2H), 2.36-2.20 (m, 5H), 2.20-2.06 (m, 3H), 2.06-1.74 (m, 4H), 1.71-1.45 (m, 6H), 1.32 (s, 3H), 1.44-1.14 (m, 3H), 1.14-0.85 (m, 10H), 0.74 (s, 3H).

ESI-MS m/z: 568.22[M+Na]⁺

### Example 77 Synthesis of Compound I-77

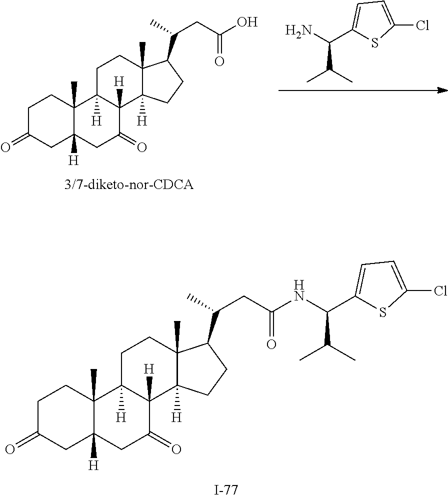

The compound I-77 was obtained by a synthetic method similar to that in Example 1 using 3,7-diketo-nor-CDCA and I-A-9-R as raw materials. ¹HNMR(CDCl₃, 400 MHz): δ 6.79-6.67 (m, 2H), 5.71 (s, 1H), 5.07-4.96 (m, 1H), 2.95-2.85 (m, 1H), 2.58-2.41 (m, 2H), 2.36-2.20 (m, 6H), 2.20-2.06 (m, 3H), 2.06-1.74 (m, 4H), 1.71-1.45 (m, 5H), 1.32 (s, 3H), 1.44-1.14 (m, 3H), 1.14-0.85 (m, 10H), 0.75 (s, 3H).

ESI-MS m/z: 568.22[M+Na]⁺

### Example 78 Synthesis of Compound I-78

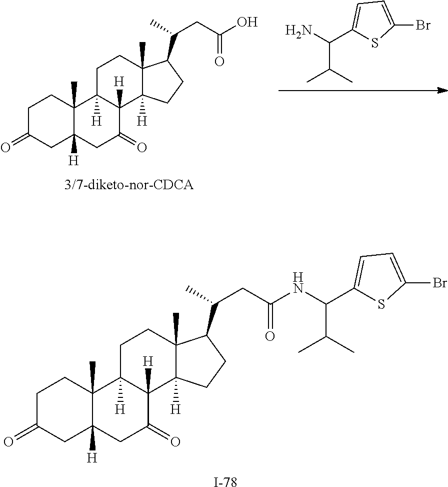

The compound I-78 was obtained by a synthetic method similar to that in Example 1 using 3,7-diketo-nor-CDCA and I-A-10 as raw materials. ¹HNMR(CDCl₃, 400 MHz): δ 6.90 (d, J=3.7 Hz, 1H), 6.73-6.66 (m, 1H), 5.75 (s, 1H), 5.09-4.99 (m, 1H), 2.95-2.86 (m, 1H), 2.58-2.40 (m, 2H), 2.35-2.21 (m, 6H), 2.21-1.74 (m, 9H), 1.74-1.55 (m, 2H), 1.56-1.25 (m, 2H), 1.32 (s, 3H), 1.26-1.11 (m, 2H), 1.10-0.91 (m, 10H), 0.75 (d, J=4.0 Hz, 3H).

ESI-MS m/z: 612.14[M+Na]⁺

### Example 79 Synthesis of Compound I-79

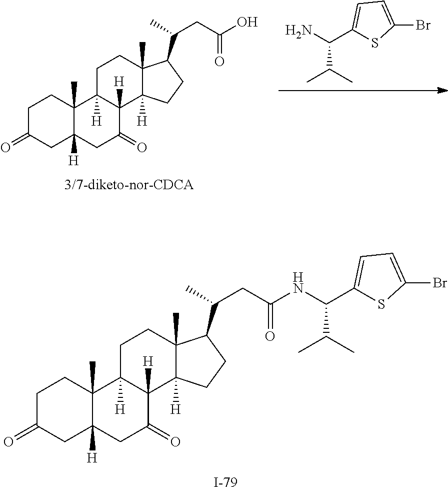

The compound I-79 was obtained by a synthetic method similar to that in Example 1 using 3,7-diketo-nor-CDCA and I-A-10-S as raw materials. ¹HNMR(CDCl₃, 400 MHz): δ 6.90 (d, J=3.7 Hz, 1H), 6.73-6.66 (m, 1H), 5.75 (s, 1H), 5.09-4.99 (m, 1H), 2.96-2.86 (m, 1H), 2.58-2.40 (m, 2H), 2.35-2.17 (m, 7H), 2.17-1.74 (m, 8H), 1.74-1.55 (m, 2H), 1.56-1.25 (m, 2H), 1.32 (s, 3H), 1.26-1.11 (m, 2H), 1.10-0.91 (m, 10H), 0.74 (s, 3H).

ESI-MS m/z: 612.14[M+Na]⁺

### Example 80 Synthesis of Compound I-80

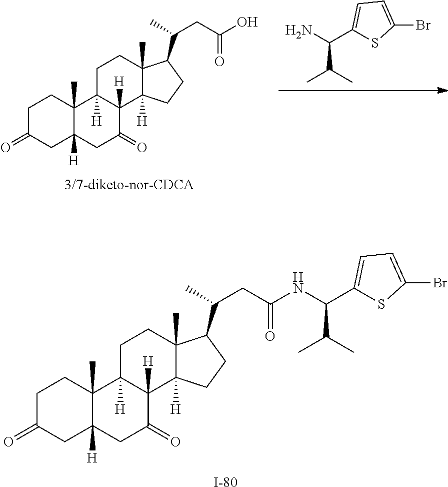

The compound I-80 was obtained by a synthetic method similar to that in Example 1 using 3,7-diketo-nor-CDCA and I-A-10-R as raw materials. ¹HNMR(CDCl₃, 400 MHz): δ 6.90 (d, J=3.8 Hz, 1H), 6.69 (d, J=3.8, 1H), 5.68 (s, 1H), 5.09-5.00 (m, 1H), 2.95-2.86 (m, 1H), 2.58-2.48 (m, 1H), 2.48-2.40 (m, 1H), 2.33-2.17 (m, 6H), 2.17-2.07 (m, 3H), 2.07-1.90 (m, 3H), 1.90-1.74 (m, 2H), 1.74-1.59 (m, 1H), 1.63-1.45 (m, 3H), 1.33 (s, 3H), 1.42-1.12 (m, 3H), 1.15-0.93 (m, 10H), 0.75 (s, 3H).

ESI-MS m/z: 612.14[M+Na]⁺

### Example 81 Synthesis of Compound I-81

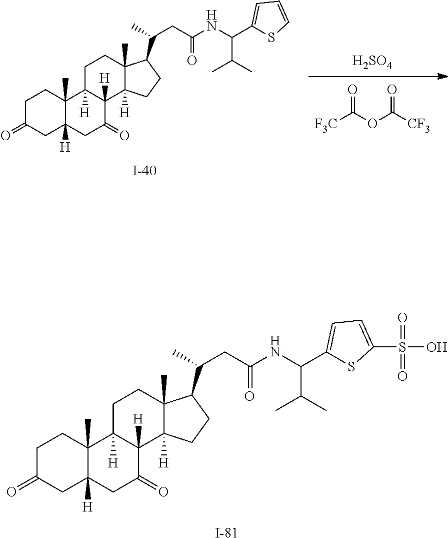

The compound I-81 was obtained by a synthetic method similar that in Example 34 using I-40 as raw materials.

ESI-MS m/z: 613.14[M+Na]⁺

### Example 82 Synthesis of Compound I-82

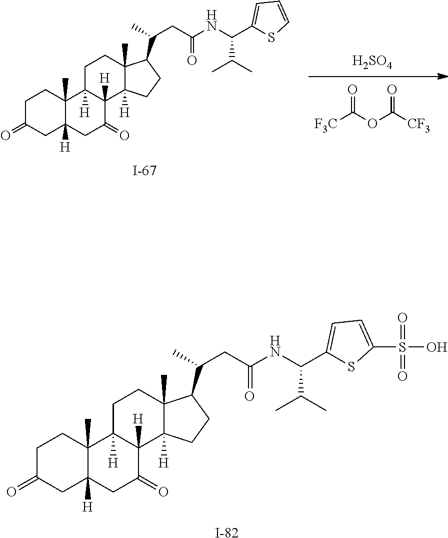

The compound I-82 was obtained by a synthetic method similar that in Example 34 using I-67 as raw materials.

ESI-MS m/z: 613.14[M+Na]⁺

### Example 83 Synthesis of Compound I-83

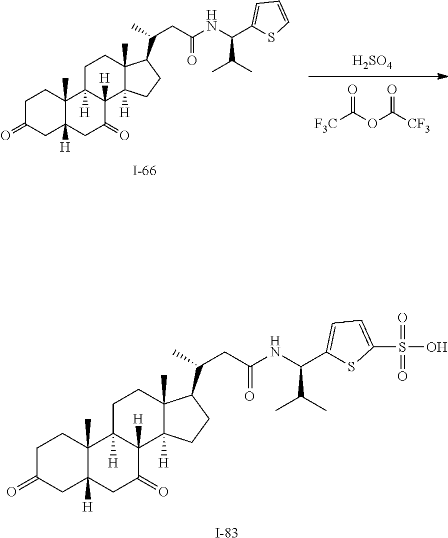

The compound I-83 was obtained by a synthetic method similar that in Example 34 using I-66 as raw materials.

ESI-MS m/z: 613.14[M+Na]⁺

### Example 84 Synthesis of Compound I-84

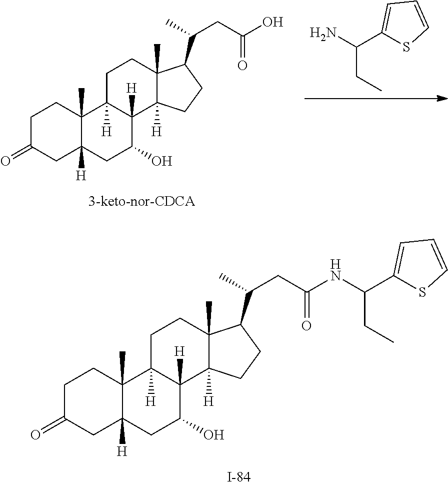

The compound I-84 was obtained by a synthetic method similar to that in Example 1 using 3-keto-nor-CDCA and I-A-1 as raw materials.

¹HNMR(CDCl₃, 400 MHz): δ 7.25-7.19 (m, 1H), 6.97 (s, 2H), 5.64 (brs, 1H), 5.25 (q, J=7.6 Hz, 1H), 3.97-3.91 (m, 1H), 3.46-3.34 (m, 1H), 2.48-2.35 (m, 2H), 2.26-1.74 (m, 10H), 1.74-1.34 (m, 10H), 1.27-1.12 (m, 4H), 1.05-0.95 (m, 9H), 0.76 (s, 3H).

ESI-MS m/z: 498.11[M−1]⁻

### Example 85 Synthesis of Compound I-85

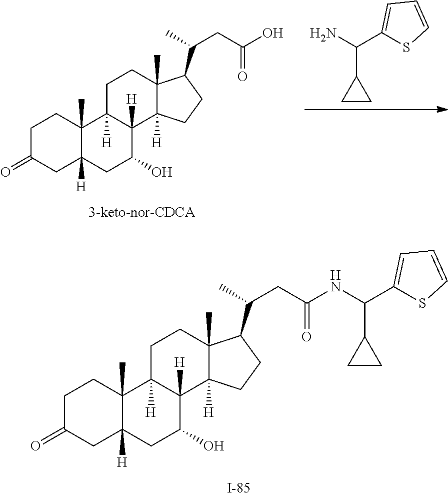

The compound I-85 was obtained by a synthetic method similar to that in Example 1 using 3-keto-nor-CDCA and I-A-5 as raw materials.

¹HNMR(CDCl₃, 400 MHz): δ 7.23 (d, J=5.1 Hz, 1H), 7.06 (s, 1H), 6.98 (t, J=4.4 Hz, 1H), 5.89-5.80 (m, 1H), 4.75 (t, J=8.9 Hz, 1H), 3.95 (s, 1H), 3.46-3.34 (m, 1H), 2.50-2.35 (m, 2H), 2.26-2.12 (m, 2H), 2.11-1.92 (m, 5H), 1.85-1.74 (m, 2H), 1.62-1.36 (m, 10H), 1.33-1.16 (m, 5H), 1.06 (d, J=6.4 Hz, 2H), 1.03 (s, 3H), 0.77 (s, 3H), 0.79-0.67 (m, 2H), 0.67-0.44 (m, 2H).

ESI-MS m/z: 410.13[M−1]⁻

### Example 86 Synthesis of Compound I-86

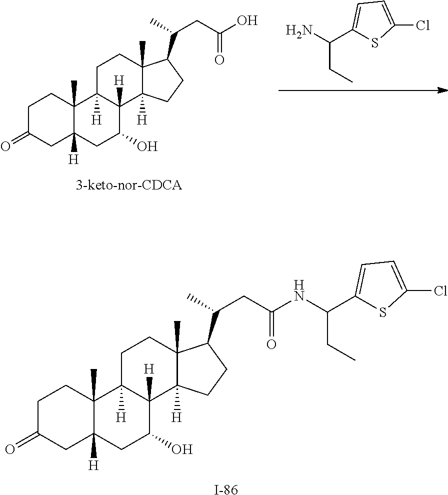

The compound I-86 was obtained by a synthetic method similar to that in Example 1 using 3-keto-nor-CDCA and I-A-6 as raw materials.

¹HNMR(d6-DMSO, 400 MHz): δ8.25 (d, J=8.3 Hz, 1H), 6.94 (d, J=3.8 Hz, 1H), 6.82-6.76 (m, 1H), 4.92-4.81 (m, 1H), 4.41-4.36 (m, 1H), 3.69 (s, 1H), 3.47-3.35 (m, 1H), 2.43-2.29 (m, 1H), 2.34-2.14 (m, 1H), 2.01-1.88 (m, 5H), 1.88-1.74 (m, 5H), 1.74-1.61 (m, 4H), 1.53-1.38 (m, 4H), 1.38-1.17 (m, 3H), 1.17-0.98 (m, 2H), 0.97-0.78 (m, 9H), 0.67 (s, 3H).

ESI-MS m/z: 532.14[M−1]⁻

### Example 87 Synthesis of Compound I-87

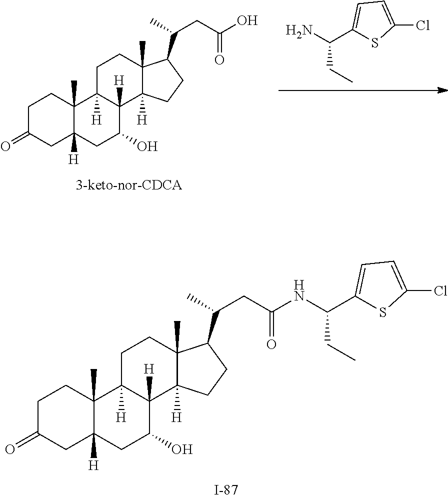

The compound I-87 was obtained by a synthetic method similar to that in Example 1 using 3-keto-nor-CDCA and I-A-6-S as raw materials.

¹HNMR(d6-DMSO, 400 MHz): δ8.25 (d, J=8.3 Hz, 1H), 6.94 (d, J=3.8 Hz, 1H), 6.80 (d, J=3.8 Hz, 1H), 4.93-4.80 (m, 1H), 4.38 (d, J=3.6 Hz, 1H), 3.69 (s, 1H), 3.45-3.35 (m, 1H), 2.43-2.29 (m, 1H), 2.35-2.15 (m, 1H), 2.01-1.88 (m, 5H), 1.88-1.74 (m, 5H), 1.74-1.61 (m, 4H), 1.53-1.38 (m, 4H), 1.38-1.17 (m, 3H), 1.17-0.98 (m, 2H), 0.97-0.78 (m, 9H), 0.67 (s, 3H).

ESI-MS m/z: 532.14[M−1]⁻

### Example 88 Synthesis of Compound I-88

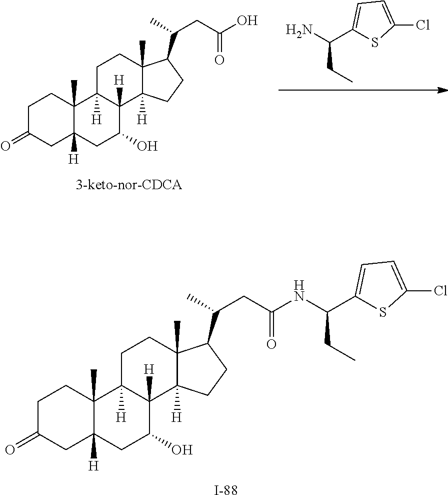

The compound I-88 was obtained by a synthetic method similar to that in Example 1 using 3-keto-nor-CDCA and I-A-6-R as raw materials.

¹HNMR(d6-DMSO, 400 MHz): δ8.25 (d, J=8.3 Hz, 1H), 6.94 (d, J=3.8 Hz, 1H), 6.79 (d, J=3.8 Hz, 1H), 4.92-4.81 (m, 1H), 4.38 (d, J=3.6 Hz, 1H), 3.69 (s, 1H), 3.47-3.35 (m, 1H), 2.43-2.29 (m, 1H), 2.34-2.14 (m, 1H), 2.01-1.88 (m, 5H), 1.88-1.74 (m, 5H), 1.74-1.61 (m, 4H), 1.53-1.38 (m, 4H), 1.38-1.17 (m, 3H), 1.17-0.98 (m, 2H), 0.97-0.78 (m, 9H), 0.67 (s, 3H).

ESI-MS m/z: 532.14[M−1]⁻

### Example 89 Synthesis of Compound I-89

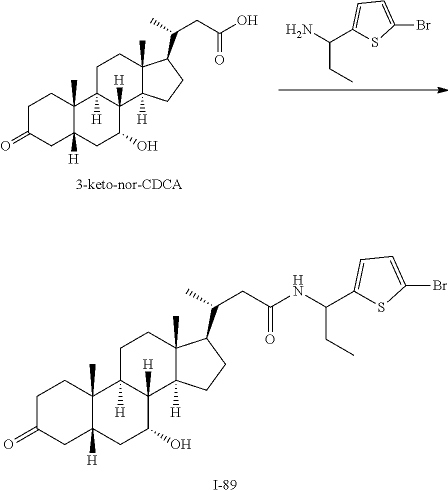

The compound I-89 was obtained by a synthetic method similar to that in Example 1 using 3-keto-nor-CDCA and I-A-7 as raw materials.

¹HNMR(CDCl₃, 400 MHz): δ6.94-6.88 (m, 1H), 6.76-6.70 (m, 1H), 5.14 (q, J=7.7 Hz, 1H), 3.94 (s, 1H), 3.46-3.33 (m, 1H), 2.49-2.35 (m, 2H), 2.27-2.11 (m, 2H), 2.11-1.71 (m, 10H), 1.70-1.12 (m, 11H), 1.05-0.87 (m, 10H), 0.76 (s, 3H).

ESI-MS m/z: 575.86[M−1]⁻

### Example 90 Synthesis of Compound I-90

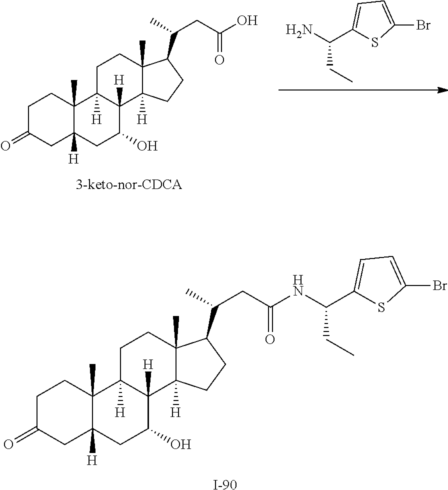

The compound I-90 was obtained by a synthetic method similar to that in Example 1 using 3-keto-nor-CDCA and I-A-7-S as raw materials.

¹HNMR(CDCl₃, 400 MHz): δ6.91 (d, J=3.8 Hz, 1H), 6.73 (d, J=3.7 Hz, 1H), 5.13 (q, J=7.7 Hz, 1H), 3.94 (s, 1H), 3.48-3.35 (m, 1H), 2.48-2.35 (m, 2H), 2.25-2.11 (m, 2H), 2.12-1.71 (m, 9H), 1.71-1.12 (m, 12H), 1.05-0.87 (m, 10H), 0.76 (s, 3H).

ESI-MS m/z: 575.86[M−1]⁻

### Example 91 Synthesis of Compound I-91

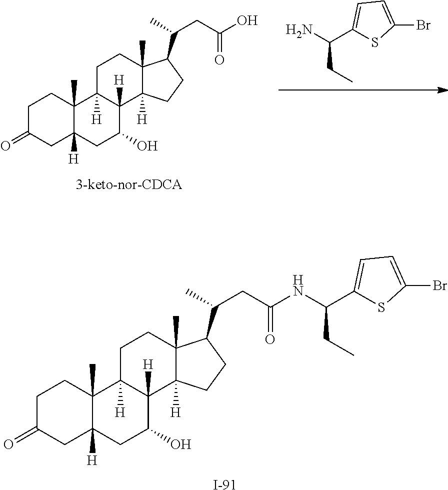

The compound I-91 was obtained by a synthetic method similar to that in Example 1 using 3-keto-nor-CDCA and I-A-7-R as raw materials.

¹HNMR(CDCl₃, 400 MHz): δ6.91 (d, J=3.8 Hz, 1H), 6.73 (d, J=3.7 Hz, 1H), 5.14 (q, J=7.7 Hz, 1H), 3.94 (s, 1H), 3.47-3.34 (m, 1H), 2.48-2.35 (m, 2H), 2.26-2.11 (m, 2H), 2.11-1.71 (m, 9H), 1.71-1.12 (m, 12H), 1.05-0.87 (m, 10H), 0.76 (s, 3H).

ESI-MS m/z: 575.86[M−1]⁻

### Example 92 Synthesis of Compound I-92

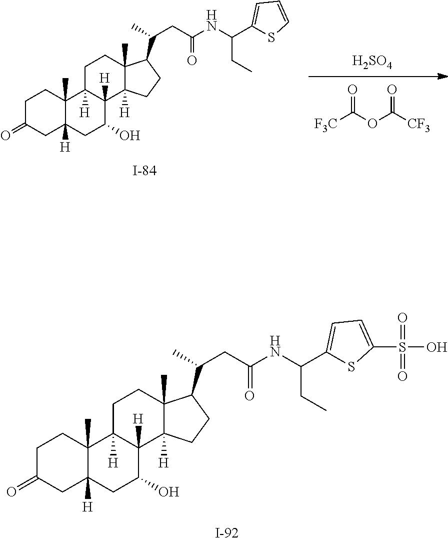

The compound I-92 was obtained by a synthetic method similar that in Example 34 using the compound I-84 as raw materials.

ESI-MS m/z: 601.14[M+Na]⁺

### Example 93 Synthesis of Compound I-93

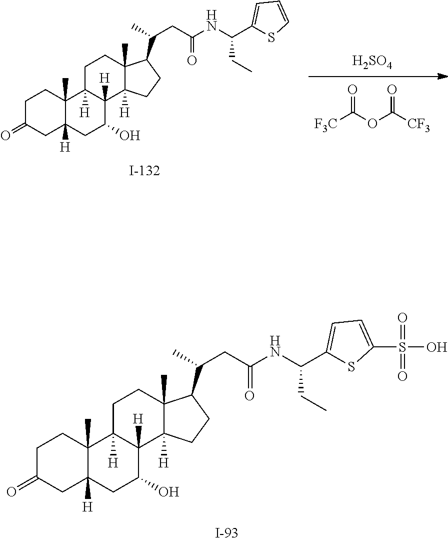

The compound I-93 was obtained by a synthetic method similar that in Example 34 using the compound I-132 as raw materials.

ESI-MS m/z: 601.14[M+Na]⁺

### Example 94 Synthesis of Compound I-94

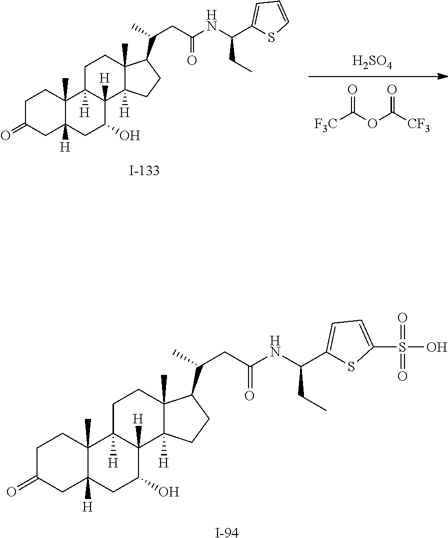

The compound I-94 was obtained by a synthetic method similar that in Example 34 using the compound I-133 as raw materials.

ESI-MS m/z: 601.14[M+Na]⁺

### Example 95 Synthesis of Compound I-95

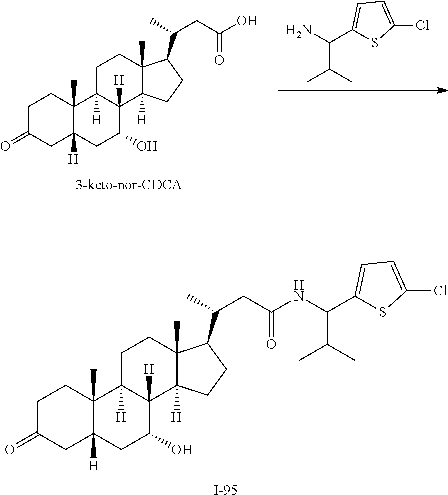

The compound I-95 was obtained by a synthetic method similar to that in Example 1 using 3-keto-nor-CDCA and I-A-9 as raw materials.

¹HNMR(CDCl₃, 400 MHz): δ 6.80-6.68 (m, 2H), 5.69 (s, 1H), 5.07-4.97 (m, 1H), 3.94 (q, J=3.0 Hz, 1H), 3.47-3.34 (m, 1H), 2.48-2.36 (m, 2H), 2.27-2.13 (m, 2H), 2.13-1.92 (m, 7H), 1.84-1.74 (m, 3H), 1.71-1.32 (m, 7H), 1.32-1.14 (m, 1H), 1.21 (s, 3H), 1.05-0.92 (m, 12H), 0.76 (s, 3H).

ESI-MS m/z: 570.24[M+Na]⁺

### Example 96 Synthesis of Compound I-96

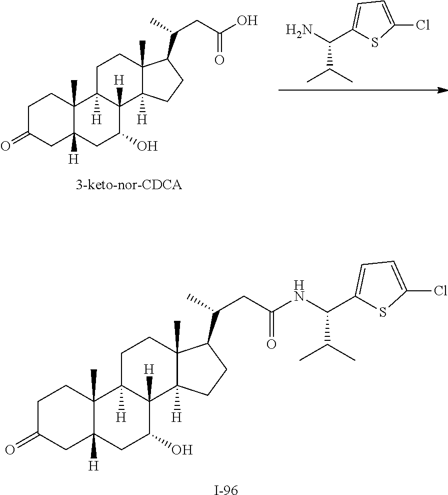

The compound I-96 was obtained by a synthetic method similar to that in Example 1 using 3-keto-nor-CDCA and I-A-9-S as raw materials.

¹HNMR(CDCl₃, 400 MHz): δ 6.78-6.68 (m, 2H), 5.69 (s, 1H), 5.06-4.96 (m, 1H), 3.94 (q, J=3.0 Hz, 1H), 3.47-3.34 (m, 1H), 2.48-2.35 (m, 2H), 2.27-2.13 (m, 2H), 2.13-1.92 (m, 7H), 1.83-1.73 (m, 3H), 1.71-1.32 (m, 7H), 1.32-1.14 (m, 1H), 1.21 (s, 3H), 1.05-0.92 (m, 12H), 0.76 (s, 3H).

ESI-MS m/z: 570.24[M+Na]⁺

### Example 97 Synthesis of Compound I-97

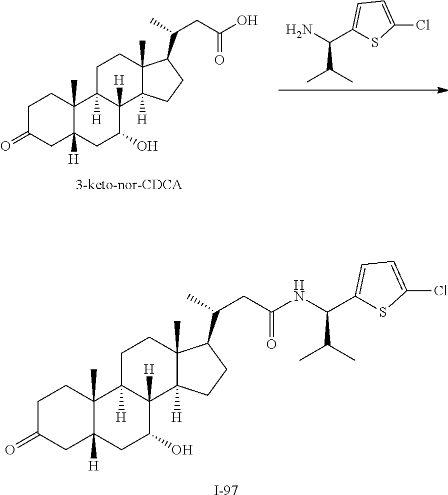

The compound I-97 was obtained by a synthetic method similar to that in Example 1 using 3-keto-nor-CDCA and I-A-9-R as raw materials.

¹HNMR(CDCl₃, 400 MHz): δ 6.79-6.68 (m, 2H), 5.69 (s, 1H), 5.06-4.97 (m, 1H), 3.94 (q, J=3.0 Hz, 1H), 3.47-3.34 (m, 1H), 2.48-2.35 (m, 2H), 2.27-2.13 (m, 2H), 2.13-1.92 (m, 7H), 1.84-1.73 (m, 3H), 1.71-1.32 (m, 7H), 1.32-1.14 (m, 1H), 1.21 (s, 3H), 1.05-0.92 (m, 12H), 0.76 (s, 3H).

ESI-MS m/z: 570.24[M+Na]⁺

### Example 98 Synthesis of Compound I-98

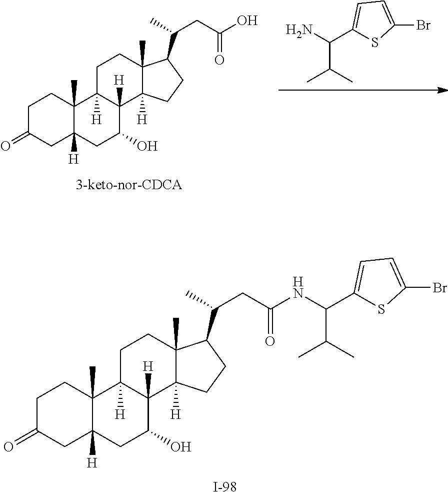

The compound I-98 was obtained by a synthetic method similar to that in Example 1 using 3-keto-nor-CDCA and I-A-10 as raw materials.

¹HNMR(CDCl₃, 400 MHz):

ESI-MS m/z: 613.13[M+Na]⁺

### Example 99 Synthesis of Compound I-99

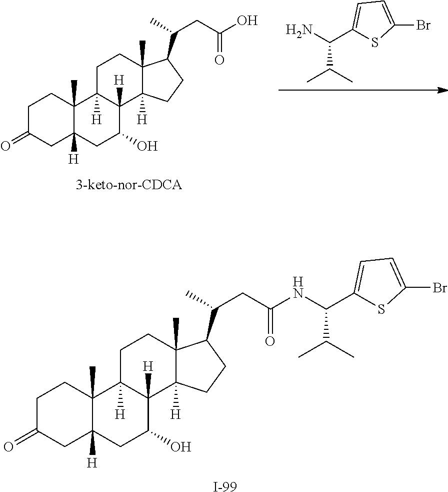

The compound I-99 was obtained by a synthetic method similar to that in Example 1 using 3-keto-nor-CDCA and I-A-10-S as raw materials.

¹HNMR(CDCl₃, 400 MHz):

ESI-MS m/z: 613.13[M+Na]⁺

### Example 100 Synthesis of Compound I-100

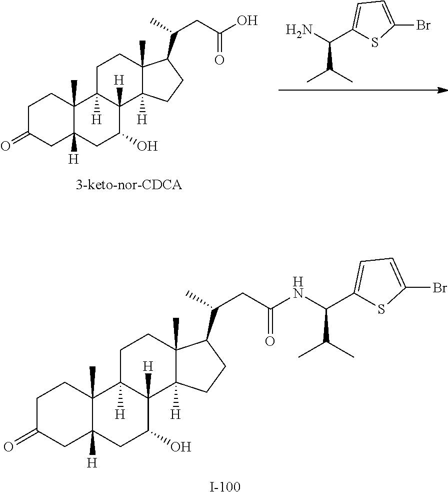

The compound I-100 was obtained by a synthetic method similar to that in Example 1 using 3-keto-nor-CDCA and I-A-10-R as raw materials.

¹HNMR(CDCl₃, 400 MHz):

ESI-MS m/z: 613.13[M+Na]⁺

### Example 101 Synthesis of Compound I-101

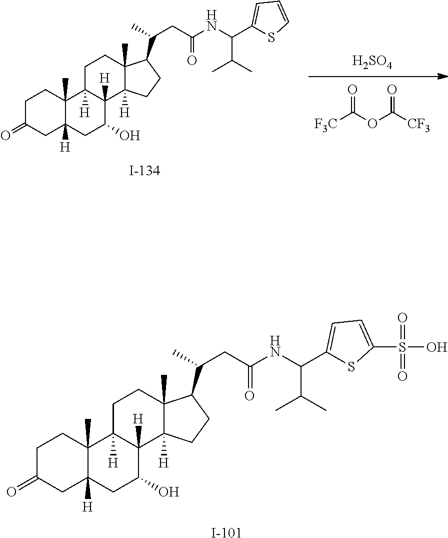

The compound I-101 was obtained by a synthetic method similar that in Example 34 using the compound I-134 as raw materials.

ESI-MS m/z: 615.15[M+Na]⁺

### Example 102 Synthesis of Compound I-102

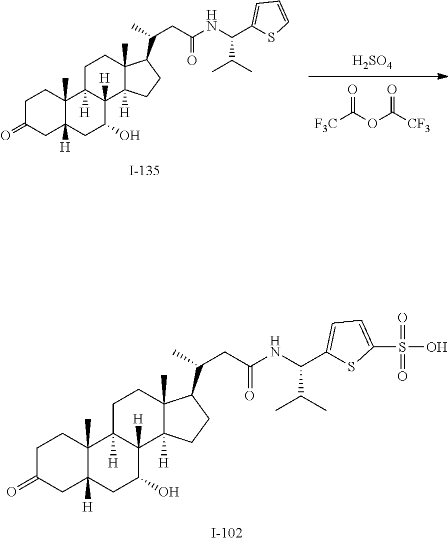

The compound I-102 was obtained by a synthetic method similar that in Example 34 using the compound I-135 as raw materials.

ESI-MS m/z: 615.15[M+Na]⁺

### Example 103 Synthesis of Compound I-103

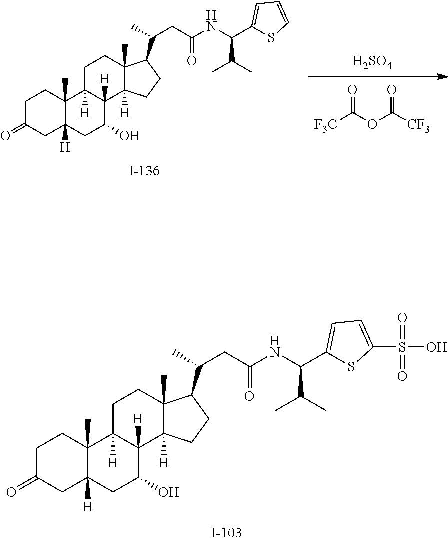

The compound I-103 was obtained by a synthetic method similar that in Example 34 using the compound I-136 as raw materials.

ESI-MS m/z: 615.15[M+Na]⁺

### Example 104 Synthesis of Compound I-104

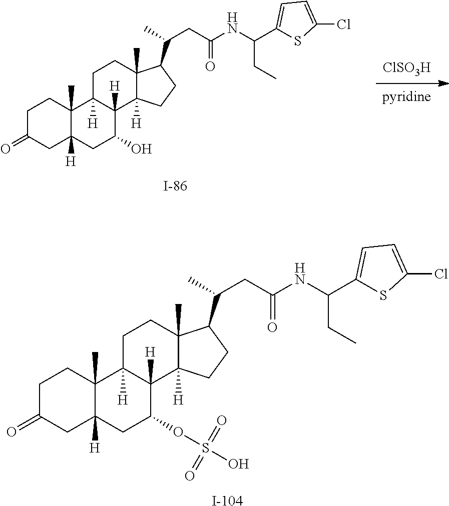

0.6 g of the compound I-86 (1.12 mmol) was added into a 100 ml single-necked flask, 30 ml of pyridine was added, a mixture was cooled in an ice-salt bath to −15° C., and 0.45 g of chlorosulfonic acid (3.86 mmol) was slowly added dropwise with stirring, the reaction system was warmed to room temperature and continued to react for 24 h. After the complete reaction was confirmed by TLC, 100 ml of saturated saline and 20 ml of 30% dilute hydrochloric acid were added, and stirred for 1 min. 70 ml of ethyl acetate and 30 ml of methanol were added, stirred for 1 min, and allowed to stand for stratification. An organic phase was retained, and an aqueous phase was extracted twice with a mixture of 35 ml of ethyl acetate and 15 ml of methanol; the organic phases were combined and dried over anhydrous sodium sulfate. The sodium sulfate was removed by filtration and the organic phase was concentrated under reduced pressure to obtain a foamy solid, namely the compound I-104.

¹HNMR(d6-DMSO, 400 MHz): δ 8.30-8.23 (m, 1H), 6.97-6.91 (m, 1H), 6.80 (d, J=3.8 Hz, 1H), 4.87 (q, J=8.0 Hz, 1H), 4.17 (s, 1H), 3.30-3.19 (m, 1H), 2.39-2.29 (m, 1H), 2.25-2.16 (m, 2H), 2.03-1.88 (m, 7H), 1.85-1.79 (m, 3H), 1.76-1.65 (m, 3H), 1.54-1.39 (m, 3H), 1.37-1.15 (m, 6H), 0.98-0.82 (m, 10H), 0.67 (s, 3H).

ESI-MS m/z: 615.15[M−1]⁻

0.1 g of the compound I-104 was dissolved in 10 ml of a mixed solution of ethanol/water (2:1), a sodium hydroxide solution was added, and the solvent was concentrated to dryness; a resulting product was redissolved with 10 ml of ethanol, 10 ml of methyl tert-butyl ether was added, a white solid was separated out, filtered with suction and dried to obtain a sodium salt I-104-1 of the compound I-104, with a structure being as follows:

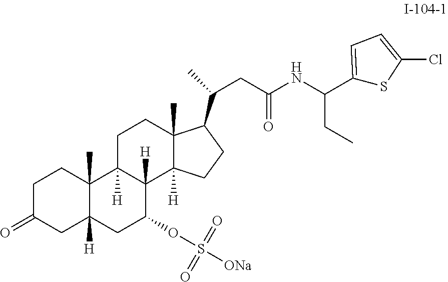

### Example 105 Synthesis of Compound I-105

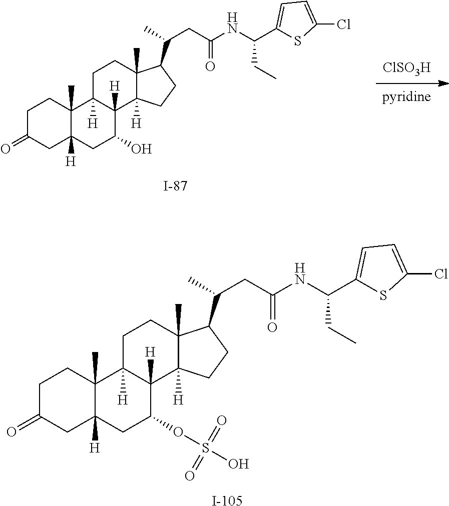

The compound I-105 was obtained by a synthetic method similar that in Example 104 using the compound I-87 as raw materials.

¹HNMR(d6-DMSO, 400 MHz): δ 8.26 (d, J=8.4 Hz, 1H), 6.94 (d, J=3.8 Hz, 1H), 6.78 (d, J=3.8 Hz, 1H), 4.87 (q, J=8.0 Hz, 1H), 4.17 (s, 1H), 3.29-3.19 (m, 1H), 2.38-2.28 (m, 1H), 2.25-2.15 (m, 2H), 2.03-1.87 (m, 7H), 1.84-1.79 (m, 3H), 1.76-1.65 (m, 3H), 1.54-1.39 (m, 3H), 1.39-1.15 (m, 6H), 0.98-0.82 (m, 10H), 0.67 (s, 3H).

ESI-MS m/z: 615.15[M−1]⁻

The compound I-105 was prepared into a sodium salt I-105-1 according to the method described in Example 104, with a structure being as follows:

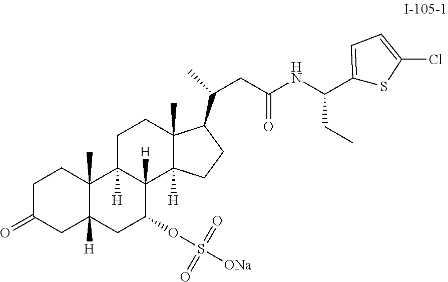

### Example 106 Synthesis of Compound I-106

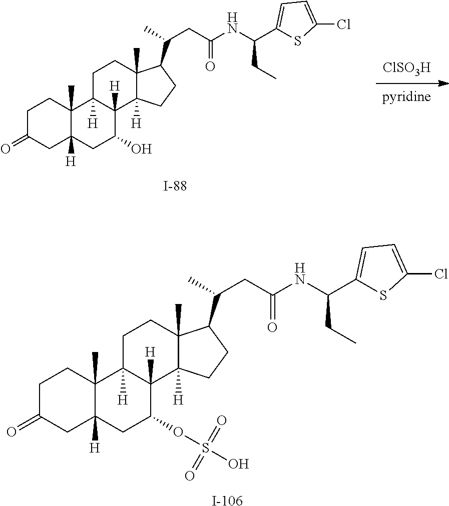

The compound I-106 was obtained by a synthetic method similar that in Example 104 using the compound I-88 as raw materials.

¹HNMR(d6-DMSO, 400 MHz): δ 8.27 (d, J=8.4 Hz, 1H), 6.94 (d, J=3.8 Hz, 1H), 6.79 (d, J=3.8 Hz, 1H), 4.87 (q, J=8.0 Hz, 1H), 4.17 (s, 1H), 3.30-3.18 (m, 1H), 2.39-2.28 (m, 1H), 2.25-2.16 (m, 2H), 2.03-1.88 (m, 7H), 1.84-1.79 (m, 3H), 1.75-1.65 (m, 3H), 1.54-1.39 (m, 3H), 1.37-1.15 (m, 6H), 0.98-0.82 (m, 10H), 0.67 (s, 3H).

ESI-MS m/z: 615.15[M−1]⁻

The compound I-106 was prepared into a sodium salt I-106-1 according to the method described in Example 104, with a structure being as follows:

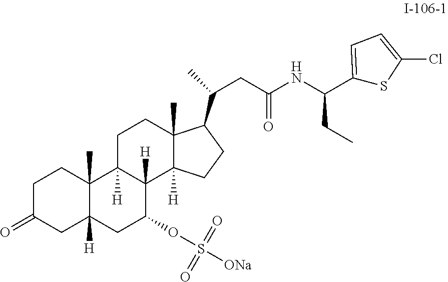

### Example 107 Synthesis of Compound I-107

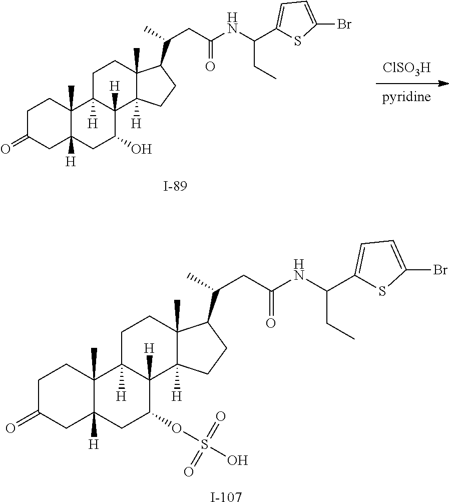

The compound I-107 was obtained by a synthetic method similar that in Example 104 using the compound I-89 as raw materials.

¹HNMR(d6-DMSO, 400 MHz): δ 8.30-8.23 (m, 1H), 7.04 (d, J=3.8 Hz, 1H), 6.80-6.74 (m, 1H), 4.92-4.84 (m, 1H), 4.17 (s, 1H), 3.30-3.18 (m, 1H), 2.27-2.17 (m, 2H), 2.04-1.90 (m, 6H), 1.86-1.76 (m, 4H), 1.73-1.68 (m, 3H), 1.49-1.43 (m, 3H), 1.38-1.16 (m, 4H), 0.96 (s, 3H), 0.92-0.84 (m, 9H), 0.67 (s, 3H).

ESI-MS m/z: 656.06[M−1]⁻

The compound I-107 was prepared into a sodium salt I-107-1 according to the method described in Example 104, with a structure being as follows:

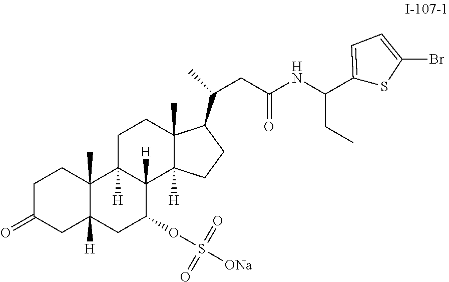

### Example 108 Synthesis of Compound I-108

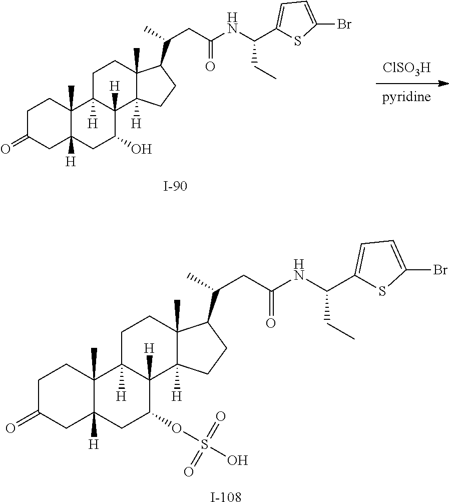

The compound I-108 was obtained by a synthetic method similar that in Example 104 using the compound I-90 as raw materials.

¹HNMR(d6-DMSO, 400 MHz): δ 8.28 (d, J=8.5 Hz, 1H), 7.04 (s, 1H), 6.77 (s, 1H), 4.92-4.84 (m, 1H), 4.17 (s, 1H), 3.31-3.19 (m, 1H), 2.26-2.16 (m, 2H), 2.04-1.90 (m, 6H), 1.87-1.75 (m, 4H), 1.75-1.67 (m, 3H), 1.48-1.43 (m, 3H), 1.37-1.17 (m, 4H), 0.96 (s, 3H), 0.92-0.84 (m, 9H), 0.66 (s, 3H).

ESI-MS m/z: 656.06[M−1]⁻

The compound I-108 was prepared into a sodium salt I-108-1 according to the method described in Example 104, with a structure being as follows:

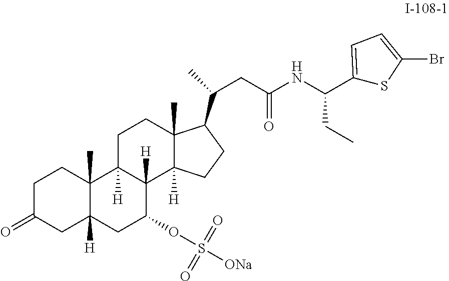

### Example 109 Synthesis of Compound I-109

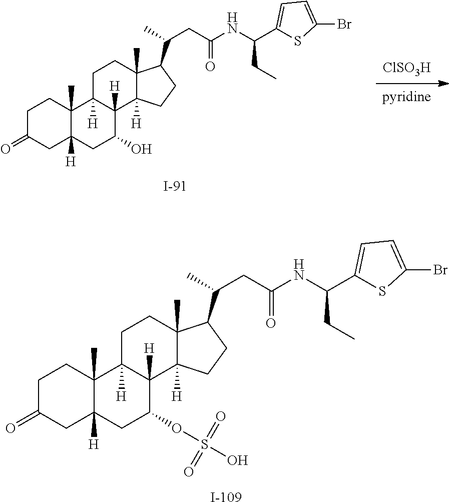

The compound I-109 was obtained by a synthetic method similar that in Example 104 using the compound I-91 as raw materials.

¹HNMR(d6-DMSO, 400 MHz): δ 8.27 (d, J=8.5 Hz, 1H), 7.04 (s, 1H), 6.77 (s, 1H), 4.92-4.84 (m, 1H), 4.17 (s, 1H), 3.30-3.18 (m, 1H), 2.25-2.17 (m, 2H), 2.04-1.90 (m, 6H), 1.87-1.76 (m, 4H), 1.73-1.68 (m, 3H), 1.48-1.43 (m, 3H), 1.37-1.16 (m, 4H), 0.96 (s, 3H), 0.92-0.84 (m, 9H), 0.67 (s, 3H).

ESI-MS m/z: 656.06[M−1]⁻

The compound I-109 was prepared into a sodium salt I-109-1 according to the method described in Example 104, with a structure being as follows:

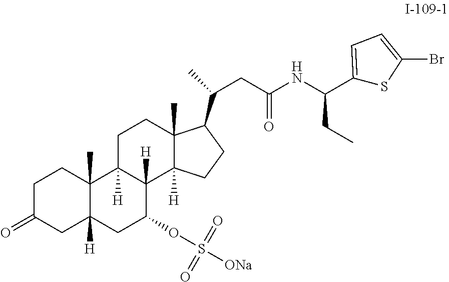

### Example 110 Synthesis of Compound I-110

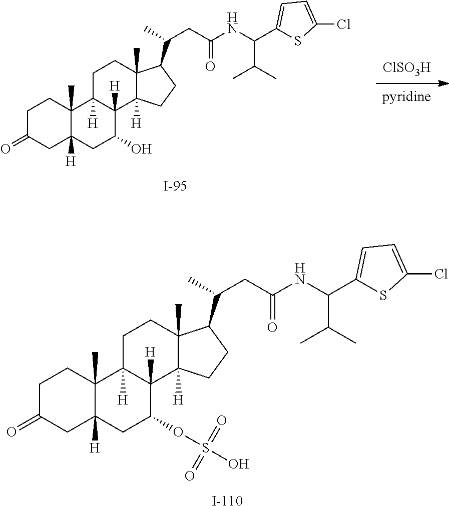

The compound I-110 was obtained by a synthetic method similar that in Example 104 using the compound I-95 as raw materials.

¹HNMR(d6-DMSO, 400 MHz): δ 8.26-8.18 (m, 1H), 6.94 (d, J=3.8 Hz, 1H), 6.82-6.76 (m, 1H), 4.82-4.73 (m, 1H), 4.17 (s, 1H), 3.31-3.18 (m, 1H), 2.40-2.67 (m, 1H), 2.25-2.16 (m, 2H), 2.11-1.88 (m, 7H), 1.91-1.65 (m, 6H), 1.65-1.15 (m, 7H), 1.14-1.02 (m, 1H), 0.96 (s, 3H), 0.93-0.85 (m, 7H), 0.83 (d, J=6.7 Hz, 3H), 0.66 (s, 3H).

ESI-MS m/z: 626.14 [M−1]⁻

The compound I-110 was prepared into a sodium salt I-110-1 according to the method described in Example 104, with a structure being as follows:

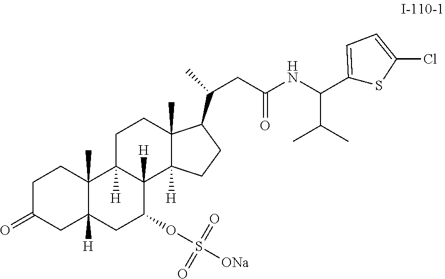

### Example 111 Synthesis of Compound I-111

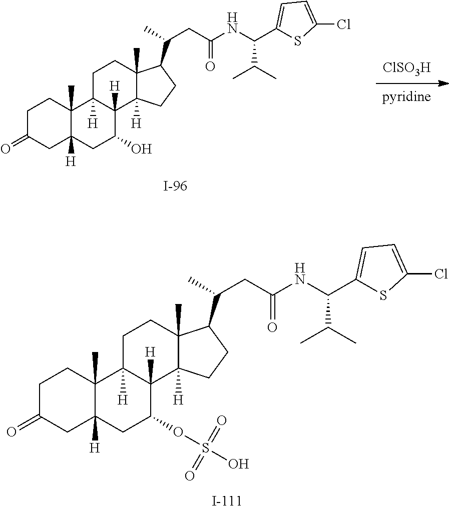

The compound I-111 was obtained by a synthetic method similar that in Example 104 using the compound I-96 as raw materials.

¹HNMR(d6-DMSO, 400 MHz): δ 8.21 (d, J=8.8 Hz, 1H), 6.94 (d, J=3.8 Hz, 1H), 6.79 (d, J=3.8 Hz, 1H), 4.81-4.71 (m, 1H), 4.17 (s, 1H), 3.31-3.18 (m, 1H), 2.40-2.67 (m, 1H), 2.25-2.16 (m, 2H), 2.11-1.88 (m, 7H), 1.91-1.78 (m, 3H), 1.78-1.65 (m, 3H), 1.65-1.38 (m, 3H), 1.38-1.02 (m, 5H), 0.96 (s, 3H), 0.93-0.85 (m, 7H), 0.83 (d, J=6.7 Hz, 3H), 0.66 (s, 3H).

ESI-MS m/z: 626.14 [M−1]⁻

The compound I-111 was prepared into a sodium salt I-111-1 according to the method described in Example 104, with a structure being as follows:

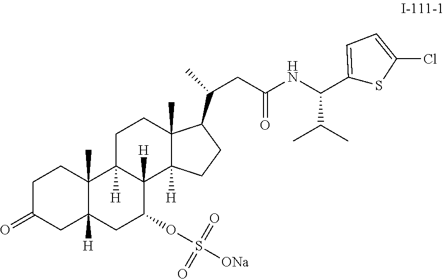

### Example 112 Synthesis of Compound I-112

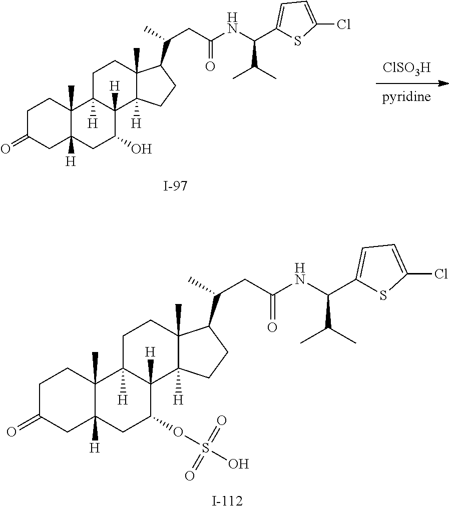

The compound I-112 was obtained by a synthetic method similar that in Example 104 using the compound I-97 as raw materials.

¹HNMR(d6-DMSO, 400 MHz): δ 8.22 (d, J=8.8 Hz, 1H), 6.94 (d, J=3.8 Hz, 1H), 6.79 (d, J=3.8 Hz, 1H), 4.78 (t, J=8.2 Hz, 1H), 4.17 (s, 1H), 3.31-3.18 (m, 1H), 2.40-2.67 (m, 1H), 2.25-2.16 (m, 2H), 2.11-1.88 (m, 7H), 1.91-1.79 (m, 3H), 1.77-1.65 (m, 3H), 1.65-1.38 (m, 3H), 1.38-1.15 (m, 4H), 1.14-1.02 (m, 1H), 0.96 (s, 3H), 0.93-0.85 (m, 7H), 0.83 (d, J=6.7 Hz, 3H), 0.66 (s, 3H).

ESI-MS m/z: 626.14 [M−1]⁻

The compound I-112 was prepared into a sodium salt I-112-1 according to the method described in Example 104, with a structure being as follows:

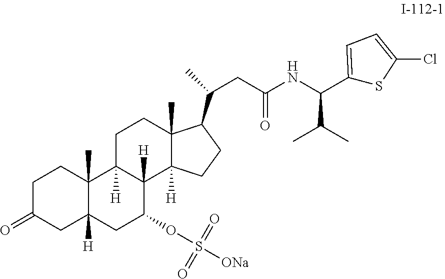

### Example 113 Synthesis of Compound I-113

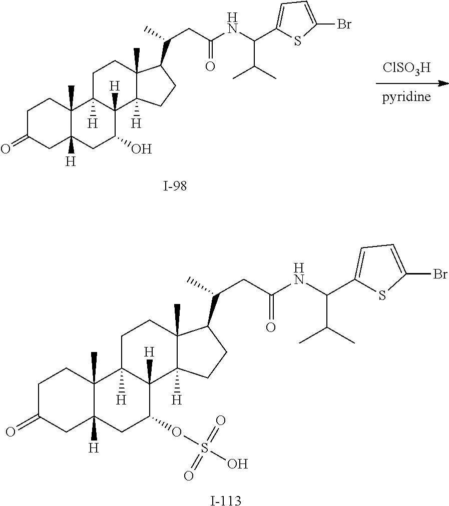

The compound I-113 was obtained by a synthetic method similar that in Example 104 using the compound I-98 as raw materials.

¹HNMR(CD₃OD, 400 MHz): δ 6.98-6.92 (m, 1H), 6.79-6.73 (m, 1H), 4.87-4.80 (m, 1H), 4.54-4.43 (m, 1H), 3.63-3.55 (m, 1H), 3.47-3.35 (m, 1H), 2.42-2.22 (m, 3H), 2.20-2.01 (m, 8H), 2.01-1.57 (m, 7H), 1.57-1.15 (m, 6H), 1.08 (s, 3H), 1.05-0.89 (m, 10H), 0.77 (s, 3H).

ESI-MS m/z: 670.15[M−1]⁻

The compound I-113 was prepared into a sodium salt I-113-1 according to the method described in Example 104, with a structure being as follows:

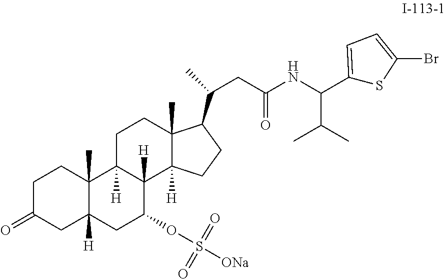

### Example 114 Synthesis of Compound I-114

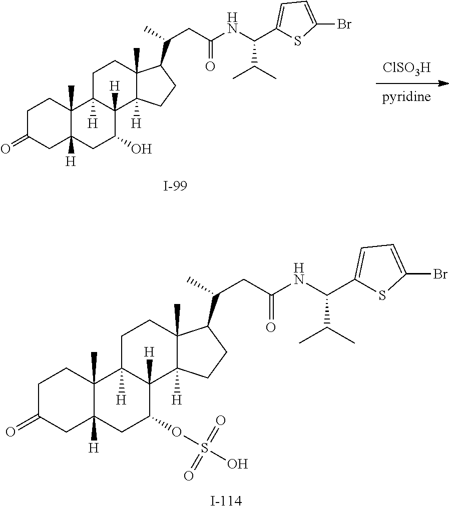

The compound I-114 was obtained by a synthetic method similar that in Example 104 using the compound I-99 as raw materials.

¹HNMR(CD₃OD, 400 MHz): δ 6.95 (d, J=3.8 Hz, 1H), 6.75 (d, J=3.8 Hz, 1H), 4.84 (d, J=8.4 Hz, 1H), 4.54-4.44 (m, 1H), 3.58 (t, J=6.5 Hz, 1H), 3.47-3.35 (m, 1H), 2.42-2.21 (m, 3H), 2.21-2.01 (m, 8H), 2.01-1.77 (m, 4H), 1.77-1.56 (m, 3H), 1.56-1.15 (m, 6H), 1.08 (s, 3H), 1.05-0.89 (m, 10H), 0.77 (s, 3H).

ESI-MS m/z: 670.15[M−1]⁻

The compound I-114 was prepared into a sodium salt I-114-1 according to the method described in Example 104, with a structure being as follows:

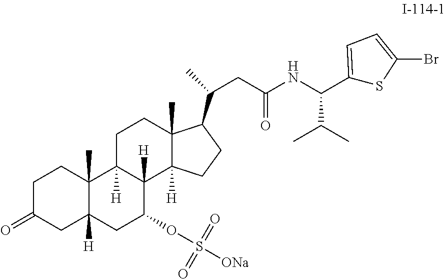

### Example 115 Synthesis of Compound I-115

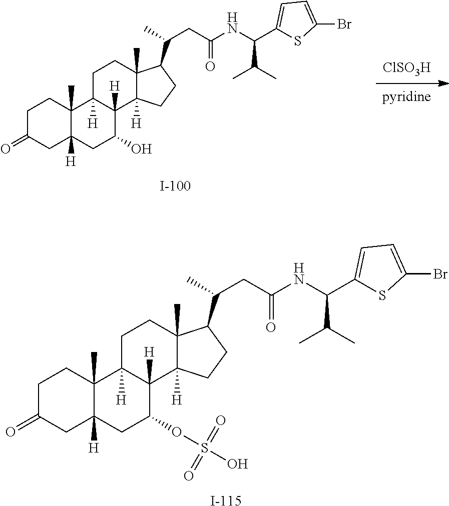

The compound I-115 was obtained by a synthetic method similar that in Example 104 using the compound I-100 as raw materials.

¹HNMR(CD₃OD, 400 MHz): δ 6.95 (d, J=3.8 Hz, 1H), 6.76 (d, J=3.8 Hz, 1H), 4.84 (d, J=8.4 Hz, 1H), 4.54-4.43 (m, 1H), 3.59 (t, J=6.5 Hz, 1H), 3.47-3.35 (m, 1H), 2.42-2.22 (m, 3H), 2.20-2.01 (m, 8H), 2.01-1.77 (m, 4H), 1.77-1.56 (m, 3H), 1.57-1.15 (m, 6H), 1.08 (s, 3H), 1.05-0.89 (m, 10H), 0.77 (s, 3H).

ESI-MS m/z: 670.15[M−1]⁻

The compound I-115 was prepared into a sodium salt I-115-1 according to the method described in Example 104, with a structure being as follows:

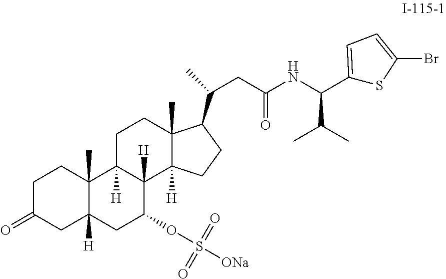

### Example 116 Synthesis of Compound I-116

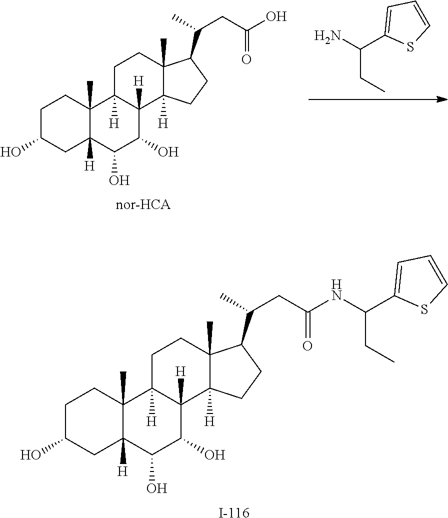

The compound I-116 was obtained by a synthetic method similar to that in Example 1 using nor-HCA and I-A-1 as raw materials.

¹HNMR(CDCl₃, 400 MHz): δ 7.26-7.19 (m, 1H), 7.00-6.95 (m, 2H), 5.74 (S, 1H), 5.28-5.19 (m, 1H) 3.86 (s, 2H), 2.39-2.20 (m, 4H), 2.10-1.80 (m, 5H), 1.74-1.69 (m, 6H), 1.57-1.45 (m, 4H), 1.43-1.28 (m, 2H), 1.32-1.14 (m, 4H), 1.12-0.94 (m, 7H), 0.92 (s, 3H), 0.66 (s, 3H).

ESI-MS m/z: 516.18[M−1]⁻

### Example 117 Synthesis of Compound I-117

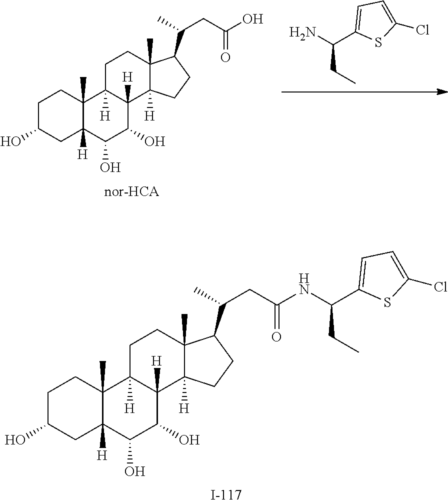

The compound I-117 was obtained by a synthetic method similar to that in Example 1 using nor-HCA and I-A-6-R as raw materials.

¹HNMR(CDCl₃, 400 MHz): δ 6.80-6.71 (m, 2H), 5.74 (s, 1H), 5.14-5.03 (m, 1H), 3.91-3.84 (m, 2H), 3.52-3.37 (m, 1H), 2.38-2.24 (m, 4H), 2.18-2.04 (m, 5H), 2.02-1.79 (m, 3H), 1.75-1.64 (m, 5H), 1.54-1.47 (m, 3H), 1.43-1.12 (m, 4H), 1.12-0.94 (m, 7H), 0.93 (s, 3H), 0.66 (s, 3H).

ESI-MS m/z: 550.22[M−1]⁻

### Example 118 Synthesis of Compound I-118

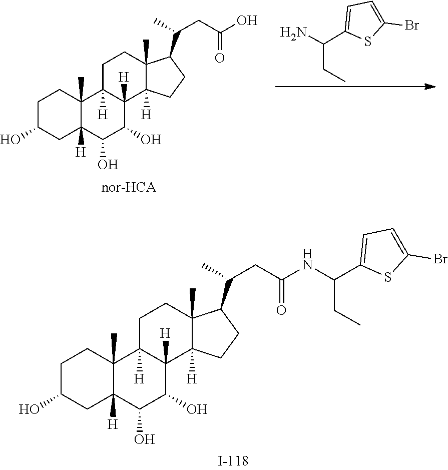

The compound I-118 was obtained by a synthetic method similar to that in Example 1 using nor-HCA and I-A-7 as raw materials.

ESI-MS m/z: 594.10[M−1]⁻

### Example 119 Synthesis of Compound I-119

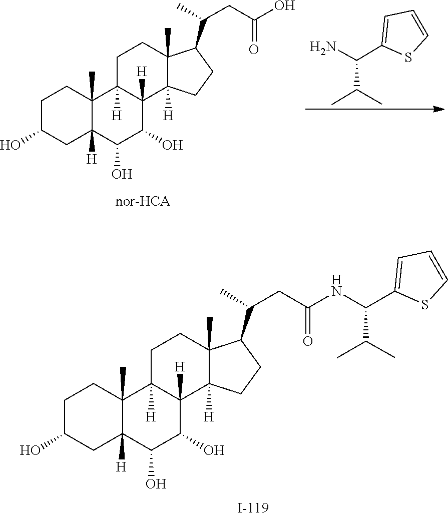

The compound I-119 was obtained by a synthetic method similar to that in Example 1 using nor-HCA and I-A-8 as raw materials.

¹HNMR(CDCl₃, 400 MHz): δ 7.24-7.16 (m, 1H), 6.99-6.91 (m, 2H), 5.89 (s, 1H), 5.18-5.01 (m, 1H), 3.90-3.84 (m, 2H), 3.52-3.41 (m, 1H), 2.47-2.06 (m, 2H), 2.09-1.87 (m, 4H), 1.89-1.79 (m, 2H), 1.78-1.63 (m, 5H), 1.53-1.46 (m, 3H), 1.40-1.21 (m, 3H), 1.24-1.12 (m, 4H), 1.13-0.89 (m, 10H), 0.92 (s, 3H), 0.65 (d, J=2.5 Hz, 3H).

ESI-MS m/z: 530.22[M−1]⁻

### Example 120 Synthesis of Compound I-120

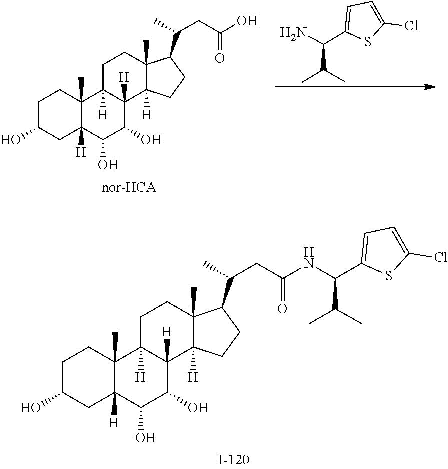

The compound I-120 was obtained by a synthetic method similar to that in Example 1 using nor-HCA and I-A-9-R as raw materials.

ESI-MS m/z: 564.16[M−1]⁻

### Example 121 Synthesis of Compound I-121

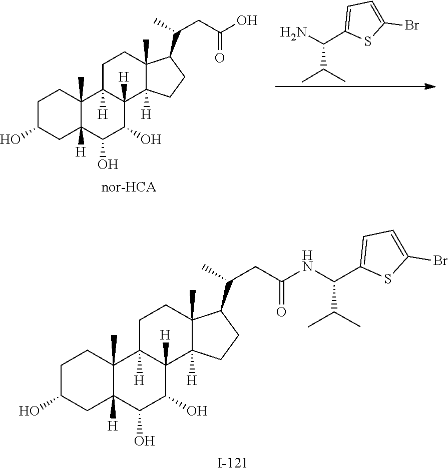

The compound I-121 was obtained by a synthetic method similar to that in Example 1 using nor-HCA and I-A-10-S as raw materials.

ESI-MS m/z: 608.21[M−1]⁻

### Example 122 Synthesis of Compound I-122

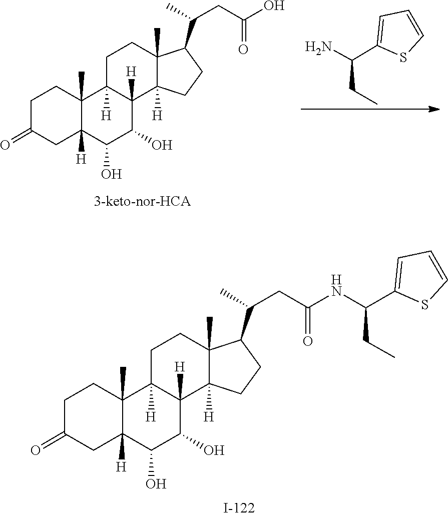

The compound I-122 was obtained by a synthetic method similar to that in Example 1 using 3-keto-nor-HCA and 1-A-1-R as raw materials.

ESI-MS m/z: 514.23[M−1]⁻

### Example 123 Synthesis of Compound I-123

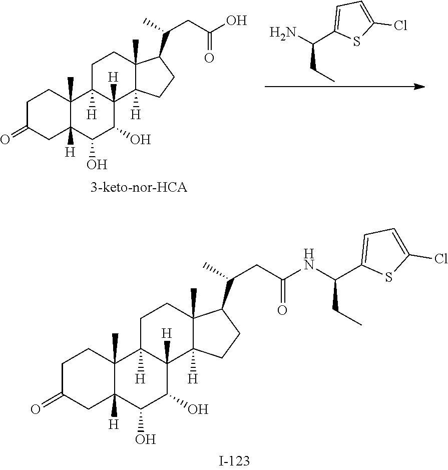

The compound I-123 was obtained by a synthetic method similar to that in Example 1 using 3-keto-nor-HCA and I-A-6-R as raw materials.

ESI-MS m/z: 548.23[M−1]⁻

### Example 124 Synthesis of Compound I-124

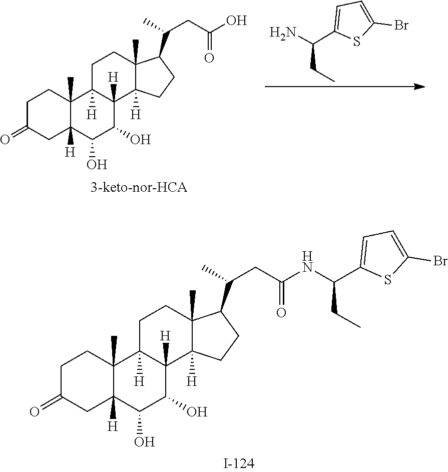

The compound I-124 was obtained by a synthetic method similar to that in Example 1 using 3-keto-nor-HCA and I-A-6-R as raw materials.

ESI-MS m/z: 592.16[M−1]⁻

### Example 125 Synthesis of Compound I-125

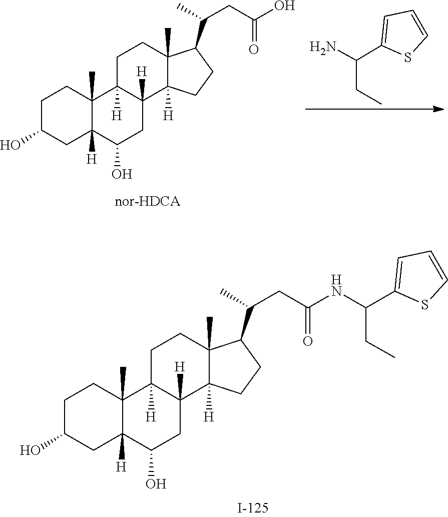

The compound I-125 was obtained by a synthetic method similar to that in Example 1 using nor-HDCA and I-A-1 as raw materials.

¹HNMR(CDCl₃, 400 MHz): δ 7.25-7.18 (m, 1H), 7.01-6.94 (m, 2H), 5.67 (s, 1H), 5.30-5.20 (m, 1H), 4.13-4.03 (m, 1H), 3.70-3.58 (m, 1H), 2.47-2.39 (m, 1H), 2.04-1.87 (m, 5H), 1.84-1.76 (m, 6H), 1.75-1.57 (m, 3H), 1.50-1.22 (m, 6H), 1.26-1.08 (m, 6H), 1.08-0.94 (m, 7H), 0.93 (s, 3H), 0.70 (d, J=3.1 Hz, 3H).

ESI-MS m/z:500.17[M−1]⁻

### Example 126 Synthesis of Compound I-126

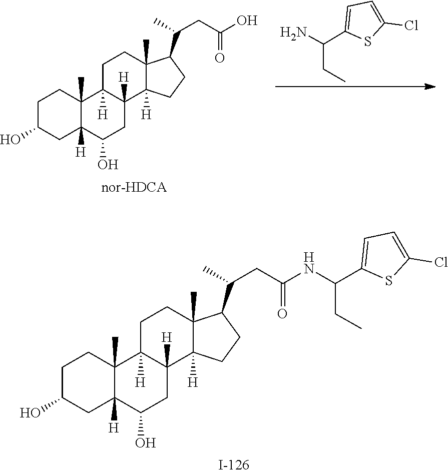

The compound I-126 was obtained by a synthetic method similar to that in Example 1 using nor-HDCA and I-A-6 as raw materials.

¹HNMR(CDCl₃, 400 MHz): δ 6.79-6.71 (m, 2H), 5.66 (s, 1H), 5.16-5.05 (m, 1H), 4.13-4.03 (m, 1H), 3.71-3.58 (m, 1H), 2.46-2.32 (m, 1H), 2.03-1.95 (m, 4H), 1.95-1.59 (m, 9H), 1.52-1.36 (m, 4H), 1.29-1.15 (m, 2H), 1.15-1.08 (m, 5H), 1.08-0.74 (m, 7H), 0.93 (s, 3H), 0.70 (d, J=2.9 Hz, 3H).

ESI-MS m/z:534.19[M−1]⁻

### Example 127 Synthesis of Compound I-127

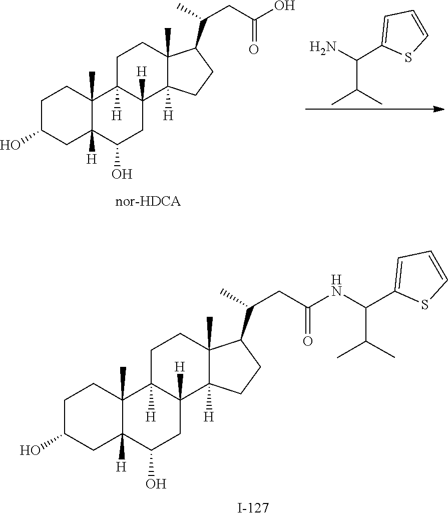

The compound I-127 was obtained by a synthetic method similar to that in Example 1 using nor-HDCA and I-A-8 as raw materials.

¹HNMR(CDCl₃, 400 MHz): δ 7.24-7.17 (m, 1H), 7.00-6.92 (m, 2H), 5.69 (s, 1H), 5.21-5.10 (m, 1H), 4.13-4.03 (m, 1H), 3.71-3.58 (m, 1H), 2.44-2.22 (m, 2H), 2.04-1.87 (m, 6H), 1.84-1.60 (m, 9H), 1.47-1.05 (m, 9H), 1.05-0.88 (m, 12H), 0.73-0.67 (m, 3H).

ESI-MS m/z:514.26[M−1]⁻

### Example 128 Synthesis of Compound I-128

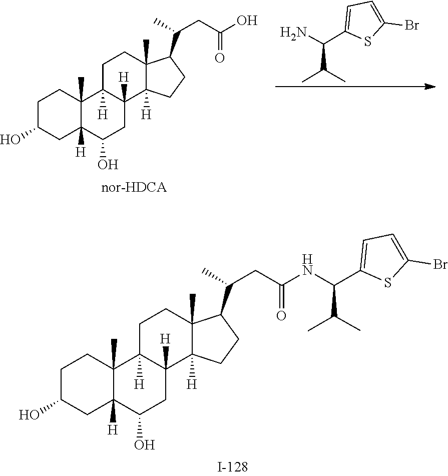

The compound I-128 was obtained by a synthetic method similar to that in Example 1 using nor-HDCA and 1-A-10-R as raw materials.

ESI-MS m/z:592.11[M−1]⁻

### Example 129 Synthesis of Compound I-129

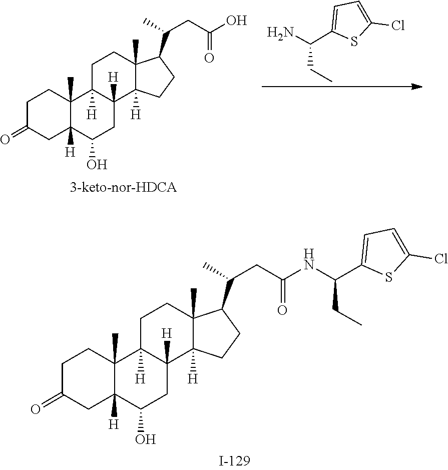

The compound I-129 was obtained by a synthetic method similar to that in Example 1 using 3-keto-nor-HDCA and I-A-6-S as raw materials.

ESI-MS m/z:531.07[M−1]⁻

### Example 130 Synthesis of Compound I-130

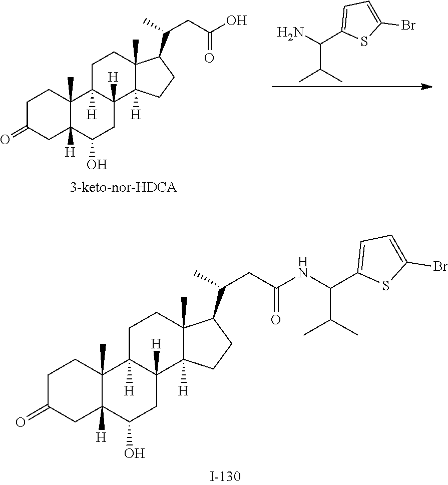

The compound I-130 was obtained by a synthetic method similar to that in Example 1 using 3-keto-nor-HDCA and I-A-10 as raw materials.

ESI-MS m/z:590.20[M−1]⁻

### Example 131 Synthesis of Compound I-131

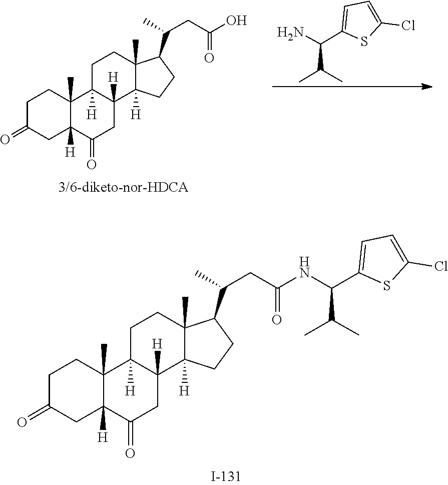

The compound I-131 was obtained by a synthetic method similar to that in Example 1 using 3,6-diketo-nor-HDCA, and I-A-9-R as raw materials.

ESI-MS m/z:544.19[M−1]⁻

### Example 132 Synthesis of Compound I-132

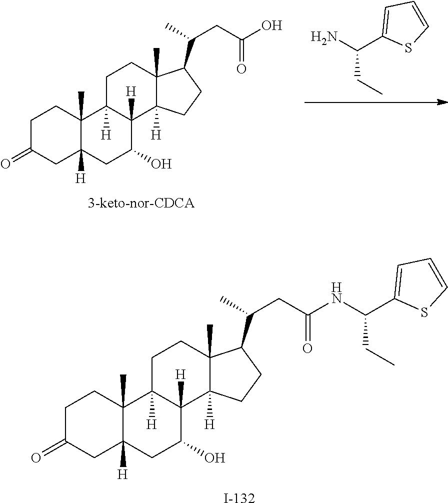

The compound I-132 was obtained by a synthetic method similar to that in Example 1 using 3-keto-nor-CDCA and I-A-1-S as raw materials.

¹HNMR(CDCl₃, 400 MHz): δ 7.25-7.19 (m, 1H), 9.00-6.94 (m, 2H), 5.64 (brs, 1H), 5.25 (q, J=7.6 Hz, 1H), 3.94 (s, 1H), 3.45-3.34 (m, 1H), 2.48-2.35 (m, 2H), 2.26-1.74 (m, 10H), 1.74-1.34 (m, 10H), 1.27-1.12 (m, 4H), 1.05-0.95 (m, 9H), 0.76 (s, 3H).

ESI-MS m/z: 498.11[M−1]⁻

### Example 133 Synthesis of Compound I-133

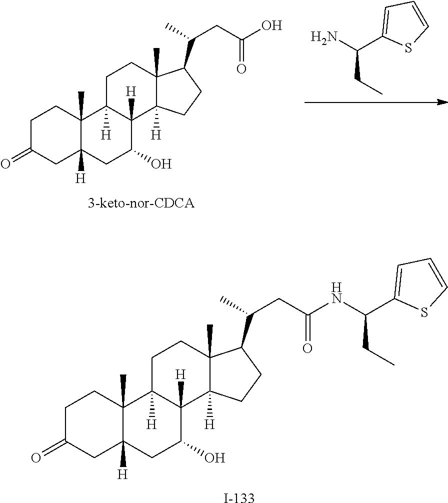

The compound I-133 was obtained by a synthetic method similar to that in Example 1 using 3-keto-nor-CDCA and I-A-1-R as raw materials.

¹HNMR(CDCl₃, 400 MHz): δ 7.25-7.19 (m, 1H), 6.96 (s, 2H), 5.64 (brs, 1H), 5.25 (q, J=7.6 Hz, 1H), 3.97-3.91 (m, 1H), 3.46-3.35 (m, 1H), 2.48-2.36 (m, 2H), 2.27-1.74 (m, 10H), 1.74-1.33 (m, 10H), 1.27-1.12 (m, 4H), 1.05-0.95 (m, 9H), 0.76 (s, 3H).

ESI-MS m/z: 498.11[M−1]⁻

### Example 134 Synthesis of Compound I-134

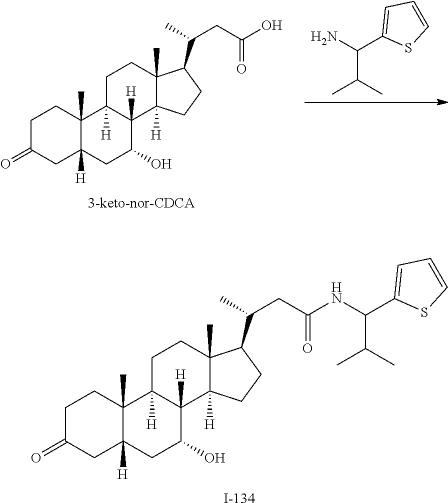

The compound I-134 was obtained by a synthetic method similar to that in Example 1 using 3-keto-nor-CDCA and I-A-8 as raw materials.

¹HNMR(CDCl₃, 400 MHz): δ 7.24-7.17 (m, 1H), 7.00-6.91 (m, 2H), 5.70 (s, 1H), 5.17 (t, J=8.1 Hz, 1H), 3.98-3.91 (m, 1H), 3.47-3.34 (m, 1H), 2.50-2.35 (m, 2H), 2.27-2.16 (m, 2H), 2.16-1.92 (m, 7H), 1.91-1.73 (m, 2H), 1.62-1.51 (m, 2H), 1.51-1.36 (m, 4H), 1.38-1.15 (m, 4H), 1.06-0.91 (m, 13H), 0.75 (d, J=4.9 Hz, 3H).

ESI-MS m/z: 512.13[M−1]⁻

### Example 135 Synthesis of Compound I-135

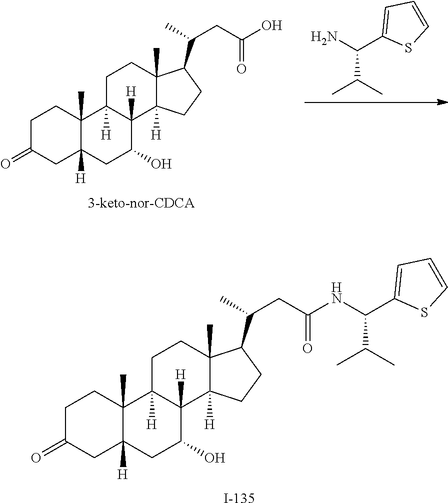

The compound I-135 was obtained by a synthetic method similar to that in Example 1 using 3-keto-nor-CDCA and I-A-8-S as raw materials.

¹HNMR(CDCl₃, 400 MHz): δ 7.24-7.16 (m, 1H), 7.00-6.90 (m, 2H), 5.70 (s, 1H), 5.21-5.12 (m, 1H), 3.98-3.90 (m, 1H), 3.47-3.34 (m, 1H), 2.50-2.35 (m, 2H), 2.26-2.16 (m, 2H), 2.16-1.91 (m, 7H), 1.91-1.73 (m, 2H), 1.62-1.38 (m, 6H), 1.38-1.15 (m, 4H), 1.06-0.91 (m, 13H), 0.75 (d, J=4.9 Hz, 3H).

ESI-MS m/z: 512.13[M−1]⁻

### Example 136 Synthesis of Compound I-136

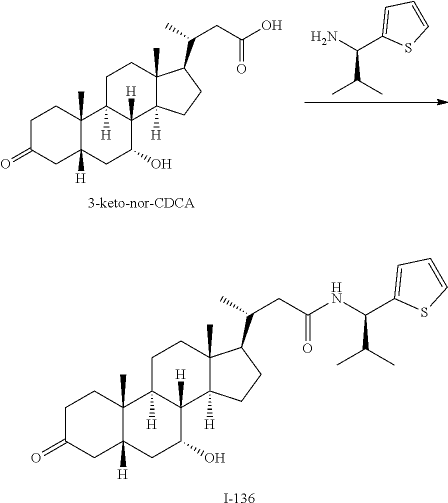

The compound I-136 was obtained by a synthetic method similar to that in Example 1 using 3-keto-nor-CDCA and I-A-8-R as raw materials.

¹HNMR(CDCl₃, 400 MHz): δ 7.25-7.18 (m, 1H), 7.01-6.91 (m, 2H), 5.71 (s, 1H), 5.17 (t, J=8.1 Hz, 1H), 3.99-3.90 (m, 1H), 3.47-3.34 (m, 1H), 2.50-2.35 (m, 2H), 2.26-2.16 (m, 2H), 2.16-1.92 (m, 7H), 1.93-1.73 (m, 2H), 1.62-1.52 (m, 2H), 1.51-1.36 (m, 4H), 1.36-1.15 (m, 4H), 1.06-0.91 (m, 13H), 0.75 (d, J=4.9 Hz, 3H).

ESI-MS m/z: 512.13[M−1]⁻

### Example 137 Synthesis of Compound I-137

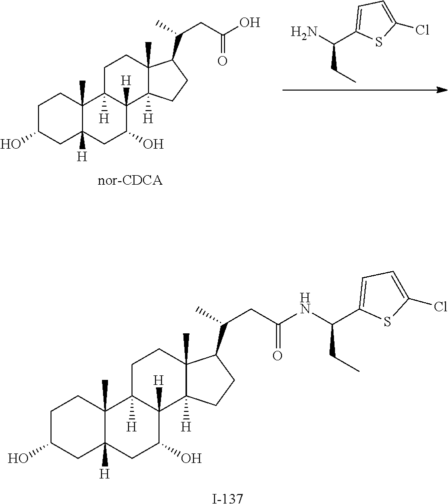

The compound I-137 was obtained by a synthetic method similar to that in Example 1 using nor-CDCA and I-A-6-R as raw materials.

¹HNMR(CDCl₃, 400 MHz): δ 6.79-6.70 (m, 2H), 5.73 (d, J=8.5 Hz, 1H), 5.10 (q, J=7.6 Hz, 1H), 3.90-3.83 (m, 1H), 3.55-3.42 (m, 1H), 2.46-2.37 (m, 1H), 2.31-2.14 (m, 1H), 2.12-1.96 (m, 4H), 1.96-1.61 (m, 7H), 1.57-1.45 (m, 3H), 1.45-1.08 (m, 10H), 1.06-0.94 (m, 7H), 0.92 (s, 3H), 0.72 (s, 3H).

ESI-MS m/z: 535.11[M−1]⁻

### Example 138 Synthesis of Compound I-138

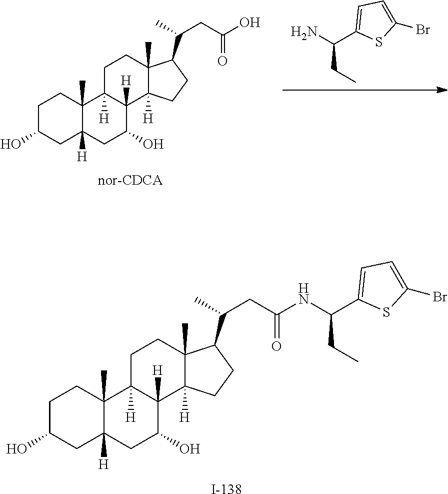

The compound I-138 was obtained by a synthetic method similar to that in Example 1 using nor-CDCA and I-A-7-R as raw materials.

¹HNMR(CDCl₃, 400 MHz): δ 6.89 (d, J=3.8 Hz, 1H), 6.75-6.69 (d, J=3.7 Hz, 1H), 5.85-5.78 (m, 1H), 5.12 (q, J=7.7 Hz, 1H), 3.90-3.82 (m, 1H), 3.54-3.42 (m, 1H), 2.45-2.33 (m, 1H), 2.29-2.14 (m, 1H), 2.09-1.92 (m, 5H), 1.92-1.62 (m, 6H), 1.57-1.45 (m, 4H), 1.45-1.09 (m, 8H), 1.06-0.94 (m, 7H), 0.92 (s, 3H), 0.71 (s, 3H).

ESI-MS m/z: 578.20[M−1]⁻

### Example 139 Synthesis of Compound I-139

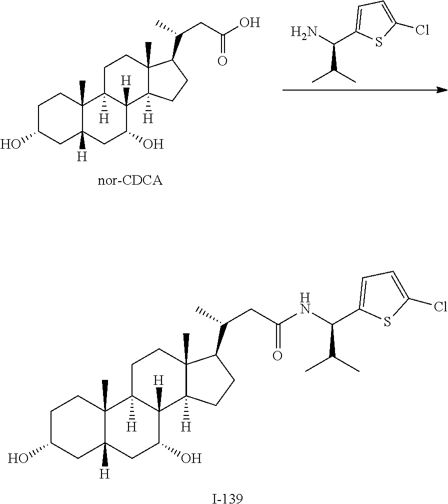

The compound I-139 was obtained by a synthetic method similar to that in Example 1 using nor-CDCA and I-A-9-R as raw materials.

ESI-MS m/z: 548.10[M−1]⁻

### Example 140 Synthesis of Compound I-140

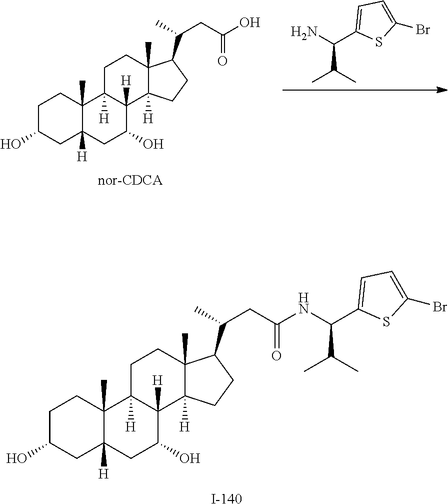

The compound I-140 was obtained by a synthetic method similar to that in Example 1 using nor-CDCA and I-A-10-R as raw materials.

ESI-MS m/z: 592.03[M−1]⁻

### Example 141 Synthesis of Compound I-141

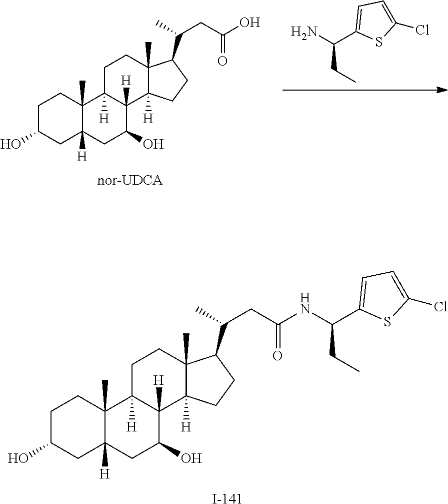

The compound I-141 was obtained by a synthetic method similar to that in Example 1 using nor-UDCA and I-A-6-R as raw materials.

ESI-MS m/z: 535.11[M−1]⁻

### Example 142 Synthesis of Compound I-142

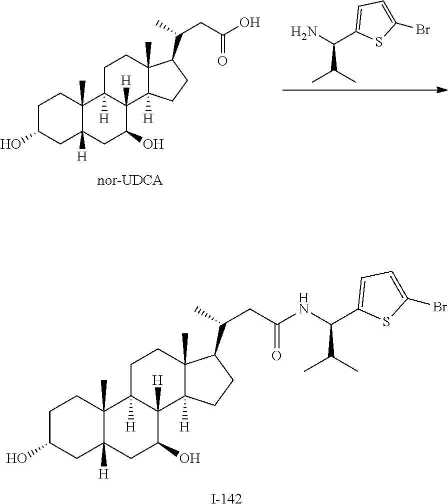

The compound I-142 was obtained by a synthetic method similar to that in Example 1 using nor-UDCA and I-A-10-R as raw materials.

ESI-MS m/z: 592.03[M−1]⁻

### Example 143 Synthesis of Compound I-143

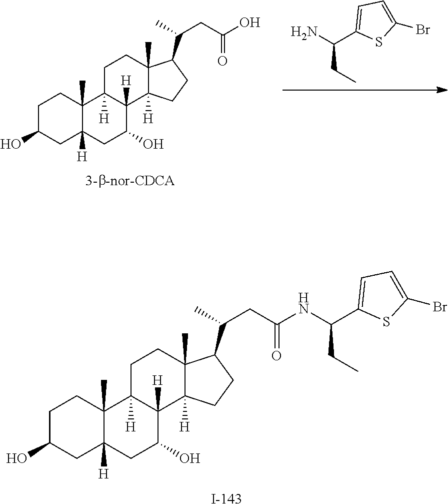

The compound I-143 was obtained by a synthetic method similar to that in Example 1 using 3-β-nor-CDCA and I-A-7-R as raw materials.

¹HNMR(CDCl₃, 400 MHz): δ 6.90 (d, J=3.7 Hz, 1H), 6.69 (d, J=3.7, 1H), 5.65-5.57 (m, 1H), 5.10-5.00 (m, 1H), 4.12-4.06 (m, 1H), 3.92-3.85 (m, 1H), 2.53-2.38 (m, 2H), 2.12-1.96 (m, 4H), 1.87-1.60 (m, 6H), 1.59-1.41 (m, 5H), 1.45-1.28 (m, 2H), 1.32-1.17 (m, 4H), 1.21-1.08 (m, 2H), 1.06-0.94 (m, 7H), 0.97 (s, 3H), 0.73 (s, 3H).

ESI-MS m/z: 578.20 [M−1]⁻

### Example 144 Synthesis of Compound I-144

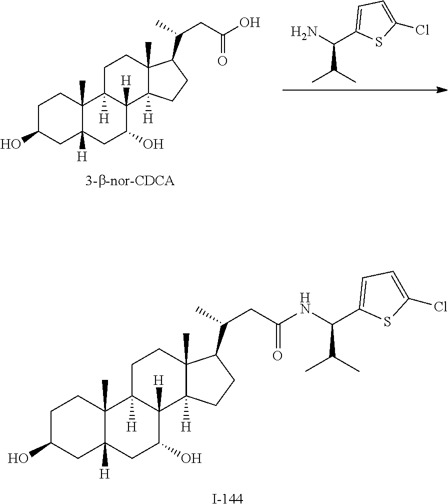

The compound I-144 was obtained by a synthetic method similar to that in Example 1 using 3-β-nor-CDCA and I-A-9-R as raw materials.

¹HNMR(CDCl₃, 400 MHz): δ 6.79-6.67 (m, 2H), 5.64-5.57 (m, 1H), 5.07-4.98 (m, 1H), 4.12-4.06 (m, 1H), 3.92-3.85 (m, 1H), 2.53-2.39 (m, 2H), 2.11-1.96 (m, 4H), 1.87-1.71 (m, 6H), 1.71-1.34 (m, 7H), 1.34-1.18 (m, 7H), 1.05-0.86 (m, 12H), 0.73 (s, 3H).

ESI-MS m/z: 548.10 [M−1]⁻

### Example 145 Synthesis of Compound I-145

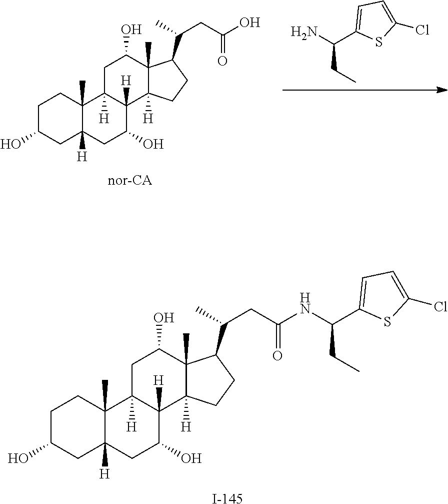

The compound I-145 was obtained by a synthetic method similar to that in Example 1 using nor-CA and I-A-6-R as raw materials.

ESI-MS m/z: 550.18[M−1]⁻

### Example 146 Synthesis of Compound I-146

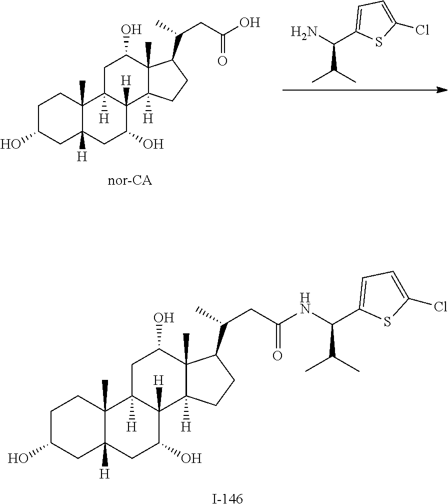

The compound I-146 was obtained by a synthetic method similar to that in Example 1 using nor-CA and I-A-9-R as raw materials.

ESI-MS m/z: 564.22[M−1]⁻

### Example 147 Synthesis of Compound I-147

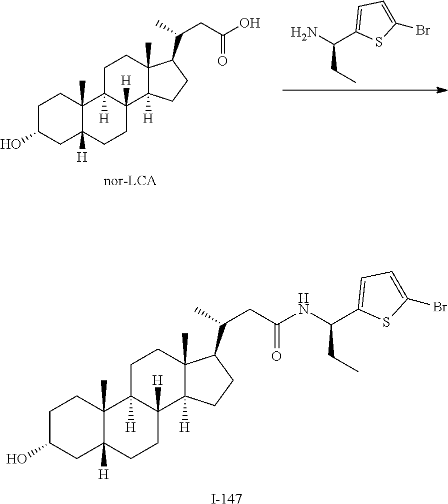

The compound I-147 was obtained by a synthetic method similar to that in Example 1 using nor-LCA and I-A-7-R as raw materials.

ESI-MS m/z: 562.12[M−1]⁻

### Example 148 Synthesis of Compound I-148

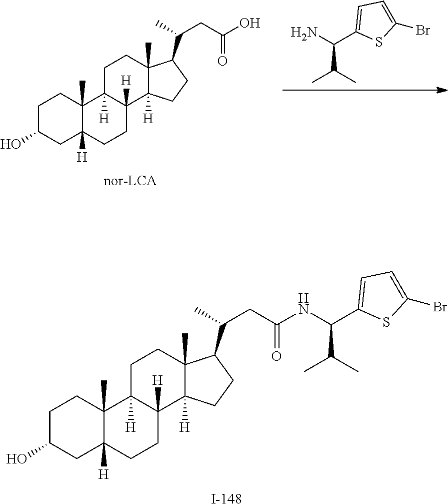

The compound I-148 was obtained by a synthetic method similar to that in Example 1 using nor-LCA and I-A-10-R as raw materials.

ESI-MS m/z: 577.12[M−1]⁻

### Example 149 Synthesis of Compound I-149

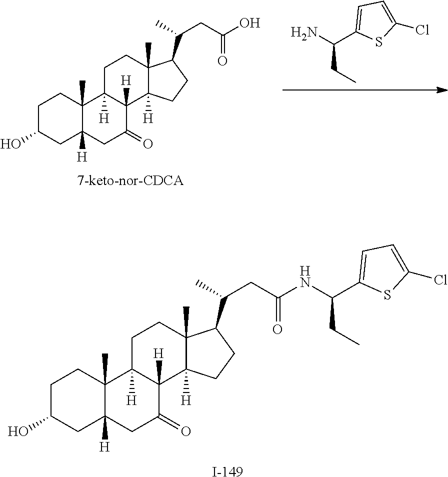

The compound I-149 was obtained by a synthetic method similar to that in Example 1 using 7-keto-nor-CDCA and I-A-6-R as raw materials.

ESI-MS m/z: 532.05[M−1]⁻

### Example 150 Synthesis of Compound I-150

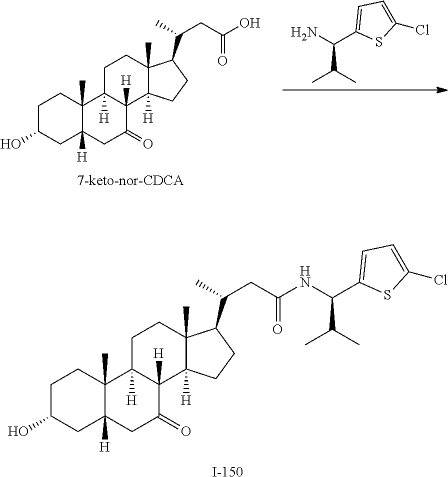

The compound I-150 was obtained by a synthetic method similar to that in Example 1 using 7-keto-nor-CDCA and I-A-9-R as raw materials.

ESI-MS m/z: 546.03[M−1]⁻

### Example 151 Synthesis of Compound I-151

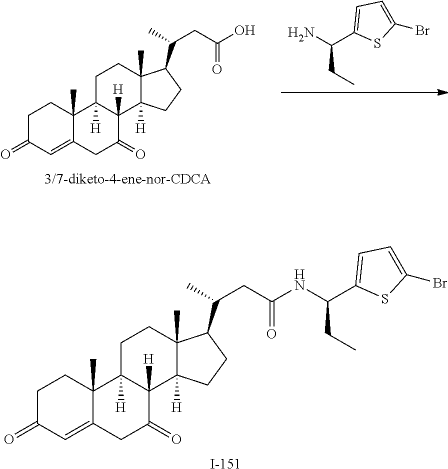

The compound I-151 was obtained by a synthetic method similar to that in Example 1 using 3,7-diketo-4-ene-nor-CDCA and I-A-7-R as raw materials.

ESI-MS m/z: 572.00[M−1]⁻

### Example 152 Synthesis of Compound I-152

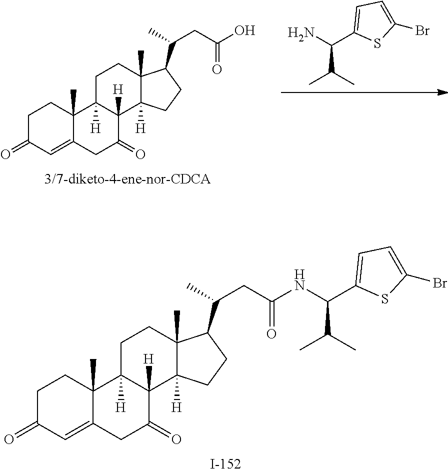

The compound I-152 was obtained by a synthetic method similar to that in Example 1 using 3,7-diketo-4-ene-nor-CDCA and I-A-10-R as raw materials.

ESI-MS m/z: 586.11[M−1]⁻

### Example 153 Synthesis of Compound I-153

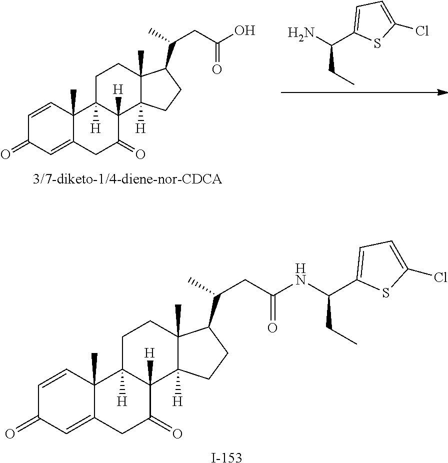

The compound I-153 was obtained by a synthetic method similar to that in Example 1 using 3,7-diketo-1,4-ene-nor-CDCA and I-A-6-R as raw materials.

ESI-MS m/z: 526.07[M−1]⁻

### Example 154 Synthesis of Compound I-154

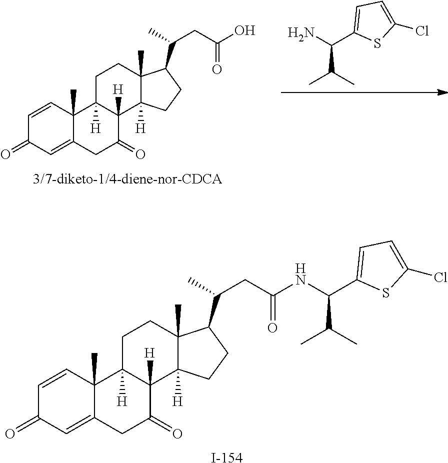

The compound I-154 was obtained by a synthetic method similar to that in Example 1 using 3,7-diketo-1,4-diene-nor-CDCA and I-A-9-R as raw materials.

ESI-MS m/z: 540.06[M−1]⁻

### Example 155 Synthesis of Compound I-155

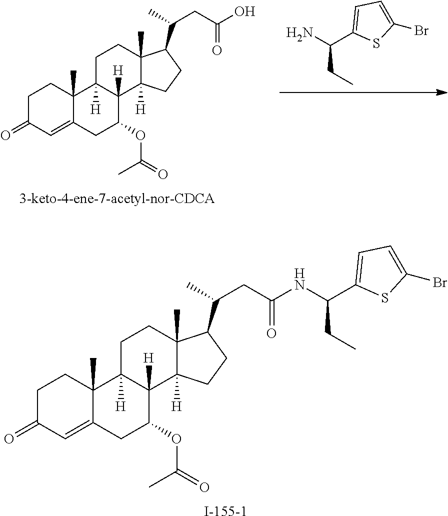

The compounds I-155-1 and I-155-2 were obtained by a synthetic method similar to that in Example 1 using 3-keto-4-ene-7-acetyl-nor-CDCA and I-A-7-R as raw materials through separation and purification by silica gel column chromatography; in which a structure of the compound I-155-1 was shown in the above reaction formula,

ESI-MS m/z: 616.22[M−1]⁻

a structure of the compound I-155-2 was shown as follows:

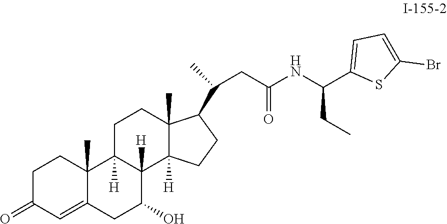

¹HNMR(CDCl₃, 400 MHz): δ 6.80-6.71 (m, 2H), 5.74 (s, 1H), 5.09 (q, J=7.7 Hz, 1H), 3.91-3.85 (m, 2H), 3.52-3.42 (m, 1H), 2.38-2.24 (m, 1H), 2.18-2.04 (m, 4H), 2.02-1.79 (m, 6H), 1.75-1.64 (m, 4H), 1.54-1.47 (m, 3H), 1.43-1.12 (m, 4H), 1.12-0.94 (m, 7H), 0.93 (s, 3H), 0.66 (s, 3H).

ESI-MS m/z: 576.46[M−1]⁻

### Example 156 Synthesis of Compound I-156

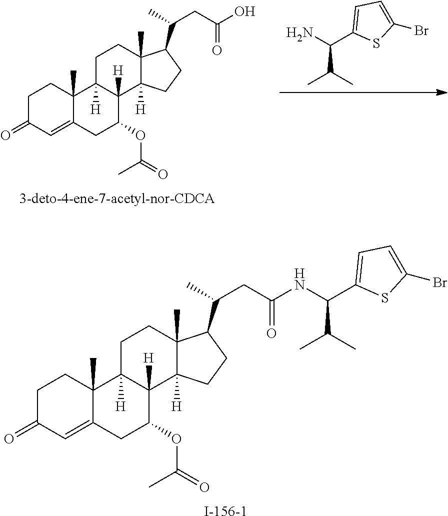

The compounds I-156-1 and I-156-2 were obtained by a synthetic method similar to that in Example 1 using 3-keto-4-ene-7-acetyl-nor-CDCA and 1-A-10-R as raw materials through separation and purification by silica gel column chromatography; in which a structure of the compound I-156-1 was shown in the above reaction formula,

ESI-MS m/z: 530.22[M−1]⁻

a structure of the compound I-156-2 was shown as follows:

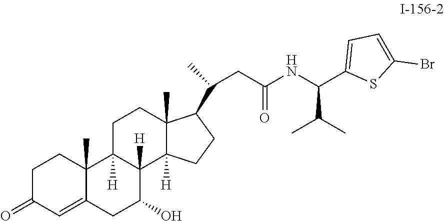

ESI-MS m/z: 576.46[M−1]−

### Example 157 Synthesis of Compound I-157

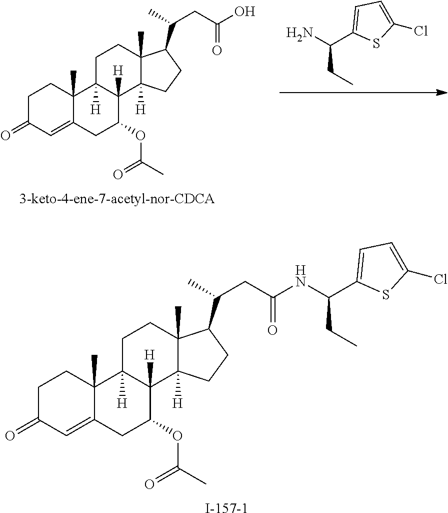

The compounds I-157-1 and I-157-2 were obtained by a synthetic method similar to that in Example 1 using 3-keto-4-ene-7-acetyl-nor-CDCA and I-A-6-R as raw materials through separation and purification by silica gel column chromatography; in which a structure of the compound I-157-1 was shown in the above reaction formula,

ESI-MS m/z: 572.11[M−1]⁻

a structure of the compound I-157-2 was shown as follows:

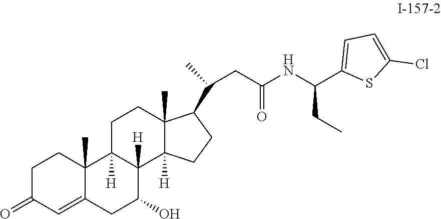

ESI-MS m/z: 530.08[M−1]⁻

### Example 158 Synthesis of Compound I-158

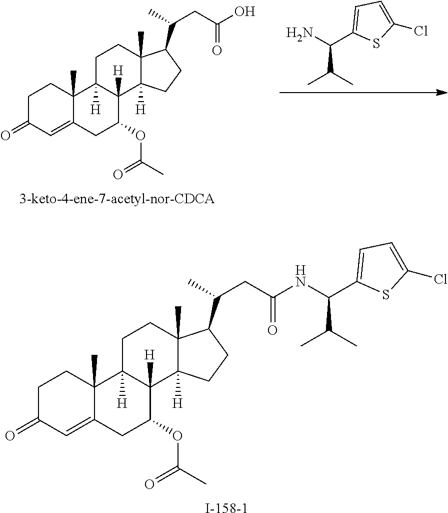

The compounds I-158-1 and I-158-2 were obtained by a synthetic method similar to that in Example 1 using 3-keto-4-ene-7-acetyl-nor-CDCA and I-A-9-R as raw materials through separation and purification by silica gel column chromatography; in which a structure of the compound I-158-1 was shown in the above reaction formula,

ESI-MS m/z: 586.06[M−1]⁻

a structure of the compound I-158-2 was shown as follows:

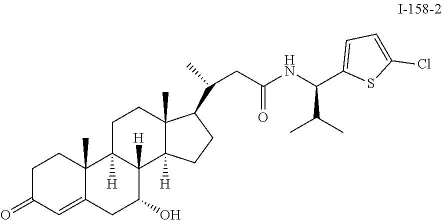

ESI-MS m/z: 544.09[M−1]⁻

### Example 159 Synthesis of Compound I-159

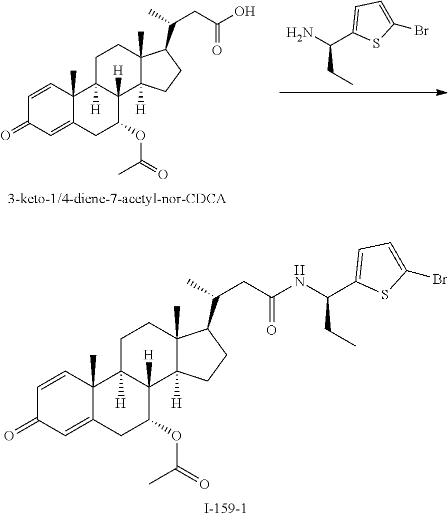

The compounds I-159-1 and I-159-2 were obtained by a synthetic method similar to that in Example 1 using 3-keto-1,4-diene-7-acetyl-nor-CDCA and I-A-7-R as raw materials through separation and purification by silica gel column chromatography; in which a structure of the compound I-159-1 was shown in the above reaction formula,

ESI-MS m/z: 614.20[M−1]⁻

a structure of the compound I-159-2 was shown as follows:

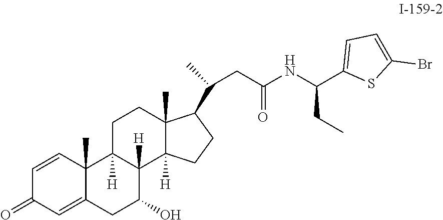

ESI-MS m/z: 572.15[M−1]⁻

### Example 160 Synthesis of Compound I-160

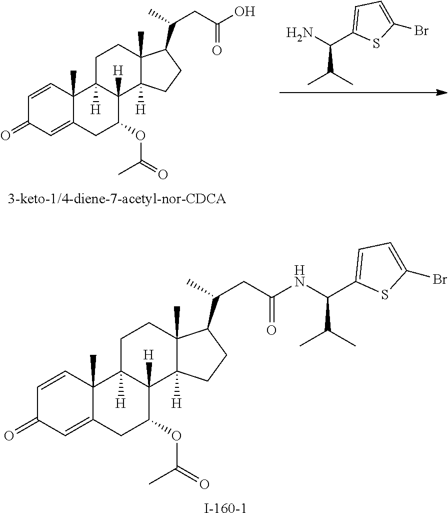

The compounds I-160-1 and I-160-2 were obtained by a synthetic method similar to that in Example 1 using 3-keto-1,4-diene-7-acetyl-nor-CDCA and I-A-10-R as raw materials through separation and purification by silica gel column chromatography; in which a structure of the compound I-160-1 was shown in the above reaction formula,

ESI-MS m/z: 628.20[M−1]⁻

a structure of the compound I-160-2 was shown as follows:

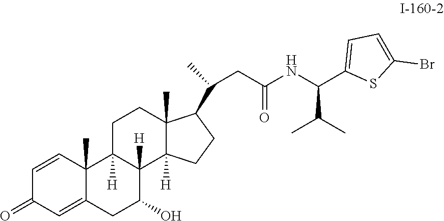

ESI-MS m/z: 586.08[M−1]⁻

### Example 161 Synthesis of Compound I-161

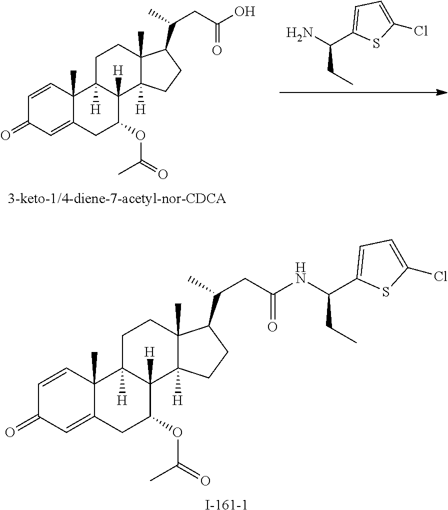

The compounds I-161-1 and I-161-2 were obtained by a synthetic method similar to that in Example 1 using 3-keto-1,4-diene-7-acetyl-nor-CDCA and I-A-6-R as raw materials through separation and purification by silica gel column chromatography; in which a structure of the compound I-161-1 was shown in the above reaction formula,

ESI-MS m/z: 570.11[M−1]⁻

a structure of the compound I-161-2 was shown as follows:

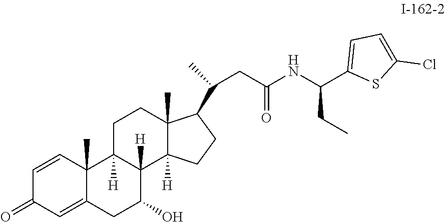

ESI-MS m/z: 528.06[M−1]⁻

### Example 162 Synthesis of Compound I-162

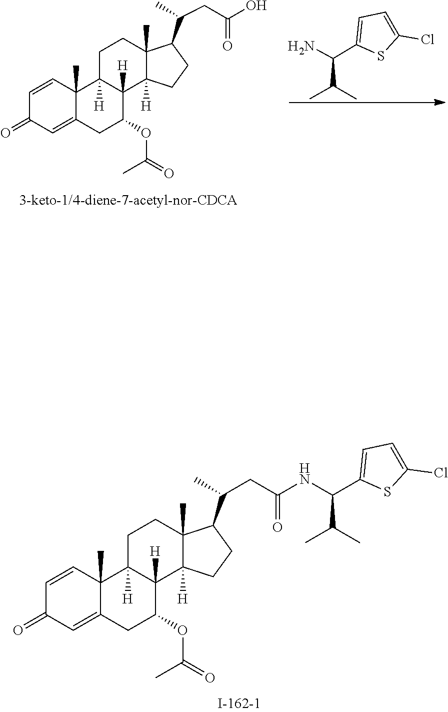

The compounds I-162-1 and I-162-2 were obtained by a synthetic method similar to that in Example 1 using 3-keto-1,4-diene-7-acetyl-nor-CDCA and I-A-9-R as raw materials through separation and purification by silica gel column chromatography; in which a structure of the compound I-162-1 was shown in the above reaction formula,

¹HNMR(CDCl₃, 400 MHz): δ 7.13-7.05 (m, 1H), 6.80-6.71 (m, 2H), 6.32-6.24 (m, 1H), 6.03 (s, 1H), 5.62-5.55 (m, 1H), 5.16-5.03 (m, 2H), 2.68-2.62 (m, 2H), 2.42-2.30 (m, 3H), 2.02 (s, 3H), 1.98-1.67 (m, 6H), 1.67-1.53 (m, 4H), 1.53-1.21 (m, 3H), 1.27 (s, 3H), 1.25-1.11 (m, 2H), 1.06-0.92 (m, 6H), 0.80 (s, 3H).

ESI-MS m/z: 584.13[M−1]⁻

a structure of the compound I-162-2 was shown as follows:

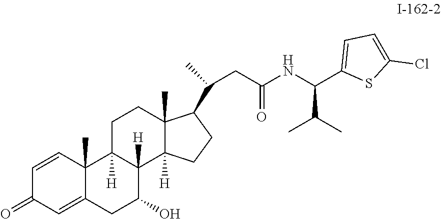

ESI-MS m/z: 542.08[M−1]⁻

### Example 163 Synthesis of Compound I-163

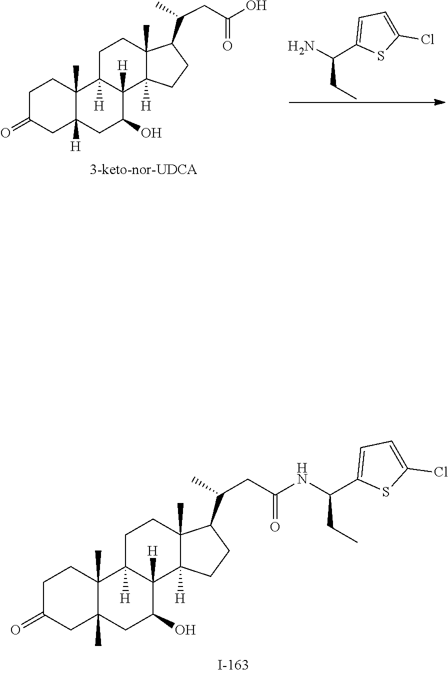

The compound I-163 was obtained by a synthetic method similar to that in Example 1 using 3-keto-nor-UDCA and I-A-6-R as raw materials.

ESI-MS m/z: 532.15[M−1]⁻

### Example 164 Synthesis of Compound I-164

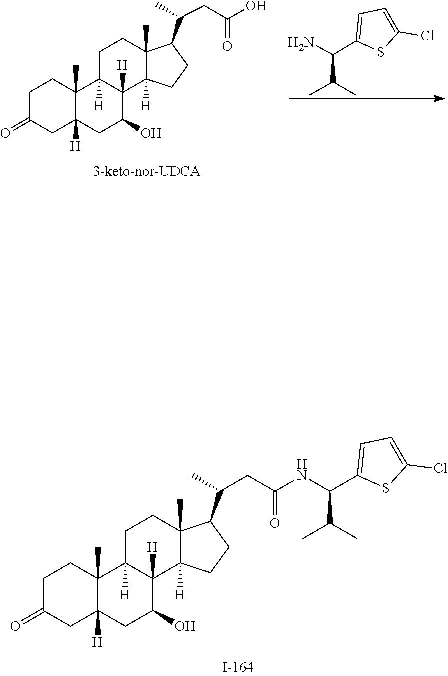

The compound I-164 was obtained by a synthetic method similar to that in Example 1 using 3-keto-nor-UDCA and I-A-9-R as raw materials.

ESI-MS m/z: 546.23[M−1]⁻

### Example 165 Synthesis of Compound I-165

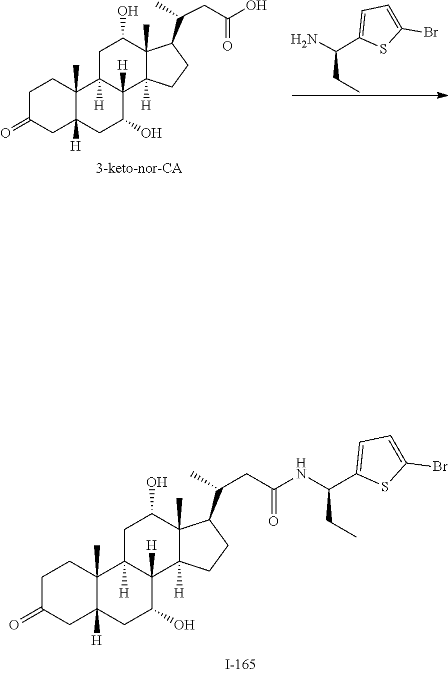

The compound I-165 was obtained by a synthetic method similar to that in Example 1 using 3-keto-nor-CA and I-A-7-R as raw materials.

ESI-MS m/z: 592.00[M−1]⁻

### Example 166 Synthesis of Compound I-166

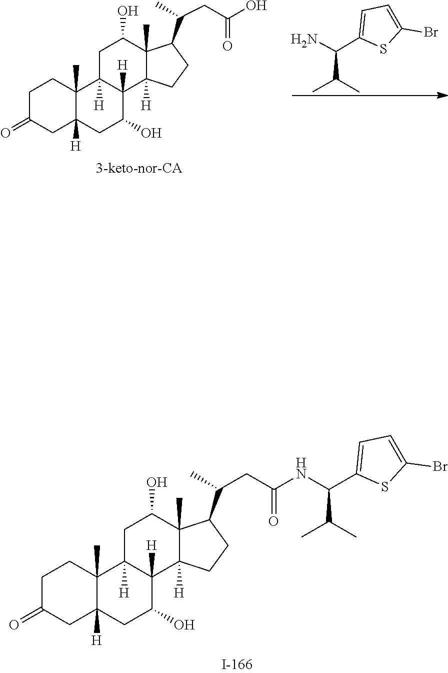

The compound I-166 was obtained by a synthetic method similar to that in Example 1 using 3-keto-nor-CA and I-A-10-R as raw materials.

ESI-MS m/z: 606.13[M−1]⁻

### Example 167 Synthesis of Compound I-167

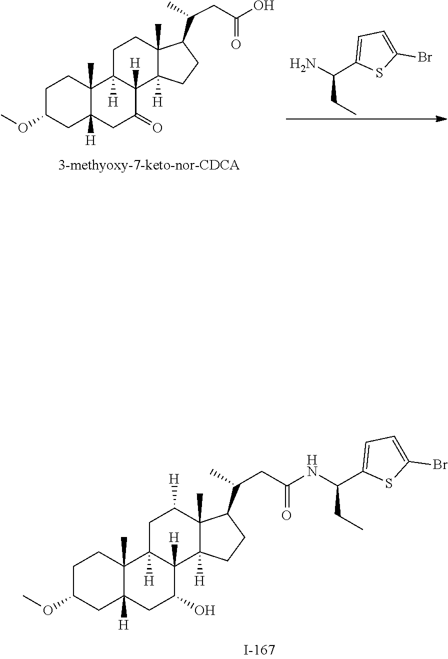

The compound I-167 was obtained by a synthetic method similar to that in Example 1 using 3-methoxy-7-keto-nor-CDCA and I-A-6-R as raw materials.

ESI-MS m/z: 546.03[M−1]⁻

### Example 168 Synthesis of Compound I-168

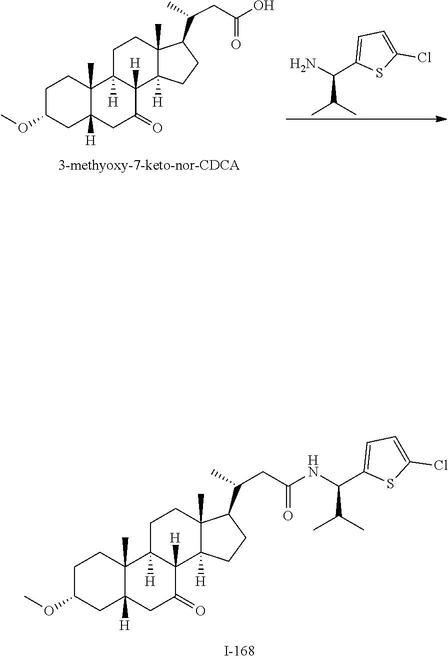

The compound I-168 was obtained by a synthetic method similar to that in Example 1 using 3-methoxy-7-keto-nor-CDCA and I-A-9-R as raw materials.

ESI-MS m/z: 560.10[M−1]⁻

### Example 169 Synthesis of Compound I-169

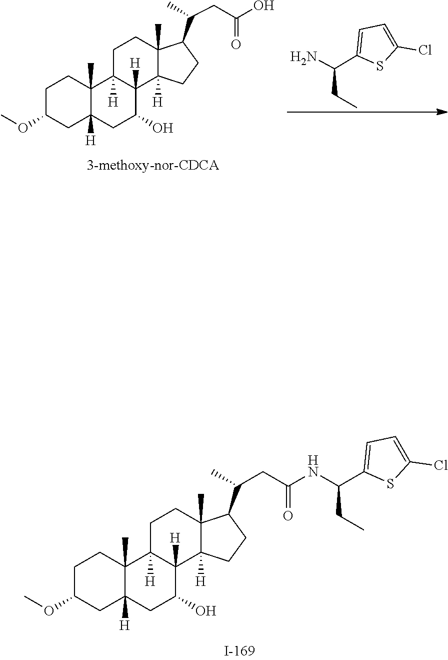

The compound I-169 was obtained by a synthetic method similar to that in Example 1 using 3-methoxy-nor-CDCA and I-A-6-R as raw materials.

ESI-MS m/z: 548.12[M−1]⁻

### Example 170 Synthesis of Compound I-170

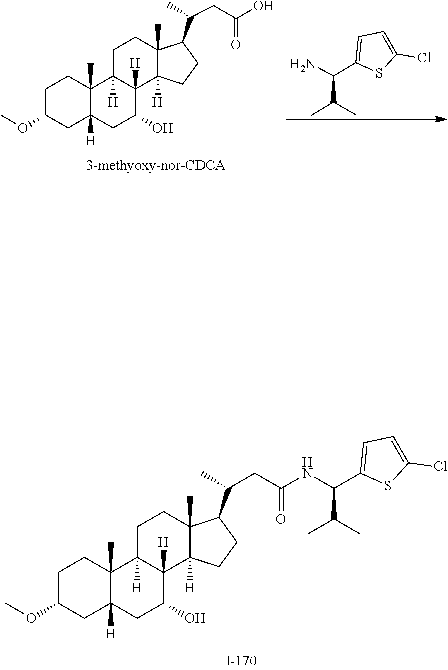

The compound I-170 was obtained by a synthetic method similar to that in Example 1 using 3-methoxy-nor-CDCA and I-A-9-R as raw materials.

¹HNMR(CDCl₃, 400 MHz): δ 6.78-6.67 (m, 2H), 5.82-5.62 (m, 1H), 5.05-4.96 (m, 1H), 3.85 (s, 1H), 3.36 (s, 3H), 3.10-2.97 (m, 1H), 2.47-2.38 (m, 1H), 2.22-1.93 (m, 4H), 1.90-1.71 (m, 6H), 1.71-1.58 (m, 2H), 1.58-1.45 (m, 3H), 1.45-1.11 (m, 9H), 1.03-0.87 (m, 9H), 0.92 (s, 3H), 0.71 (d, J=1.6 Hz, 3H).

ESI-MS m/z: 462.20[M−1]⁻

### Example 171 Synthesis of Compound I-171

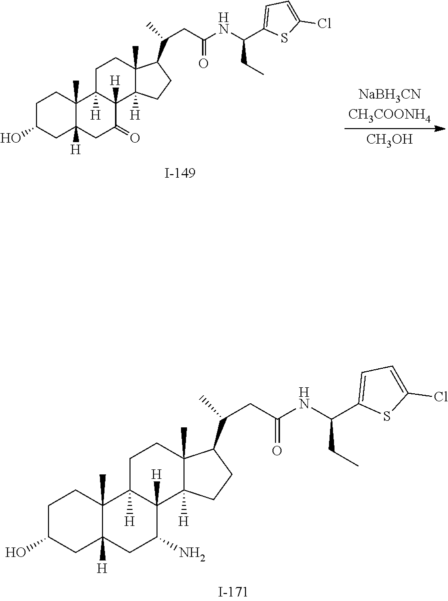

0.5 g of the compound I-149 was dissolved in 10 ml of methanol, 1.5 g of ammonium acetate and 0.75 g of sodium cyanoborohydride were added, and a reaction was conducted under reflux in an oil bath for 16 h until the raw materials disappeared completely; in the reaction system, 50 ml of a 3% potassium carbonate aqueous solution and 50 ml of ethyl acetate were added, and the reaction system was stirred fully for stratification; an aqueous phase was extracted twice with 15 mL of ethyl acetate, and organic layers were combined and washed twice with 20 mL of water; the organic layer was dried with 5 g of anhydrous sodium sulfate, filtered to remove the sodium sulfate, and a filtrate was concentrated to dryness to obtain a pale yellow solid, namely the compound I-171.

ESI-MS m/z: 533.06[M−1]⁻

### Example 172 Synthesis of Compound I-172

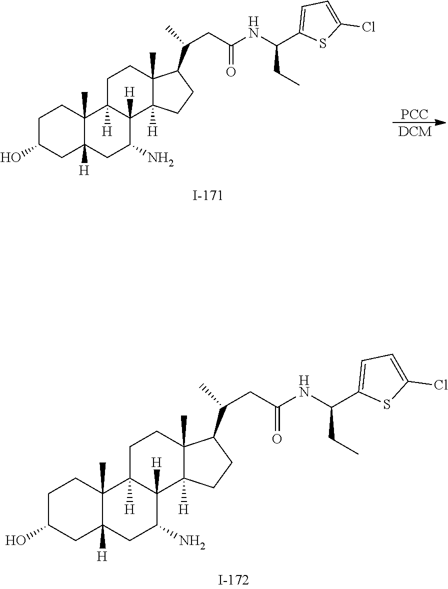

0.2 g of the compound I-171 was dissolved in 5 ml of dichloromethane, and 0.16 g of PCC was dissolved in 5 ml of dichloromethane; a dichloromethane solution of the PCC was added dropwise to a dichloromethane solution of the compound I-171 at room temperature, and a reaction was conducted by stirring at room temperature for 3 h until the raw materials disappeared completely; after concentration, a product was separated by column chromatography to obtain the compound I-172.

ESI-MS m/z: 531.19[M−1]⁻

### Example 173 Synthesis of Compound I-173

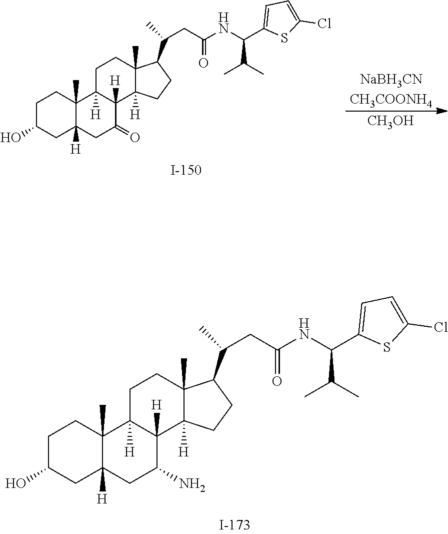

The compound I-173 was obtained by a synthetic method similar that in Example 171 using the compound I-150 as raw materials.

ESI-MS m/z: 547.16[M−1]⁻

### Example 174 Synthesis of Compound I-174

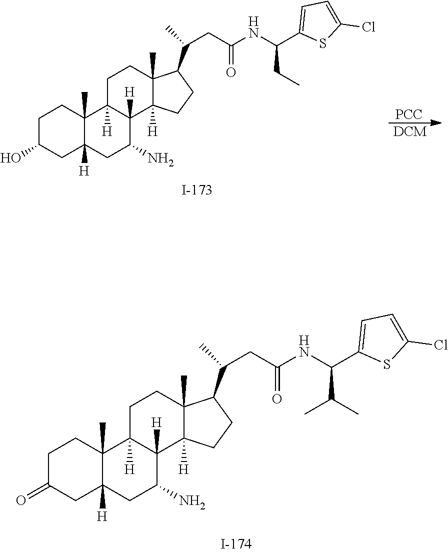

The compound I-174 was obtained by a synthetic method similar that in Example 172 using the compound I-173 as raw materials.

ESI-MS m/z: 545.10[M−1]⁻

### Example 175 Synthesis of Compound I-175

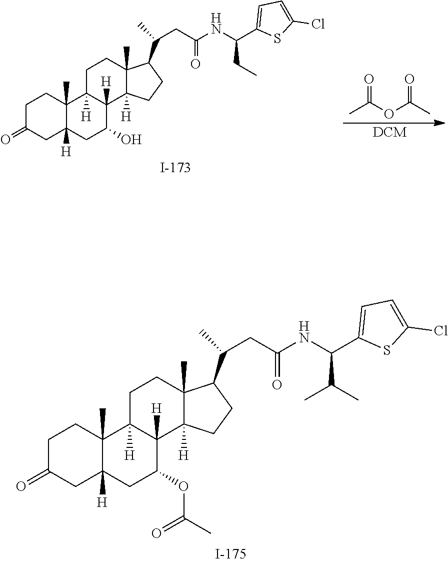

0.5 g of the compound I-88 was dissolved in 5 ml of dichloromethane, 0.3 g of triethylamine and 0.1 g of DMAP were added, and a temperature was lowered to 0° C. to 4° C. in an ice bath; 0.2 g of acetic anhydride was added dropwise, the ice bath was removed, and a reaction was continued for 3 h until the raw materials disappeared completely. 40 ml of dichloromethane and 30 ml of water were added to the system, stirred well and allowed to stand for stratification; an aqueous layer was extracted twice with 15 ml of dichloromethane, and organic layers were combined, washed twice with 10 ml of 0.1 M hydrochloric acid, and then once with water; the organic layer was dried with 5 g of anhydrous sodium sulfate for 20 min, the sodium sulfate was removed by filtration, and a filtrate was concentrated to dryness. An obtained product was refined by silica gel column chromatography to obtain the compound I-175.

ESI-MS m/z: 574.10[M−1]⁻

### Example 176 Synthesis of Compound I-176

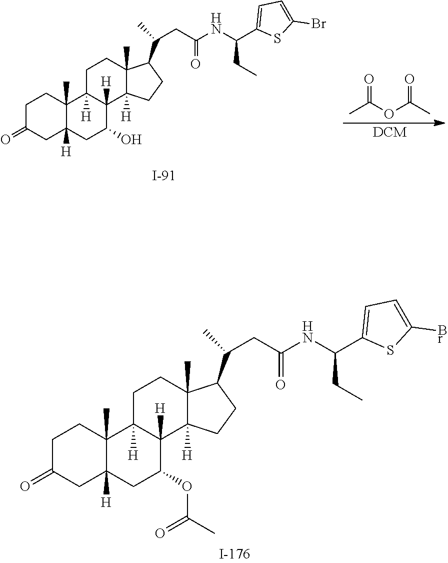

The compound I-176 was obtained by a synthetic method similar in Example 175 using the compound I-91 as raw materials.

ESI-MS m/z: 618.12[M−1]⁻

### Example 177 Synthesis of Compound I-177

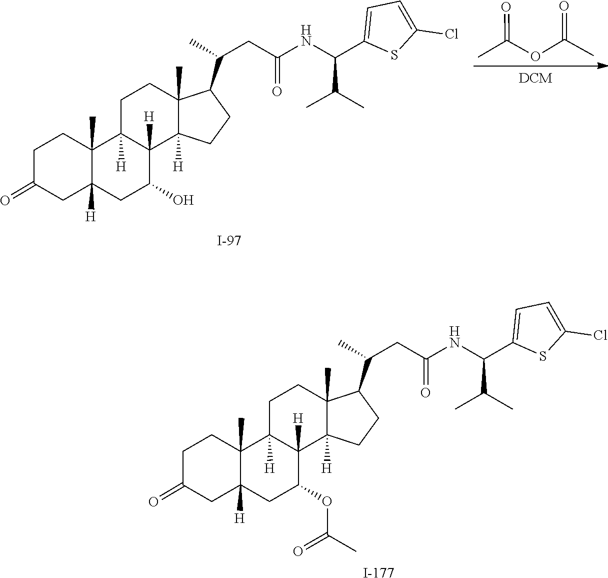

The compound I-177 was obtained by a synthetic method similar in Example 175 using the compound I-97 as raw materials.

ESI-MS m/z: 588.24[M−1]⁻

### Example 178 Synthesis of Compound I-178

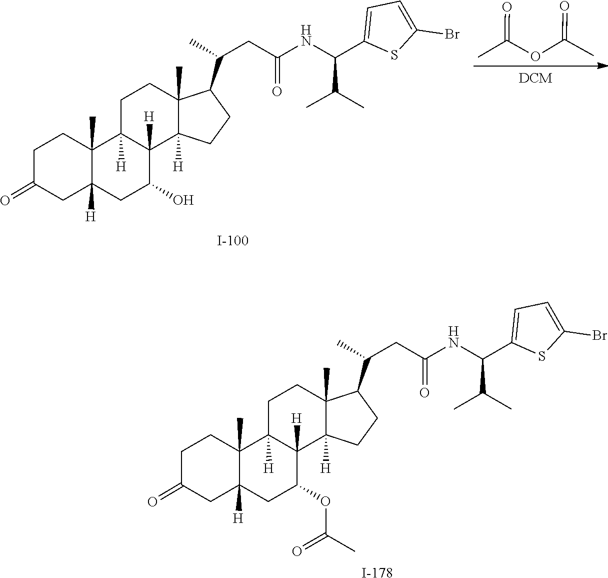

The compound I-178 was obtained by a synthetic method similar in Example 175 using the compound I-100 as raw materials.

ESI-MS m/z: 632.06[M−1]⁻

### Example 179 Synthesis of Compound I-179

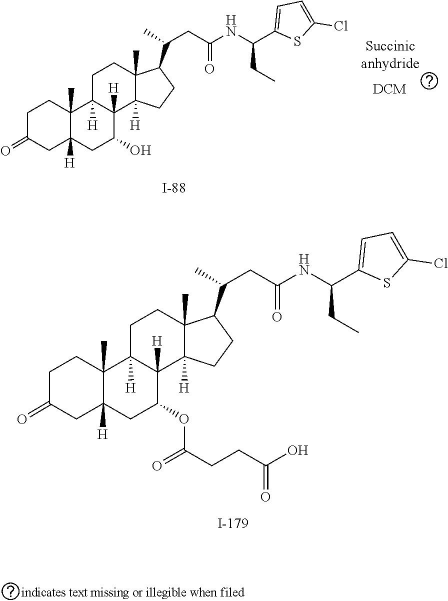

0.5 g of the compound I-88 was dissolved in 5 ml of dichloromethane, and 0.25 g of pyridine, 0.1 g of DMAP, and 0.2 g of succinic anhydride were added; a reaction was conducted at 40° C. by reflux for 10 h, until the raw materials were reacted completely. 40 ml of dichloromethane and 30 ml of water were added to the system, stirred well and allowed to stand for stratification; an aqueous layer was extracted twice with 15 ml of dichloromethane, and organic layers were combined, washed twice with 10 ml of 0.1 M hydrochloric acid, and then once with water; the organic layer was dried with 5 g of anhydrous sodium sulfate for 20 min, the sodium sulfate was removed by filtration, and a filtrate was concentrated to dryness. An obtained product was refined by silica gel column chromatography to obtain the compound I-179.

ESI-MS m/z: 632.11[M−1]⁻

### Example 180 Synthesis of Compound I-180

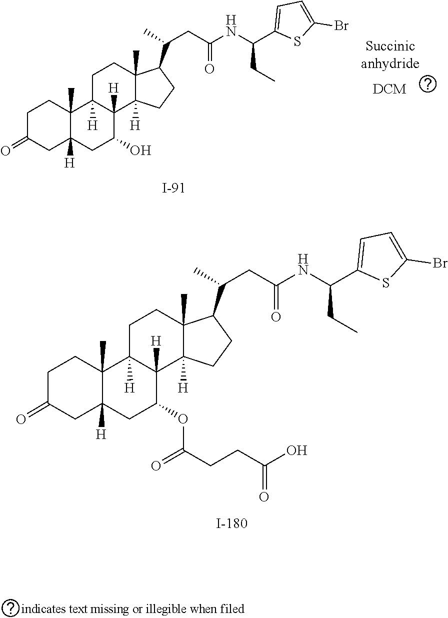

The compound I-180 was obtained by a synthetic method similar in Example 179 using the compound I-91 as raw materials.

ESI-MS m/z: 676.08[M−1]⁻

### Example 181 Synthesis of Compound I-181

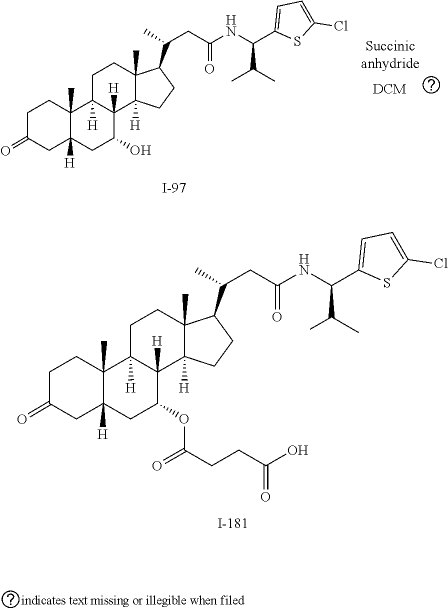

The compound I-181 was obtained by a synthetic method similar in Example 179 using the compound I-97 as raw materials.

¹HNMR(CDCl₃, 400 MHz): δ 6.79-6.68 (m, 2H), 6.27 (s, 1H), 5.08-4.97 (m, 2H), 3.03-2.92 (m, 1H), 2.80-2.50 (m, 3H), 2.50-2.35 (m, 4H), 2.26-2.18 (m, 3H), 2.16-1.78 (m, 9H), 1.73-1.40 (m, 4H), 1.40-1.13 (m, 5H), 1.05 (s, 3H), 1.13-0.86 (m, 10H), 0.72 (s, 3H).

ESI-MS m/z: 646.12[M−1]⁻

### Example 182 Synthesis of Compound I-182

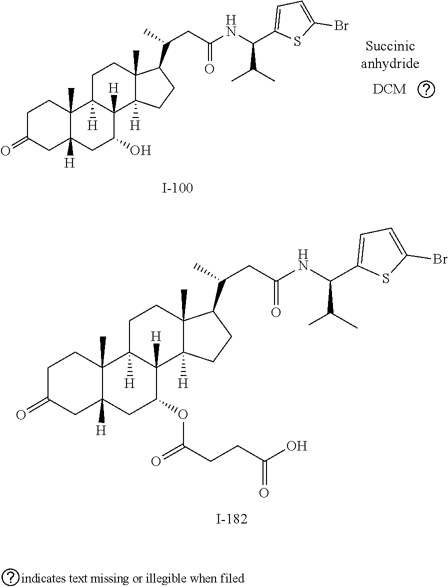

The compound I-182 was obtained by a synthetic method similar in Example 179 using the compound I-100 as raw materials.

ESI-MS m/z: 690.11[M−1]⁻

### Example 183 Synthesis of Compound I-183

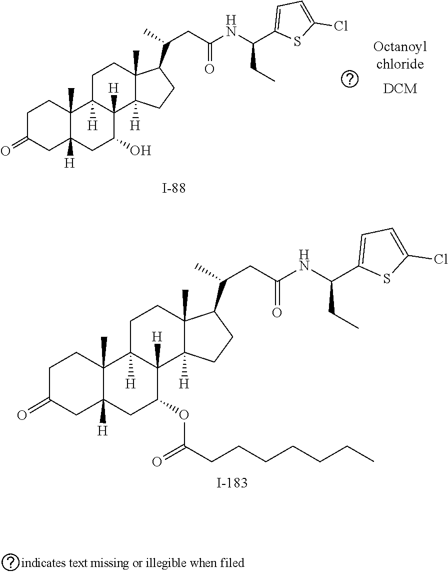

0.5 g of the compound I-88 was dissolved in 5 ml of methylene chloride, 0.5 g of pyridine was added, and a reaction system was cooled in an ice-water bath to 5° C.; 0.25 g of octanoyl chloride was dissolved in 3 ml of dichloromethane, slowly added dropwise to the reaction system, the ice-water bath was removed and the reaction system was warmed naturally to room temperature; a reaction was conducted at 40° C. by heating reflux for 16 h, until the raw materials were reacted completely. 40 ml of dichloromethane and 30 ml of water were added to the system, stirred well and allowed to stand for stratification; an aqueous layer was extracted twice with 15 ml of dichloromethane, and organic layers were combined, washed twice with 10 ml of 0.1 M hydrochloric acid, and then once with water; the organic layer was dried with 5 g of anhydrous sodium sulfate for 20 min, the sodium sulfate was removed by filtration, and a filtrate was concentrated to dryness. An obtained product was refined by silica gel column chromatography to obtain the compound I-183.

ESI-MS m/z: 658.18[M−1]⁻

### Example 184 Synthesis of Compound I-184

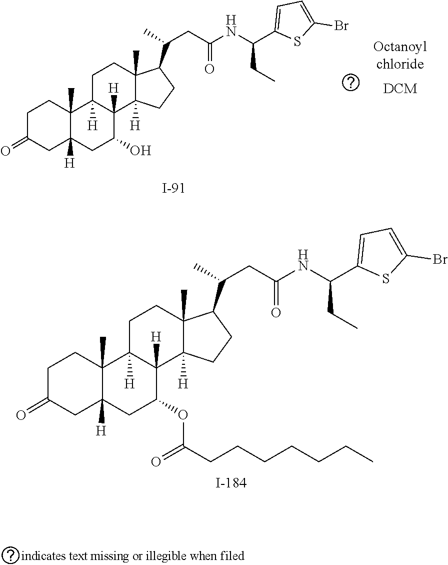

The compound I-184 was obtained by a synthetic method similar in Example 183 using the compound I-91 as raw materials.

ESI-MS m/z:702.13[M−1]⁻

### Example 185 Synthesis of Compound I-185

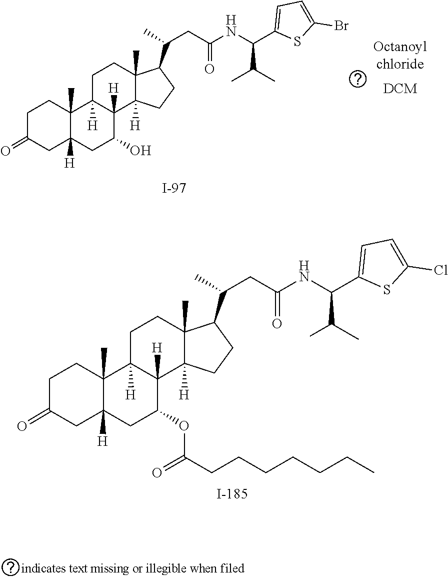

The compound I-185 was obtained by a synthetic method similar in Example 183 using the compound I-97 as raw materials.

ESI-MS m/z:672.19[M−1]⁻

### Example 186 Synthesis of Compound I-186

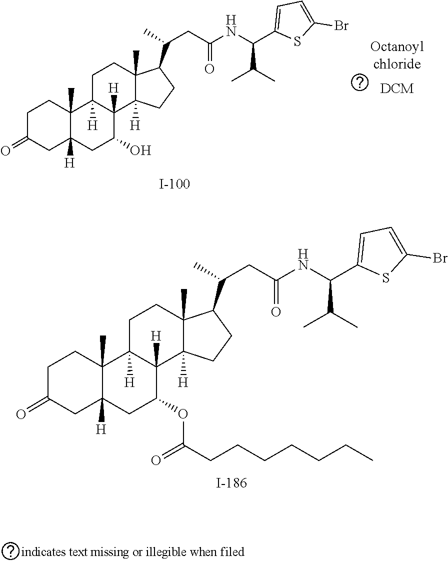

The compound I-186 was obtained by a synthetic method similar in Example 183 using the compound I-100 as raw materials.

ESI-MS m/z:716.24[M−1]⁻

### Example 187 Synthesis of Compound I-187

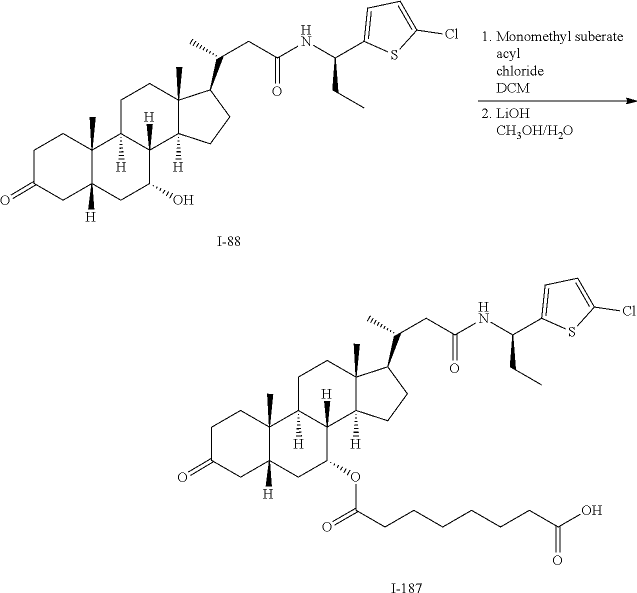

0.3 g of monomethyl suberate was dissolved in 5 ml of THF, 0.2 g of pyridine was added, 0.2 g of thionyl chloride was added dropwise at room temperature, a reaction was continued for 30 min, and an oily substance was concentrated to dryness; the oily substance was redissolved in 5 ml of dichloromethane to obtain a dichloromethane solution of monomethyl suberate acyl chloride. 0.5 g of the compound I-88 was dissolved in 5 ml of dichloromethane, 0.2 g of triethylamine was added, and a temperature was lowered to 5° C. in an ice-water bath; the dichloromethane solution of monomethyl suberate acyl chloride obtained in the previous step was slowly added dropwise into the reaction system, and the ice-water bath was removed and raised to room temperature; a reaction was conducted at 40° C. by heating reflux for 16 h, until the raw materials were reacted completely. 40 ml of dichloromethane and 30 ml of water were added to the system, stirred well and allowed to stand for stratification; an aqueous layer was extracted twice with 15 ml of dichloromethane, and organic layers were combined, washed twice with 10 ml of 0.1 M hydrochloric acid, and then once with water; the organic layer was dried with 5 g of anhydrous sodium sulfate for 20 min, and concentrated to dryness. An obtained compound was dissolved in 10 ml of methanol, 0.1 g of lithium hydroxide was dissolved in 2 ml of water, added to the above reaction system, and a reaction was conducted for 6 h until all raw materials disappeared. The methanol was concentrated and evaporated; an aqueous layer was extracted once with 10 mL of ethyl acetate, adjusted to a pH value of 6 with 1 M hydrochloric acid, and extracted three times with 30 mL of ethyl acetate; organic layers were combined, backwashed once with water, dried over 5 g of anhydrous sodium sulfate, filtered to remove the sodium sulfate, and a filtrate was concentrated to dryness. An obtained crude product was refined by silica gel column chromatography to obtain the compound I-187.

¹HNMR(CDCl₃, 400 MHz): δ 6.79-6.72 (m, 2H), 5.90-5.80 (m, 1H), 5.17-5.05 (m, 1H), 5.03-4.97 (m, 1H), 3.06-2.94 (m, 1H), 2.49-2.19 (m, 6H), 2.16-1.78 (m, 5H), 1.70-1.56 (m, 10H), 1.55-1.41 (m, 1H), 1.40-1.34 (m, 7H), 1.34-1.08 (m, 8H), 1.08-0.94 (m, 7H), 1.05 (s, 3H), 0.74 (s, 3H).

ESI-MS m/z:688.15[M−1]⁻

### Example 188 Synthesis of Compound I-188

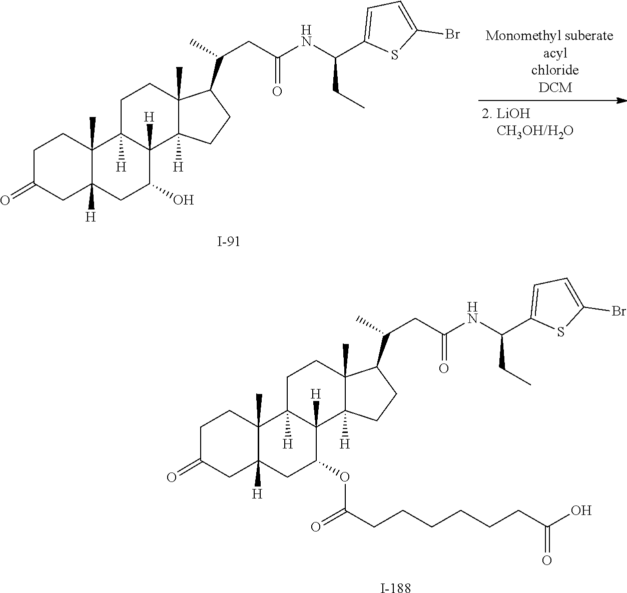

The compound I-188 was obtained by a synthetic method similar in Example 187 using the compound I-91 as raw materials.

ESI-MS m/z:732.10[M−1]⁻

### Example 189 Synthesis of Compound I-189

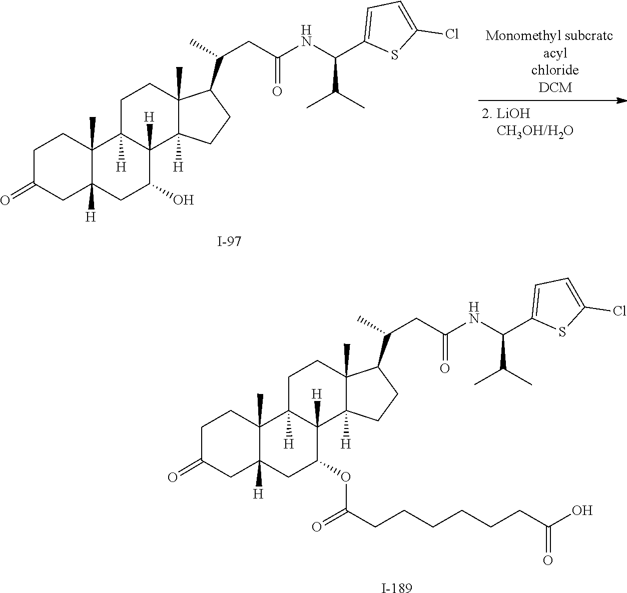

The compound I-189 was obtained by a synthetic method similar in Example 187 using the compound I-97 as raw materials.

ESI-MS m/z:702.17[M−1]⁻

### Example 190 Synthesis of Compound I-190

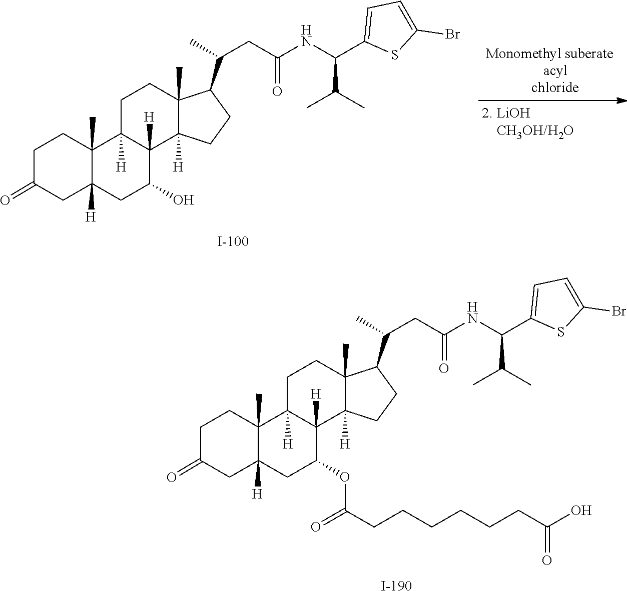

The compound I-190 was obtained by a synthetic method similar in Example 187 using the compound I-100 as raw materials.

ESI-MS m/z:746.12[M−1]⁻

### Example 191 Synthesis of Compound I-191

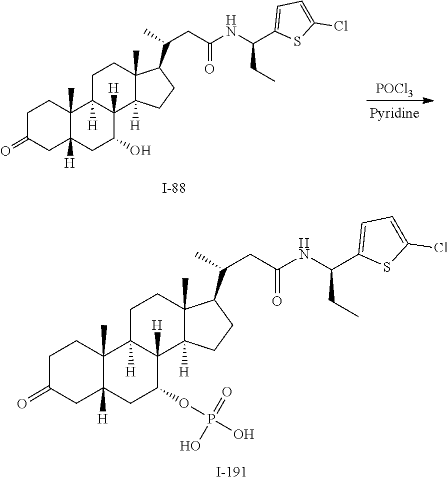

0.5 g of the compound I-88 was dissolved in 10 ml of anhydrous pyridine, and a reaction system was cooled in an ice-water bath to 5° C., and phosphorus oxychloride was added dropwise under stirring; after stirring for 2 h, ice water was added dropwise and stirred overnight; after extraction with ethyl acetate and concentration, a pale yellow solid was precipitated, and then purified by silica gel column chromatography to obtain the compound I-191.

ESI-MS m/z:635.03[M+Na]⁺

The compound I-191 was dissolved with 2 equivalents of a IN sodium hydroxide solution, stirred for 30 min, and treated with acetone to precipitate an off-white solid, to obtain a sodium salt I-191-1 of the compound I-191, with a structure being as follows:

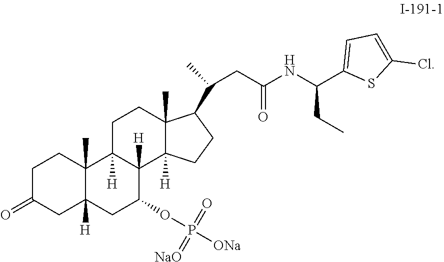

### Example 192 Synthesis of Compound I-192

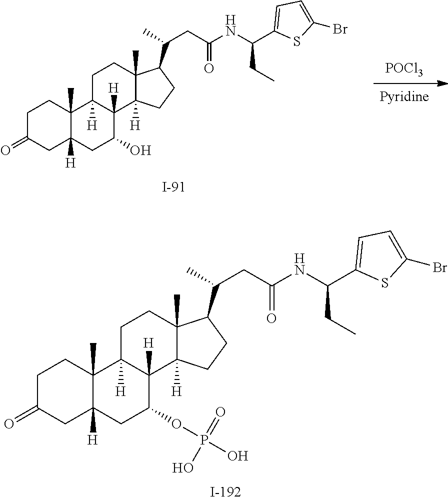

The compound I-192 was obtained by a synthetic method similar in Example 191 using the compound I-91 as raw materials.

ESI-MS m/z:679.18[M+Na]⁺

In a similar manner to Example 191, a sodium salt I-192-1 of the compound I-192 was obtained, with a structure being as follows:

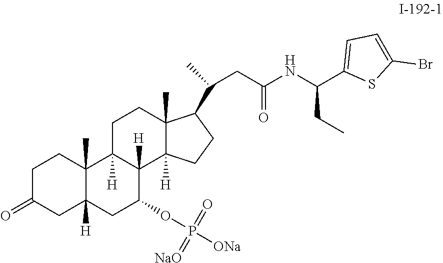

### Example 193 Synthesis of Compound I-193

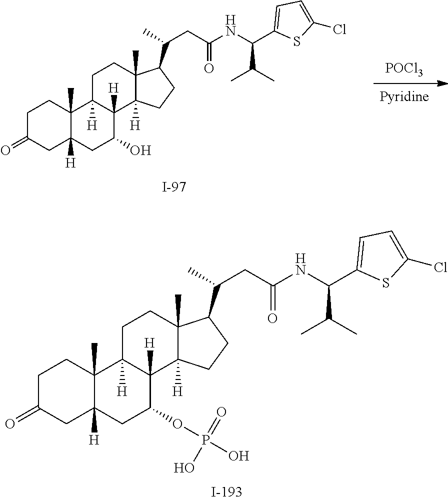

The compound I-193 was obtained by a synthetic method similar in Example 191 using the compound I-97 as raw materials.

ESI-MS m/z:649.05[M+Na]⁺

In a similar manner to Example 191, a sodium salt I-193-1 of the compound I-193 was obtained, with a structure being as follows:

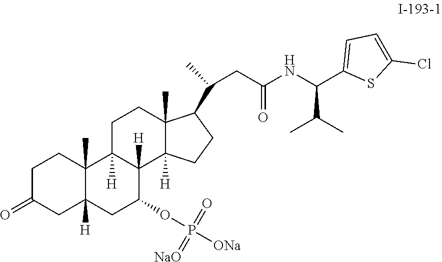

### Example 194 Synthesis of Compound I-194

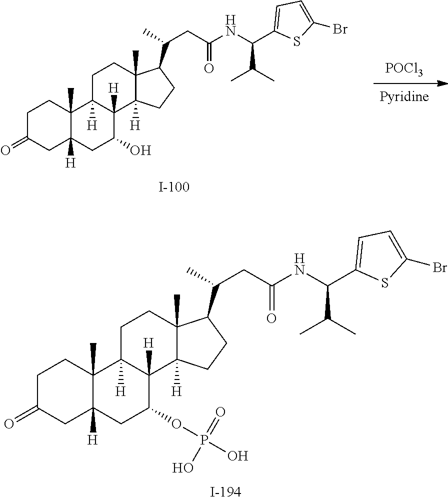

The compound I-194 was obtained by a synthetic method similar in Example 191 using the compound I-100 as raw materials.

ESI-MS m/z:692.99[M+Na]⁺

In a similar manner to Example 191, a sodium salt I-194-1 of the compound I-194 was obtained, with a structure being as follows:

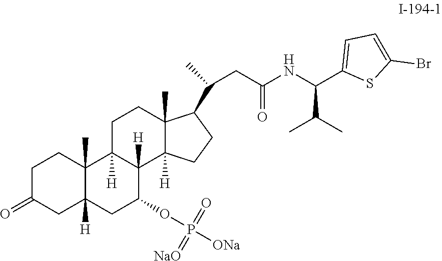

### Example 195 Synthesis of Compound I-195

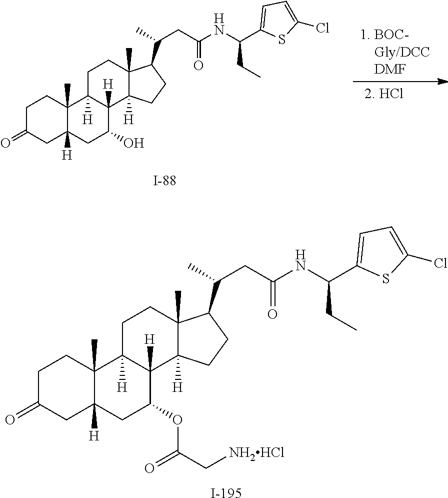

0.5 g of the compound I-88 was dissolved in 5 ml of DMF, 0.15 g of BOC-glycine was added, a reaction system was cooled by ice bath to 0° C., 0.3 g of dicyclohexylcarbodiimide (DCC) was slowly added, and a reaction was continued for 4 h until the raw materials all disappeared. 60 ml of ethyl acetate and 30 ml of water were added to the system, stirred well and allowed to stand for stratification; an aqueous layer was extracted twice with 15 mL of ethyl acetate; organic layers were combined and washed once with water, and the organic layer was dried with 5 g of anhydrous sodium sulfate for 20 min, filtered to remove the sodium sulfate, and a filtrate was concentrated to dryness. An obtained concentrate was deprotected from BOC with hydrochloric acid gas to obtain the compound I-195.

ESI-MS m/z:625.07[M−1]⁻

### Example 196 Synthesis of Compound I-196

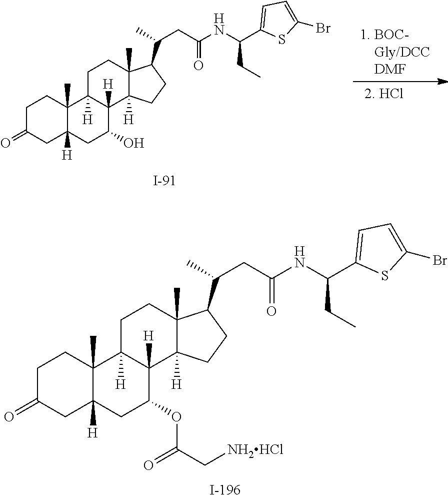

The compound I-196 was obtained by a synthetic method similar in Example 195 using the compound I-91 as raw materials.

ESI-MS m/z:669.00[M−1]⁻

### Example 197 Synthesis of Compound I-197

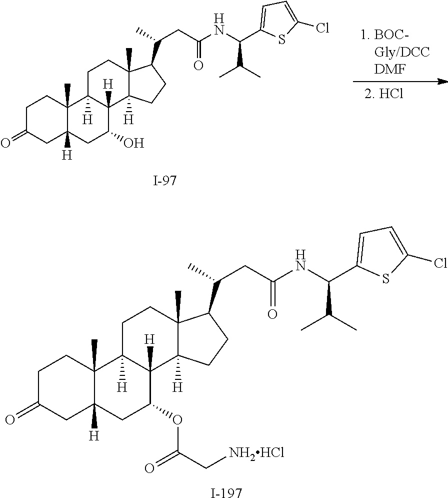

The compound I-197 was obtained by a synthetic method similar in Example 195 using the compound I-97 as raw materials.

ESI-MS m/z:603.11[M−1]⁻

### Example 198 Synthesis of Compound I-198

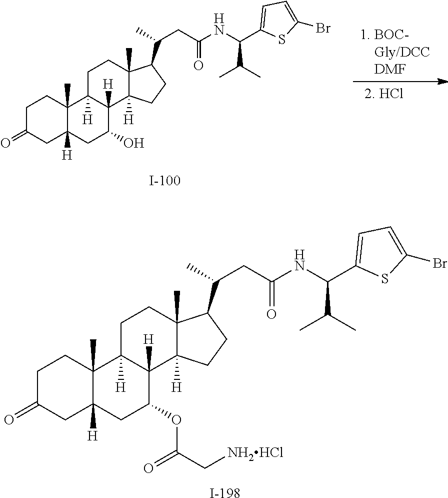

The compound I-198 was obtained by a synthetic method similar in Example 195 using the compound I-100 as raw materials.

ESI-MS m/z:647.06[M−1]⁻

### Example 199 Synthesis of Compound I-199

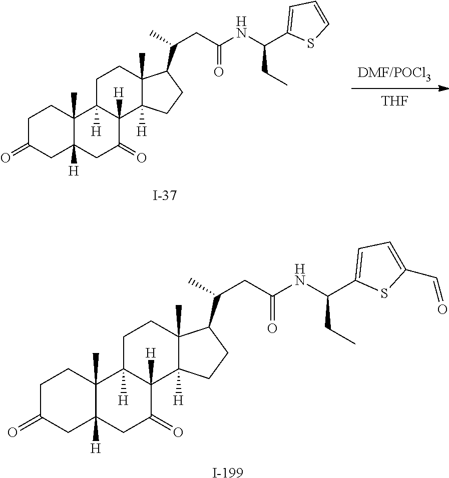

0.5 g of the compound I-37 was dissolved in 10 ml of tetrahydrofuran, 0.2 g of DMF was added, a reaction system was cooled by ice bath to 0° C., and phosphorus oxychloride was slowly added dropwise, and the reaction was continued for 4 h until the raw materials are reacted completely. 60 ml of ethyl acetate and 30 ml of water were added to the system, stirred well and allowed to stand for stratification; an aqueous layer was extracted twice with 15 mL of ethyl acetate; organic layers were combined and washed once with water, and the organic layer was dried with 5 g of anhydrous sodium sulfate for 20 min, filtered to remove the sodium sulfate, and a filtrate was concentrated to dryness. An obtained crude product was refined by silica gel column chromatography to obtain the compound I-199.

ESI-MS m/z:524.09[M−1]⁻

### Example 200 Synthesis of Compound I-200

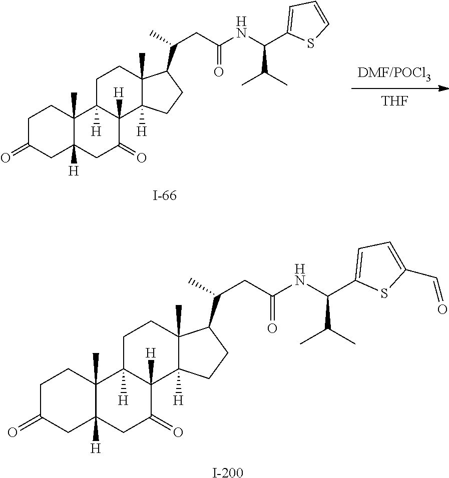

The compound I-200 was obtained by a synthetic method similar in Example 199 using the compound I-66 as raw materials.

ESI-MS m/z:538.11 [M−1]⁻

### Example 201 Synthesis of Compound I-201

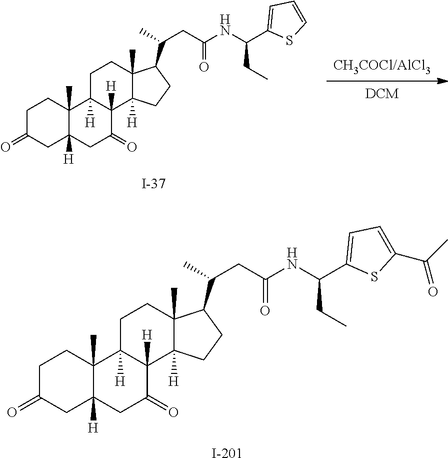

0.5 g of the compound I-37 was dissolved in 10 ml of dichloromethane, 0.3 g of aluminum chloride was added, a reaction system was cooled by ice bath to 0° C., a dichloromethane solution of acetyl chloride was slowly added dropwise, and the reaction was continued for 4 h until the raw materials disappeared completely. 40 ml of dichloromethane and 30 ml of water were added to the system, stirred well and allowed to stand for stratification; an aqueous layer was extracted twice with 15 ml of dichloromethane, and organic layers were combined, washed once with water; the organic layer was dried with 5 g of anhydrous sodium sulfate for 20 min, the sodium sulfate was removed by filtration, and a filtrate was concentrated to dryness. An obtained crude product was refined by silica gel column chromatography to obtain the compound I-201.

¹HNMR(CDCl₃, 400 MHz): δ 7.56 (d, J=3.9 Hz, 1H), 7.04-6.96 (m, 1H), 5.89 (s, 1H), 5.28-5.17 (m, 1H), 2.90 (dd, J=12.8, 5.6 Hz, 1H), 2.53 (s, 3H), 2.57-2.39 (m, 3H), 2.30-2.14 (m, 5H), 2.15-1.76 (m, 7H), 1.72-1.44 (m, 4H), 1.32 (s, 3H), 1.30-1.09 (m, 4H), 1.09-0.90 (m, 8H), 0.74 (d, J=3.8 Hz, 3H).

ESI-MS m/z:538.11 [M−1]⁻

### Example 202 Synthesis of Compound I-202

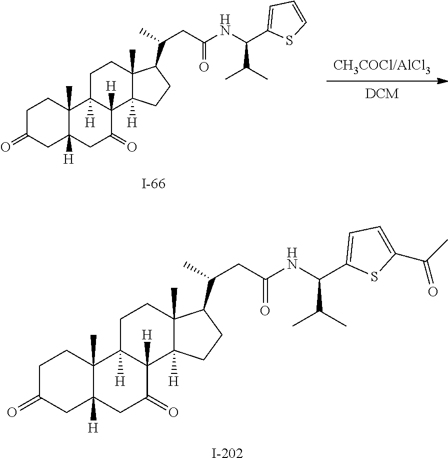

The compound I-202 was obtained by a synthetic method similar in Example 201 using the compound I-66 as raw materials.

ESI-MS m/z:552.22 [M−1]⁻

### Example 203 Synthesis of Compound I-203

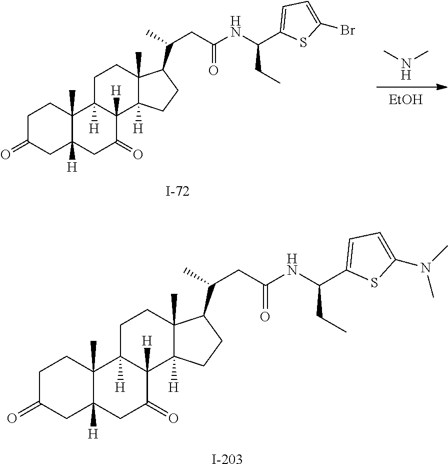

0.5 g of the compound I-72 was dissolved in 10 ml of ethanol, a 40% aqueous solution of dimethylamine was added, and a mixture was heated to reflux overnight until the raw materials were reacted completely. 60 ml of ethyl acetate was added to the system, stirred well and allowed to stand for stratification; an aqueous layer was extracted twice with 15 mL of ethyl acetate; organic layers were combined and washed once with water; the organic layer was dried with 5 g of anhydrous sodium sulfate for 20 min, the sodium sulfate was removed by filtration, and a filtrate was concentrated to dryness. An obtained crude product was refined by silica gel column chromatography to obtain the compound I-203.

ESI-MS m/z:539.14 [M−1]⁻

### Example 204 Synthesis of Compound I-204

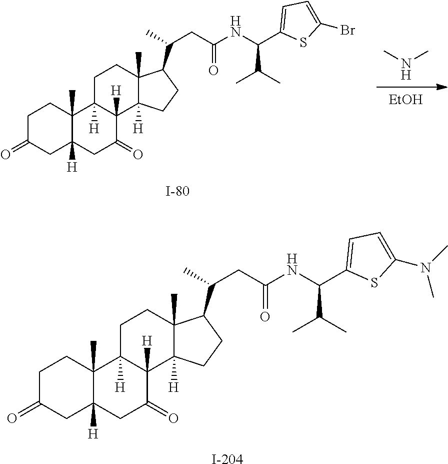

The compound I-204 was obtained by a synthetic method similar in Example 203 using the compound I-80 as raw materials.

ESI-MS m/z:553.15 [M−1]⁻

### Example 205 Synthesis of Compound I-205

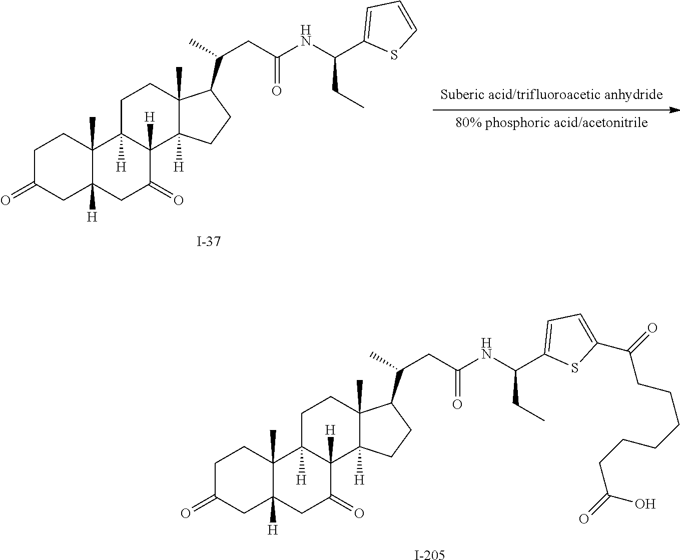

0.5 g of the compound I-37 was dissolved in 10 ml of acetonitrile, 0.2 g of suberic acid, 0.5 g of trifluoroacetic anhydride, and 0.15 g of an 80% phosphoric acid aqueous solution were added; a reaction was conducted by heating at 50° C. for 3 h to 4 h until the raw materials disappeared completely. 60 ml of ethyl acetate and 30 ml of water were added to the system, stirred well and allowed to stand for stratification; an aqueous layer was extracted twice with 15 mL of ethyl acetate; organic layers were combined and washed once with water, and the organic layer was dried with 5 g of anhydrous sodium sulfate for 20 min, filtered to remove the sodium sulfate, and a filtrate was concentrated to dryness. An obtained crude product was refined by silica gel column chromatography to obtain the compound I-205.

ESI-MS m/z:562.18 [M−1]⁻

### Example 206 Synthesis of Compound I-206

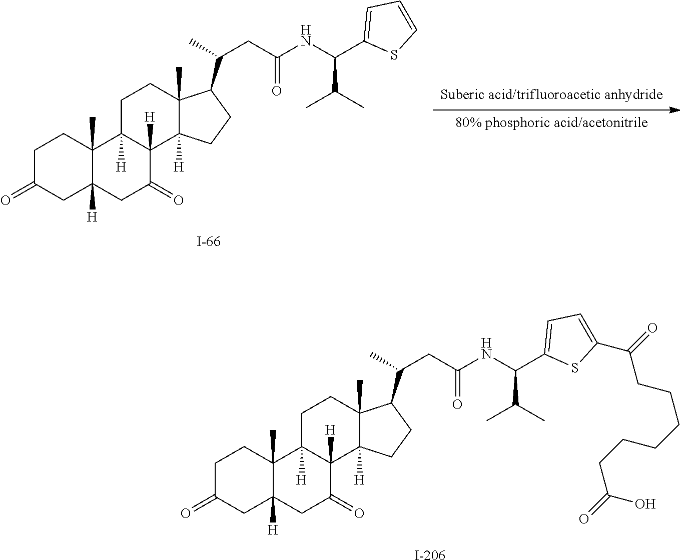

The compound I-206 was obtained by a synthetic method similar in Example 205 using the compound I-66 as raw materials.

ESI-MS m/z:666.19 [M−1]⁻

### Example 207 Preparation of Pharmaceutical Composition Tablet of the Compounds in the Present Disclosure

A pharmaceutical composition tablet of the compound I-70 included the following components in parts by weight: 1 part of the compound I-70, 0.1 parts to 0.3 parts of lactose, 0.4 parts to 0.2 parts of starch, 0.008 parts to 0.014 parts of sodium carboxymethyl starch, an appropriate amount of povidone K30, 0.01 parts to 0.05 part of magnesium stearate, and 0.5 parts of 40% ethanol; a tablet was prepared according to the above ratio, to obtain the pharmaceutical composition tablet of the compound I-70, in which each tablet contained 50 mg to 1,500 mg of the compound I-70.

By the same method as above, pharmaceutical composition tablets were prepared for the compounds I-72, I-74, I-77, I-80, I-88, I-91, I-97, I-100, I-106, I-109, I-112, and I-115.

### Example 208 Preparation of Pharmaceutical Composition Capsule of the Compounds in the Present Disclosure

A pharmaceutical composition capsule of the compound I-115 included the following components: 300 g of the compound I-115, 193 g of microcrystalline cellulose, 7 g of micropowder silica gel, which were 500 g in total, and a 2 # hollow capsule; alternatively, 1,200 g of the compound I-115, 279 g of the microcrystalline cellulose, 21 g of the micropowder silica gel, which were 1,500 g in total, and a 00 # hollow capsule. The method of preparation was as follows:

a, the compound I-115, the microcrystalline cellulose and the micropowder silica gel were mixed to obtain a mixed powder; and

b, the mixed powder was sieved through a 120-mesh sieve, filled into capsules and sealed, to obtain 1000 capsules in total.

Each capsule included 300 mg or 1,200 mg of the compound I-115.

By the same method as above, pharmaceutical composition capsules were prepared for the compounds I-70, I-72, I-74, I-77, I-80, I-88, I-91, I-97, I-100, I-106, I-109, and I-112.

The beneficial effects of the compounds in present disclosure were verified by the following experimental examples.

Experimental Example 1 Compounds of the present disclosure inhibiting germination of C. difficile spores

(1) Experimental Method

According to a method disclosed in “The Journal of Infectious Diseases. 2013; 207: 1498-504”, spores of ATCC BBA1870, ATCC 43255 and ATCC 630 strains were obtained by culture and purification, and stored at 4° C.

Spore preparation: the C. difficile spores stored at 4° C. were heat-activated for 30 min at 65° C., centrifuged at 9,400 g for 2 min, and washed three times to remove any self-germinating spores. The spores were resuspended in a BHIS medium, and an absorbance value at 580 nm was detected by a microplate reader; a spore density was adjusted, such that 100 μl of the spores in a 96-well microplate had an optical density (OD) value of around 1.0.

Control drug: three cholic acid derivatives, a compound CamSA, a compound 20β (prepared by the method of “Journal of Medicinal Chemistry. 2017, 60 (8): 3451-3471”), and a compound 12a (prepared by the method of “Journal of Medicinal Chemistry. 2018, 61, 6759-6778”), were used as control drugs.

Compound preparation: the compound of the present disclosure, and the positive control drugs CamSA, 20β, and 12a were dissolved in DMSO, to prepare a stock solution with a concentration of 10 mM (prepared for immediate use). The compound was diluted appropriately for use, such that the compound had a detection concentration range of 0.125 μM to 256 μM, with a total of 11 concentration gradients of two-fold dilution.

The detection was conducted in a 96-well cell culture plate at a constant temperature of 37° C.; a final volume of each well of the 96-well plate was 200 and a buffer was the BHIS medium; 150 μl of spores and drugs were added to the well in advance, and 50 μl of 4 mg/ml sodium taurocholate was added; OD₅₈₀ was detected every 1 min immediately using a microplate reader, for continuously 60 min. A germination agent control group and a non-germination group were set up; after the detection, a final OD value of each well was compared with an initial value, and compared with the germination agent group to calculate a germination rate and an inhibition rate. A formula for the germination rate was:

$$Germination = \frac{\begin{matrix}1 - Fin OD value of experimental \\ group / Initial OD value of experimental group\end{matrix}}{\begin{matrix}1 - Fin OD value of germination group / Fina \\ OD value of germination group\end{matrix}} \times 100 \% .$$

A formula for the inhibition rate was:

Inhibition rate=100%−germination rate.

A minimum concentration that completely inhibits spore germination was an MIC value of the compound, and results were recorded in Table 1.

(2) Experimental Results

TABLE 1
Inhibition of compounds of the present disclosure on germination of C. difficile spores

| | MIC (μM) | | | | MIC (μM) | | |
|---|---|---|---|---|---|---|---|
| Compound | 1870 | 43255 | 630 | Compound | 1870 | 43255 | 630 |
| I-1 | 32 | 32 | 32 | I-2 | 16 | 8 | 8 |
| I-3 | 8 | 8 | 4 | I-4 | 32 | 16 | 8 |
| I-5 | 16 | 16 | 32 | I-6 | 64 | 64 | 64 |
| I-7 | 16 | 16 | 16 | I-8 | 4 | 2 | 4 |
| I-9 | 8 | 8 | 8 | I-10 | 32 | 32 | 32 |
| I-11 | 64 | 64 | 64 | I-12 | 128 | 256 | 256 |
| I-13 | 16 | 16 | 16 | I-14 | 4 | 4 | 4 |
| I-15 | 32 | 64 | 32 | I-16 | 256 | 256 | 256 |
| I-17 | 64 | 32 | 32 | I-18 | 32 | 8 | 8 |
| I-19 | 64 | 32 | 32 | I-20 | 64 | 32 | 32 |
| I-21 | 8 | 8 | 4 | I-22 | 8 | 8 | 8 |
| I-23 | 4 | 2 | 2 | I-24 | >256 | >256 | >256 |
| I-25 | 2 | 1 | 1 | I-26 | 0.5 | 1 | 0.5 |
| I-27 | 2 | 2 | 2 | I-28 | >256 | >256 | >256 |
| I-29 | 1 | 1 | 1 | I-30 | 2 | 2 | 1 |
| I-31 | 8 | 8 | 8 | I-32 | 0.5 | 0.5 | 0.5 |
| I-33 | 0.5 | 1 | 0.5 | I-34 | 64 | 64 | 64 |
| I-35 | 0.5 | 0.5 | 0.5 | I-36 | 8 | 8 | 4 |
| I-37 | 0.25 | 0.25 | 0.25 | I-38 | 2 | 2 | 1 |
| I-39 | 1 | 0.5 | 0.5 | I-40 | 4 | 2 | 2 |
| I-41 | 0.5 | 0.5 | 0.5 | I-42 | 2 | 1 | 1 |
| I-43 | 64 | 32 | 32 | I-44 | 256 | >256 | >256 |
| I-45 | >256 | >256 | >256 | I-46 | >256 | >256 | >256 |
| I-47 | >256 | >256 | >256 | I-48 | 0.5 | 0.5 | 0.5 |
| I-49 | >256 | >256 | >256 | I-50 | 0.25 | 0.25 | 0.25 |
| I-51 | 0.5 | 0.5 | 0.5 | I-52 | 16 | 16 | 16 |
| I-53 | 32 | 32 | 32 | I-54 | >256 | >256 | 256 |
| I-55 | 64 | 32 | 32 | I-56 | 16 | 16 | 16 |
| I-57 | 32 | 32 | 16 | I-58 | 8 | 4 | 4 |
| I-59 | 128 | 64 | 64 | I-60 | 2 | 2 | 2 |
| I-61 | 128 | 128 | 128 | I-62 | 256 | 128 | 128 |
| I-63 | >256 | 256 | 256 | I-64 | >256 | >256 | >256 |
| I-65 | 128 | 128 | 128 | I-66 | 0.25 | 0.25 | 0.25 |
| I-67 | >256 | >256 | >256 | I-68 | 1 | 2 | 1 |
| I-69 | 8 | 16 | 8 | I-70 | 0.25 | 0.5 | 0.25 |
| I-71 | 32 | 16 | 16 | I-72 | 1 | 1 | 0.5 |
| I-73 | >256 | >256 | >256 | I-74 | 16 | 16 | 16 |
| I-75 | 0.5 | 0.5 | 0.5 | I-76 | >256 | >256 | >256 |
| I-77 | 0.25 | 0.25 | 0.25 | I-78 | 0.5 | 1 | 0.5 |
| I-79 | 8 | 8 | 4 | I-80 | 0.125 | 0.125 | 0.125 |
| I-81 | 32 | 32 | 32 | I-82 | >256 | 256 | >256 |
| I-83 | 16 | 16 | 16 | I-84 | 1 | 1 | 1 |
| I-85 | 0.5 | 0.5 | 0.5 | I-86 | 0.5 | 0.5 | 0.5 |
| I-87 | 256 | 128 | 128 | I-88 | 0.25 | 0.25 | 0.125 |
| I-89 | 0.5 | 0.5 | 0.5 | I-90 | >256 | >256 | >256 |
| I-91 | 0.25 | 0.25 | 0.25 | I-92 | 16 | 32 | 16 |
| I-93 | >256 | >256 | >256 | I-94 | 8 | 16 | 8 |
| I-95 | 0.25 | 0.25 | 0.25 | I-96 | 4 | 4 | 4 |
| I-97 | 0.125 | 0.125 | 0.125 | I-98 | 0.5 | 0.5 | 0.5 |
| I-99 | >256 | 256 | 256 | I-100 | 0.25 | 0.25 | 0.25 |
| I-101 | 32 | 32 | 32 | I-102 | >256 | >256 | >256 |
| I-103 | 16 | 16 | 16 | I-104 | 1 | 1 | 1 |
| I-105 | >256 | >256 | >256 | I-106 | 0.5 | 0.25 | 0.5 |
| I-107 | 1 | 1 | 1 | I-108 | >256 | >256 | >256 |
| I-109 | 0.5 | 0.25 | 0.5 | I-110 | 1 | 0.5 | 1 |
| I-111 | >256 | >256 | >256 | I-112 | 0.5 | 0.25 | 0.5 |
| I-113 | 1 | 0.5 | 1 | I-114 | >256 | >256 | >256 |
| I-115 | 0.5 | 0.25 | 0.5 | I-116 | 16 | 16 | 16 |
| I-117 | 8 | 8 | 4 | I-118 | 16 | 8 | 8 |
| I-119 | 32 | 32 | 32 | I-120 | 16 | 16 | 16 |
| I-121 | >256 | >256 | >256 | I-122 | 128 | 128 | 128 |
| I-123 | 64 | 64 | 64 | I-124 | 64 | 64 | 64 |
| I-125 | 2 | 2 | 2 | I-126 | 2 | 2 | 1 |
| I-127 | 8 | 8 | 4 | I-128 | 2 | 2 | 1 |
| I-129 | 4 | 2 | 2 | I-130 | 4 | 4 | 4 |
| I-131 | 2 | 2 | 2 | I-132 | >256 | 128 | 256 |
| I-133 | 2 | 2 | 2 | I-134 | 2 | 2 | 2 |
| I-135 | >256 | >256 | >256 | I-136 | 1 | 1 | 1 |
| I-137 | 1 | 1 | 1 | I-138 | 1 | 1 | 1 |
| I-139 | 2 | 1 | 1 | I-140 | 1 | 1 | 1 |
| I-141 | 32 | 32 | 32 | I-142 | 32 | 32 | 32 |
| I-143 | 8 | 4 | 4 | I-144 | 4 | 2 | 2 |
| I-145 | 32 | 32 | 16 | I-146 | 16 | 16 | 16 |
| I-147 | 32 | 16 | 16 | I-148 | 16 | 16 | 16 |
| I-149 | 2 | 2 | 2 | I-150 | 2 | 2 | 1 |
| I-151 | 0.5 | 1 | 0.5 | I-152 | 1 | 1 | 0.5 |
| I-153 | 0.5 | 0.5 | 0.5 | I-154 | 1 | 0.5 | 0.5 |
| I-155 -1 | 0.25 | 0.25 | 0.25 | I-155-2 | 0.25 | 0.25 | 0.25 |
| I-156 -1 | 0.5 | 0.5 | 0.5 | I-156-2 | 0.25 | 0.5 | 0.25 |
| I-157 -1 | 0.5 | 0.5 | 0.25 | I-157-2 | 0.25 | 0.5 | 0.25 |
| I-158 -1 | 0.5 | 0.5 | 0.5 | I-158-2 | 0.5 | 0.5 | 0.5 |
| I-159 -1 | 0.5 | 0.5 | 0.5 | I-159-2 | 0.5 | 0.5 | 0.5 |
| I-160 -1 | 0.5 | 0.5 | 0.5 | I-160-2 | 0.5 | 0.5 | 0.5 |
| I-161 -1 | 0.5 | 0.5 | 0.25 | I-161-2 | 0.25 | 0.5 | 0.25 |
| I-162 -1 | 0.25 | 0.5 | 0.5 | I-162-2 | 0.5 | 0.5 | 0.5 |
| I-163 | 32 | 32 | 32 | I-164 | 32 | 32 | 32 |
| I-165 | 64 | 64 | 32 | I-166 | 64 | 32 | 32 |
| I-167 | 256 | 128 | 128 | I-168 | 128 | 128 | 128 |
| I-169 | 256 | 256 | 128 | I-170 | 128 | 128 | 128 |
| I-171 | 32 | 32 | 32 | I-172 | 32 | 32 | 32 |
| I-173 | 32 | 16 | 16 | I-174 | 32 | 32 | 32 |
| I-175 | 1 | 1 | 0.5 | I-176 | 0.5 | 0.5 | 0.5 |
| I-177 | 0.5 | 1 | 0.5 | I-178 | 1 | 0.5 | 0.5 |
| I-179 | 2 | 2 | 2 | I-180 | 2 | 2 | 1 |
| I-181 | 2 | 2 | 2 | I-182 | 2 | 2 | 2 |
| I-183 | 256 | 128 | 128 | I-184 | 128 | 128 | 128 |
| I-185 | 128 | 128 | 128 | I-186 | 256 | 256 | 256 |
| I-187 | 0.125 | 0.125 | 0.125 | I-188 | 0.25 | 0.25 | 0.25 |
| I-189 | 0.25 | 0.25 | 0.125 | I-190 | 0.25 | 0.25 | 0.25 |
| I-191 | 64 | 64 | 64 | I-192 | 64 | 64 | 64 |
| I-193 | 64 | 64 | 64 | I-194 | 64 | 64 | 64 |
| I-195 | 256 | >256 | 256 | I-196 | >256 | 256 | 256 |
| I-197 | >256 | 256 | 256 | I-198 | >256 | >256 | 256 |
| I-199 | 16 | 16 | 16 | I-200 | 32 | 32 | 16 |
| I-201 | 32 | 32 | 32 | I-202 | 32 | 32 | 32 |
| I-203 | 32 | 32 | 16 | I-204 | 32 | 32 | 32 |
| I-205 | 128 | 128 | 128 | I-206 | 256 | 128 | 128 |
| 20β | 64 | 64 | 64 | CamSA | >256 | >256 | >256 |
| 12a | 4 | 4 | 4 | | | | |

As can be seen from Table 1, the compounds of the present disclosure may effectively inhibit the germination of C. difficile spores; the inhibition effects of most compounds on germination of C. difficile spores were better than that of the control compound CamSA. In particular, the MIC values of many compounds such as I-32, I-35, I-37, I-41, I-48, I-50, and I-51 on the germination of various C. difficile spores were all as low as 1 μM, showing a particularly significant inhibitory activity against spore germination.

In conclusion, the present disclosure provides a compound represented by formula (I). The compound is capable of effectively inhibiting germination of C. difficile spores with a significant bacteriostatic activity. The compound has an excellent prospect for use in preparation of a drug for preventing and/or treating a C. difficile-caused infectious disease, recurrence of the C. difficile-caused infectious disease, or a complication of the C. difficile-caused infectious disease.

1. A compound represented by formula (I), or a salt or a stereoisomer thereof: 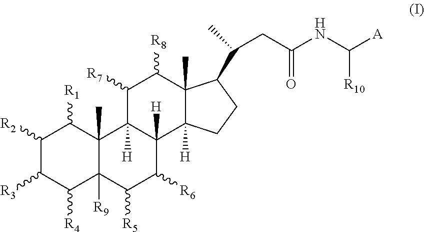
wherein R₁, R₂, R₃, R₄, R₅, R₆, R₇, and R₈ are independently selected from the group consisting of H, —OH, C₁₋₅ alkoxy, —NH₂, ═O, ═S, ═NOH, —NHSO₃H or a salt thereof, —OSO₃H or a salt thereof, —OPO(OH)₂ or a salt thereof, —OCO(CH₂)ₘCOOH or a salt thereof, an amino acid ester group or a salt thereof, and —OCO(CH₂)ₘCH₃; m is an integer ranging from 0 to 9; and the amino acid ester group is a residue with a hydrogen atom removed from carboxyl on an amino acid; or
R₁ and R₂, R₅ and R₆, and/or R₇ and R₈ are ligated to form a double bond;
R₉ is selected from H, or R₉ is ligated with R₄ or R₅ to form a double bond;
R₁₀ is selected from H, linear or branched C₁₋₁₃ alkyl, —(CH₂)ₙCOOH, —(CH₂)ₙ₊₁NH₂, and —(CH₂)ₙ-B; and n is an integer ranging from 0 to 4;
A and B are independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl;
cycloalkyl comprises cyclopropyl, cyclobutyl, cyclopentyl, and cyclohexyl; and [symbol]
represents a single bond or a double bond.

2. The compound, or the salt or the stereoisomer thereof according to claim 1, wherein the compound of formula (I) has a structure represented by formula (IIA): 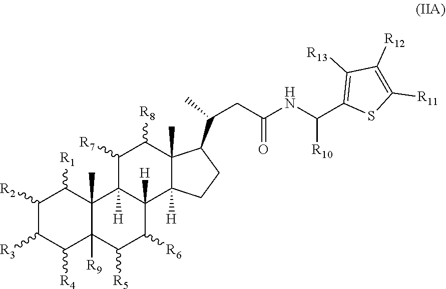
R₁, R₂, R₃, R₄, R₅, R₆, R₇, and R₈ are independently selected from the group consisting of H, —OH, C₁₋₅ alkoxy, —NH₂, ═O, ═S, ═NOH, —NHSO₃H or a salt thereof, —OSO₃H or a salt thereof, —OPO(OH)₂ or a salt thereof, —OCO(CH₂)ₘCOOH or a salt thereof, an amino acid ester group or a salt thereof, and —OCO(CH₂)ₘCH₃; and m is an integer ranging from 0 to 9; or
R₁ and R₂, R₅ and R₆, and/or R₇ and R₈ are ligated to form a double bond;
R₉ is selected from H, or R₉ is ligated with R₄ or R₅ to form a double bond;
R₁₀ is selected from H, linear or branched C₁₋₄ alkyl, —(CH₂)ₙCOOH, —(CH₂)ₙ₊₁NH₂, and —(CH₂)ₙ-B; and n is an integer ranging from 0 to 4;
B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl;
cycloalkyl comprises cyclopropyl, cyclobutyl, cyclopentyl, and cyclohexyl; and
R₁₁, R₁₂, and R₁₃ are independently selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

3. The compound, or the salt or the stereoisomer thereof according to claim 2, wherein the compound of formula (IIA) has a structure represented by formula (IIIA-1) or (IIIA-2): 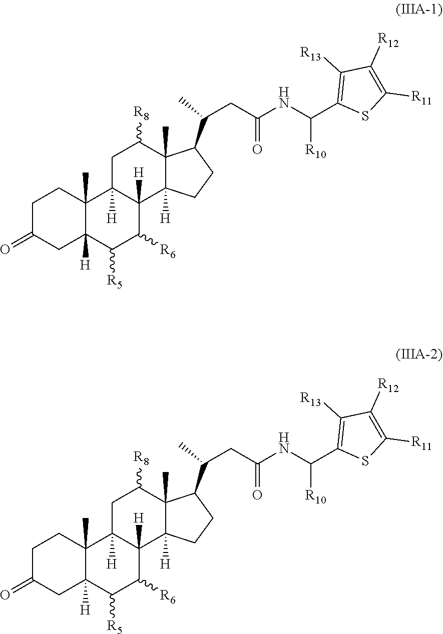
R₅, R₆, and R₈ are independently selected from the group consisting of H, —OH, —NH₂, —OSO₃H or a salt thereof, —OPO(OH)₂ or a salt thereof, —OCO(CH₂)ₙCOOH or a salt thereof, an amino acid ester group or a salt thereof, and —OCO(CH₂)ₘCH₃; and m is an integer ranging from 0 to 9;
R₁₀ is selected from H, linear or branched C₁₋₄ alkyl, —(CH₂)ₙCOOH, —(CH₂)ₙ₊₁NH₂, and —(CH₂)ₙ-B; and n is an integer ranging from 0 to 4;
B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;
cycloalkyl comprises cyclopropyl, cyclobutyl, cyclopentyl, and cyclohexyl; and
R₁₁, R₁₂, and R₁₃ are independently selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

4. The compound, or the salt or the stereoisomer thereof according to claim 3, wherein the compound of formula (IIIA-11 has a structure represented by formula (IVA-1): 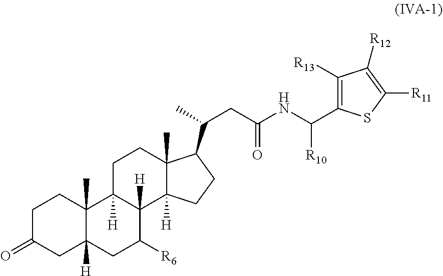
R₆ is selected from the group consisting of —OH, —NH₂, —OSO₃H or a salt thereof, —OPO(OH)₂ or a salt thereof, —OCO(CH₂)ₘCOOH or a salt thereof, an amino acid ester or a salt thereof, and —OCO(CH₂)ₘCH₃; the amino acid ester group is an α-amino acid ester group; and m is an integer ranging from 0 to 9;
R₁₀ is selected from H, linear or branched C₁₋₄ alkyl, —(CH₂)ₙCOOH, —(CH₂)ₙ₊₁NH₂, and —(CH₂)ₙ-B; and n is an integer ranging from 0 to 4;
B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;
cycloalkyl comprises cyclopropyl, cyclobutyl, cyclopentyl, and cyclohexyl; and
R₁₁, R₁₂, and R₁₃ are independently selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

5. The compound, or the salt or the stereoisomer thereof according to claim 3, wherein the compound of formula (IIIA-1) has a structure represented by formula (VA-1): 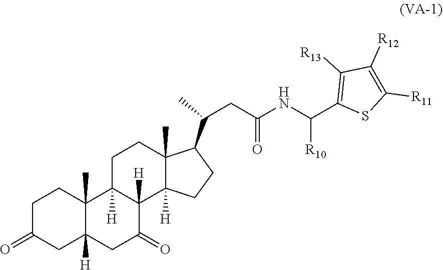
R₁₀ is selected from H, linear or branched C₁₋₄ alkyl, —(CH₂)ₙCOOH, —(CH₂)ₙ₊₁NH₂, and —(CH₂)ₙ-B; and n is an integer ranging from 0 to 4;
B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;
cycloalkyl comprises cyclopropyl, cyclobutyl, cyclopentyl, and cyclohexyl; and
R₁₁, R₁₂, and R₁₃ are independently selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

6. The compound, or the salt or the stereoisomer thereof according to claim 3, wherein the compound of formula (IIIA-1) has a structure represented by formula (VIA-1): 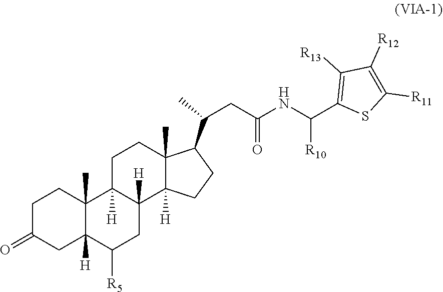
R₅ is selected from the group consisting of —OH, —NH₂, —OSO₃H or a salt thereof, —OPO(OH)₂ or a salt thereof, —OCO(CH₂)ₘCOOH or a salt thereof, an amino acid ester or a salt thereof, and —OCO(CH₂)ₘCH₃; the amino acid ester group is an α-amino acid ester group; and m is an integer ranging from 0 to 9;
R₁₀ is selected from H, linear or branched C₁₋₄ alkyl, —(CH₂)ₙCOOH, —(CH₂)ₙ₊₁NH₂, and —(CH₂)ₙ-B; and n is an integer ranging from 0 to 4;
B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;
cycloalkyl comprises cyclopropyl, cyclobutyl, cyclopentyl, and cyclohexyl; and
R₁₁, R₁₂, and R₁₃ are independently selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

7. The compound, or the salt or the stereoisomer thereof according to claim 3, wherein the compound of formula (IIIA-1) has a structure represented by formula (VIIA-1): 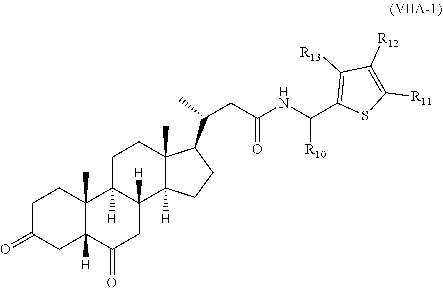
R₁₀ is selected from H, linear or branched C₁₋₄ alkyl, —(CH₂)ₙCOOH, —(CH₂)ₙ₊₁NH₂, and —(CH₂)ₙ-B; and n is an integer ranging from 0 to 4;
B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;
cycloalkyl comprises cyclopropyl, cyclobutyl, cyclopentyl, and cyclohexyl; and
R₁₁, R₁₂, and R₁₃ are independently selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

8. The compound, or the salt or the stereoisomer thereof according to claim 3, wherein the compound of formula (IIIA-1) has a structure represented by formula (VIIIA-1): 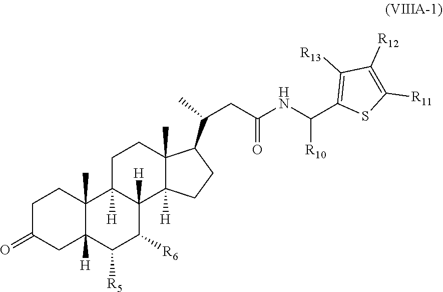
R₅ and R₆ are selected from the group consisting of —OH, —NH₂, —OSO₃H or a salt thereof, —OPO(OH)₂ or a salt thereof, —OCO(CH₂)ₙCOOH or a salt thereof, an amino acid ester or a salt thereof, and —OCO(CH₂)ₘCH₃; the amino acid ester group is an α-amino acid ester group; and m is an integer ranging from 0 to 9;
R₁₀ is selected from H, linear or branched C₁₋₄ alkyl, —(CH₂)ₙCOOH, —(CH₂)ₙ₊₁NH₂, and —(CH₂)ₙ-B; and n is an integer ranging from 0 to 4;
B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;
cycloalkyl comprises cyclopropyl, cyclobutyl, cyclopentyl, and cyclohexyl; and
R₁₁, R₁₂, and R₁₃ are independently selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

9. The compound, or the salt or the stereoisomer thereof according to claim 3, wherein the compound of formula (IIIA-1) has a structure represented by formula (IXA-1): 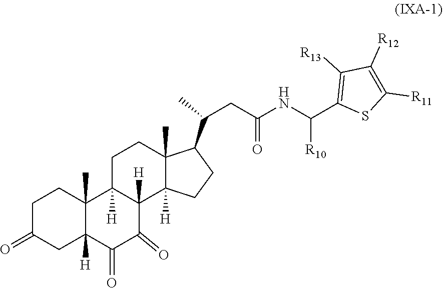
R₁₀ is selected from H, linear or branched C₁₋₄ alkyl, —(CH₂)ₙCOOH, —(CH₂)ₙ₊₁NH₂, and —(CH₂)ₙ-B; and n is an integer ranging from 0 to 4;
B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;
cycloalkyl comprises cyclopropyl, cyclobutyl, cyclopentyl, and cyclohexyl; and
R₁₁, R₁₂, and R₁₃ are independently selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

10. The compound, or the salt or the stereoisomer thereof according to claim 3, wherein the compound of formula (IIIA-1) has a structure represented by formula (XA-1): 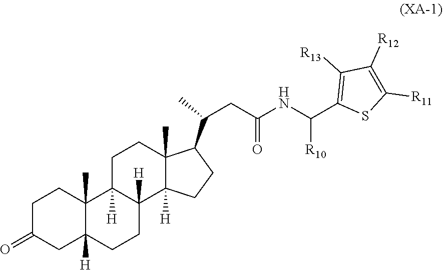
R₁₀ is selected from H, linear or branched C₁₋₄ alkyl, —(CH₂)ₙCOOH, —(CH₂)ₙ₊₁NH₂, and —(CH₂)ₙ-B; and n is an integer ranging from 0 to 4;
B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;
cycloalkyl comprises cyclopropyl, cyclobutyl, cyclopentyl, and cyclohexyl; and
R₁₁, R₁₂, and R₁₃ are independently selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

11. The compound, or the salt or the stereoisomer thereof according to claim 4, wherein the compound of formula (IVA-1) has a structure represented by formula (XIA-1) or (XIA-2): 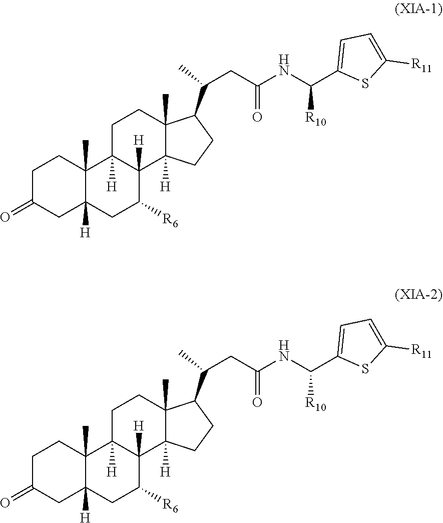
R₆ is selected from the group consisting of —OH, —NH₂, —OSO₃H or a salt thereof, —OPO(OH)₂ or a salt thereof, —OCO(CH₂)ₘCOOH or a salt thereof, an amino acid ester or a salt thereof, and —OCO(CH₂)ₘCH₃; the amino acid ester group is an α-amino acid ester group; and m is an integer ranging from 0 to 9;
R₁₀ is selected from the group consisting of H, C₁₋₄ alkyl, and cyclopropyl;
R₁₁ is selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

12. The compound, or the salt or the stereoisomer thereof according to claim 5, wherein the compound of formula (VA-1) has a structure represented by formula (XIIA-1) or (XIIA-2): 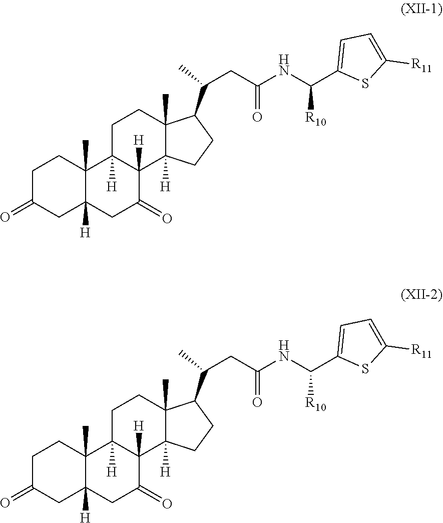
R₁₀ is selected from the group consisting of H, C₁₋₄ alkyl, and cyclopropyl;
R₁₁ is selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

13. The compound, or the salt or the stereoisomer thereof according to claim 2, wherein the compound of formula (IIA) has a structure represented by formula (IIIB): 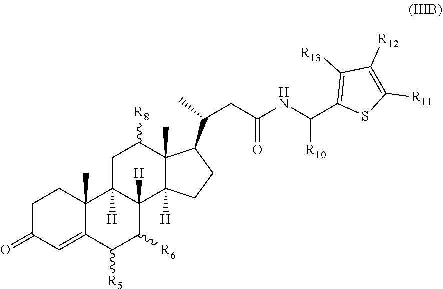
R₅, R₆, and R₈ are independently selected from the group consisting of H, —OH, C₁₋₅ alkoxy, —NH₂, ═O, ═S, ═NOH, —NHSO₃H or a salt thereof, —OSO₃H or a salt thereof, —OPO(OH)₂ or a salt thereof, —OCO(CH₂)ₘCOOH or a salt thereof, an amino acid ester group or a salt thereof, and —OCO(CH₂)ₘCH₃; and m is an integer ranging from 0 to 9; or
R₁₀ is selected from H, linear or branched C₁₋₄ alkyl, —(CH₂)ₙCOOH, —(CH₂)ₙ₊₁NH₂, and —(CH₂)ₙ-B; and n is an integer ranging from 0 to 4;
B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;
cycloalkyl comprises cyclopropyl, cyclobutyl, cyclopentyl, and cyclohexyl; and
R₁₁, R₁₂, and R₁₃ are independently selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

14. The compound, or the salt or the stereoisomer thereof according to claim 2, wherein the compound of formula (IIA) has a structure represented by formula (IIIC): 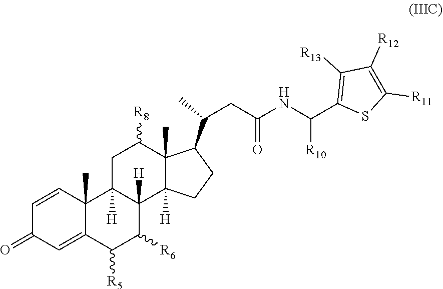
R₅, R₆, and R₈ are independently selected from the group consisting of H, —OH, C₁₋₅ alkoxy, —NH₂, ═O, ═S, ═NOH, —NHSO₃H or a salt thereof, —OSO₃H or a salt thereof, —OPO(OH)₂ or a salt thereof, —OCO(CH₂)ₘCOOH or a salt thereof, an amino acid ester group or a salt thereof, and —OCO(CH₂)ₘCH₃; and m is an integer ranging from 0 to 9; or
R₁₀ is selected from H, linear or branched C₁₋₄ alkyl, —(CH₂)ₙCOOH, —(CH₂)ₙ₊₁NH₂, and —(CH₂)ₙ-B; and n is an integer ranging from 0 to 4;
B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;
cycloalkyl comprises cyclopropyl, cyclobutyl, cyclopentyl, and cyclohexyl; and
R₁₁, R₁₂, and R₁₃ are independently selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

15. The compound, or the salt or the stereoisomer thereof according to claim 13, wherein the compound of formula (IIIB) has a structure represented by formula (IVB-1), (IVB-2), or (IVB-3): 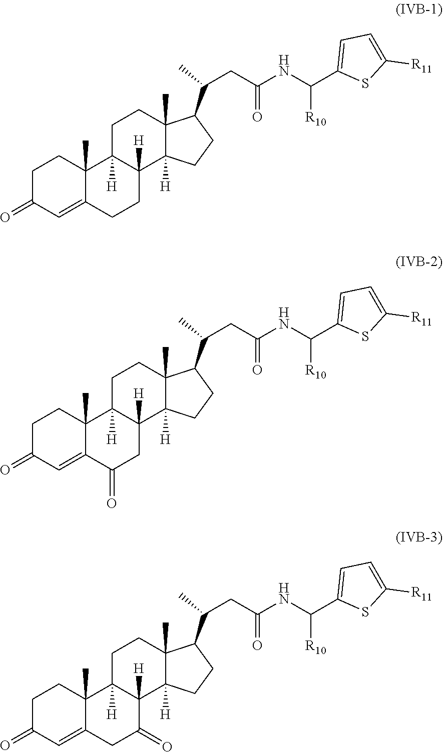
R₁₀ is selected from H, linear or branched C₁₋₄ alkyl, —(CH₂)ₙCOOH, —(CH₂)ₙ₊₁NH₂, and —(CH₂)ₙ-B; and n is an integer ranging from 0 to 4;
B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;
cycloalkyl comprises cyclopropyl, cyclobutyl, cyclopentyl, and cyclohexyl; and
R₁₁ is selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

16. The compound, or the salt or the stereoisomer thereof according to claim 13, wherein the compound of formula (IIIB) has a structure represented by formula (VB-1): 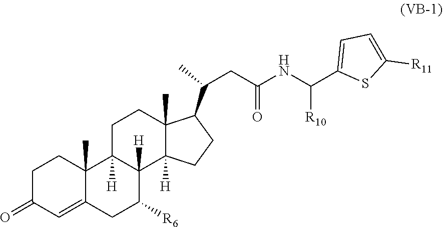
R₆ is selected from the group consisting of —OH, C₁₋₅ alkoxy, —NH₂, ═O, ═S, ═NOH, —NHSO₃H or a salt thereof, —OSO₃H or a salt thereof, —OPO(OH)₂ or a salt thereof, —OCO(CH₂)ₘCOOH or a salt thereof, an amino acid ester group or a salt thereof, and —OCO(CH₂)ₘCH₃; the amino acid ester group is an α-amino acid ester group; and m is an integer ranging from 0 to 9; or
R₁₀ is selected from H, linear or branched C₁₋₄ alkyl, —(CH₂)ₙCOOH, —(CH₂)ₙ₊₁NH₂, and —(CH₂)ₙ-B; and n is an integer ranging from 0 to 4;
B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;
cycloalkyl comprises cyclopropyl, cyclobutyl, cyclopentyl, and cyclohexyl; and
R₁₁ is selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

17. The compound, or the salt or the stereoisomer thereof according to claim 14, wherein the compound of formula (IIIC) has a structure represented by formula (IVC-1), (IVC-2), or (IVC-3): 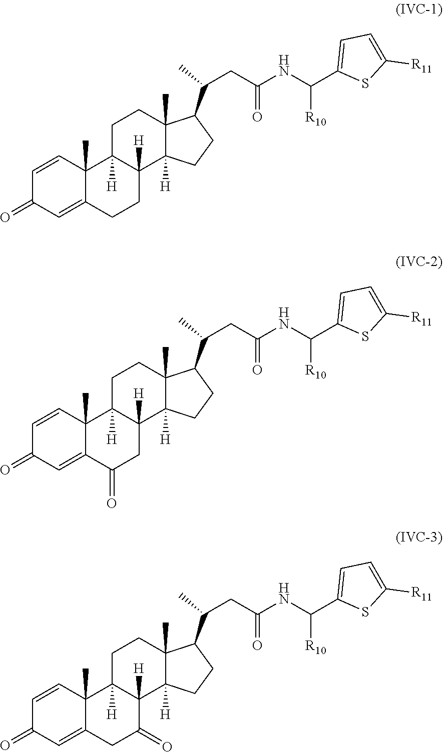
R₁₀ is selected from H, linear or branched C₁₋₄ alkyl, —(CH₂)ₙCOOH, —(CH₂)ₙ₊₁NH₂, and —(CH₂)ₙ-B; and n is an integer ranging from 0 to 4;
B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;
cycloalkyl comprises cyclopropyl, cyclobutyl, cyclopentyl, and cyclohexyl; and
R₁₁ is selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

18. The compound, or the salt or the stereoisomer thereof according to claim 14, wherein the compound of formula (IIIC) has a structure represented by formula (VC-1): 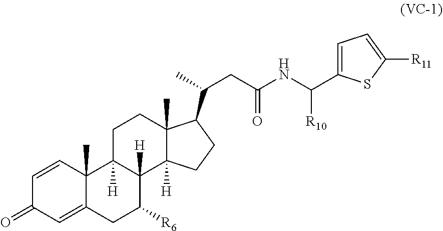
R₆ is selected from the group consisting of —OH, C₁₋₅ alkoxy, —NH₂, ═O, ═S, ═NOH, —NHSO₃H or a salt thereof, —OSO₃H or a salt thereof, —OPO(OH)₂ or a salt thereof, —OCO(CH₂)ₘCOOH or a salt thereof, an amino acid ester group or a salt thereof, and —OCO(CH₂)ₘCH₃; the amino acid ester group is an α-amino acid ester group; and m is an integer ranging from 0 to 9; or
R₁₀ is selected from H, linear or branched C₁₋₄ alkyl, —(CH₂)ₙCOOH, —(CH₂)ₙ₊₁NH₂, and —(CH₂)ₙ-B; and n is an integer ranging from 0 to 4;
B is independently selected from the group consisting of substituted or unsubstituted phenyl, thienyl, pyrrolyl, furyl, imidazolyl, oxazolyl, thiazolyl, pyridyl, pyrimidinyl, pyrazinyl, benzothienyl, indolyl, benzothiazolyl, and cycloalkyl; and a substituent on B is selected from the group consisting of halogen, methyl, and methoxy;
cycloalkyl comprises cyclopropyl, cyclobutyl, cyclopentyl, and cyclohexyl; and
R₁₁ is selected from the group consisting of H, —OH, —NH₂, linear or branched C₁₋₃ alkyl, C₃₋₆ N heterocyclyl, —NO₂, —CHO, sulfo, F, Cl, Br, I, —O(CH₂)ₙ₂CH₃, —(CH₂)ₙ₂COOH, —N[(CH₂)ₙ₂CH₃]₂, —COCH₃, and —CO(CH₂)ₙ₃COOH; and n2 is an integer ranging from 0 to 3 and n3 is an integer ranging from 1 to 6.

19.-24. (canceled)

25. The compound, or the salt or the stereoisomer thereof according to claim 1, wherein the compound is selected from the following compounds:

| No. | Compound structure |
|---|---|
| I-1 | 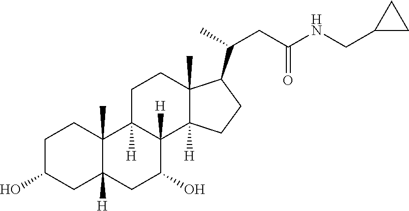 |
| I-2 | 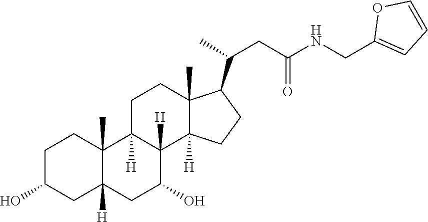 |
| I-3 | 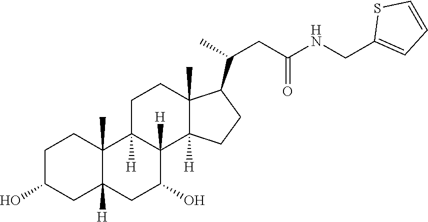 |
| I-4 | 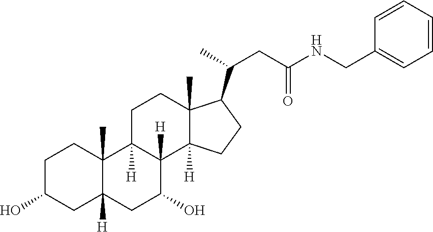 |
| I-5 | 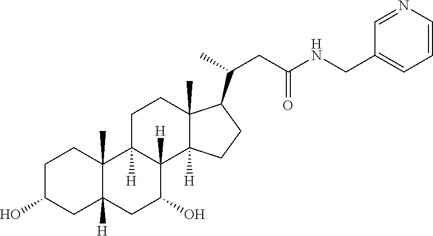 |
| I-6 | 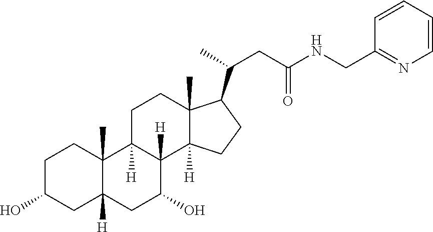 |
| I-7 | 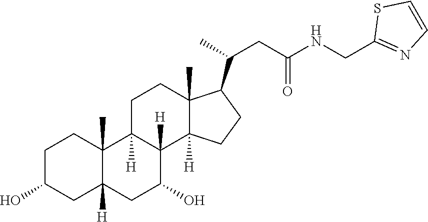 |
| I-8 | 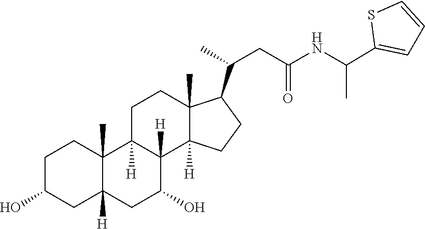 |
| I-9 | 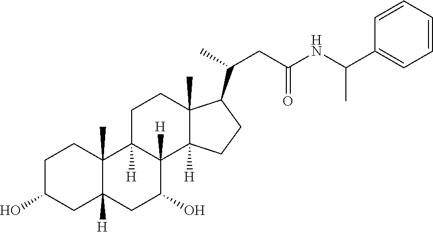 |
| I-10 | 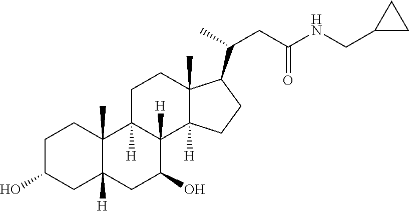 |
| I-11 | 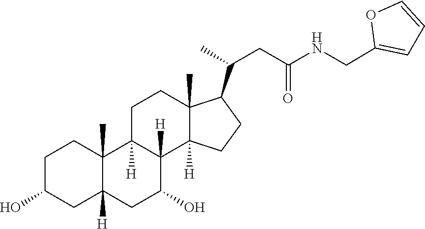 |
| I-12 | 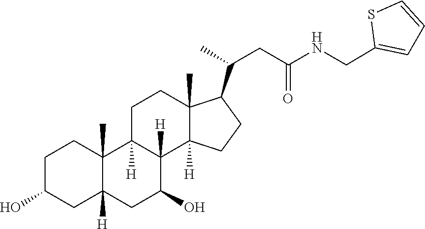 |
| I-13 | 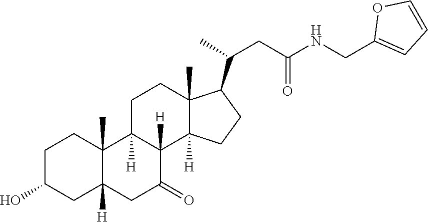 |
| I-14 | 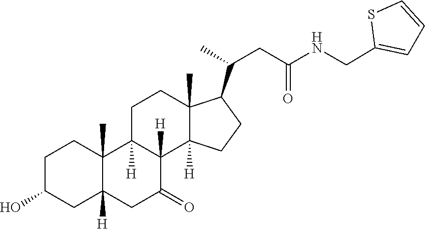 |
| I-15 | 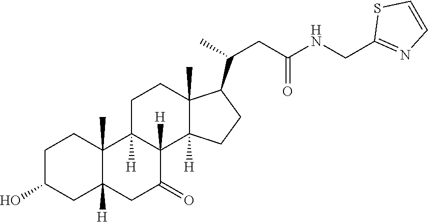 |
| I-16 | 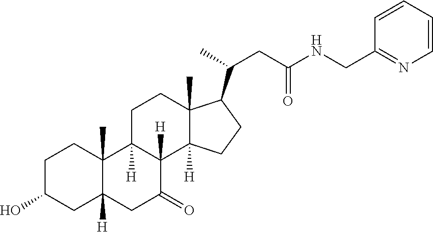 |
| I-17 | 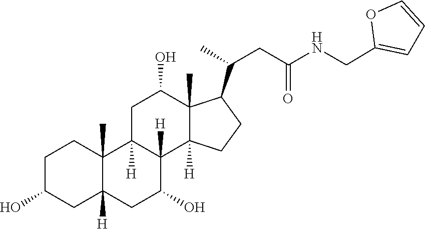 |
| I-18 | 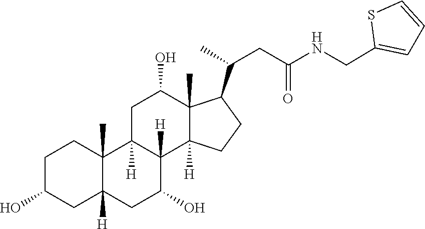 |
| I-19 | 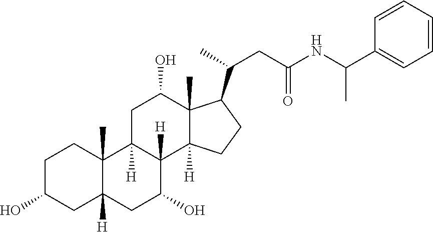 |
| I-20 | 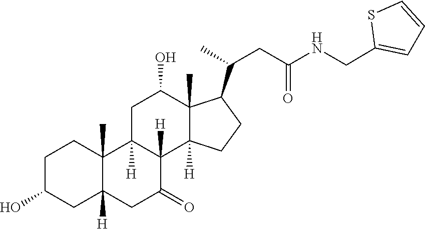 |
| I-21 | 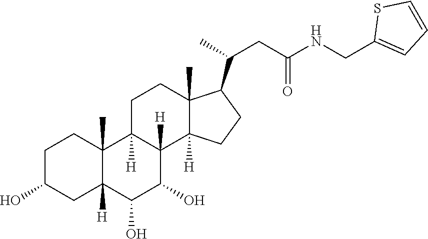 |
| I-22 | 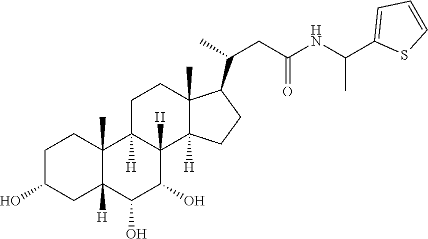 |
| I-23 | 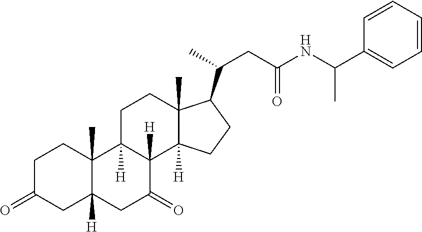 |
| I-24 | 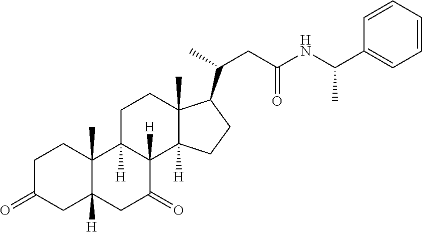 |
| I-25 | 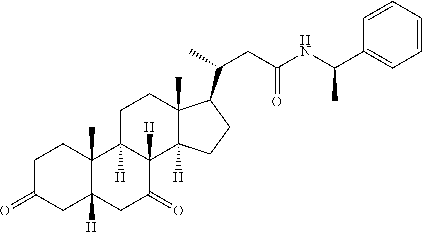 |
| I-26 | 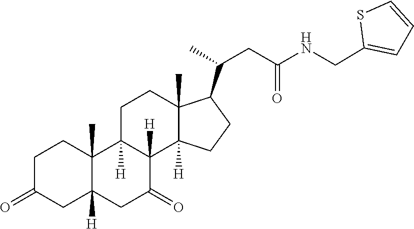 |
| I-27 | 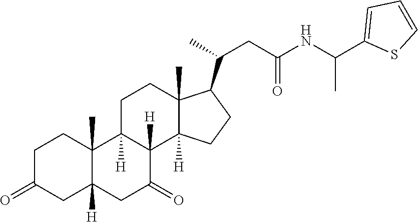 |
| I-28 | 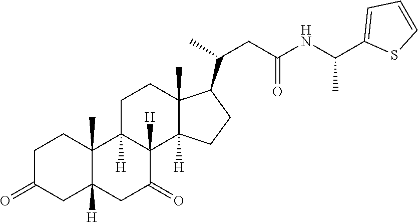 |
| I-29 | 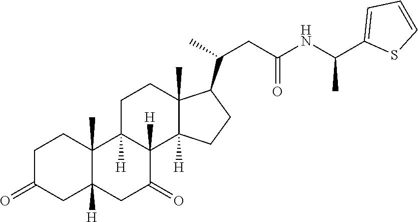 |
| I-30 | 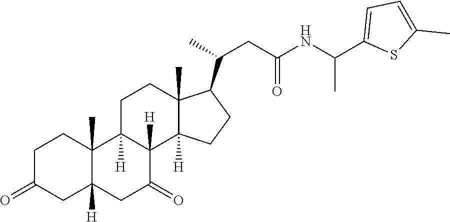 |
| I-31 | 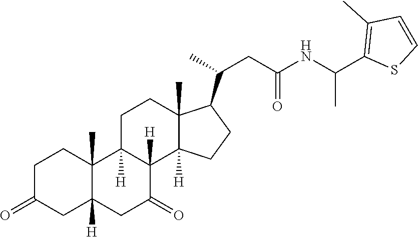 |
| I-32 | 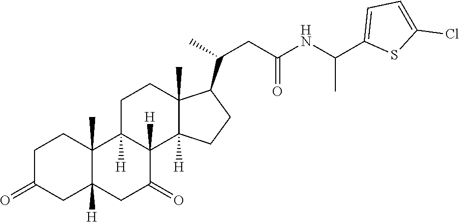 |
| I-33 | 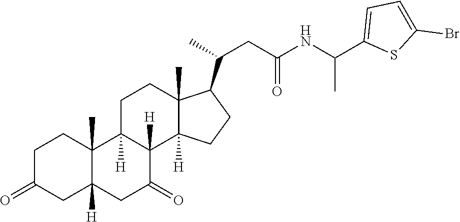 |
| I-34 | 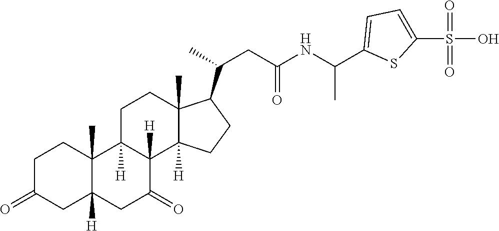 |
| I-35 | 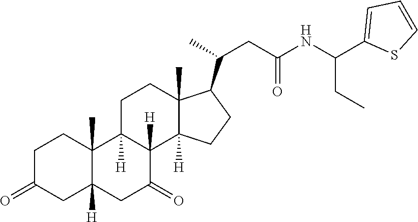 |
| I-36 | 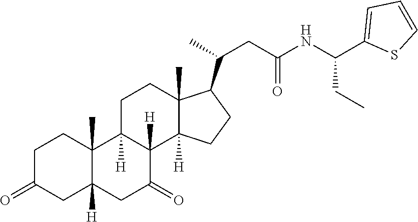 |
| I-37 | 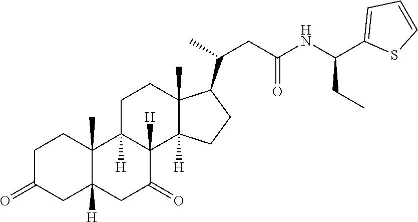 |
| I-38 | 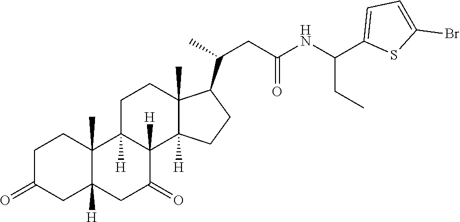 |
| I-39 | 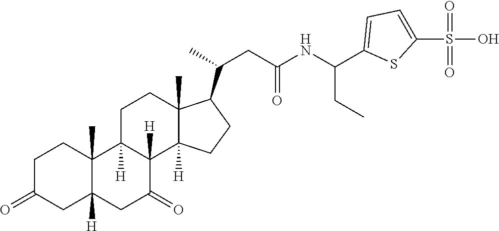 |
| I-40 | 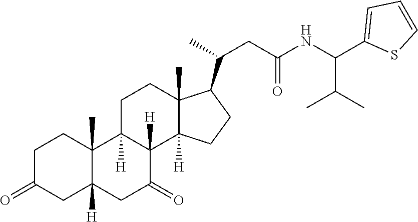 |
| I-41 | 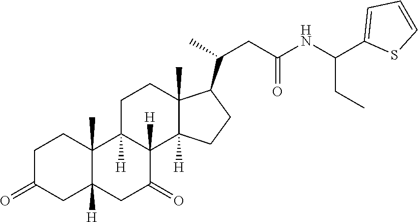 |
| I-42 | 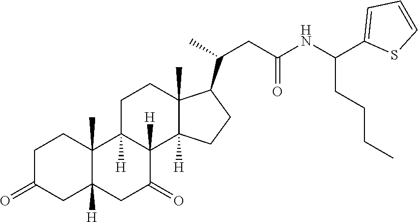 |
| I-43 | 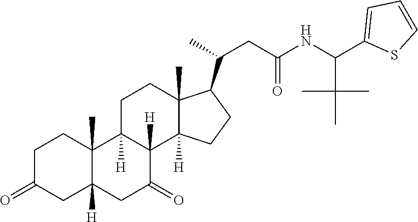 |
| I-44 | 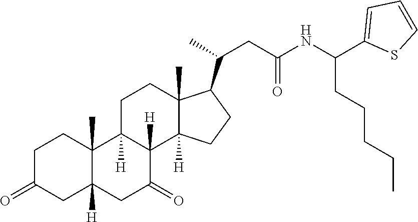 |
| I-45 | 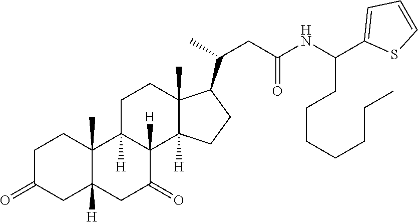 |
| I-46 | 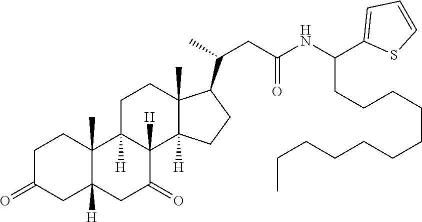 |
| I-47 | 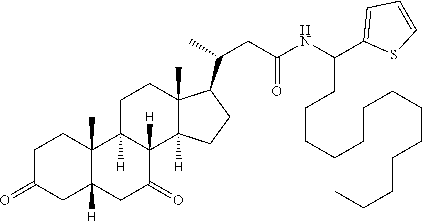 |
| I-48 | 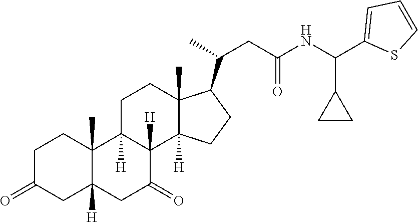 |
| I-49 | 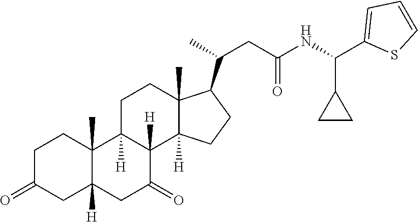 |
| I-50 | 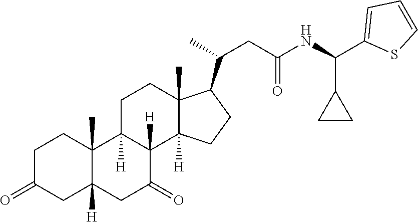 |
| I-51 | 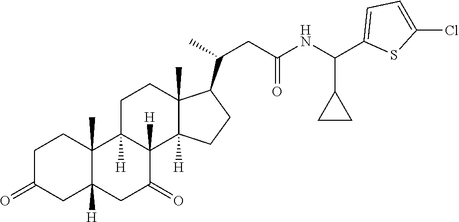 |
| I-52 | 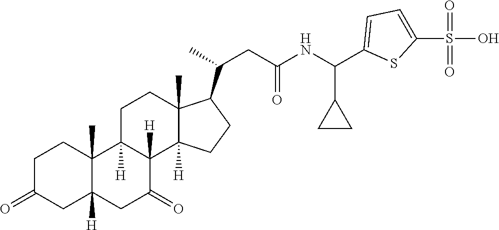 |
| I-53 | 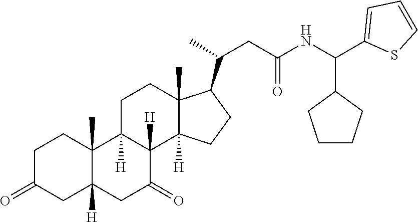 |
| I-54 | 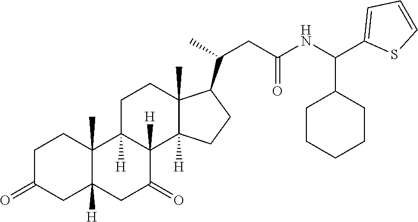 |
| I-55 | 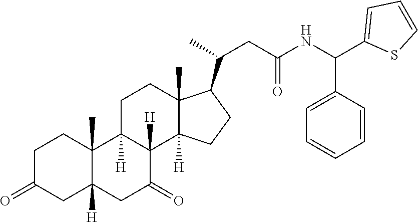 |
| I-56 | 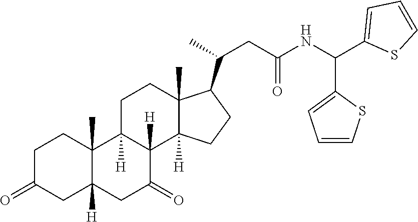 |
| I-57 | 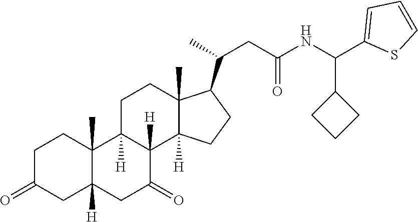 |
| I-58 | 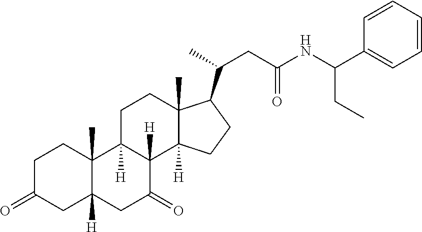 |
| I-59 | 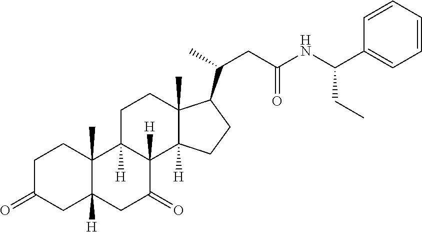 |
| I-60 | 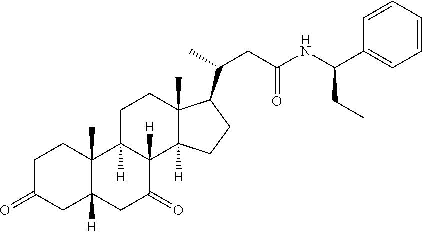 |
| I-61 | 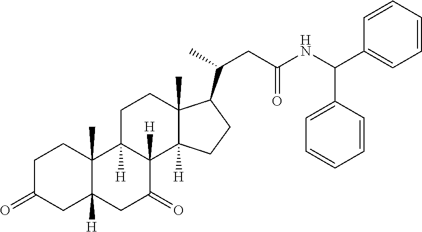 |
| I-62 | 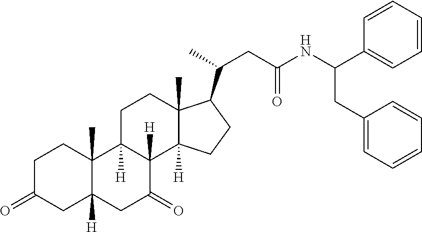 |
| I-63 | 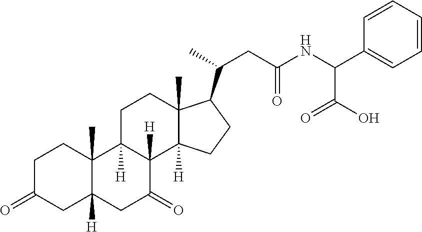 |
| I-64 | 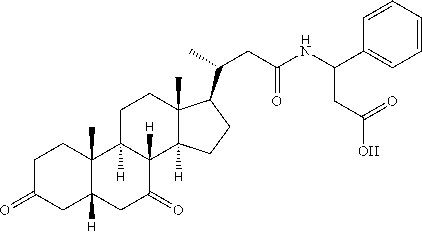 |
| I-65 | 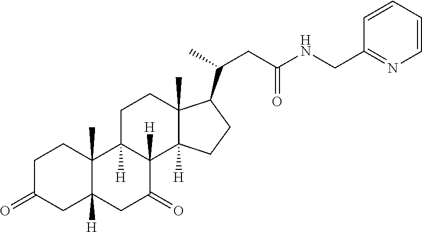 |
| I-66 | 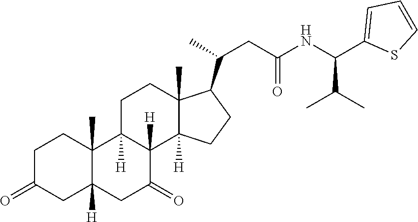 |
| I-67 | 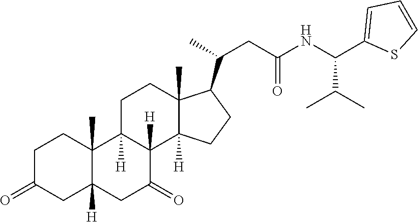 |
| I-68 | 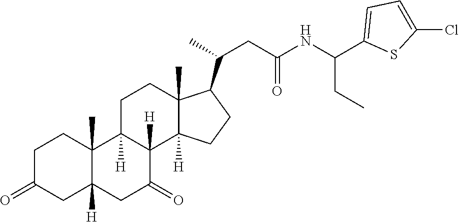 |
| I-69 | 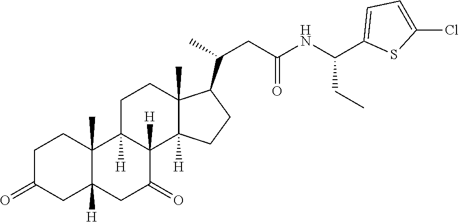 |
| I-70 | 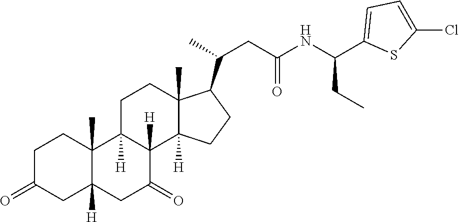 |
| I-71 | 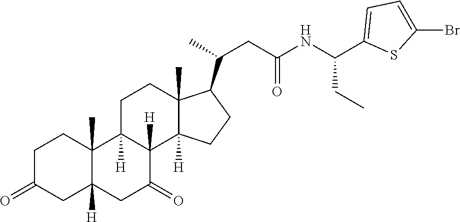 |
| I-72 | 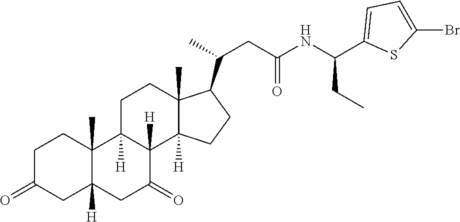 |
| I-73 | 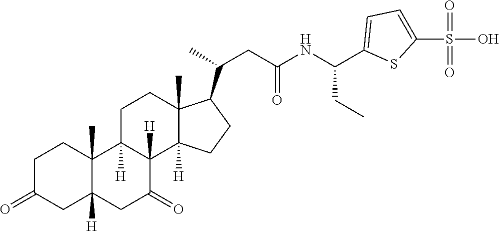 |
| I-74 | 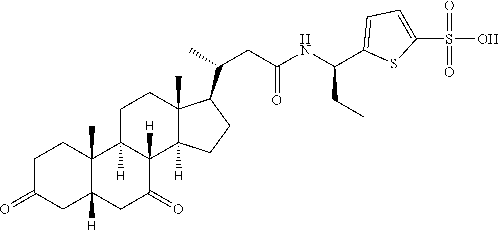 |
| I-75 | 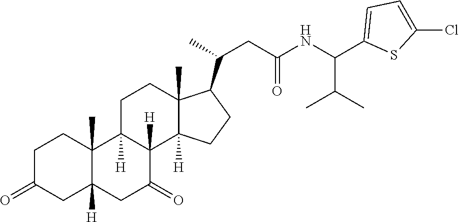 |
| I-76 | 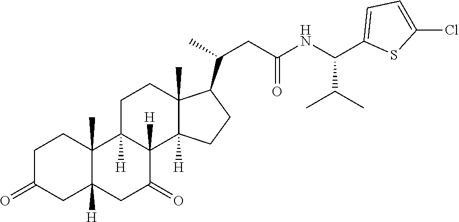 |
| I-77 | 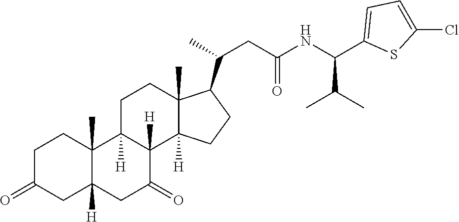 |
| I-78 | 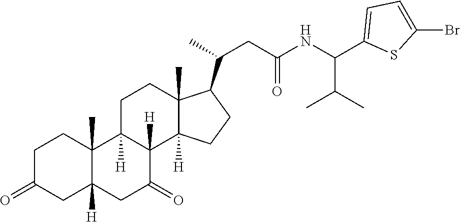 |
| I-79 | 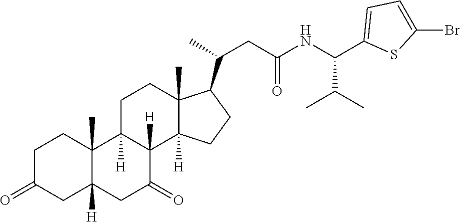 |
| I-80 | 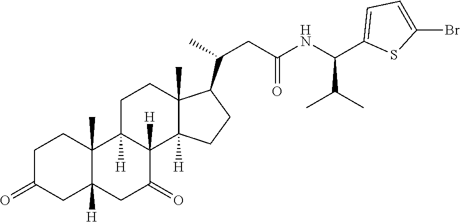 |
| I-81 | 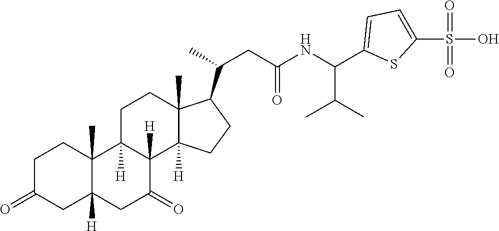 |
| I-82 | 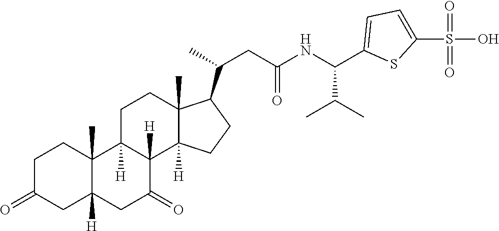 |
| I-83 | 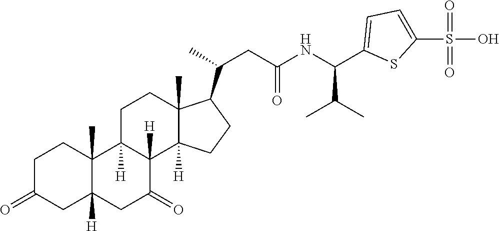 |
| I-84 | 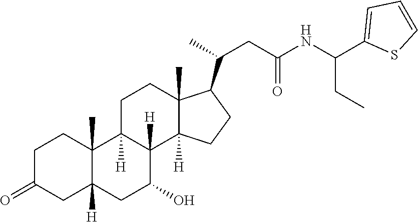 |
| I-85 | 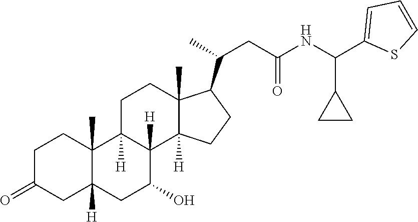 |
| I-86 | 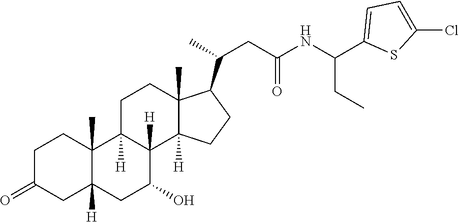 |
| I-87 | 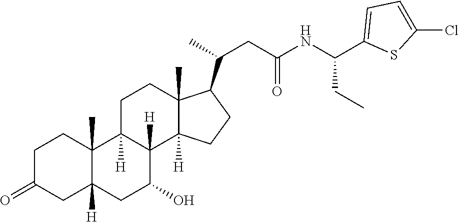 |
| I-88 | 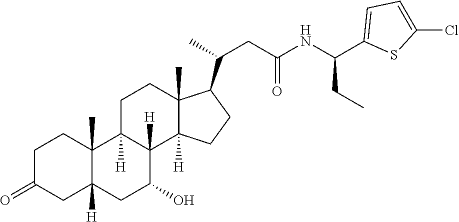 |
| I-89 | 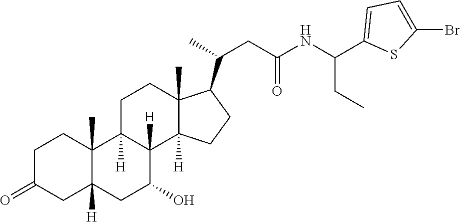 |
| I-90 | 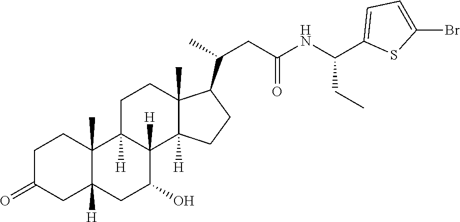 |
| I-91 | 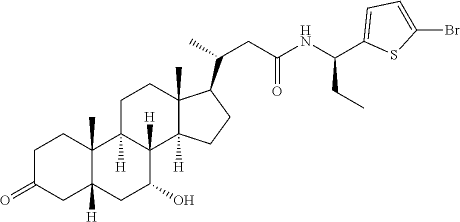 |
| I-92 | 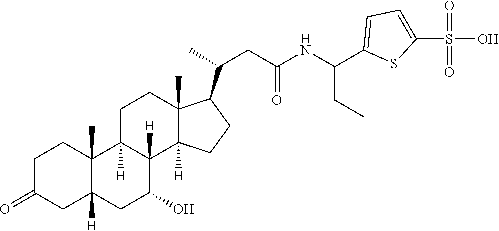 |
| I-93 | 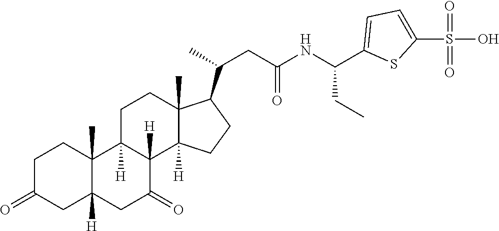 |
| I-94 | 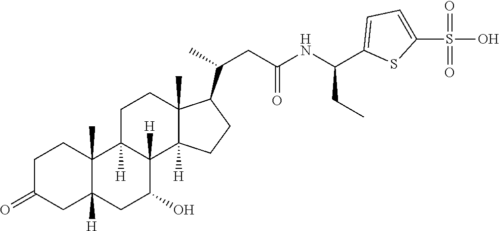 |
| I-95 | 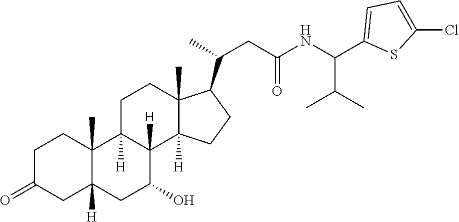 |
| I-96 | 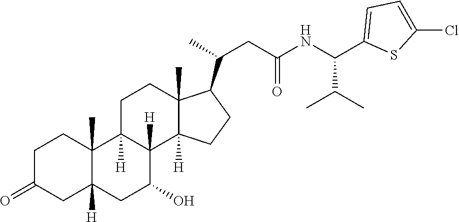 |
| I-97 | 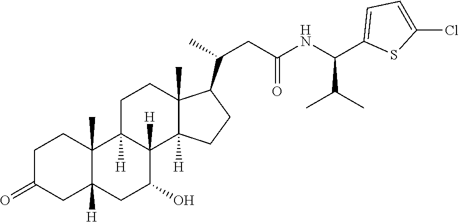 |
| I-98 | 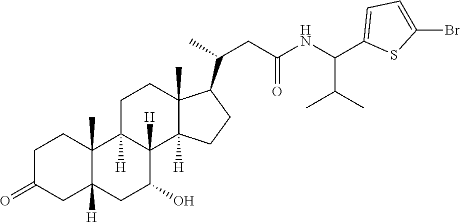 |
| I-99 | 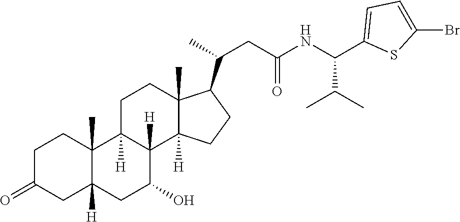 |
| I-100 | 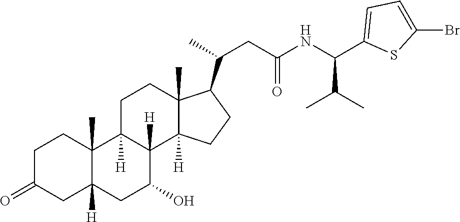 |
| I-101 | 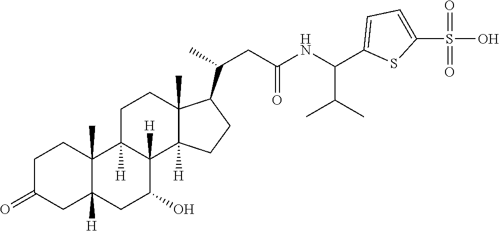 |
| I-102 | 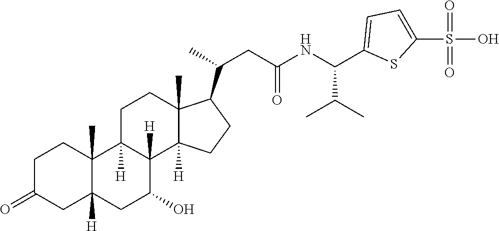 |
| I-103 | 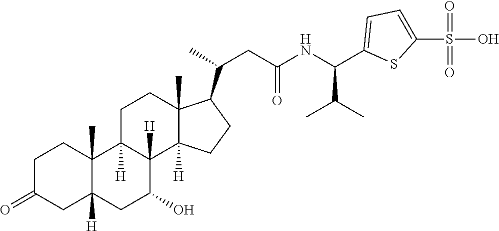 |
| I-104 | 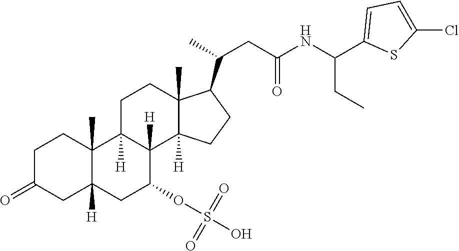 |
| I-104-1 | 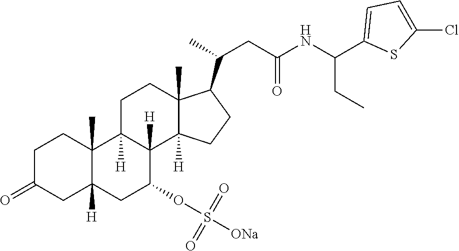 |
| I-105 | 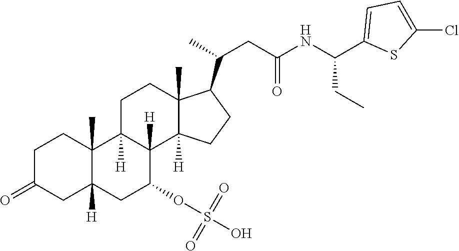 |
| I-105-1 | 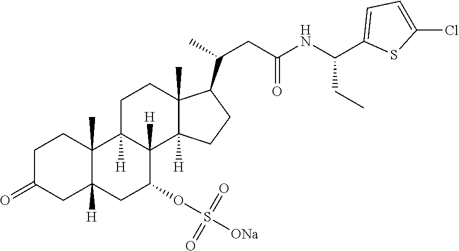 |
| I-106 | 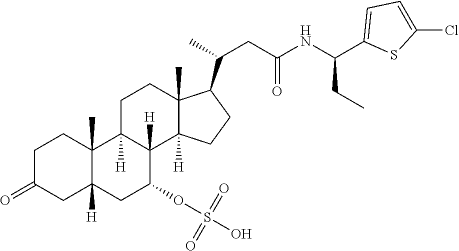 |
| I-106-1 | 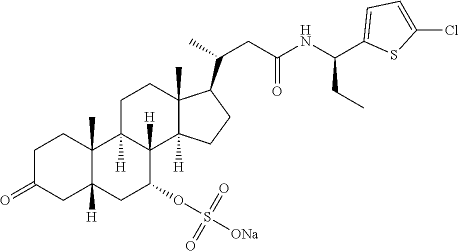 |
| I-107 | 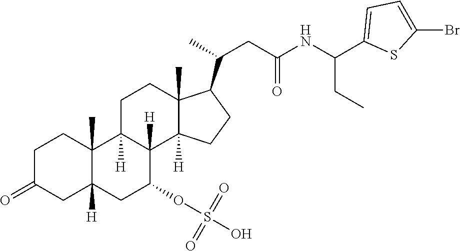 |
| I-107-1 | 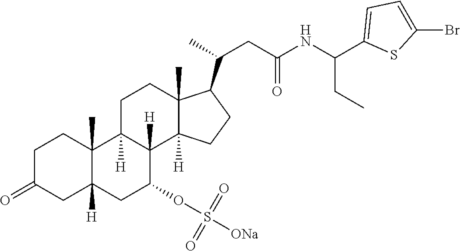 |
| I-108 | 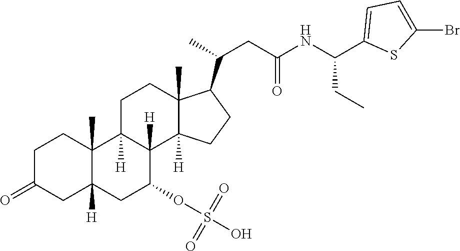 |
| I-108-1 | 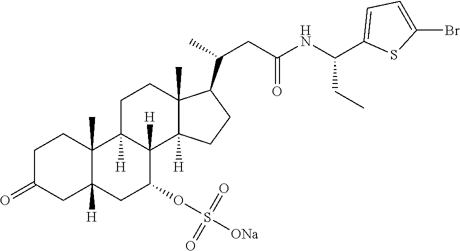 |
| I-109 | 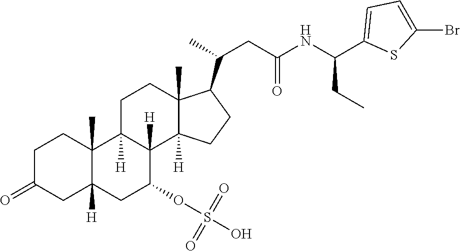 |
| I-109-1 | 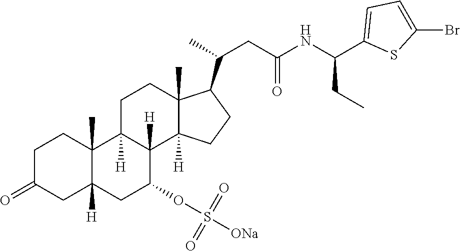 |
| I-110 | 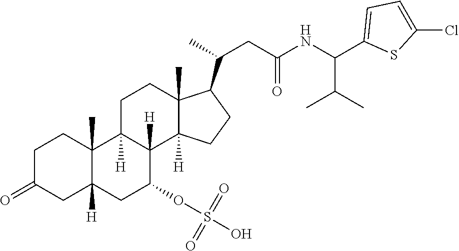 |
| I-110-1 | 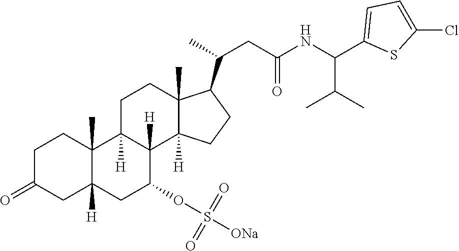 |
| I-111 | 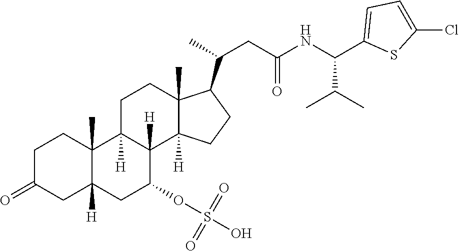 |
| I-111-1 | 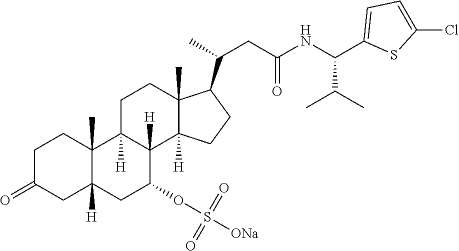 |
| I-112 | 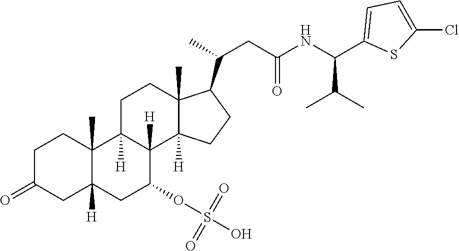 |
| I-112-1 | 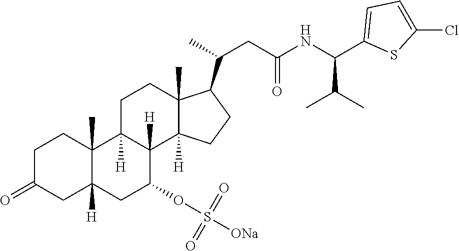 |
| I-113 | 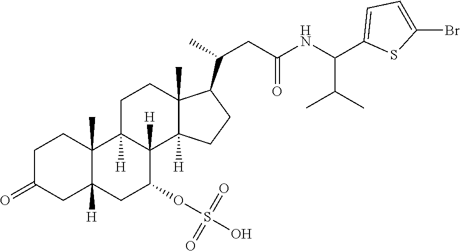 |
| I-113-1 | 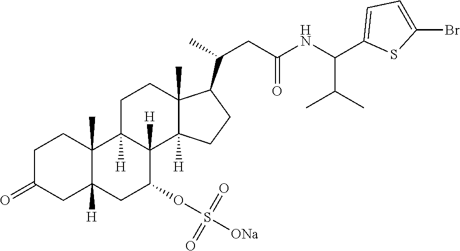 |
| I-114 | 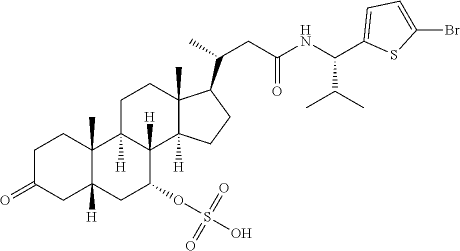 |
| I-114-1 | 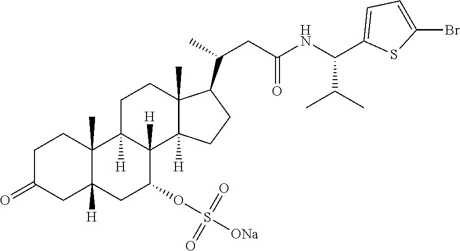 |
| I-115 | 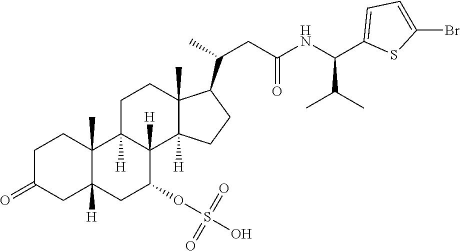 |
| I-115-1 | 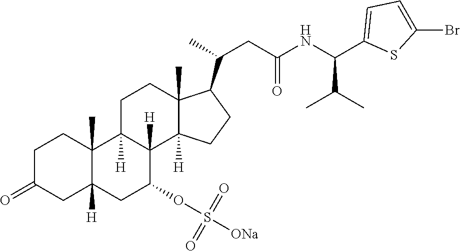 |
| I-116 | 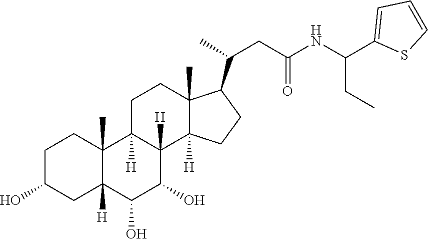 |
| I-117 | 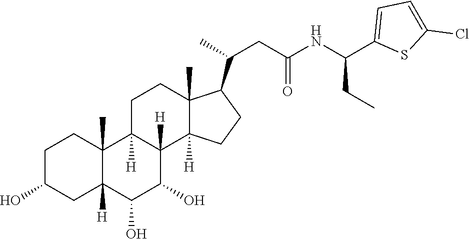 |
| I-118 | 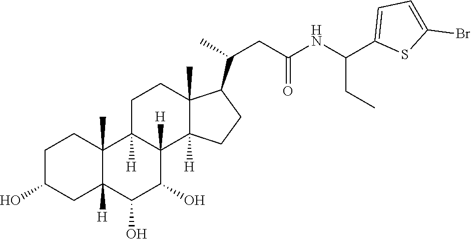 |
| I-119 | 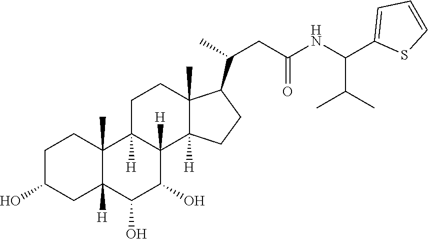 |
| I-120 | 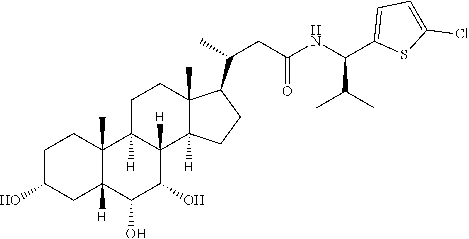 |
| I-121 | 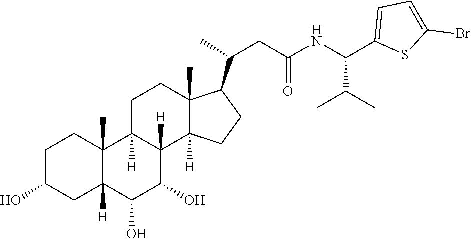 |
| I-122 | 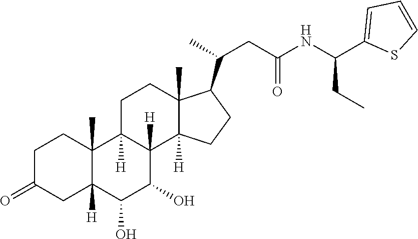 |
| I-123 | 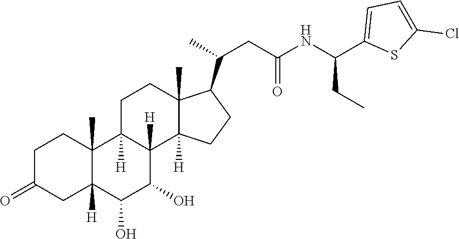 |
| I-124 | 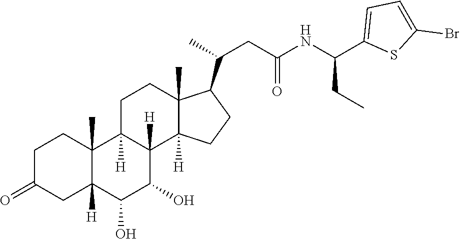 |
| I-125 | 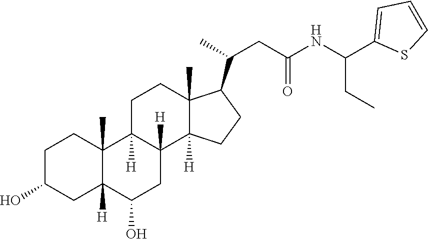 |
| I-126 | 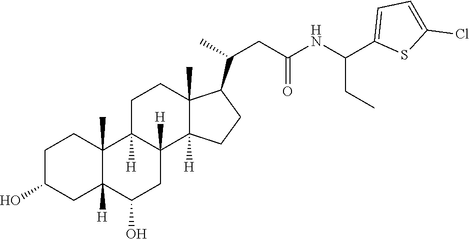 |
| I-127 | 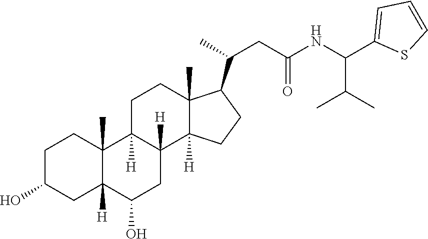 |
| I-128 | 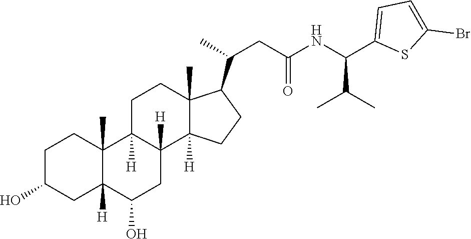 |
| I-129 | 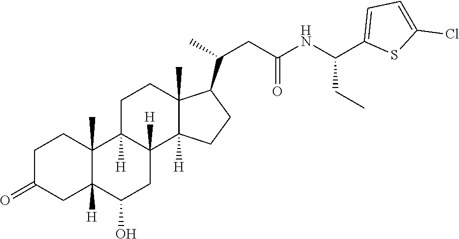 |
| I-130 | 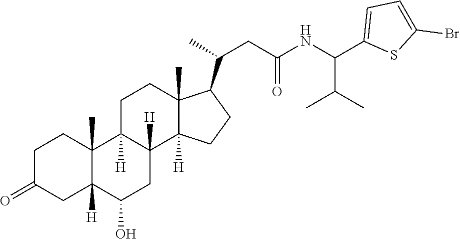 |
| I-131 | 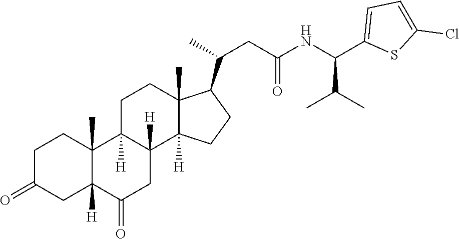 |
| I-132 | 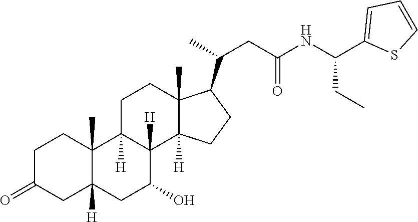 |
| I-133 | 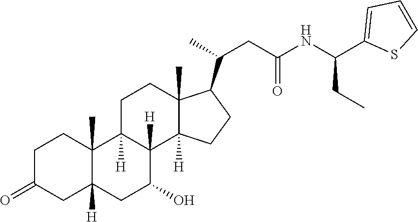 |
| I-134 | 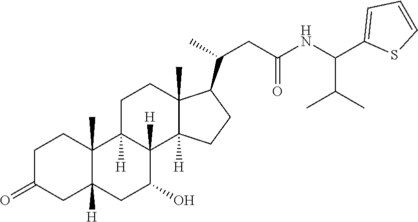 |
| I-135 | 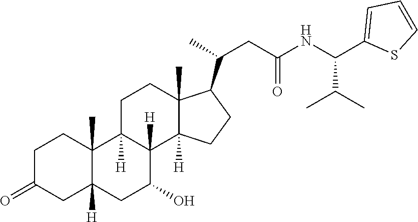 |
| I-136 | 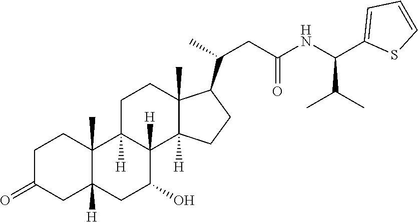 |
| I-137 | 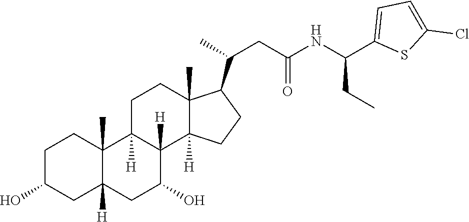 |
| I-138 | 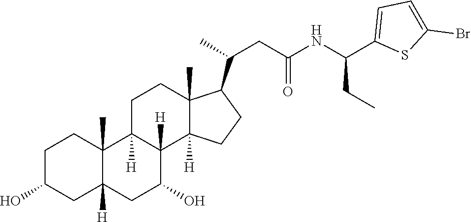 |
| I-139 | 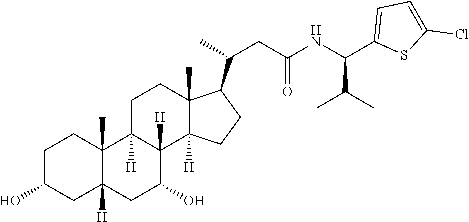 |
| I-140 | 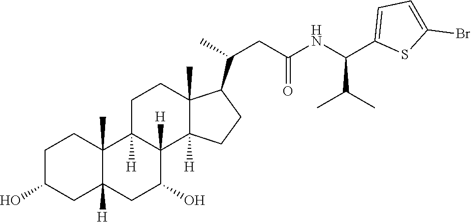 |
| I-141 | 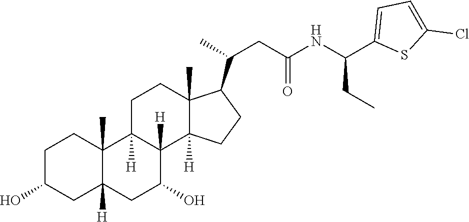 |
| I-142 | 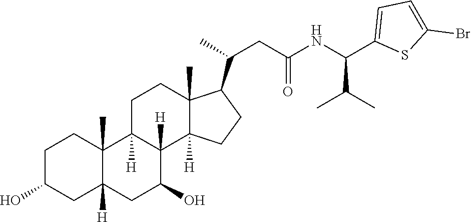 |
| I-143 | 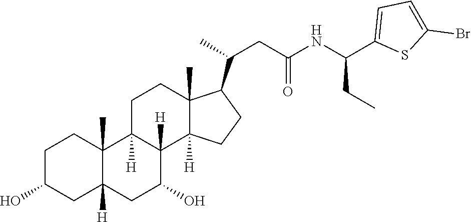 |
| I-144 | 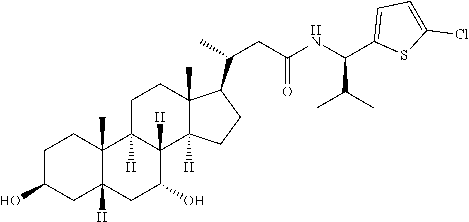 |
| I-145 | 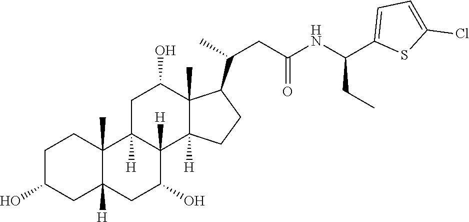 |
| I-146 | 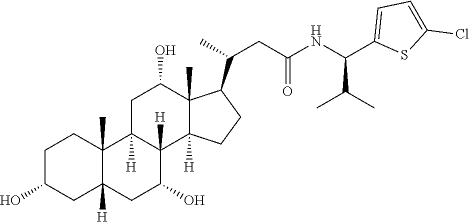 |
| I-147 | 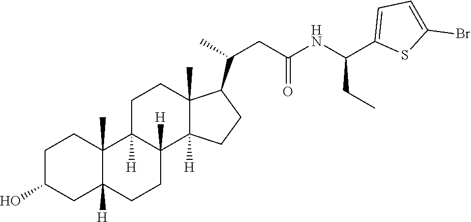 |
| I-148 | 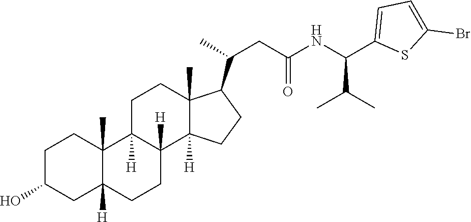 |
| I-149 | 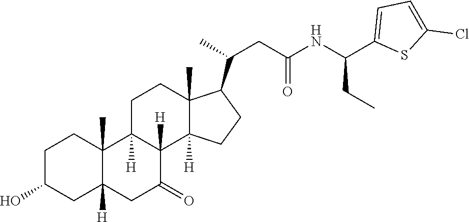 |
| I-150 | 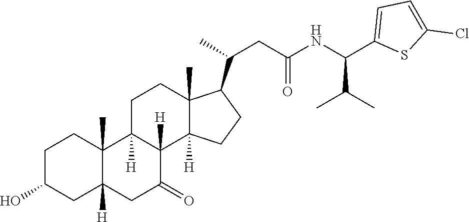 |
| I-151 | 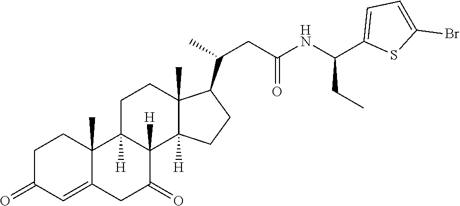 |
| I-152 | 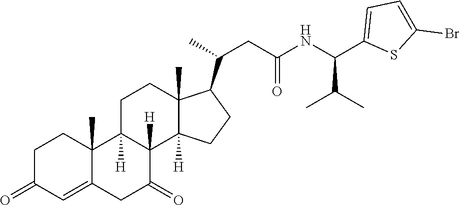 |
| I-153 | 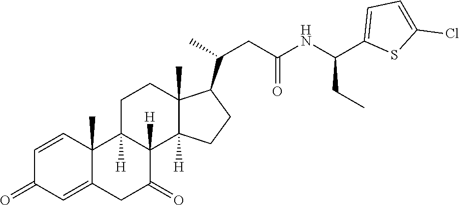 |
| I-154 | 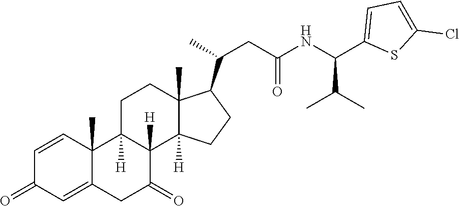 |
| I-155-1 | 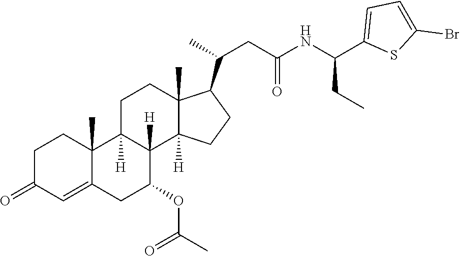 |
| I-155-2 | 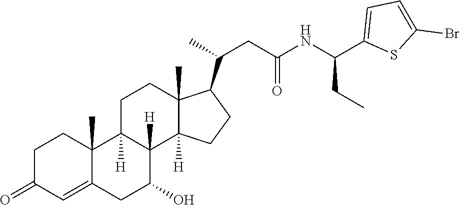 |
| I-156-1 | 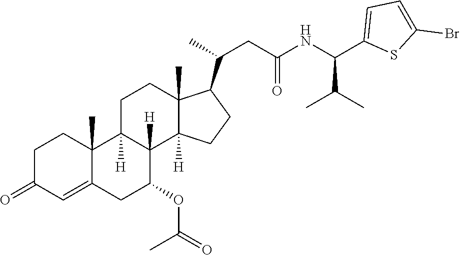 |
| I-156-2 | 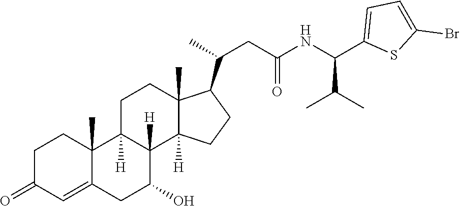 |
| I-157-1 | 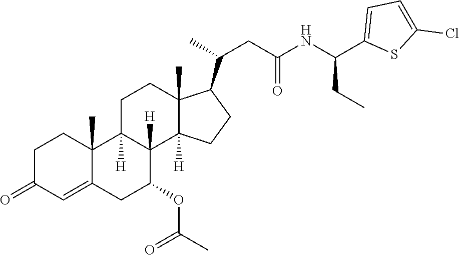 |
| I-157-2 | 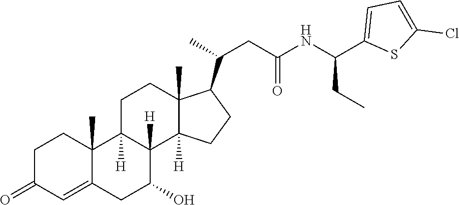 |
| I-158-1 | 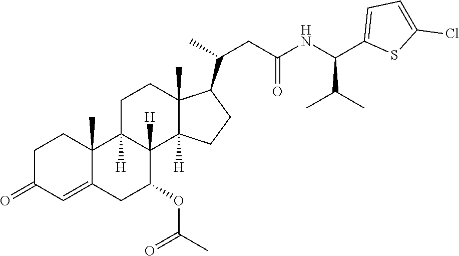 |
| I-158-2 | 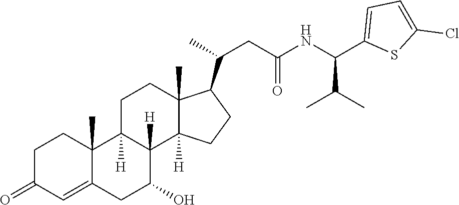 |
| I-159-1 | 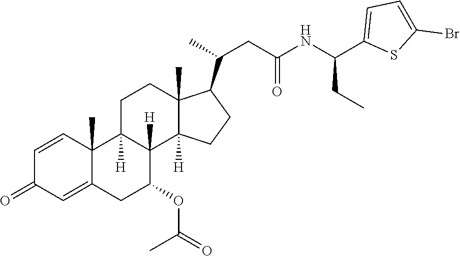 |
| I-159-2 | 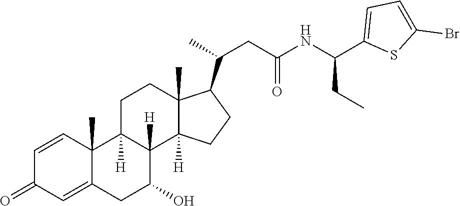 |
| I-160-1 | 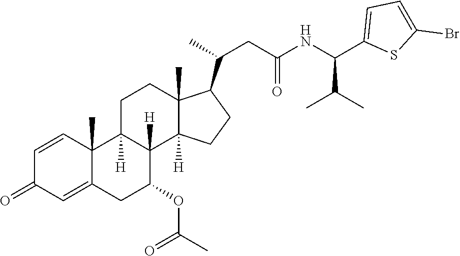 |
| I-160-2 | 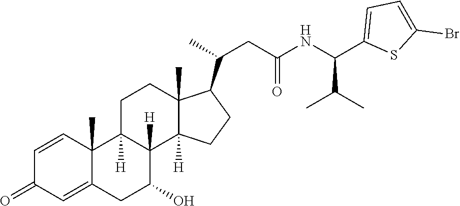 |
| I-161-1 | 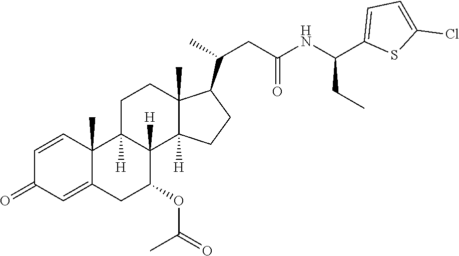 |
| I-161-2 | 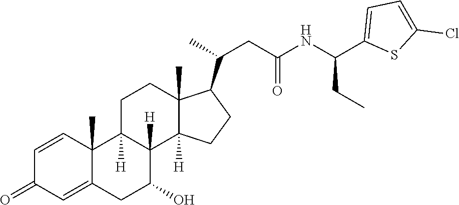 |
| I-162-1 | 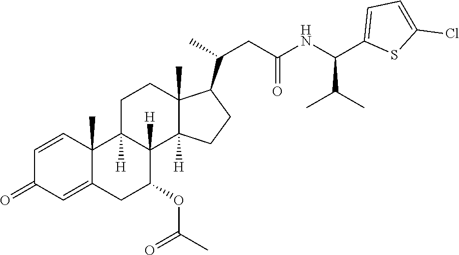 |
| I-162-2 | 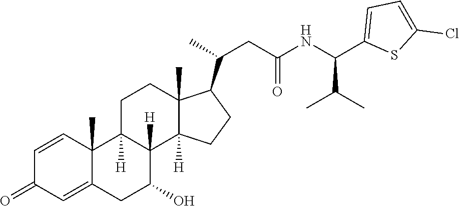 |
| I-163 | 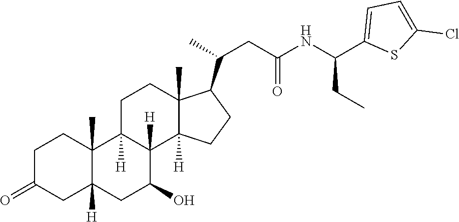 |
| I-164 | 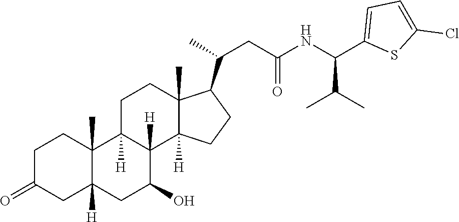 |
| I-165 | 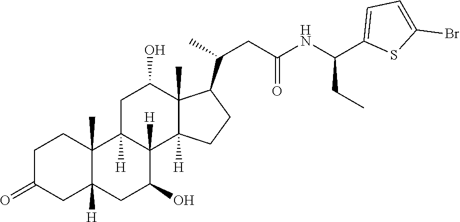 |
| I-166 | 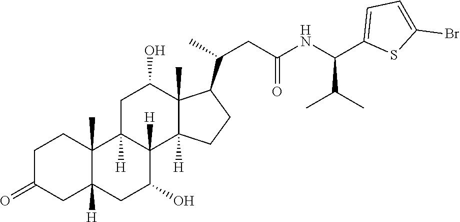 |
| I-167 | 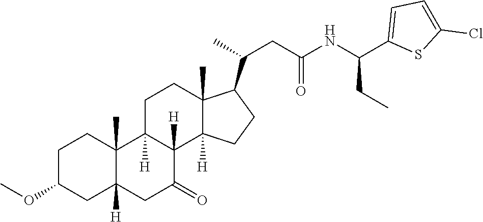 |
| I-168 | 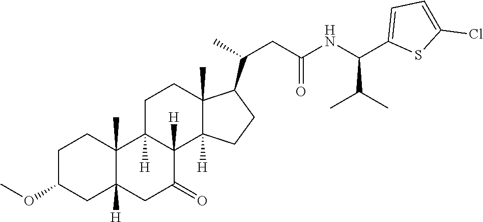 |
| I-169 | 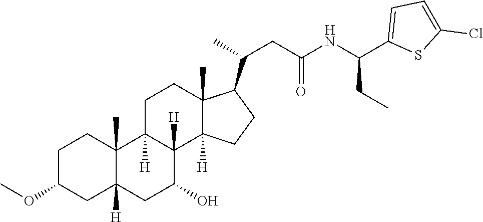 |
| I-170 | 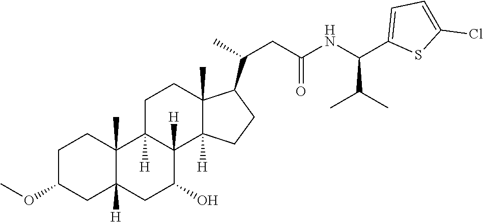 |
| I-171 | 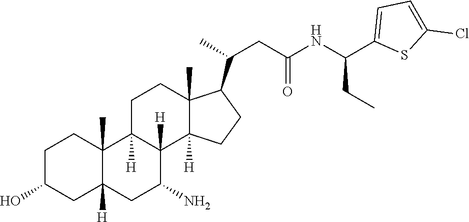 |
| I-172 | 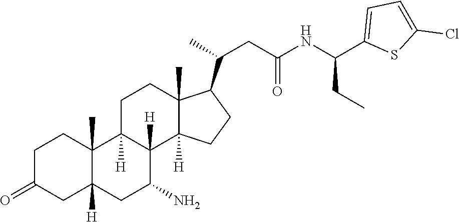 |
| I-173 | 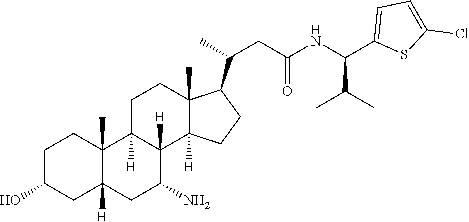 |
| I-174 | 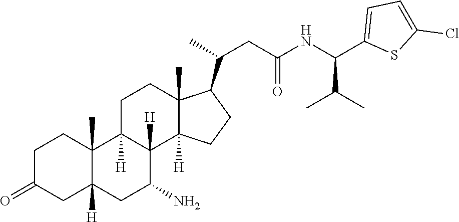 |
| I-175 | 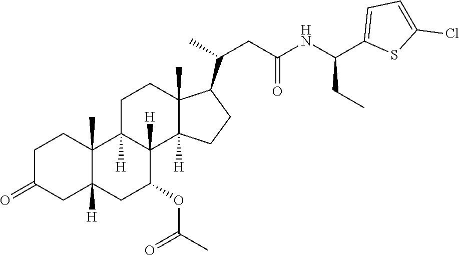 |
| I-176 | 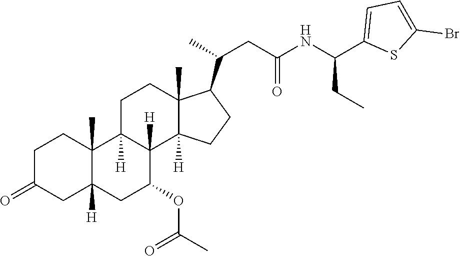 |
| I-177 | 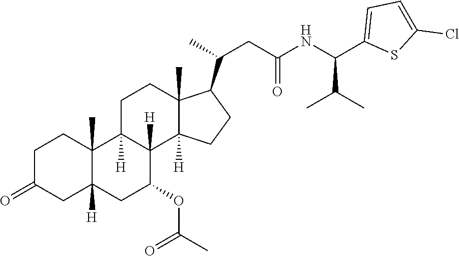 |
| I-178 | 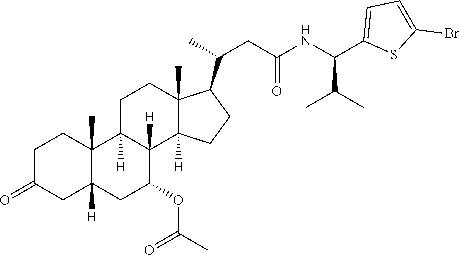 |
| I-179 | 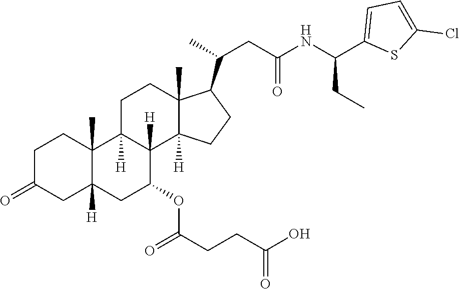 |
| I-180 | 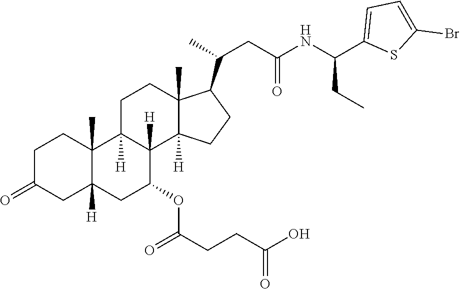 |
| I-181 | 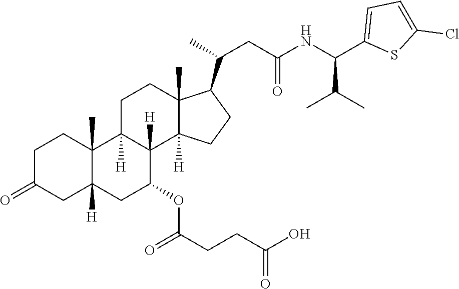 |
| I-182 | 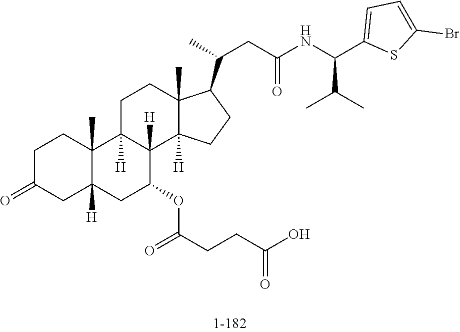 |
| I-183 | 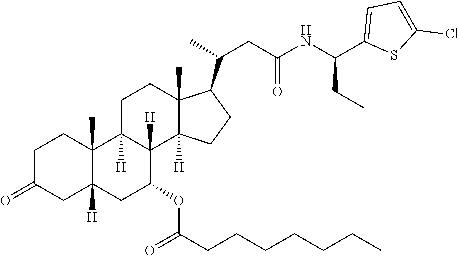 |
| I-184 | 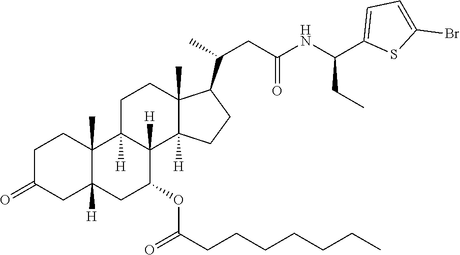 |
| I-185 | 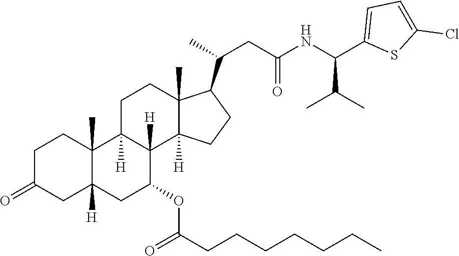 |
| I-186 | 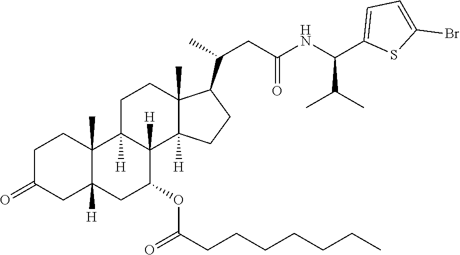 |
| I-187 | 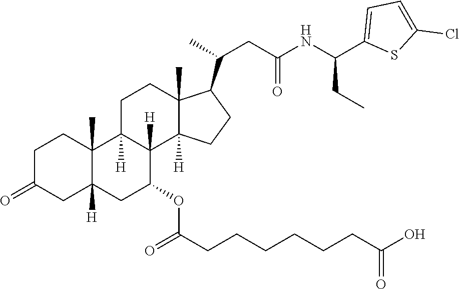 |
| I-188 | 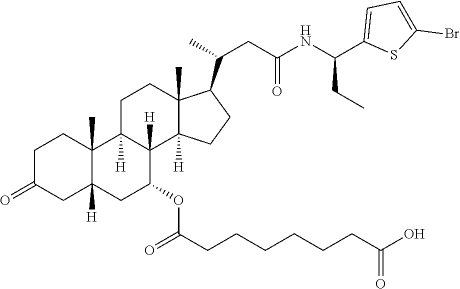 |
| I-189 | 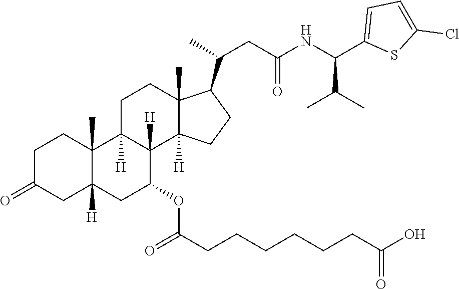 |
| I-190 | 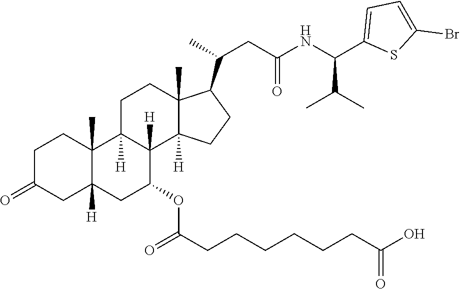 |
| I-191 | 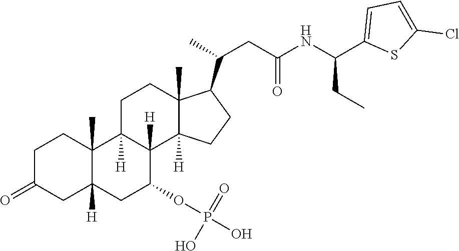 |
| I-191-1 | 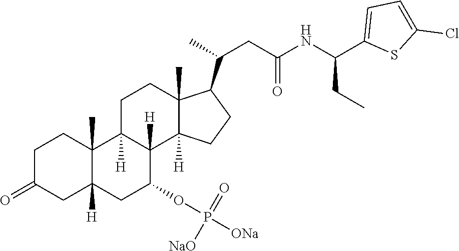 |
| I-192 | 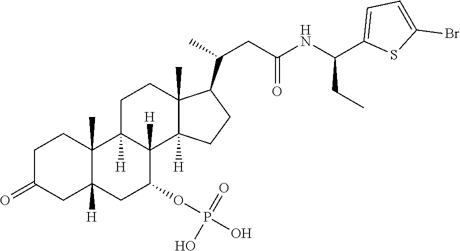 |
| I-192-1 | 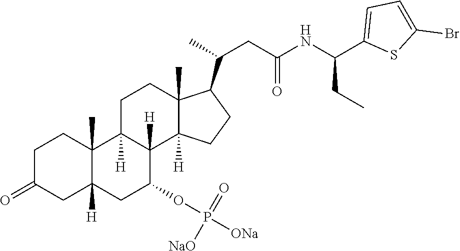 |
| I-193 | 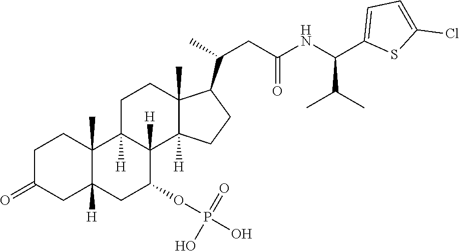 |
| I-193-1 | 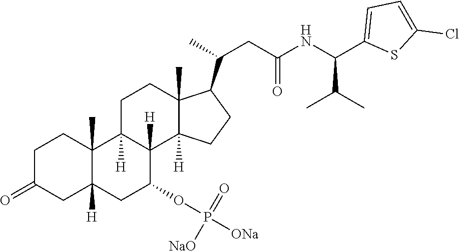 |
| I-194 | 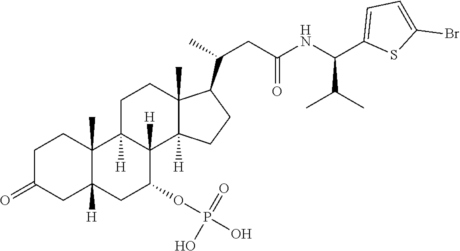 |
| I-194-1 | 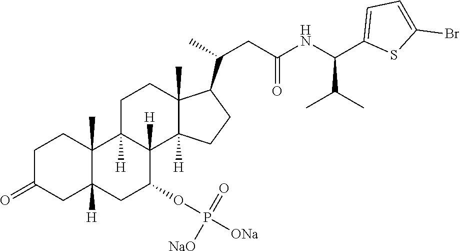 |
| I-195 | 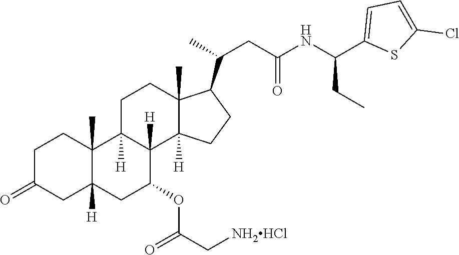 |
| I-196 | 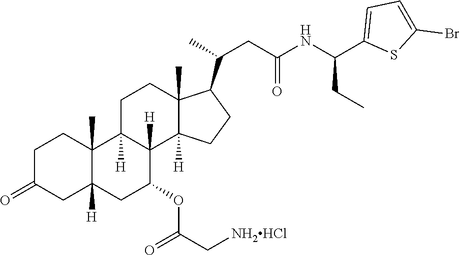 |
| I-197 | 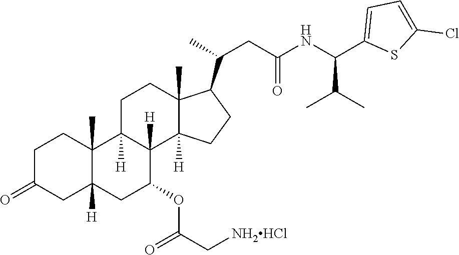 |
| I-198 | 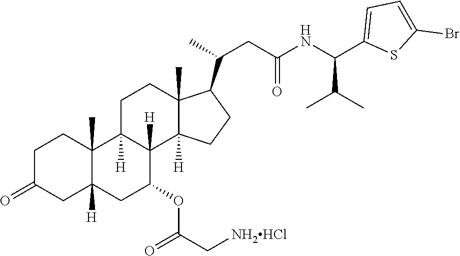 |
| I-199 | 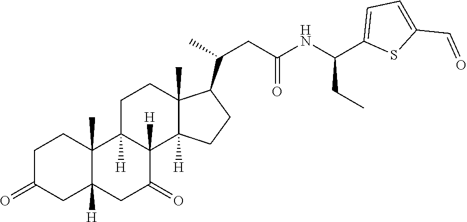 |
| I-200 | 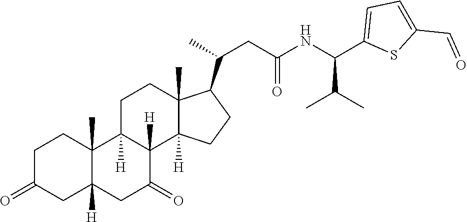 |
| I-201 | 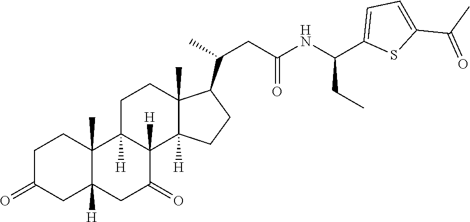 |
| I-202 | 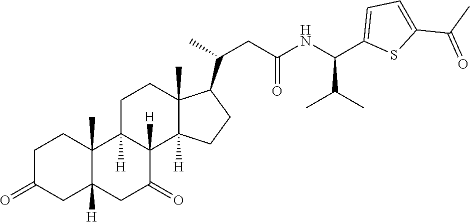 |
| I-203 | 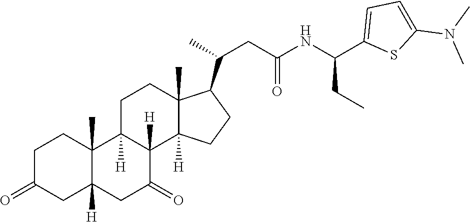 |
| I-204 | 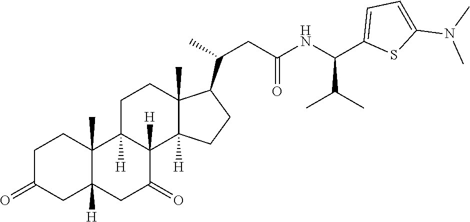 |
| I-205 | 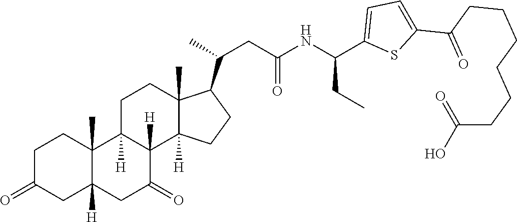 |
| I-206 | 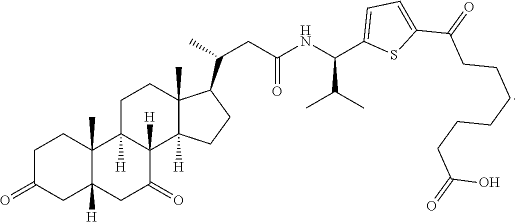 |

26. A method for inhibiting germination of C. difficile spores, comprising administering the compound, or the salt or the stereoisomer thereof according to claim 1.

27.-34. (canceled)
